SEQUENCE LISTING (9 sequences)

SEQ ID NO: 1 - protein, 462 aa, Homo sapiens
MLLLARCLLLVLVSSLLVCSGLACGPGRGFGKRRHPKKLTPLAYKQFIPNVAEKTLGASGRYEGKISRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLSPGDRVLAADDQGRLLYSDFLTFLDRDDGAKKVFYVIETREPRERLLLTAAHLLFVAPHNDSATGEPEASSGSGPPSGGALGPRALFASRVRPGQRVYVVAERDGDRRLLPAAVHSVTLSEEAAGAYAPLTAQGTILINRVLASCYAVIEEHSWAHRAFAPFRLAHALLAALAPARTDRGGDSGGGDRGGGGGRVALTAPGAADAPGAGATAGIHWYSQLLYQIGTWLLDSEALHPLGMAVKSS

SEQ ID NO: 2 - DNA, 1576 bp, Homo sapiens
gcgaggcagccagcgagggagagagcgagcgggcgagccggagcgaggaagggaaagcgcaagagagagcgcacacgcacacacccgccgcgcgcactcgcgcacggacccgcacggggacagctcggaagtcatcagttccatgggcgagatgctgctgctggcgagatgtctgctgctagtcctcgtctcctcgctgctggtatgctcgggactggcgtgcggaccgggcagggggttcgggaagaggaggcaccccaaaaagctgacccctttagcctacaagcagtttatccccaatgtggccgagaagaccctaggcgccagcggaaggtatgaagggaagatctccagaaactccgagcgatttaaggaactcacccccaattacaaccccgacatcatatttaaggatgaagaaaacaccggagcggacaggctgatgactcagaggtgtaaggacaagttgaacgctttggccatctcggtgatgaaccagtggccaggagtgaaactgcgggtgaccgagggctgggacgaagatggccaccactcagaggagtctctgcactacgagggccgcgcagtggacatcaccacgtctgaccgcgaccgcagcaagtacggcatgctggcccgcctggcggtggaggccggcttcgactgggtgtactacgagtccaaggcacatatccactgctcggtgaaagcagagaactcggtggcggccaaatcgggaggctgcttcccgggctcggccacggtgcacctggagcagggcggcaccaagctggtgaaggacctgagccccggggaccgcgtgctggcggcggacgaccagggccggctgctctacagcgacttcctcactttcctggaccgcgacgacggcgccaagaaggtcttctacgtgatcgagacgcgggagccgcgcgagcgcctgctgctcaccgccgcgcacctgctctttgtggcgccgcacaacgactcggccaccggggagcccgaggcgtcctcgggctcggggccgccttccgggggcgcactggggcctcgggcgctgttcgccagccgcgtgcgcccgggccagcgcgtgtacgtggtggccgagcgtgacggggaccgccggctcctgcccgccgctgtgcacagcgtgaccctaagcgaggaggccgcgggcgcctacgcgccgctcacggcccagggcaccattctcatcaaccgggtgctggcctcgtgctacgcggtcatcgaggagcacagctgggcgcaccgggccttcgcgcccttccgcctggcgcacgcgctcctggctgcactggcgcccgcgcgcacggaccgcggcggggacagcggcggcggggaccgcgggggcggcggcggcagagtagccctaaccgctccaggtgctgccgacgctccgggtgcgggggccaccgcgggcatccactggtactcgcagctgctctaccaaataggcacctggctcctggacagcgaggccctgcacccgctgggcatggcggtcaagtccagctgaagccggggggccgggggaggggcgcgggagggggcg

SEQ ID NO: 3 - DNA, 16410 bp, Homo sapiens
agctccatgccaggcccagccgggcagacgggacccaaggcacacaggcatcaagcaggactggggagcgtccaggggctggggggccaagcagacggagcatccggaggctggggagtgtccgcgggctgggggagcagcagggggcttggagctggcagttgcacccacatctccccctcccacgttcgctcctgggttttgggatccccatcagggaagtcagcaaaggagaaattctgatttgagtgctcgggagtgaggcctcagcgcggcctgtcggctttttctcagcccccaggtgaggcccatgtgcgcaagggtttaatgcattaactgtctggggcacctgcctcttaactaggctccaagatggaagcaatgatggtcttgtggaaggatctctaggtagctggggtgcaaggagcagcatgcagacgcagggtcccttcctccctgggcacgaggccacctgcccacactgtgcctccctttccaactcccaaatgtagcttcaggctcccctgacaacggtgctagagcactttgaaactgtacagaggcacacacctgaggcattatttagacctaagaagaggtgtccattacttaaaatgaagacgaagtgactcaacaaaggcaggcaatggtctaaagagagaaaatcagctcaaaggcacacagcaacatagttcaggctgcacgttcacatccgagctgatcgcctgccttaggatgtctacttcgacagagctggctgtgtgatctttccagcgtccccagaaactgcagggtcctcatctctcagagcctgcagccttcatgcatgtgagccgggtctccacggggctccacggggctccacggggctccacggggcttggtgtgcaggcggacaggcatgagggcaggaatcccagtcaccaaagtgtctcctgcaccctgcttcacagagtggcccaggccgggaagaagccaggctgcacccacagtcatggggcctgtggcagcttgggggtggccccgtgcccgctgcacagcactactgagaaacgctgcacagtgggcacccagcgtagctggctccacatgccagccatggtcccccgaggccctgtgtgtcttggcagctgcctcccagcgggcctgccctcagcgctggtgccatgaggatgtggctgcaggagtccagagagccctgcagccaccccaaagctcctgggctttggcacggagaggacagcattgcctcaatccacctgcagccaggccacaatcagggtgctgtgcagagtgtggctggatttcctgggctatccagagcctgagccctccaggaagctggagactcacatccccagggcccggggatggggaggaccatggagttagggaaagggggctaaggtgcagaatccctcccaaattcaaaaggccctggatgcaatcacaagagaactggagtgcagaagttccaccggccaaaacagggaaaatccagaaagaagtggagactggatggctggatggaaagagattgcgtggaattactataaggaagaaaaatcaaaggggagaaggacatgaaagattagaggccaaccccagagactagaaacagaggcagcacctggagatgccaggcaggcgggctccataactgcagagcccagcgggtacaaatctgggaaaggaaagaggggatgaggggggcgcagaaactgctgacgggaccaggggaggatgaacgaaggaggctgcctggcttccgggcctcccgacctgggactgcggggatttcccaaaccggccaagcccgagccgaaggcagagtgaggaggccatctagcggcaggggccggaaggaggggcaggggcagtgcccaggagggagcctgggggctggggcagaagaggaggcagcttctgtcctcagcagggatgctgggcaaggggtggaggtgggggagggtgggggagggaaagctggaggaaggagcgagggagggatgcgcgtgaccccaaggaagagaggcagagcccaggtagatgaataacggagccccagcatgtccggatgtctggccttgtcagggaggctggtgtttggagcctctgctggtgcggggagggggccaaatggtctgggggggggggggaaggcaaagagatagagccacctggcctctggaggggaaggcagtgctgggccgtggggagcaggtgggatggaaggacagggagtgaagggcgggagggcagagcagcagcttctctggggggctcagtcagcagcctggtctataactgggattgctttctgcgccgagctcccctcaacccatgcagggggcaggccctctgaacttaggcgtgggggcaggctttctggcccatcattatttcactctggaggatcggccagtctacaaggggggcggcccctgaggtgctctccagggtgtctagggctgtctggagatggaggggagggagggggcctcacgccatcccttagggcagggaggtttcaggaaatgccctgagtcacccaagctcaatgccaagccacgtcttctccctgcgctctgctaagagggagctgaccgcacctgggggcaccccacgtccagctgtgcccacaaggaactctgtgcagctcaggcttttgcttccaaaagcacaccccacccaccctgccccttgcagaggtgagctgtgtgcacatgccacctcccggcagggctgcacacccaatctctcaggccccagggtggcaacccgtgtggggggccttctgcctgcatttggaagggagtaagtagcggggaaagacagggggaggaggagggactggggcaagaggagatgccaagggtggaaggcagggcagtcaaaagtttgcaaatgtcccactgggccaggtcgctggtccagcccaggcaccacccagtgatgtgacatccacatgctgtgcaggtagacacatcaaacaggccggcctcctctggagagaacacgggtcctgttttgtgtctgtttttactgggaacgggacaggcagcgcagacacaccgcctggagaatccctgggctcagctgagctccagctccgctcagtggaggctgccggggcctggtcaagtggacgctgctcccccacggtgctctcctccagggatcacgattcgtcacaaaccatggaaaggaacaagaaagagaggcgggaaggggagcccgggtcaacctccccaaaggcacccaggccaccgagtcttgttaacgctcagcctctcatgcccaagacagatgtgtcctaagatgctcctctccttctctcccccaccacacacacaaaaagttgatctgcaaagggaacttctggaaacttccatcgtttgagggctgagcttcacaggggtcctctccccctccactccaagggcccggggtcaagggagagtgtttaacttcacagggccacatttgcagcatctgatgactcctcagggtcaggatttcccctccacgtaaataaccctcactctctaatcctatgctagaggaatcattgcacatgagtgggatggcgattgagaaggggaagggcatgatctgtttttttttttttaaaaaaaaaaagaaaagcagggttgagagggagcttcaaattttattttcctggtttacttaggacttttgagtttttttcctttttctgcaaaaagtgaggtgattatagactcaaagattgccacctcctgggcactgtgccctctttctaggaagcccctcgttaaccagaacactgttcccccagggcccctgaccccttgggctgcagcccctaggactggtggctcctgtgcctgaagactgctggcctgctgagcccccacacaaggccagctttgcaaggagcctgaattccacattcagctcacccctggctctgcactttgctctgatcgatcgtgggggttgtgtgagcacagcttcgcccactcgcctgccccccccccaacaccctgcaatggcctccacacacccagcgcacacactggcccccactctctcccctgtgctggggtgcagcccccagacctgctttgcccatccaccctgacgcccgcagttcactctgtaactgcccttgagcacgcctccatcccatttgcagagctctacaaaaaaaaaaatgtattaattgaagcacaataaaagagcagtatttttacatttttctaaggcacttaaagcatttcaaataccagtgctttaagttatggtcactcataattatcctcggtccacagaggctgagtcttcaggaggggctcagtcctctttcttcaaaatgcagggagagccagcctgcactataaagtccctggggaagaaaaattaaataacagctgggcagctgtgcccctcaaatgtgtgagcgactctgtgcttgatgactgaagcttgcgggggtttaaactgtcctttaaaaacttaagttggttttaactgaagcacctaccattccagggaaagtcagggggaaacgcaaggatgtacacaaagccgggagtaactgctgttaccacaagacatgcgctcctccctcccgcccacctttatcttaggaaagatttttttttttttaactcgaatccttcaggtgaaaataaagcaacagcagcaacagaaaaaaaaaacgtagtcttcttcagggttaacatcagaagacaagcttgtgcggctttggccaatcagatgcgcccctggcagctataatatagtgctaaaggcagcctctctcacaagctctccaggcttgctaccatttaaaatcagactctttttgtcttttgattgctgtctcgcgacccaactccgatgtgttccgttatcagcggccggcagcctgccattccagcccctgtctgggtggggagcggtggagagtcccccgcagccgcggcgggcaaggttatataggaagagaaagagcgaggcagccagcgagggagagagcgagcgggcgagccggagcgaggaagggaaagcgcaagagagagcgcacacgcacacacccgccgcgcgcactcgcgcacggacccgcacggggacagctcggaagtcatcagttccatgggcgagatgctgctgctggcgagatgtctgctgctagtcctcgtctcctcgctgctggtatgctcgggactggcgtgcggaccgggcagggggttcgggaagaggaggcaccccaaaaagctgacccctttagcctacaagcagtttatccccaatgtggccgagaagaccctaggcgccagcggaaggtatgaagggaagatctccagaaactccgagcgatttaaggaactcacccccaattacaaccccgacatcatatttaaggatgaagaaaacaccggagcggacaggctgatgactcaggtaggaacccagcgccggggcgtggaatgtgtggctttccagggggttacgagaagccgaacacttccagacttaactctgtttgctcttcgggcagataggaaggtgatttcacccgctccttccccacccacctgcccgccccccatctcttcctcttcctggaggagaatggaggtcaagggtccagctggagaagtttagggtgtggtgggggtgaggacggtaacagacgtggttcattatggccctgatttgatgagtcttgctacaatggccttccccatcctacctctgccctggcttgtaacttggggagaccttcactttgggggcgtcggccctttccaagtcaggagtggaaatggaaggagaggctgggaatccccctcccacaaacatgaagtggtctcctggtactgtacgaacgaacgaacgtagccttgggcattggagctcagagcccccacgtttcccgttgcctctgtggttttctttcccaccactacccccaccctgcacctccccaccaaagaattctcaactggaaaagccaggaggcggttctgacaaaaggcaggggctccaggggagactccgcccgtccctgggtggctggctgtatcgcagagctggctttgcgattgcgtgtccgcaattgtgcccatcagagtgtgaatgtattgatatttctttaaggatgctctttcgttcttccaagcccgaggtaccttaggggagggacttagaacttattggcattgcatcactttagttttcaacctgcttgcataagaattaagagcgaataaatattagtgtggggggaggggaagctaagcaaaatatgaattcctctctctctccccacctcctttgagatttctgagctgccaatctcccagccaattctagactttctgaaactccatgcacgtataactgaagccagaaatgggtttccttgcaaatataggtcaacatcctttttattgccctattaaaatattcaagtcctacctttagggctaggtgcgtacagcggctgatggagtggcgctggtggggcgcaagtgcagggggagggtactgacggcagagagagaggagctacctccgtgccgccctgcttcccgacccgattcccaggcttgcttgaggccgagaaaggcgaggggcaggcaaggtagcctgctccagctgtcggaagggagaggaatgggaaatggtcctgatttccttgctctccctcatctgctcccgaccaccttaaatctggaccgcgagtgtggacgcgcgcgccagtgccagacagcagcgcgatccacaattaactctgcacgggccatggggtgcccgttgcgtgcagctggctggagggagttctccggctagcccgaggcgcccatcctctcgtcaccctcactccccgcggaggaggggccttgccagggtccctcggaacccgagaggagggaggcactgcggagagagcggcgggggcgtggatacccgaggtcccagagccagagtgggtcagcttctgccctgctctgcgggaggccaataccgcagaaggggtcctgggctcgcacaccttcccagggcttgggccttgcagccctgctgcaaagctgcaagcgcacagagccgcgcagcgaggcagacgcctgcagccccacttactcccgggttatcgatcccccgcggctagggtttcagtgcgcgaggggctgggctggagccgccgggctctgctgctccacgcgcgggagcgcaggcaccgcaggagctaacagcagcgccgggctcgctgtagtgtccccggcggcgggggcgcggagatgggggcgcccgcgcaggggccggggcgcatcgcgggctccgccggcctgccctgggacgcgcccccatccccagtccgccgcctgcctggcctctaggcctccgcgtcccagccggagcccccagcccgggggcctccccccgacccccgtccgccctgccgggggacgcagggcccagcggccccgcgcccggccactctcgccgccgcgcgcacaggcagcatttgaacttctgaccttctgtcaacttccctcagacgaacgaaacgaaaacaaatacttttttccttgggcagtggctattcccgttcccaacacaaaaggagggggaaggacggcccaagtgggggttgggtgaggagagccaggccgggattatcaggcagaccccacaaaggtcccctaaaaccgaggggggtaggggctggcagtctgtgaggtatccccggttgatccctcccctaccttccttctcccgattccaggagttaagggtgggggaggaagggatggggaaggcggaggctcgggtgctgagggcaggggcggggtgcaggaggcggcaggggagccccaggccggcgggaggtttggggagcctgctcggccgccctcattttaaataaccacctaggctctgccccaggtgcgtgaccctcttcttctgtctccctccctgtctctgggtctctaatgtgactgccgcccaagtccctcaaccatggcgagatcgtccccagtggaactttcggagcagttccggaacgcaggagctgccggttaatattaacccgggagaggaaagcgcagacagacacgctctccccgcgcgggcctaggtgccaggcgagggtgctggcggccagggggctcctaaggggcaggaggccagagggccggatctgaagcctggagtggggtcccgagccgctacactaaatagatttaatgtgcgctctggggccgccaggaaagacgctcaggtatggggttggggaggggctgttccaccaagcttgggggaaaggacagtggagagaggtgcgtttaggggctggggctgtcttgaagctgggacgcccccgcccccgcgctgggggaagcccaccggctgctggcggtgacactcgccggcgcggctcgcagatcagggaggtaggcgggagctcaggcgtggggaacaacttggcctccgccgacacaaagcccggccccggcggccctgctgggcttcacggtggctgcacagagtcgggcttgattcgcggcacacgacccaatgaattaataaccggcctgggcttcccggctttgcctgcgacaatcccgcccagcgcgggcggaggagaggccgccagccgaggccgcgcggagcccgggccggaggagggcgcaaggggcgggggcgccaactccagcaaccctcggcctccgcccctcactcgcgcagccacctcccgtcgcggcccggctggacccgggtctccctgcccggggtcctccatgcctgcccaagtggcgcagctcacagagctgggggccaggtcatcctcaccctgccgccctctccctggctgccctcctgggaagctgtttaaagcttcttcggcacagccccaggggagggagctgcggtggggtggggggcttgcatgggggtccctgtgcgtgttggtggtgtgcgcctgcgcgcaacgggcctcacatcatagctctacactgaccctggtttactgattgattttcatgtaaaacgcgttcaatcctcaagatgacctcactcaaactctgcccttccgacttttttttttaactgctggcaggcccacaaacatgcaggcactgacctgttaccagggcggcccccagccctaccccacccccagttgttgcatgttgaactctacaaccatattactgggttttattgctgccagatacacaggacttttcctgttgcgcaatttgtcacgtccccttaaagcgccgcagcagtggggccagcgtcctcgccccaccctctcgaaagagtccccccaacccacgctacagttagggccctggatagaagctgtccctccatggcgacaaccagactccaagcagagcatccttccagactggaggaggttagaggtcagccccgccctctgcagaagtcaccttgaaattgcccctcggcctccacttggcgcagcttcttgggggatgccaccatcgtcatctgtgccagttccccctctttaaatcccatgtcccaccagcagcagcagggtaaacatccaggaagcaagtcagtgcccccacaaacacacacagtggattcaactgctttctgtcgcatccttatctgagggtgaccccagaattccaggggaacccccacaatctgaatcccaggtaaccccgtctgcatctgcctagtcagtgtttcctgcctcctcccaggcaacttcctgggaaactccccaggcggaggactccgagacctcaggccttcctgtctcccctccccctcctctcaaacccctccccctccctccacctcttcagtttgctcttcaaacttgctggacgccattctatgctggggccaagaacacaagagcggaggaagggaacaggttaaagaaaacaagaaacacaatcagaccacagaaaagccaggcagaaaagggttcgacgggcaaaaagaatgtggctgtccagataaagaatgtctgtcccggccccggcctgtgctgcaagtggcaactcacctagccgcctgccacccaggctcccgcccaccgcgcagccccgccagcggcttctcgcctcccctctgcctcggatagggttagggcctgaggtaaataaatgcaaggccttcaattctccaagcagtgcgcagtgcatttttctttatttctgggaacttgcgcccaggtctctgtcaggcctgctgtgagggattctacgcggggagaaggtggaggctgcgcaggtggagaaaggggccccagaaggggggctagaagtggagggcaacgtgggggcggggcgggtatcccagagggtgcccctggagggtcctgtagttgatgtcttaaacatgcaggtcacttgtttcagagaaactttatttgcttcttaggcctcgctaggagcatcggctgtttcaggacctggagaaaggcccccagctctaccctgagaggacgtgctcctccacgctcctccgcaaatgctgtccctcttccccagcccagggcccggctcttcggtgtgtctgggccattccaacccccgtctccccacctctccgcatggccctcgcgccttgagactgggcagggcaggctgatggaggggccgggaggggtggcggttgcccaggctaacgtgtccgtcggtgggggtccccttgtcttcgcagaggtgtaaggacaagttgaacgctttggccatctcggtgatgaaccagtggccaggagtgaaactgcgggtgaccgagggctgggacgaagatggccaccactcagaggagtctctgcactacgagggccgcgcagtggacatcaccacgtctgaccgcgaccgcagcaagtacggcatgctggcccgcctggcggtggaggccggcttcgactgggtgtactacgagtccaaggcacatatccactgctcggtgaaagcaggtaagctggccctggccccccggatccgacccaaggaaggccattggcgcacctcggcttgattcaagagaaaaagaaacctggggggaggctgagggccaggagcagggtcgctgggcgatgactgcgtttccgcggtggaacctgccctgtgaggtgccggcccctcgaaatcacccctacctttgaggccacagagcccaaggttctccatgccccgagatggggtcctgtggcttcctgcccgcttctggagcccccactgcagggggtgggaaagcgtgactgggggaggggcgctaggcccttccaggcgagggaagacagccctgcgcggttagccaggtctgggcgagctccttcctctcgtttagggcttaagaaccaaccgcccccacccgctatcccaagcgcaggggtgtctatcctgccccggagcccgcgtcctggctcctccccgccgggcgcccgtggatcctaagctgcctttggggagaggcctggtgggcggcagtaaacccaggggcaaccacctccagcatctggaggcggcgcgcccggagcctgcgttcctactgggagccgggccgggacgccctgggcggcgggcaggccccgaaacgccggcccgagtcggcgcgaggctgtcttctctgggcctgcaacgccacacgctgttgccggcgaggaacagccgtggaggaggcgccatcgcgcgcacgcaaacctccggcccgaggctgtgtgcacagcgctcttctccgcccgcataaattggcacgtttagcaaagccgttcacggtgaatttcggggaaactctgccttcctcaacccccttccaggcttccctacttgtctcctaaattccatgttaatggcactatgttagtaggaaaacactgttaaggtgtcaaggcacacttgtaggtaaaggctagagtggcttctcgtccccacagaaagcaaaggcgtggagcgggggcggcaggggcgggtgtgcggcccggagagctcccggctgcaggcaggcaggaggcggcgcccccacctcgcgggctcggcggcggcccctgggcccagggcgccccctgcgcaaaacctcctccccggctccctgcccgcggggtccccctagcgggggtctccggaggcctcctcccaagtgagcagcgctaatccatcccccggatcgcgccgggagagcggagccgcggcgcgggagccgctcattggcattctgagcacacgggcgggggcgcggggcgcagcgtgtcaagccgggccgtgcgactcgacgactcgggctcgccagcgcccggggtcgcattccggggggctacggagggcctccaacggccagccccgcacttcatgccagagaaaccgatgagaagattaaaagccccctgtaattccagcaggaagattctttctggcaatctctatttgcaaaaagcatgatcccggagattggaatgcaaagaagacggccctccccgccctcctccccggccccctgcgctccgccccaacttcaattattgtcctggggacagtgagcctcagagagcgacagagggctcgagaaagcgggtagtcaaggggccttgagacccggcgcttccagcgctccgaacaggccccgccatttaaaattcaaatacacatcttgagtgcttggaagagaggcctggctgtgcaaatagtgcttgtgaattgcacacggggtgggggggggttgcacctgagcaaatagggagggggaggcccgcgagctggggagagagtgagctgagaacagggaggggagaaaatggaagtgtccccttccaagagtgtctcctgtttatcccagaaatcacaatgacaatgctgggccctttattggattttaattagaaaatccacacaagcctcggattttcacacctcggccaatctctggaatgtttgtccagttgctacaactactgcagctatttttcactccccgcccccgcccctccgcaggcccacgccgaggcgcggcagggtgctgcgggcaggcgggcaggcgggcaggcgggccaggggtttccgccgcgcagcccgggtgctgagtgcgcgagcaggcgccgcgccccgcgccggggcgggagggaaggagggtgcgcccggcgcccgcgggagctcaaggaggcttcctgaggaatccaagtgcagagcaaacaccctctggatggattcgcggcgaggccgggtgtgtgcggagctgggggtggggttggaggaaggcggaaggaaagagtgtcaccggcctctgcaggaaacgccagccaacctctgtgaccgccagcccagacttagagagtcgttaaggaatgtgtcggaatcctgtccctggggcagtggggttgggggagggaggtgtgtgcgggacccgcctggaatcaatcgccccgccccgcgccttgcgcacccctggcctaggagcgcgggcaccaagcgtgcgccctcctccccgagacgcgcctccctctcggaactcaatgccctgtcctctcttctttcccttctcctcacccgcagagaactcggtggcggccaaatcgggaggctgcttcccgggctcggccacggtgcacctggagcagggcggcaccaagctggtgaaggacctgagccccggggaccgcgtgctggcggcggacgaccagggccggctgctctacagcgacttcctcactttcctggaccgcgacgacggcgccaagaaggtcttctacgtgatcgagacgcgggagccgcgcgagcgcctgctgctcaccgccgcgcacctgctctttgtggcgccgcacaacgactcggccaccggggagcccgaggcgtcctcgggctcggggccgccttccgggggcgcactggggcctcgggcgctgttcgccagccgcgtgcgcccgggccagcgcgtgtacgtggtggccgagcgtgacggggaccgccggctcctgcccgccgctgtgcacagcgtgaccctaagcgaggaggccgcgggcgcctacgcgccgctcacggcccagggcaccattctcatcaaccgggtgctggcctcgtgctacgcggtcatcgaggagcacagctgggcgcaccgggccttcgcgcccttccgcctggcgcacgcgctcctggctgcactggcgcccgcgcgcacggaccgcggcggggacagcggcggcggggaccgcgggggcggcggcggcagagtagccctaaccgctccaggtgctgccgacgctccgggtgcgggggccaccgcgggcatccactggtactcgcagctgctctaccaaataggcacctggctcctggacagcgaggccctgcacccgctgggcatggcggtcaagtccagctgaagccggggggccgggggaggggcgcgggagggggcggggcggggcagcaacagcaacgcaaagcaaaaagacactcggaaaaggcgcacgaaccagactgagttataataagaataagaataataaagtaggacagtccaaagtagactctaaggaaacaaggaccccgggaagttttgttgttgtgcttagttgatatatattttttgaatttttttggttattgttttattttggttattttttcctcctctcctggctatttatttgtttggtatgaatagatgttttaaataatatgaaccggaccttcaagagccttaaatagtttgtttcttggataatttattatgatcatgtgaactgtactcacgggggaaagattattttgtgaggccaagcaacctgctcagagtctatttttctacatgtccctcgtcccggctgtcagaaagcaaacctctgtccccgccatccctcccttccatctcctgcttctcagcaagtgcaaactcaaacgtgcttcatgggggtccacaaattatatttttatacacagaattgtaaattagattttttgagagatcaatacttaactgaattacatttcatttttgaaatagtgtaaaatatgaaaatatattattttaatttaactagtttccgatgtaacagccatctcctctgttgtctttatggtttcatattccctttgtattcaccattttgccacattcttggaagccaagactgttacacacacaacatacacacacacttttctttttcttttcttttcttttttttttttttttttttttttggacaaactggaagaactgttatttttaacttcaaagaatttattagaaaataatattttttaaaagcgcacatagtgacgagcccacgaggatggagcctgcagtttgtacagagaaaacaaaggatgtttttgcattaataaattgagaaataacgctgtaaatttactaaaatgtatttttgaatattttgtaatagttttatagaaataaaatgtgccatgcacagaacgcttagtatctaataactaagaattcctgttgcagccctttttcttttagtttttggcttctttgtttgggaaacctagcgctgacaggagctcccaatattctatttagatgtgttgcttatactgtgaactcagactgactgacttaggtaaccaaagtaggcacacaaaccaacttgtttcttctcatggctggagaaacctcagctctgtttgtatttattgtgtgaagctctttctacactcccaaaggttgggagtgccattcgtggggactgtgctgtccccctcaccccaccccttaactagcaaagcctgatgtgtaagcccagaggaggtggcctggggatggccaccaacaaccttctcttaggactcctgtgtgtggttgctgggcagacccctggcctgaccctgtttgtgcttctctgatctgtagggaggaggctcattgctgctttctgaggtcctgttgtgtcccttccagaaagcatccttagaagagaggacccggtttgatcttctctgaagctcctatcctcttttcttgagggtccccttaaatctgagtggcaagcccacagtggcgagggccagtcgctggaatgtcggggctgctgttgaggtcctcaggctacactggttgtctgtgtcacatggcggtcgggcgtggactttgatccctgttgagagagcatgctgctgtgggcgctgggctggggaaggtccttccatgcttcatgctgccttggaggtttggccaagagggtctctgcccttggcggttgtcagggtcccaggtggaggactactcccagttctttggcttacggaaggtgtctgccatgctttgtgctttgggccgaatcgtctgagcaggctgggccttggaagagttgcgctgcatgagcttggggcctcccagcagctgcagcctgtgtagaaggtggtccaggcttagggaacaggagtgaacaga

SEQ ID NO: 4 - protein, 396 aa, Homo sapiens
MALLTNLLPLCCLALLALPAQSCGPGRGPVGRRRYARKQLVPLLYKQFVPGVPERTLGASGPAEGRVARGSERFRDLVPNYNPDIIFKDEENSGADRLMTERCKERVNALAIAVMNMWPGVRLRVTEGWDEDGHHAQDSLHYEGRALDITTSDRDRNKYGLLARLAVEAGFDWVYYESRNHVHVSVKADNSLAVRAGGCFPGNATVRLWSGERKGLRELHRGDWVLAADASGRVVPTPVLLFLDRDLQRRASFVAVETEWPPRKLLLTPWHLVFAARGPAPAPGDFAPVFARRLRAGDSVLAPGGDALRPARVARVAREEAVGVFAPLTAHGTLLVNDVLASCYAVLESHQWAHRAFAPLRLLHALGALLPGGAVQPTGMHWYSRLLYRLAEELLG

SEQ ID NO: 5 - DNA, 1971 bp, Homo sapiens
gtccggagtccgggcgttctggcagcaccagagtcctgcagcagagacgggctgggctggcactcacccacgaccccccttccctgctatgatggtttgtcagtagcaggtcctagacaccccccgcccgctcccggggcacgtgggcagagctagcagcagccaggtgcacggagccacaagagctctagggcactctgggggcagctgtgcctgctgccctctctggtgcctgtggagatgcctaactgacaggagagttagtgaggacaagaacgctcccttttggccgaggtccgctgtatccatggctctcctgaccaatctactgcccctgtgctgcttggcacttctggcgctgccagcccagagctgcgggccgggccgggggccggttggccggcgccgctatgcgcgcaagcagctcgtgccgctactctacaagcaatttgtgcccggcgtgccagagcggaccctgggcgccagtgggccagcggaggggagggtggcaaggggctccgagcgcttccgggacctcgtgcccaactacaaccccgacatcatcttcaaggatgaggagaacagtggagccgaccgcctgatgaccgagcgttgtaaggagcgggtgaacgctttggccattgccgtgatgaacatgtggcccggagtgcgcctacgagtgactgagggctgggacgaggacggccaccacgctcaggattcactccactacgaaggccgtgctttggacatcactacgtctgaccgcgaccgcaacaagtatgggttgctggcgcgcctcgcagtggaagccggcttcgactgggtctactacgagtcccgcaaccacgtccacgtgtcggtcaaagctgataactcactggcggtccgggcgggcggctgctttccgggaaatgcaactgtgcgcctgtggagcggcgagcggaaagggctgcgggaactgcaccgcggagactgggttttggcggccgatgcgtcaggccgggtggtgcccacgccggtgctgctcttcctggaccgggacttgcagcgccgggcttcatttgtggctgtggagaccgagtggcctccacgcaaactgttgctcacgccctggcacctggtgtttgccgctcgagggccggcgcccgcgccaggcgactttgcaccggtgttcgcgcgccggctacgcgctggggactcggtgctggcgcccggcggggatgcgcttcggccagcgcgcgtggcccgtgtggcgcgggaggaagccgtgggcgtgttcgcgccgctcaccgcgcacgggacgctgctggtgaacgatgtcctggcctcttgctacgcggttctggagagtcaccagtgggcgcaccgcgcttttgcccccttgagactgctgcacgcgctaggggcgctgctccccggcggggccgtccagccgactggcatgcattggtactctcggctcctctaccgcttagcggaggagctactgggctgagcgtcccaggcatagaaacctcgaagcgcccgaggaaacgagggcctgctggctgagatatggggctgtgggcagcagacgatgccgactggaagggagggagagggagggggagggagaaaatggggctatgcctggcttaggggcaacctcgtactgagaggaggtgataccgggtcctaggtaggtagggctgatggtgcgcactccttaaagaggactatttagcctctcccccagtgcttcagccccacccgataattttattttatttcttatttataaattgtaatataatgattcccttgcccagcttgccaaggtgaggggctggggcatggcattaacactgtctgacacagccagccagtctgacatctgacgttttcctgcaaggtgggctaataaagggaaggctttcaggagcttactggaaaaaaaaaaaaaaaaaaaaaaaaaaaaaaaaaaaaaaa

SEQ ID NO: 6 - DNA, 12399 bp, Homo sapiens
cacctgaggtcaggatttcgagaccagcctgagcaacatggagaaaccccatctctactaaaaatacaaaattagccaggtgtggtggtgcatgcctgtagtcccagctactcgggaagctgaggcaggagaatcacttgaacccgggaggcaaaggttgcggtgagctgagctcacgccattgcactccagcctgggcaacaagagcgaatcttcatctcaaaaaaaaaaaaaaaaagttcaagagtgtaactagttcaccccaaggtagtgtgtgagctgcagaggtaacagccgccctcctacctccccctaaataccacatcatcctctgtattttatatgtgatttcatatttaagtcagttctggggccaggcctggtggctcacgtctgtaatctcacactttgggaggccaaggtggacagatcagcttgaggccaggaggtcaagaccggcctagccaacatggtgaaaccccatctttatgaaaaatataaaaattagccaggtatggtggcacatgcctgtaatctcagctgaggcatgagaattgctgagcttgggaggtggctgaggcatgagaattgcttgagcttgggaggtggaggttgcagcgagccaagatggtaccactgcactccagcctgggtgacagagcgagactctgtctcagaaaaaaaaaaaaaaaaaaatcagctgtgagccaggcctggtggttcacgcctgtaatcctagcactttgggaggccaaggtgggaggatcacttgaggccaggagtcctagaccagcttgagcaatatagtgagaccccatctctaccaaaaaaaaaaaaaaaagtgtttttaattagcacacctatagtcctagctatgtgtgagactgaggcaggaggattgcttgggcccaggagtttgaggctgcagtgagctaagactgtgccactgcactccaacctgggcaacagagcaagaccctgtcttaagacaaaaccaaattaaacaaaacaaaaaacagctctatttcattcttaacgttatattatttccactctaccatttgtatttatgccaggagaatttgtaagaaagtaggtttgtgggtcttccagttagttcctttagggtggatgcaggtaagaatatttaaaggtggagagcagagttttttgatttatgataatattatgaggggaccagtaaatgttagaatagtgcctggcacagacagtgctttatgtgtgttgtctcatttaattcttaggaaactgaggcacagaaaatttaaggaaattgccccagatcacgcagctagttaggggtggagttgggatatgaaccaaagcagcctgatgagtatgagcgcccagcaaccacactgttctgtctactaaaaaaaacttggcaaactgcttattccaggcccaggaacaagtagtaggccctgggagttcctggggacaccaggagccagaggaggcctcactggaggagaagataccagactctggaagcgaagttgaagcaagcctagctgctggcacttagaacagtcccgaactgctcttgagacagcttgggtggtagaattgggagaggccaacctatcagttgcagtgttgaaatgtggccaccaggaggcagcagaaagttgggctggtagcagaaaagggcctggggagcccacctctcatcctggcctttgtgcttcagggctcactcgctttgacctgctgggtgactttggacgcttcaactggctgggcaatttctacattgtgttcctctacaacgcagcctttgcaggcctcaccacactctgtctggtgaagaccttcactgcagctgtgcgggcagagctgatccgggcctttggtgagagggagtgagggtgcaggctagtggggggtggtaatcatctaggacactctggaacatggatcaccccagcaggggagagggtgtgcttcctgctttgtgggtaacgtacctcatccacacattctgtggcttggctgcctggtccctaacggactggacctttgacttgcttttgactgcccatctcctcttctccagggctggacagactgccgctgcccgtctccggtttcccccaggcatctaggaagacccagcaccagtgacctccagctgggggtgggaaggaaaaaactggacactgccatctgctgcctaggcctggagggaagcccaaggctacttggacctcaggacctggaatctgagagggtgggtggcagaggggagcagagccatctgcactattgcataatctgagccagagtttgggaccaggacctcctgcttttccatacttaactgtggcctcagcatggggtagggctgggtgactgggtctagcccctgatcccaaatctgtttacacatcaatctgcctcactgctgttctgggccatccccatagccatgtttacatgatttgatgtgcaatagggtggggtaggggcagggaaaggactgggccagggcaggctcgggagatagattgtctcccttgcctctggcccagcagagcctaagcactgtgctatcctggaggggctttggaccacctgaaagaccaaggggatagggaggaggaggcttcagccatcagcaataaagttgatcccagggtttgctttgtttttttaaagcacctgtctctgtgtatatttgaacagccttgctcccaccagcccggcgtctgcccagtaagagaagcagaggggtcctgcccaccatccttttccttccccttcatctatattattaccatcctttggggacttggatctggtgttaggccatgcagttgggcagtgggaaactcctccatcctcagtcttgcttaaagctggcttgtcccctgactcctccttatattcatggccttgtttctgtaccttgagtattgggggtaggacatgggcataggagtaggctaacctccccgatacactcataccttcagccctatcctccctggcttaactattggatatagagtaaaataagtggctggctgacaaggattttcatttcctacctgagagaagaatctagctttttactagaggccatcagtttcttgaaaagcccatgtgctatgttgggagtaagagcgcaaaacttaggcttatacagattgtgtattgcacagcttccagggacaccattcacctcatagtcattgtgtggatagttactatgatagttttctaataaatgtcagtcatcttgaggaaggggtgctttttttttcttttttcttcttcttttttttttttttttttttgtgagacaaggtctggctctatcaccctggctggagtgcagtggtgcaatctcagctcactgcaacctccgcattctgggctcaagccattcttccagctcaggctccagaatagctaggacaacagacacatgctaccatgcctggctaatttttgtatttttagtagagatggggttttgccatgttgcctgggctggtcttgaactcctgagctcaagtgatccacctaccttggactccccaaatgatggaattacaggggttagccacaacacccagccatcttttttttgggggggggggtcctcactctgtcacccaggttagagtgcagtggcatgatcttagctcactctacagccttgaactcttgcacccaagtgatcctccttcctcagcctcctgagtacctaagactacaggcatgtaccactacctcccactaattaattttttttttttgaaacggggtgtcactatgttgaccaagttggtcttcaactcatggcctcaagcagtccttgcctcgcccccaaagtgttgggattacaggagtgagccactgtgcctggcctggttttttctgatgtacacaaagttgctatgagtactagccatagagtgtggtgaggggagggtgaatatgaaacacaaagtgtagattccacaaatgcttgcaaagcacccttctgtagagtaggcacttaaccttggagatacatgataaatatgtgtgtgcttaagattgctttgtctagtaaggtgccagataattctaatactgttctaacactgcagttaagtaatggttctcgagggaacacaggtgggcagtgttgggacatcaaggaaggcttcttagaagaaatgacttctaaacaaaagaaattggacgtggtgcctctgagatgagatctgagtggctgaaaatggagccccgggagtccagagagggagaaagctggagaggtgcctagatggacggttaagggaagtgatcctcacctcttacaaagacttttgagtgtccactgcccttcagcagcttccaactgagaagtcattcatttgaaaaacagcagctattagatgcctggcaaagaagagaaggggaaaaaagttggaagtcctaaatgagacaataagtaccaagctctgttcctggttctggggaagttccaaaggagactgcaaagttagtagagaaatgggcccctggtatcctcacctcccagtcctgcttaatgctgaatgtgaggagagatggaagtcggggagatctggggaggactacagagaagggaggctctggttgtatgcccaaagggcttggctggtcttgggcagaaattggaacccatacttcctggatgacagcggagggcatggcctgccagggtaggctttgagagagtaaaaattccaattcaagtatcagaggagggtggtttggttccaagtttaagcttggaggtagaaggatgtttaggcagccaaggactgtaaagctacaagtatttcctaggcaggtgtctgcctggtttccaggatgggacacacttcagtcgcaaaatggaccagcagtgaccggctagatcccccgcccgggtgggagggggtgggacggtcttgggagaagggcggagggcggggccaggggccacttaaggcggagtccggagtccgggcgttctggcagcaccagagtcctgcagcagagacgggctgggctggcactcacccacgaccccccttccctgctatgatggtttgtcagtagcaggtcctagacaccccccgcccgctcccggggcacgtgggcagagctagcagcagccaggtgcacggagccacaagagctctagggcactctgggggcagctgtgcctgctgccctctctggtgcctgtggagatgcctaactgacaggagagttagtgaggacaagaacgctcccttttggccgaggtccgctgtatccatggctctcctgaccaatctactgcccctgtgctgcttggcacttctggcgctgccagcccagagctgcgggccgggccgggggccggttggccggcgccgctatgcgcgcaagcagctcgtgccgctactctacaagcaatttgtgcccggcgtgccagagcggaccctgggcgccagtgggccagcggaggggagggtggcaaggggctccgagcgcttccgggacctcgtgcccaactacaaccccgacatcatcttcaaggatgaggagaacagtggagccgaccgcctgatgaccgaggtaaggagggcctctccctacctgcgccggggcactgccagctcctccgttttcctatggcagcccaggtgggtgagtccagcttcacctacagagagtttagccttagagatttcccttgtcaccagccccaggggggggaaaaatccttgcagtctggacagagtaattaggacggctcccgactagaatagtctgggatggggaggggaggagggactgactagtgagagtgaagctggtagaggcagcgagggcggggaaaggaggatggcggcaggcgagacgggaaatggctgaggcgtggaccgggggtcagccacccagccaggcaggaagggctggggtgagctgggggccaggagctggctaggtagcagacagaagcctggctcctgacctgattgtagaaaagctctgtgggccgaaaagaagagactgaaaagggctgggacaggggtggccaagggtcaggtcaccagaccatctacctgaggaggggctgggcagacattgcttttggcatgggaaacgtgtcctagaaccaggacacatctattctagggtctattctagggctatcctagggtctaaacgataatcttggggttaaaaagtaatcctcagtgagaataacagatggacccacctgagaatcagagggctcctaagtcttccaagctctgcctatctcaggggctttccttggaattacataattaggataaattcaactaccatttttgagcattggaaccacctagagtgaggtggaagagaaggaccacctctggcgagtgatttattttttattttttgtagagacggggtcttgccatgttgcctaggctagtcttgaactcctgagctcaagggatcctcccacctcggcctcccaaagtgctggattacaggtgtgagccacttcatccagcctggccagtgatcttgaatgttagaactgaaagaaaacttgagagatgacattcctccaaccctccccacccccactgccaccctcattttacatctgagagctggacttcccccaggaggaaaagcaatttaaactctcacaatttaaagtggcatggctgggactagaattcaggactcctgattctaagccctatatactttccattacaccacctaattgtgtatggagtggggaggtggggggagagggaggggtacggatttcctctgattaattgccatagttttccaggttactttgttaggaaaacctgtccagggaagaaataattccttttgcttctgccctgaagccttcctcctacctagagatgggaaaaggggcattccttctttcctccttggccgtggaacattgtggccctttcctctggttgccacctgcagcagagtgggcagaacaagggacaggctcaggaggggctccggcaggcctgggagccccctcagtcttacttatctcccacaccctggctgcaaaaggcctccattgagttggctctgcactgggctgctgcccccgcaggtcagggcctcggggcctccttaggggacgaaacgacaaagttggaaggagctaccacttttcctgttttccccccacccaaccccaactctgcggggtggaggagactagaggagaggggaaaagtgtctttctgttaggagaaaaccaagtccgaggtgagaggtcggaggggaaagaaaaaaaggaagctgtccctctgcgcctcctcccgcggtgtccctgctatggtgcgcgccaaggtctcccgctcccaggctggagcggccaagataactgtggtctcactcccccaggtgcaggcttccagccgcagcttcttttcctcttctccgctctcaccctgcagggggctccccagatccgcacaaggagaccgttattctcaggtgctactcccacgcccttctgggctgcatctagagcagagatgggagagataggggctctccggactggcagagccttagctggaactgggaagactggacgcgcggggggaggggacctacccggacggagcagggattgggggcgcggctttctgccctttcctctccctgagccggggcggctgaaaagcgcctttgtggccggagctgctgagtccgcttctcccagcgcgttcccggcctgggctcaggattctcgctcggttaagtcatcccggataatggccattgtacttcgcgcaacccccgccaccccaatcctttttccagcagccttgatcccgcacagctcctcttgggcacctctctggggggcacccagagcacgaaggggttaatgctaagggcccaaaggaggaggaggaaggaaggaaggaagcgatgcccggtgccagggaaggcaatgaaacaaaagatgaatcaggcggagggaggggagccggtgcctggggcgggggccaggctcaaacaggggctctccgggagcaaccaggccctccctgaccacgcttccattcgccctcctaactctgtccttctcccaggttgagccgctgtccctgaaccaataggggtgagggacgggcattaaaaatggggaaggagctaggtatgggcaccccctctcaacctcagaggtgagcccacagttctccaacgacgtcctgggaacagagtgcagattggtgactaccatcgactcagattctcatttctccaggacactcaaaagctgggaggggtaagagtagggccggaataacaaagaatcaaccctcctatccaggtcccttttgccccaagggtggtggtggggagactgactcctttattccccgccagcgttgtaaggagcgggtgaacgctttggccattgccgtgatgaacatgtggcccggagtgcgcctacgagtgactgagggctgggacgaggacggccaccacgctcaggattcactccactacgaaggccgtgctttggacatcactacgtctgaccgcgaccgcaacaagtatgggttgctggcgcgcctcgcagtggaagccggcttcgactgggtctactacgagtcccgcaaccacgtccacgtgtcggtcaaagctggtacagtaggaggagggtggtaaaatccgcccaaagggggctccctccgggaaactgagtctgcagtagtactgttgccaccctaggggagaccaagcgggctttagtgttgaaaaccacccgtccaggccgaggcggtgcctcacgcctctaatcccagcactttgggaggccaaggagggcggatcacttgaggtcaggagttcgagatcagcctagccaacatggtgaaatcccgtctctattataaatacgaaaattagttgggcgtggtggtgtatgcctgtaatcccggctactcgtgaggctgaggcacgagaatcacttgaacccgggaggtagaagttgcagtgagccgagatggtgccactgcactccatctactgggcgacagagcgagattctatctcaaaatacatacatacatacatacatacatacatacatacatacatacatagaaaagagaagaaaaaggaaaagaaaagaaagaaaaaccgcccttccttttctgcccaggttggggccagtcagattaacggaaaattgtgttgtccatgactggcgatatctgcgctggccttgagaatcgttctggaaccgctggtcttgattcaatcctccctgtggttgttccctgcagataactcactggcggtccgggcgggcggctgctttccgggaaatgcaactgtgcgcctgtggagcggcgagcggaaagggctgcgggaactgcaccgcggagactgggttttggcggccgatgcgtcaggccgggtggtgcccacgccggtgctgctcttcctggaccgggacttgcagcgccgggcttcatttgtggctgtggagaccgagtggcctccacgcaaactgttgctcacgccctggcacctggtgtttgccgctcgagggccggcgcccgcgccaggcgactttgcaccggtgttcgcgcgccggctacgcgctggggactcggtgctggcgcccggcggggatgcgcttcggccagcgcgcgtggcccgtgtggcgcgggaggaagccgtgggcgtgttcgcgccgctcaccgcgcacgggacgctgctggtgaacgatgtcctggcctcttgctacgcggttctggagagtcaccagtgggcgcaccgcgcttttgcccccttgagactgctgcacgcgctaggggcgctgctccccggcggggccgtccagccgactggcatgcattggtactctcggctcctctaccgcttagcggaggagctactgggctgagcgtcccaggcatagaaacctcgaagcgcccgaggaaacgagggcctgctggctgagatatggggctgtgggcagcagacgatgccgactggaagggagggagagggagggggagggagaaaatggggctatgcctggcttaggggcaacctcgtactgagaggaggtgataccgggtcctaggtaggtagggctgatggtgcgcactccttaaagaggactatttagcctctcccccagtgcttcagccccacccgataattttattttatttcttatttataaattgtaatataatgattcccttgcccagcttgccaaggtgaggggctggggcatggcattaacactgtctgacacagccagccagtctgacatctgacgttttcctgcaaggtgggctaataaagggaaggctttcaggagcttactggaaaacacttccattcagagggtgattcacctccagagagggggataaaggtggccaacctgagttaacagcttacttccggctccccctccttcctcagcagggtctttgggacccttttcctcccttcagaaggggcatcccaacccataagcaacagagcccacaagagtcgtacccttttccaggctgctgtttccgtctcccatccagagaggaccagcatttcgctcctgcggctgaagggtccagggttgtcccaggtgggaaagactgtgcacacctcttctctgtttataggggctccagcaggggctcaggcccctatgagtactgatactcaaagcatctagtcccctattgttagccagtagtcctggtccccatcctaccttggtggcagggcctggatggggtggatgttttttctgatatgccactgggccaactgatgatccatgggaaacctgggtaataatttgcaatcgtcgcatagtcatggcctgaaaaaggaaggggggcacagtaagaggctggaatatttccttactgaagggctgctcagagagagagaatggattcagcctgggtgtatgtgggggaagtagattccctaaacaagggcatatcagcattacaacccgagctcccctcctgatcgctggccctgattgttgtttctactggcttggtcttcagcgcctcctcctccctctcccattatcaccagctctacttccctagcctgggtgtttttggaacctttaggttccaacgcagtagacacctggaatccaggcaggcaggtgcccctgagttaggaatggaagctctggaggaagctgggctgctccagaagggaagtgtcttgggtggaggtgaggcggggaggaggaaatagtcgggggttgttggtaccacccggagctcgcccttgccagtagccgctgtgcgctcccatcgcacagggtccctgctatacaccgccctctggtggccttcaagagaattctaggcctatgcatatggttctctgaaacggaaggcaagcggtctcagacatggaagggtgtactggacctgcgcaaaggagcccgctcccaggagggagagatgatgggtgggccttgttaagctgccagcgctgagtcacacatatcaggagctgtcaaggagtccacctgcttctccttgggcaagtcgttccaaaccatggcacttttacatcactcctgcctagtttagctctgtggttcctggaagctgcagatgccctactagtatacctgtagtagcccagacttccttatgtatcaaagttgccgcctcatgtccctcaccctcatgacccttgagttcaattgtcaaggaggtgaaagcataatagggttccaggtagtgcttgataaaggcaatatttggaagcaggtcaagtttacctttggtgtccttccacccactgctgccctcaacccacactgaggctctgttgccacctttgtttttccgggtctcagaaacacatcccaagctgtggacctttcctactttctaaacctgcccttgtctctttttatcagatctggtgccttcctgtcatgtcaccacaggtgatgactaatccgataaatgaattagttgtcctatgttgatgggctccttggttttactgcccttcccaggtaatcaggtaatgcatatatatatacatgtattttttaaagagacaggatctcgctctgttgcccacgctggagtgcagtggtgtgaccttggtcccctgtagcttcaacctcctgggctcaagcaatcctcccacctcagcctctcaagtagctgggactacaggtgcatgccgctatgcctggctaatgtttgtattttctgtagagacagagtcttgctacgttgcccaggctggtcttgaactcctgggctcccgcctttgccccacaaagtgctggaattacaggtgtgagcccctgtgcccagcagtaatacatat

SEQ ID NO: 7 - protein, 411 aa, Homo sapiens
MSPARLRPRLHFCLVLLLLLVVPAAWGCGPGRVVGSRRRPPRKLVPLAYKQFSPNVPEKTLGASGRYEGKIARSSERFKELTPNYNPDIIFKDEENTGADRLMTQRCKDRLNSLAISVMNQWPGVKLRVTEGWDEDGHHSEESLHYEGRAVDITTSDRDRNKYGLLARLAVEAGFDWVYYESKAHVHCSVKSEHSAAAKTGGCFPAGAQVRLESGARVALSAVRPGDRVLAMGEDGSPTFSDVLIFLDREPHRLRAFQVIETQDPPRRLALTPAHLLFTADNHTEPAARFRATFASHVQPGQYVLVAGVPGLQPARVAAVSTHVALGAYAPLTKHGTLVVEDVVASCFAAVADHHLAQLAFWPLRLFHSLAWGSWTPGEGVHWYPQLLYRLGRLLLEEGSFHPLGMSGAGS

SEQ ID NO: 8 - DNA, 2074 bp, Homo sapiens
atcagcccaccaggagacctcgcccgccgctcccccgggctccccggccatgtctcccgcccggctccggccccgactgcacttctgcctggtcctgttgctgctgctggtggtgccggcggcatggggctgcgggccgggtcgggtggtgggcagccgccggcgaccgccacgcaaactcgtgccgctcgcctacaagcagttcagccccaatgtgcccgagaagaccctgggcgccagcggacgctatgaaggcaagatcgctcgcagctccgagcgcttcaaggagctcacccccaattacaatccagacatcatcttcaaggacgaggagaacacaggcgccgaccgcctcatgacccagcgctgcaaggaccgcctgaactcgctggctatctcggtgatgaaccagtggcccggtgtgaagctgcgggtgaccgagggctgggacgaggacggccaccactcagaggagtccctgcattatgagggccgcgcggtggacatcaccacatcagaccgcgaccgcaataagtatggactgctggcgcgcttggcagtggaggccggctttgactgggtgtattacgagtcaaaggcccacgtgcattgctccgtcaagtccgagcactcggccgcagccaagacgggcggctgcttccctgccggagcccaggtacgcctggagagtggggcgcgtgtggccttgtcagccgtgaggccgggagaccgtgtgctggccatgggggaggatgggagccccaccttcagcgatgtgctcattttcctggaccgcgagcctcacaggctgagagccttccaggtcatcgagactcaggaccccccacgccgcctggcactcacacccgctcacctgctctttacggctgacaatcacacggagccggcagcccgcttccgggccacatttgccagccacgtgcagcctggccagtacgtgctggtggctggggtgccaggcctgcagcctgcccgcgtggcagctgtctctacacacgtggccctcggggcctacgccccgctcacaaagcatgggacactggtggtggaggatgtggtggcatcctgcttcgcggccgtggctgaccaccacctggctcagttggccttctggcccctgagactctttcacagcttggcatggggcagctggaccccgggggagggtgtgcattggtacccccagctgctctaccgcctggggcgtctcctgctagaagagggcagcttccacccactgggcatgtccggggcagggagctgaaaggactccaccgctgccctcctggaactgctgtactgggtccagaagcctctcagccaggagggagctggccctggaagggacctgagctgggggacactggctcctgccatctcctctgccatgaagatacaccattgagacttgactgggcaacaccagcgtcccccacccccgtcgtggtgtagtcatagagctgcaagctgagctggcgaggggatggttgttgacccctctctcctagagaccttgaggctggcacggcgactcccaactcagcctgctctcactacgagttttcatactctgcctcccccattggggagggcccattccatccatcttaggcccctttgggtgggcttgcgcctcagttgatgctgctaaattccctgggagccagcatggatctggctggacccgatgctgtccagaactgggaaggccacaggggtggggcagccatcccggccattctgaggtatgacattcctccccggccacactcctcaagacacatccagagactgttgctgtctgtgggcagagttctgtgttctggccaatgtgaccgtagtgccggggactgggggaggtgggttggatgtgcttgccacccccccggctaagctcccccttctgctgaaccatgatccccaccccctccgccggtcagtctcccataccttatttattggagtggagggggaagcccatgggagaattttggggatgttttggtcttttcttccttttgtaataaaaattatttaagttgttagagccaaa

SEQ ID NO: 9 - DNA, 13097 bp, Homo sapiens
tagaagtcagagtgacaccattctgttggggaagtggtcagtcaaatgaagtataaggtgttaaaagcccaagaagaaggtaacagttctagaaggaggaaaaacagtttcttcgatttaggaaggaaaatgattgacttgaacagaggtggacaacccagcatgcttgccctgggaatcattcaccaagagactgttaccgtggagtcaacaagggcctggattccaactccagctccctattggacaagctctgaacctgagtttctgcatttgtaaaatcaagataatagtgtgaacttcacaaggtttttgtgaaggttttgtgagacgatattagggtttcctctctttcttagatcatctaacttggctcttcatcattactaggccatcatctttgcttgtaccactcccctttcctgagtatcctcttcattcttttcagtcattcccttccctccttcagaactgattttatccaagagatctaaccccaggctaatcatttcttatttccatattctcctagaccaaattcatctaatctgttagcctatctagtttataatgcattattctgtattcatttatgagtagcatttgtggctttgtaattgtcttttattttcgtgatttgtctttaaggttaaatagtaaattcctcaaggacaactgtatcttctacttttctgtttgcattctgtgttcttatgtgcctattatactacagcatcaaggtagatattccctaatagaaagagatagcggctgctaatgaccactgctgtcttgggtcaccacacatgagcctccttatgtattccccctagatgagtaataggaatcagaaaacttgctgctgtccctaagtgaacccagcagaggataaaagtgaattataagtaacatttgtactttaaagcttatatgttttaaccctttcaacaactctatgaggtatgtgatattattttcatgttatagatgagaaaatggaagttcagagtcatgtctaaggtcacacaggtagaaggtagcaaagatggtgttcaggcatgggtttgccacctccaggtctactgtttctactgagccagggttgctgtgcactgggtcactggagggtgttggactaactgcattcctgaaacttgtctggttgtggcttaccacccttggatttagagctggattgatctggtcaagtggcaggattaggagaaaaaatactagttgactatagaagtatggacctatgcagtatgtatgtggatgtgaagatatgctatagaatatcatgtatatactataagtacaatgtatttctactatatagtgttaaattaagattgactaaaaatactaacatggtagtctactaagtagatatttaaattatgttctattatgtatttccctccatttctaacctatttttttaactttcacttttctaaacaaaaaattaatcatattatccaaagcaatagataatttgttctcttcaaaatagagctgattgaaggatagaagagatctaaaggtgaaagaagaaagtgattttaccagcaggcccaggagggtcgagcccagtgaggctcgatcactgcatctctctcctgataatgaggtcacatgtcccaggctgtatgccactggagcttccatagtggcatactctgcccccgtccaattttaatactgctgacagaggctggcaatgatcagaaaaagaaccaggccttgtttgataggtctaaggagctactagcccagtttaaaagccacccaacacctactgaacagctaagaacataggcagccaagaaatgacccggatactgaggccccaggttggatttaaagtgctccaggtcactgctgtagaaattatggtgctcccagtctccatccttcctccactgaaggaggttggactgatgttgacccaaatttgagattcagatcttggagcaccattatgtcctgagaaaagagtatagctttggagtgcaaaagctctgggttcaaattttagctctgggaccccaggcaagttacttggcatctagatcttggagttataaatttgtaaaatgagaataatgtcatgcacagaggattattgtggagattttactaattgtgtaaagcacctggcagagagaaaagacttgatacacgctggtctccctctgcttctccagcacacaccctaatcactgttcctaaattagcctggcagaggagggtctggtcttggtgaagcccagtctcaaccaacgaggtggctctgctgcagaggcacacctgatggcaagctatgggagtacttatagcataaaaacttatttgacatggatcagatctttaccagatttgtatttaataggttagaattacagtctgagagaggagaaatgagaggaagaatggaaaagctaataaatggttataaattagaacctctgagagaacttaaagaaattgtcttttacttggagaaaagaagattgaaagcaatttaacaactcttcaagacaatgcatgaaggataatcagcaggataatggccctaatccctaaggccagaaaaagaggaacgagttgaatttgcattagagagggagggagatagattaaatttaaggcgaaatttctctagaagaatattattaaacagtgggataggtgaccgcagaagcctatggaattttctttcatggaactccataaaaacagagagcccctgtttggtttgtgagaggtactgaccctgttccagaaatctgaggtagaggggcaggagggaaaggggatggagaatatcgcagagatagttcttgagccctggaaaatacaagttaaagcacttggaaggctttcagggaaaagaagcaacattcatctttgactaggaaacagaatctgaatgctaatgcaagagagaacatgcggggtggggagaaggtgcgggcaaaggggaggaaaggaggcgatccattttgtttccacattgcaggcaccactccactcctagtggagcaatcctaaatttctttctgtgggcctcagtttccttttctgtaatatcaagcgcttagattaagcactctcttgagtctcatccaatcctgaatttgtgtttataaaaccgatccagagagtgtgagtaaatctgtgaacagctacttcagccccgaacccagtggtgttgagtggactggtcctgcacagagggacaatgcacctgcatgggcaaaaactggggctgggccagagtcatgggcttcaatccctgcttttcagccactagcaagttgttcatcttctctgaacttcaatatcttcattcgtaaaatgtggacagtaatagctcctttggccgtttcgagtttaaattagataatgcaagtagaagacatagtacaataatatctaataactaccatgttcaggaatataagtaccaataaaatggtacctattgtattaatagcatttgagtgagaatttttaagtctttttatttctcatcttttcatttctacctttattggatttgtagaatctattacagtcaaatcatatagtttggtgttgtggtggtgatgttttttgtttgctttttaacacacttgcctttacttgttttggcagaagggatcctaattcattcagctttctcgaatgtcatataaaaacacataacacatgttcacgttcctagttgtttctacatattctctgctcttttagatgtgtaatatagaaccaagtaattatttagcagttccggtggctttgcatcattttggcattcataccaagcccccgttactgagatctttgctgcccctctttaacatggtcagtcggggttcctcggtattctgttgacgtcaccattttagctacccttctctgcatattgacctatccctttgcaacttcatattgtcactttcaacagtgtttccaacttaaaaccccctatttgactcttcaaaaggctagactccccatccccagtttgacaccgacaggcaggctgtgggatgtgcaccaggttgatacagaacccagctccaccaagctgaaagggcgcgtcgcccgggccagggtggggcaccaggttatgagtggcctcctgccttttgggtttgcttccccgcaggggacaccgtaggcggctgtgcggtccggccactgccccccgcccctcgcgtccggccgcgccgtgggttgcggtctccgtgggggtggcagctcctgtccgggaggtttgcgcctggcggggcaccccagagccgcgaagagccggtaggcgaggccagggcggggtggggcgcgtccaggcggggagggcaaactcgggcagcgcagggggcgcagagggcagcgggcgggcggacgcgcggcggaggcgcgagcgggacgagggctggctagtgccggggccgcccgcccgagggggaggaggctgtgctgcccttgctgcaggttcgctgtcgagcgcacaggaggcagggacatgggtagggtgcggtctgcgcggggcccgagcccggatctcttccatttcccctctcactcggccccgggctgcgccgcagacggcagcagctcccgctccgcccgagccgcctgaccgccgggccggggtgctaaccgcggggccctgcagcccgccggcccggccagcccagcccagcccggcggcccgcagccccgccgcccgccgccccccgccgccgccgcgttgccaaaacaaacgggccggcctatttattggcggccggcgagcccggcagctcagagtcgaggcgccgagggggacagcgcgccgccaccagctcgggccctgggcccccgccccgcacttgagtcccgccggccctggccgcaccacgccgcccatggcgcccccgcctggagccccccggagccacccggacgcctgagcccccgcagcgctcccgtcgacgcgcctgcccatcagcccaccaggagacctcgcccgccgctcccccgggctccccggccatgtctcccgcccggctccggccccgactgcacttctgcctggtcctgttgctgctgctggtggtgccggcggcatggggctgcgggccgggtcgggtggtgggcagccgccggcgaccgccacgcaaactcgtgccgctcgcctacaagcagttcagccccaatgtgcccgagaagaccctgggcgccagcggacgctatgaaggcaagatcgctcgcagctccgagcgcttcaaggagctcacccccaattacaatccagacatcatcttcaaggacgaggagaacacaggcgccgaccgcctcatgacccaggtgagcgcgatccgccctaccagcgcgccgccaccgaagcacggccacctgcccggccctacccaccccctggcacattttctcgactggctggcacgctccctggcacctgcccctctcgccaagccccgctgcctcctgcccaagatgggctggccaggggtccaggcaggagtgtcggcatccccaagcccggaggagactgctagaagagctccccgctgccaagccccacagcccagcaggttggcggcggctcctctgccccaaagtactggcagctcagccagacgctgcacccggaggtacctctacccggctgcctcctgcgcctccaggacccacgcagtccctcccggcctaaccttctgcacagctgtgcccctttcattttgaaacttttcggcacccctgaggcaaagaatctctgggtgcgtagggtcaccacggcctttcctccctgtgcgctcttggttctacgggctgccccttccaggagcagcgtcccggctgggtctggggttaagaacagcgaggaagagttgaatcgggaggcggaggtgggaaaaggaacctgcccagcgagcccacgtgtctgtccggagagctgaggggtgctactgcggtggaagggtgggttattggggccttcgggctctgggactgagcttaggggattcctgggcttggtggtggggacgggaaggagtggaaaagaggggctgagatgccgagggaggcagggaccagggctgacgctagtctctgcatgctggagtaggacccgcgctccacccaagcttagaggcgcaaattccttcccagcagccaggtccgcgaggggagagaccttcacccccttccttcggggtctcccgaggcttccccctgaaagcaggcccctggctttctagatccgttgggaagagcagctagactcagggccctcagcatgcaccctggcgccggggctggccggccagtgtcggggccgaaggttggctggtggccgggccgggccgggcgccgaggaatgcagataaggatccgggccacgggctgggcaatcattgacagcgcacgcccgggctctggacccgagagggggcggtggcagtgagggagagaggcggggaaccagggctgcaggcgggcataggtgcggggctagaagcctgacggcagggtctacgaaggagcaagcggagctccctccctgggcagtaagcacccccagccaggctgtgcaccagcgaggacctgcgaggccctgggttgtagctgctattggctcctaggaaggctgagtccaagggtctgtgaggcccgggctctgcagggtgggggtaggttgagacttgggctgcccacacttgttggcgcctcgaatctcagagtctcagagtcgaggactggtggaagggcctttcccctcagaccaaggccctggccccagcttcttctcagagtcagctgcacagtctcttgggctctcaggacccaggcacaccaggaggcagcaaccaaggatgaccgcttcggtccttcccccagggcgcggctctaccccaactccacgtgaggaggatggtggagctggctccctgagatcagaagcttggtttatggagttccttcctacggtataggggcggggcaccggtgcagaggtcctggaggtctgggattgtcagccccttttgcgccttgcagaggaagacgttgcatgtggccccccattgggcggccctggcgctaatccgccggtttctggttccctggccctgtcatctctggcggggttgtgggccggtttaagattataagagggtcccggcgggaatcctcggtggtgctcgagggatccaggcaggccggcgattcccctccaacacaccccctgaagctaccgagacgctctgcacctgccgcgcctccagagcctgtcgtccccaggcctcagccctgaggctgcggtgacacctactgggcacctcgggccttgcacgcctcgcaggccggccgcaggcagtcggctctgctgccactcctccctcccggccgctggctggccctcctgccttggaggcacagagggaccgggcgctgaaaggtgtagtcctgtctgtggagggatggatacccggagctgaagacagcacaggagagaggggacctccacctaccagtgaaggcgccgccagggcgcttgcctccatgcctcctccagatttgtgataccgccgtgcggtccagatccccgggggcggctgtatgctagggagggagcacttcagggccctctgttcctaagggagagggacagcaccctcccgggcttccagctcccttgggtgtggcgggagagttctgggcgaggctgcgcctacacctgcacctcggccggctcctccggctgatttcgctctggttgatttcgcgcacattcctctcccagcgctgcaaggaccgcctgaactcgctggctatctcggtgatgaaccagtggcccggtgtgaagctgcgggtgaccgagggctgggacgaggacggccaccactcagaggagtccctgcattatgagggccgcgcggtggacatcaccacatcagaccgcgaccgcaataagtatggactgctggcgcgcttggcagtggaggccggctttgactgggtgtattacgagtcaaaggcccacgtgcattgctccgtcaagtccggtgagccgccgccggggggctgggcccggggccggggccgcagggcatgggcaggaggagtagtgccgagaaggtgaagagcccgcccggggatggaggcagggatccgggggaagaggacatcagcggcgctctccaaggctgctcctgaccgtgactgcctgctcatcccatgcctggcagtgtcatggtaggagcctagggcgatagggccctgtcccagggctgctgggtgctgacctggtggcccacagtccagagcctgccacaggaggtgcaagacagagcaagctggacagatggaggaagccaggctgccagacaaggcctccccaccccacctggcacagaacaaggcgggtggtggggccaggagccggggtatgcccagacacctgcatgcctgaccaggagcccagaggctcagaacccaccacccagctgcaggtcagccctccctggggcgcaggcgtccccaacctctccccctgtgccctgaagctggacattttggggcaaagccagccaggagagccagactaaacaaagtggactcaataaagtggattttcccagattggccgcattgttgatcagaattggcaggcactggggcctcacccctccctcaagtcttgtcagcaccctgtcacctacactgaacccagccatggaggctccaaatggttcctgtgtggctcccagccaagccccacctggctgccggctcagcctttcacactggcagcgtgtaatagttgcactctcatagacccgcacactgagcccaggcccacattttgatattcctgaccctaccctgacccacaaccacaccctgaaccttccactcaatacttcccctgccaactgtgtctactgcaaggcccatccacccttcactgcaaacattgacccacgacaaccacttccattcattactcctgacccccaaacactgttctccagctcacctgctgatgccgcagtgttctgtccctgcacagggactcagcatgtgaactgaagatggtgactgtgtcctcgcccctggcctgcccctgacccaaacattcactccgaggctgcccaggagtctgcctttgccccactgggtgggctcctctcacatccctacattcaaaggtccagctctgttacagaacctggggagtcagcctgagagggcaatttttgggaagtgtctgagcaaggggtgagaaataacagaacatctaggcctcggaggagctaagcaacctcccccttccatgaggtagccctttccttctcatccactgggtcttggagccccactatgctccttggcaaggtattaggctggggaggggggtcacagaagatcacattggagtaaggggatgggggcagtgaagtatgggttcaagcctgcataggaagtggggggcaggagctgggatatggtgacgggggctctctgggagcaagaggagctctggctggctggggtggccaagggagggtcgttgtgaccaggaggctgagctgcacagcagtaacaccttcatgtccctttctcccacagagcactcggccgcagccaagacgggcggctgcttccctgccggagcccaggtacgcctggagagtggggcgcgtgtggccttgtcagccgtgaggccgggagaccgtgtgctggccatgggggaggatgggagccccaccttcagcgatgtgctcattttcctggaccgcgagcctcacaggctgagagccttccaggtcatcgagactcaggaccccccacgccgcctggcactcacacccgctcacctgctctttacggctgacaatcacacggagccggcagcccgcttccgggccacatttgccagccacgtgcagcctggccagtacgtgctggtggctggggtgccaggcctgcagcctgcccgcgtggcagctgtctctacacacgtggccctcggggcctacgccccgctcacaaagcatgggacactggtggtggaggatgtggtggcatcctgcttcgcggccgtggctgaccaccacctggctcagttggccttctggcccctgagactctttcacagcttggcatggggcagctggaccccgggggagggtgtgcattggtacccccagctgctctaccgcctggggcgtctcctgctagaagagggcagcttccacccactgggcatgtccggggcagggagctgaaaggactccaccgctgccctcctggaactgctgtactgggtccagaagcctctcagccaggagggagctggccctggaagggacctgagctgggggacactggctcctgccatctcctctgccatgaagatacaccattgagacttgactgggcaacaccagcgtcccccacccccgtcgtggtgtagtcatagagctgcaagctgagctggcgaggggatggttgttgacccctctctcctagagaccttgaggctggcacggcgactcccaactcagcctgctctcactacgagttttcatactctgcctcccccattggggagggcccattccatccatcttaggcccctttgggtgggcttgcgcctcagttgatgctgctaaattccctgggagccagcatggatctggctggacccgatgctgtccagaactgggaaggccacaggggtggggcagccatcccggccattctgaggtatgacattcctccccggccacactcctcaagacacatccagagactgttgctgtctgtgggcagagttctgtgttctggccaatgtgaccgtagtgccggggactgggggaggtgggttggatgtgcttgccacccccccggctaagctcccccttctgctgaaccatgatccccaccccctccgccggtcagtctcccataccttatttattggagtggagggggaagcccatgggagaattttggggatgttttggtcttttcttccttttgtaataaaaattatttaagttgttagagccacagagtccaggatgctcacagatagtttggagccctggaaggttgtgtccagggttttctcccctcctcggagaggcctggctcaccttcctccaaggacaaaaactccaggcctcccaccctcactgcaggtccattcccacccagtagctctgcaaagcccctacagagagctgtgctctgcacagggcaggaccttcccacactgacctcaccatttaccccaaacaaggatggacccgagagagaacagagttgactagtccatggtcaccttactcatctataaccatgcccttgtgctctcagggcctgggcttctgactcttccctgttggtaatggaccacagtgtgaaaagattctgtaaaattcacgttgtcttgtgggctattctgacttctcggacaggtcagagatccctaatctagcagttctcaaatgtcaggatcacctgggaagcttgtttaaagatgattcctggccccacttccaggattccacttttttttttttttttttttttgagacagagtcttactctattggccaggctggagtgcagtagcgcaatcttggctcactgcaatctctgcctcctgggttcacaccattctcctgcctcagcctcctgagtaggtgggactacaggcacacgccaccatgcctggctaatttttatttgtatttttactggagacggggtttcaccgtgttagccaggatggtctcgatctcatgtccttgtgatccacccacctcagcctcccaaagtgctgggattacaggcaggagccaccacgcccggcccaggattccactttaatagatccagtgcaaagcccaggagcttagttttgcaaacaccccaaacatggcagatggtccatgggccacactcaagaactgctgcttagtccagaggaggcctggtttactgccaagacagcatgacccccttctttactttccctccatctctccatcccaaggcaagtttttctatactcgctatagataagctgagattctaggtccagtggtacaaaattttcaggagttttgtgagtcagttgaaataggcacagtggccatagaactctggaaactggcagagactaccaattagggtttccccccaccttggggagccagttgttgaagtgtgccactgcccaggctctgcccttttacacagatgtctgtgttgaaagtggtggggatgactgttcagactcttagtttaccctgtcctcacatccactgaagaaaatgccagagaaaaccattattctggggtctgtgtcatctggagaaatagggccccactggctgtactgataactgctatcatttttgatgactaaccattgctaggcattattctagatctttacactcagtaggccacttagtgctcataatgaccctatgtggtgtgtactttatttaatattaccactttccagaaaagaacacagaggcacagggaggttaagaaacttgcccaggagtacacaagaagcgacagaagcagaatttgaatccttccagactggctcttgggtccattgtttttatccccaagcttttcccctggggaactcaggttctctctggtaggtcgcactcagcatttgaggtctggaaatctgctttgtcaacacatctagagtggacccatctctctgtggcagactcttactgaccaggctcacagggaccaaaatgttcatgaactcattcacagtgagaaataacaggccaaggtcaagatgctcttgtccattcattcacacattctctccatgtagacattctacctgggagatcagattgtggtgacaagttgttggtgagtgtggatgcatgcagtagatgactctccagctcatggaggaaggagtggggggatgcctctcctgctggggttgggcacatgtggtccagcagacacagagccatttggtgtccttagacctttctgttctagatgggcct

(19) United States
(12) Patent Application Publication
(10) Pub. No.: US 2018/0110785 A1
(43) Pub. Date: Apr. 26, 2018

(54) METHODS AND COMPOSITIONS FOR AMELIORATING SYMPTOMS ASSOCIATED WITH CHROMOSOMAL ABNORMALITIES

(71) Applicant: Robert I. HENKIN, Bethesda, MD (US)

(72) Inventor: Robert I. HENKIN, Bethesda, MD (US)

(21) Appl. No.: 15/327,190

(22) PCT Filed: Jul. 16, 2015
(86) PCT No.: PCT/US2015/040776

§ 371 Date: Jan. 18, 2017

Related U.S. Application Data
(60) Provisional application No. 62/026,292, filed on Jul. 18, 2014.

(51) Int. Cl. A61K 31/522; A61P 43/00; A61P 25/00

(52) U.S. Cl. CPC A61K 31/522; A61P 25/00; A61P 43/00

(57) ABSTRACT

Disclosed herein are methods for diagnosing a subject with one or more symptoms associated with chromosomal abnormalities. Also disclosed herein are methods and compositions for ameliorating and/or treating a subject for one or more symptoms associated with chromosomal abnormalities.

The present application claims priority to U.S. Provisional Patent Application No. 62/026,292, filed Jul. 18, 2014, the contents of which are hereby incorporated by reference in its entirety.

### BACKGROUND OF THE DISCLOSURE

Chromosomal abnormalities can lead to a various phenotypes. Different chromosomal abnormalities have different symptoms, including unusual features, poor growth, intellectual disability, learning disabilities, behavioral problems, and problems with organ systems such as digestive disorders or heart defects.

There is a need in the art for methods for ameliorating and/or preventing one or more symptoms associated with chromosomal abnormalities.

### INCORPORATION BY REFERENCE

All publications, patents, and patent applications herein are incorporated by reference in their entireties. In the event of a conflict between a term herein and a term in an incorporated reference, the term herein controls.

### SUMMARY OF THE DISCLOSURE

Described herein is a method for ameliorating one or more symptoms associated with one or more chromosomal abnormalities in a subject or subject in need thereof comprising treating the subject or subject in need thereof with one or more activators of the hedgehog signaling pathway.

The one or more chromosomal abnormalities can be trisomy 21. The one or more chromosomal abnormalities can be trisomy 18. The one or more chromosomal abnormalities can be trisomy 13.

The one or more symptoms that can be ameliorated are one or more cognitive symptoms. The one or more cognitive symptoms can be selected from intellectual disability, speaking disability, mental illness, autism, depression, anxiety, epileptic seizures, dementia, or any combination thereof. For example, the one or more cognitive symptoms can be an intellectual disability. The one or more cognitive symptoms can also be dementia.

The one or more symptoms can be one or more physical symptoms. For example, the physical symptoms can be selected from the group consisting of stunted growth, umbilical hernia, increased skin on the neck, low muscle tone, narrow roof of mouth, flat head, flexible ligaments, large tongue, abnormal outer ears, flattened nose, separation of first and second toes, abnormal teeth, slanted eyes, shortened hands, short neck, obstructive sleep apnea, bent fifth finger tip, brushfield spots in the iris, cataracts, keratonconus, glaucoma, hearing problems, otitis media with effusion, poor Eustachian tube function, single transverse palmar crease, protruding tongue, congenital heart disease, strabismus, congenital hypothyroidism, diabetes, duodenal atresia, pyloric stenosis, Meckel diverticulum, imperforate anus, celiac disease, gastroesophageal reflux disease, early menopause, infertility, undescended testicles, and any combination thereof.

The subject or subject in need thereof can be treated at birth. In some cases, the subject or subject in need thereof can be treated post-partum.

The one or more hedgehog activators can be orally administered to the subject or subject in need thereof in a composition comprising a nutrient. The subject or subject in need thereof can be treated in utero. The subject or subject in need thereof can be a fetus. The one or more hedgehog activators can be indirectly administered to the subject or subject in need thereof by administering the one or more hedgehog activators to an adult human, where the subject or subject in need thereof can be inside of the adult human.

The subject or subject in need thereof can be continuously treated after birth. The subject or subject in need thereof can also be continuously treated during post-partum.

The one or more hedgehog activators can be orally administered to the subject or subject in need thereof in a composition comprising a nutrient. The subject or subject in need thereof can be continuously treated in utero. The subject or subject in need thereof can be a fetus. The one or more hedgehog activators can be indirectly administered to the subject or subject in need thereof by administering said one or more hedgehog activators to an adult human, where the subject or subject in need thereof is inside of the adult human.

The one or more activators of the hedgehog signaling pathway can increase levels of Sonic Hedgehog (SHH) or can be an isolated SHH. For example, an isolated SHH can be a purified natural or purified recombinant SHH. A purified recombinant SHH can have at least about 70% homology to SEQ ID. No. 1, 2, or 3. A purified recombinant SHH can have at least about 95% homology to SEQ ID. No. 1, 2, or 3. The purified recombinant SHH can comprise at least 10 amino acids of SEQ ID. No. 1, 2, or 3.

The one or more activators of the hedgehog signaling pathway can increase levels of Dessert Hedgehog (DHH) or can be an isolated DHH. For example, an isolated DHH can be a purified natural or purified recombinant DHH. A purified recombinant DHH can have at least about 70% homology to SEQ ID. No. 4, 5, or 6. A purified recombinant DHH can have at least about 95% homology to SEQ ID. No. 4, 5, or 6. The purified recombinant DHH can comprise at least 10 amino acids of SEQ ID. No. 4, 5, or 6.

The one or more activators of the hedgehog signaling pathway can increase levels of Indian Hedgehog (IHH) or an isolated IHH. For example, an isolated IHH can be a purified natural or purified recombinant IHH. A purified recombinant IHH can have at least about 70% homology to SEQ ID. No. 7, 8, or 9. A purified recombinant IHH can have at least about 95% homology to SEQ ID. No. 7, 8, or 9. The purified recombinant IHH can comprise at least 10 amino acids of SEQ ID. No. 7, 8, or 9.

The one or more activators of the hedgehog signaling pathway can be selected from the group consisting of cyclic adenosine monophosphate activator, cyclic guanosine monophosphate activator, and any combination thereof. The one or more activators of the hedgehog signaling pathway can be selected from the group consisting of ELND005, a drug that decreases myo-inositol, RG1662, Picrotoxin, a GABA blocked drug, PTZ, Nicotine, Green tea extract, a Nerve growth factor, introducing a XIST gene, theophylline, riociguat, forskolin, phosphodiesterase inhibitor, and any combination thereof. The one or more activators of the hedgehog signaling pathway can comprise theophylline. The one or more activators of the hedgehog signaling pathway can comprise a phosphodiesterase inhibitor. The phosphodiesterase inhibitor can comprise cilostazol or rolipram.

The one or more activators of the hedgehog signaling can be at least one composition or dosage unit. The at least one composition or dosage unit can be steroid-free. The at least one composition or dosage unit can be an intranasal composition or dosage unit. The at least one composition or dosage unit can be an oral composition or dosage unit.

The methods described herein can comprise performing a diagnostic test. The diagnostic test can comprises an invasive test. For example, the invasive test can be selected from the group consisting of: amniocentesis, chorionic villus sampling, embryoscopy, fetoscopy, percutaneous umbilical cord blood sampling, and any combination thereof. In some cases, the invasive test can be amniocentesis. The invasive test can also be chorionic villus sampling. The method can also comprise performing a karyotype. The diagnostic test can be a non-invasive test. For example, the non-invasive test can be selected from the group consisting of analysis of fetal cells in maternal blood, analysis of cell-free fetal DNA in maternal blood, preimplantation genetic diagnosis, external examination, ultrasound detection, analysis of fetal heartbeat, a non-stress test, transcervical retrieval of trophoblast cells, maternal serum screening, and any combination thereof. The non-invasive test can be analysis of cell-free fetal DNA in maternal blood. The diagnostic test can also detect a fetus having one or more abnormalities. The one or more abnormalities can comprise one or more chromosomal abnormalities.

Disclosed herein are also methods of prophylactically preempting one or more symptoms associated with one or more chromosomal abnormalities in a subject or subject in need thereof comprising administering to a human one or more activators of the hedgehog signaling pathway. The method can also comprise assessing if said human is or will become pregnant prior to administering to said human one or more activators of the hedgehog signaling pathway.

The method can also comprise ascertaining the age of said human. For example, the human can be greater than 35 years of age at conception. The human can also be between 15 and 45 years of age at conception.

Also disclosed herein is a method of normalizing cerebellar structure of a subject or a subject in need thereof comprising administering to the subject or the subject in need thereof a drug. Further disclosed herein is a method of normalizing hippocampal function of a subject or a subject in need thereof comprising administering to the subject or the subject in need thereof a drug. The drug can be selected from the group consisting of theophylline, riociguat, forskolin, a selective PDE inhibitor, a non-selective PDE inhibitor, and any combination thereof.

Also disclosed herein is a method of treating one or more chromosomal abnormalities in a subject or a subject in need thereof comprising treating the subject or the subject in need thereof with one or more drugs selected from the group consisting of theophylline, riociguat, forskolin, a selective PDE inhibitor, a non-selective PDE inhibitor, and any combination thereof; and where the treating results in at least one of the following phenotypes selected from the group consisting of normalized cerebellar structure, normalized hippocampal function, normalized cerebellar area, normalized hippocampal area, increased cellular proliferation within the cerebellum, increased cellular proliferation within the hippocampus, increased number of cells within the cerebellum, increased number of cells within the cerebellum, increased cerebellar volume, increased hippocampal volume, increased cerebellar area, increased hippocampal area and any combination thereof. The drugs or one or more activators of the hedgehog signaling pathway can be directly administered into the brain. The drugs or one or more activators of the hedgehog signaling pathway can be administered orally. The drugs or one or more activators of the hedgehog signaling pathway can be administered intranasally.

Disclosed herein is a method for ameliorating one or more symptoms associated with one or more chromosomal abnormalities in a subject or subject in need thereof comprising treating said subject or subject in need thereof with one or more activators of the hedgehog signaling pathway.

Disclosed herein is a method for ameliorating one or more symptoms associated with one or more chromosomal abnormalities in a subject or subject in need thereof comprising treating the subject or subject in need thereof with one or more PDE inhibitors.

The one or more chromosomal abnormalities can be trisomy 21. The one or more chromosomal abnormalities can be trisomy 18. The one or more chromosomal abnormalities can be trisomy 13.

The one or more symptoms that can be ameliorated are one or more cognitive symptoms. The one or more cognitive symptoms can be selected from intellectual disability, speaking disability, mental illness, autism, depression, anxiety, epileptic seizures, dementia, or any combination thereof. For example, the one or more cognitive symptoms can be an intellectual disability. The one or more cognitive symptoms can also be dementia.

The one or more symptoms can be one or more physical symptoms. For example, the physical symptoms can be selected from the group consisting of stunted growth, umbilical hernia, increased skin on the neck, low muscle tone, narrow roof of mouth, flat head, flexible ligaments, large tongue, abnormal outer ears, flattened nose, separation of first and second toes, abnormal teeth, slanted eyes, shortened hands, short neck, obstructive sleep apnea, bent fifth finger tip, brushfield spots in the iris, cataracts, keratonconus, glaucoma, hearing problems, otitis media with effusion, poor Eustachian tube function, single transverse palmar crease, protruding tongue, congenital heart disease, strabismus, congenital hypothyroidism, diabetes, duodenal atresia, pyloric stenosis, Meckel diverticulum, imperforate anus, celiac disease, gastroesophageal reflux disease, early menopause, infertility, undescended testicles, and any combination thereof.

The subject or subject in need thereof can be treated at birth. In some cases, the subject or subject in need thereof can be treated post-partum.

The one or more PDE inhibitors can be orally administered to the subject or subject in need thereof in a composition comprising a nutrient. The subject or subject in need thereof can be treated in utero. The subject or subject in need thereof can be a fetus. The one or more PDE inhibitors can be indirectly administered to the subject or subject in need thereof by administering the one or more PDE inhibitors to an adult human, where the subject or subject in need thereof can be inside of the adult human.

The subject or subject in need thereof can be continuously treated after birth. The subject or subject in need thereof can also be continuously treated during post-partum.

The one or more PDE inhibitors can be orally administered to the subject or subject in need thereof in a composition comprising a nutrient. The subject or subject in need thereof can be continuously treated in utero. The subject or subject in need thereof can be a fetus. The one or more PDE inhibitors can be indirectly administered to the subject or subject in need thereof by administering said one or more PDE inhibitors to an adult human, where the subject or subject in need thereof is inside of the adult human.

### BRIEF DESCRIPTION OF THE DRAWINGS

FIG. 1 shows accuracy rates for detecting trisomy 21 and 18 with a Harmony Prenatal Test, which can be used in some embodiments herein.

FIG. 2 shows a trisomy 21 (i.e., Down syndrome) karyotype.

FIG. 3 shows a trisomy 18 (i.e., Edwards syndrome) karyotype.

FIG. 4 shows a trisomy 13 (i.e., Patau syndrome) karyotype.

FIG. 5 shows an XO (i.e., Turner syndrome) karyotype.

FIG. 6 shows an XXY (i.e., Klinefelter syndrome) karyotype.

FIG. 7 shows an example of a karyotype of several chromosomal translocations.

FIG. 8 illustrates an exemplary course of events related to a method of diagnosing, treating, and/or ameliorating one or symptoms or chromosomal abnormalities.

FIG. 9 depicts a computer system useful for displaying, storing, retrieving, or calculating diagnostic results from a level of one or more biomarkers associated with one or symptoms of chromosomal abnormalities; displaying, storing, retrieving, or calculating raw data from biomarker analysis; or displaying, storing, retrieving, or calculating any sample or subject information useful in the diagnostic methods disclosed herein.

FIG. 10 reproduces FIG. 1 from Ishita Das et al., “Hedgehog Agonist Therapy Corrects Structural and Cognitive Deficits in a Down Syndrome Mouse Model,” Science Translational Medicine, Vol. 5, Issue 201; p. 201ra120 (2013) (herein referred to as “Ishita Das et al.”): An injection of SAG at P0 normalizes cerebellar morphology in adult Ts65Dn mice.

FIG. 11 reproduces FIG. 2 from Ishita Das et al.: Cerebellar LTD is minimally different between Ts65Dn and euploid mice.

FIG. 12 reproduces FIG. 3 from Ishita Das et al.: SAG corrects performance of trisomic mice in tasks dependent on hippocampus.

FIG. 13 reproduces FIG. 4 from Ishita Das et al.: SAG rescues attenuated TBS-LTP in hippocampal slices from Ts65Dn mice.

FIG. 14 reproduces FIG. 5 from Ishita Das et al.: SAG partially rescues attenuated NMDA-EPSCs in hippocampal slices from Ts65Dn mice.

FIG. 15 reproduces FIG. 51 from Ishita Das et al.: SAG had mitogenic activity in primary GCP cultures.

FIG. 16 reproduces FIG. S2 from Ishita Das et al.: Dentate gyrus is not affected by SAG treatment.

FIG. 17 reproduces FIG. S3 from Ishita Das et al.: SAG treatment does not affect open-field performance.

FIG. 18 reproduces FIG. S4 from Ishita Das et al.: SAG treatment does not improve Y maze performance in Ts65Dn mice.

FIG. 19 reproduces FIG. S5 from Ishita Das et al.: SAG treatment normalizes search strategies of Ts65Dn mice in the MWM.

FIG. 20 reproduces Table 51 from Ishita Das et al.: Cerebellar morphological measurements.

FIG. 21 reproduces Table S2 from Ishita Das et al.: Dentate gyms granule cell number at P6 (hematoxylin-stained).

FIG. 22 reproduces Table S3 from Ishita Das et al.: BrdU-positive cells in the DG at P6.

FIG. 23 reproduces Table S4 from Ishita Das et al.: Electrophysiological measurements from cerebellar Purkinje cells.

FIG. 24 reproduces Table S5 from Ishita Das et al.: Open-field activity.

FIG. 25 reproduces Table S6 from Ishita Das et al.: Total number of entries and percent alternation in Y maze.

FIG. 26 reproduces Table S7 from Ishita Das et al.: Latency in the MWM visible platform test (seconds).

FIG. 27 reproduces Table S8 from Ishita Das et al.: Latency in the MWM hidden platform test (seconds).

FIG. 28 reproduces Table S9 from Ishita Das et al.: Time spent in the correct quadrant in the MWM probe test (seconds).

FIG. 29 reproduces Table S10 from Ishita Das et al.: Strategy scores of animals in hidden platform test, by day and trial number.

FIG. 30 reproduces Table S11 from Ishita Das et al.: Relationship between FV amplitude and fEPSP slope.

FIG. 31 reproduces Table S12 from Ishita Das et al.: Paired pulse ratio.

FIG. 32 reproduces Table S13 from Ishita Das et al.: TBS-LTP enhanced by SAG in slices from Ts65Dn mice.

FIG. 33 reproduces Table S14 from Ishita Das et al.: Current-voltage relationship.

FIG. 34 reproduces Table S15 from Ishita Das et al.: NMDA/AMPA ratio.

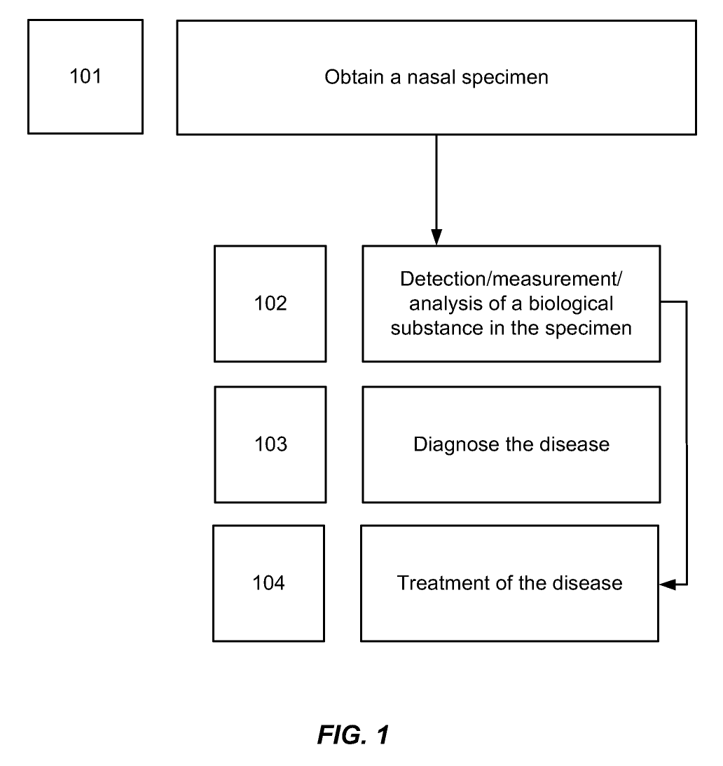

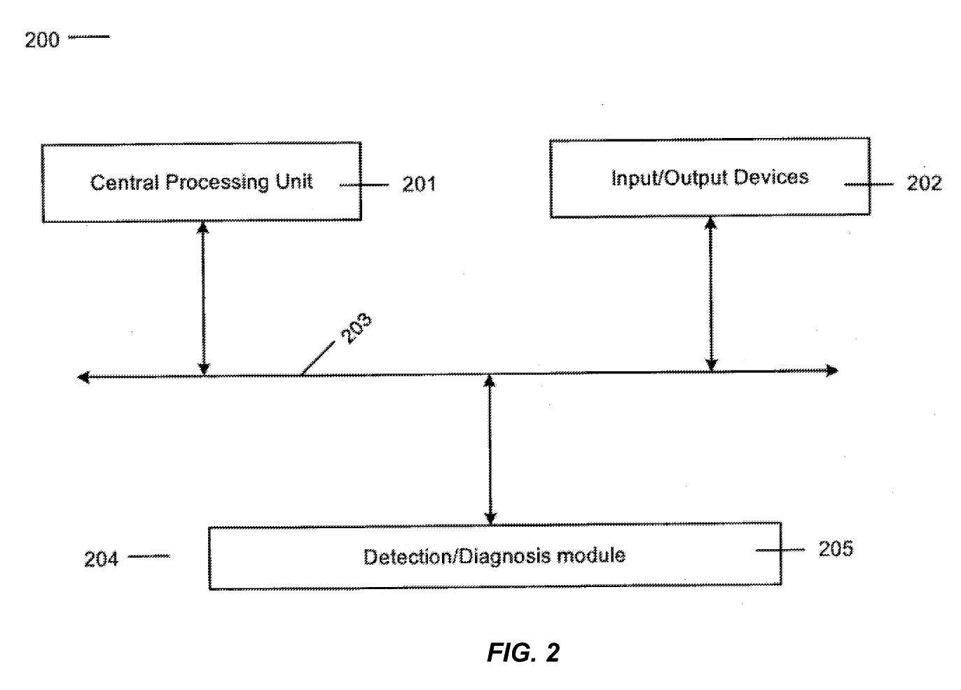

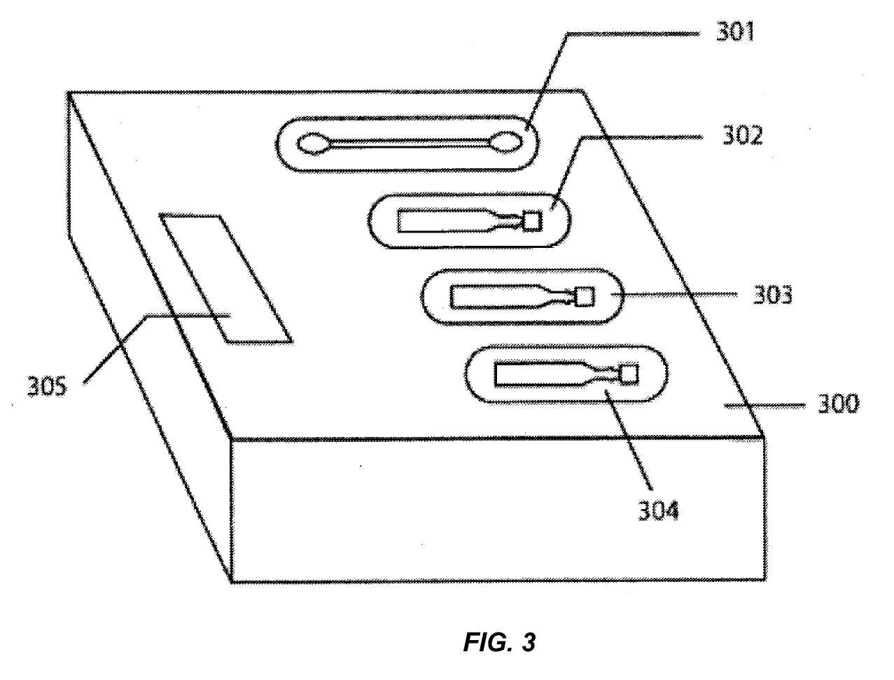

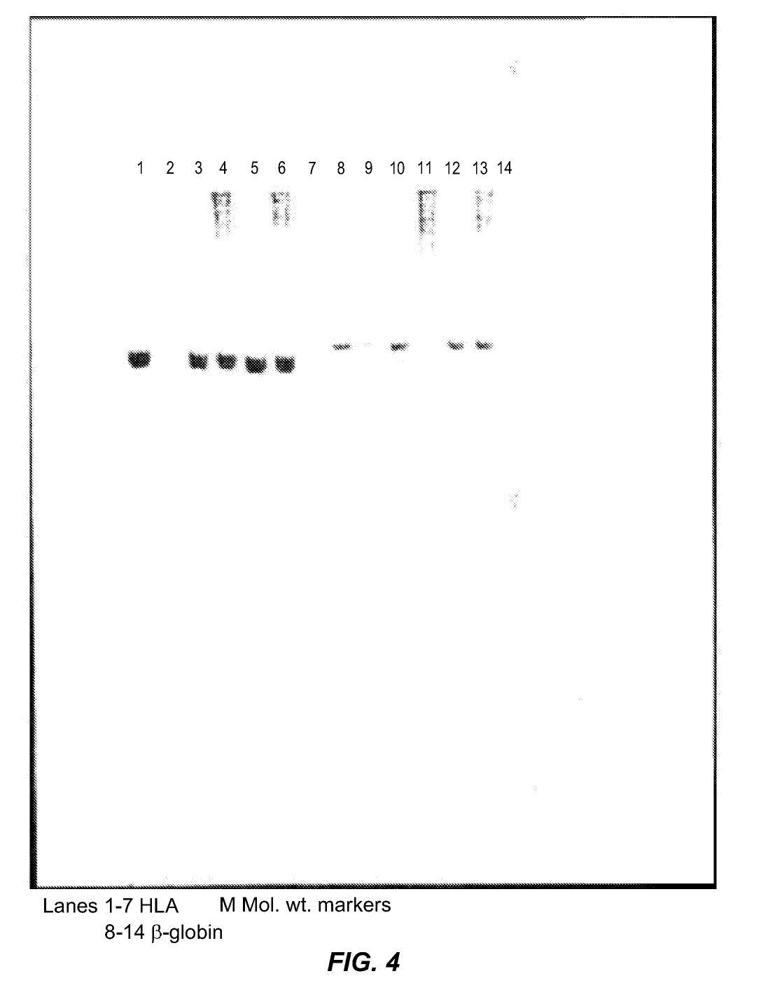

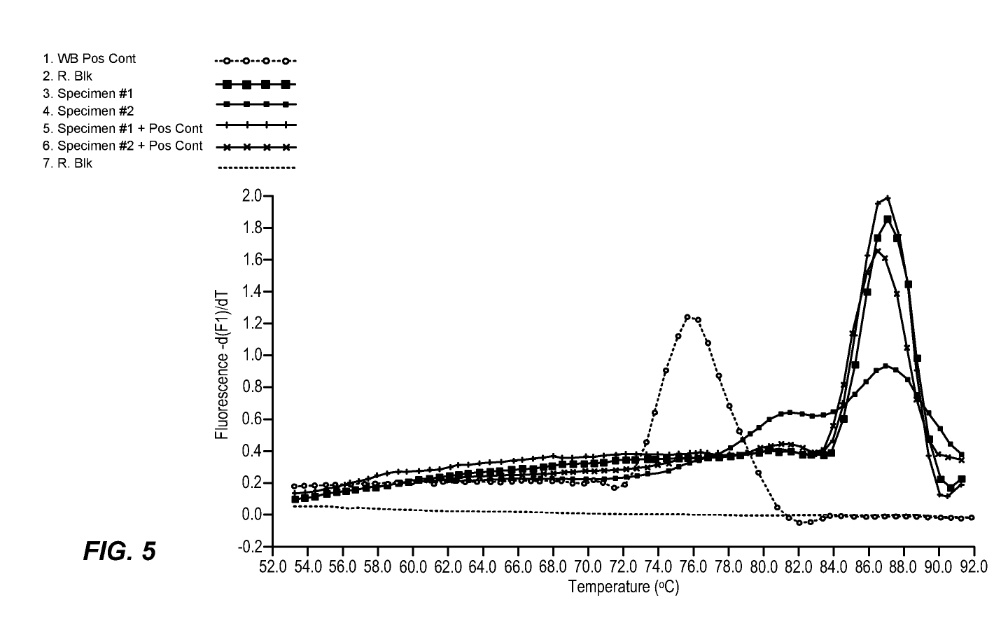

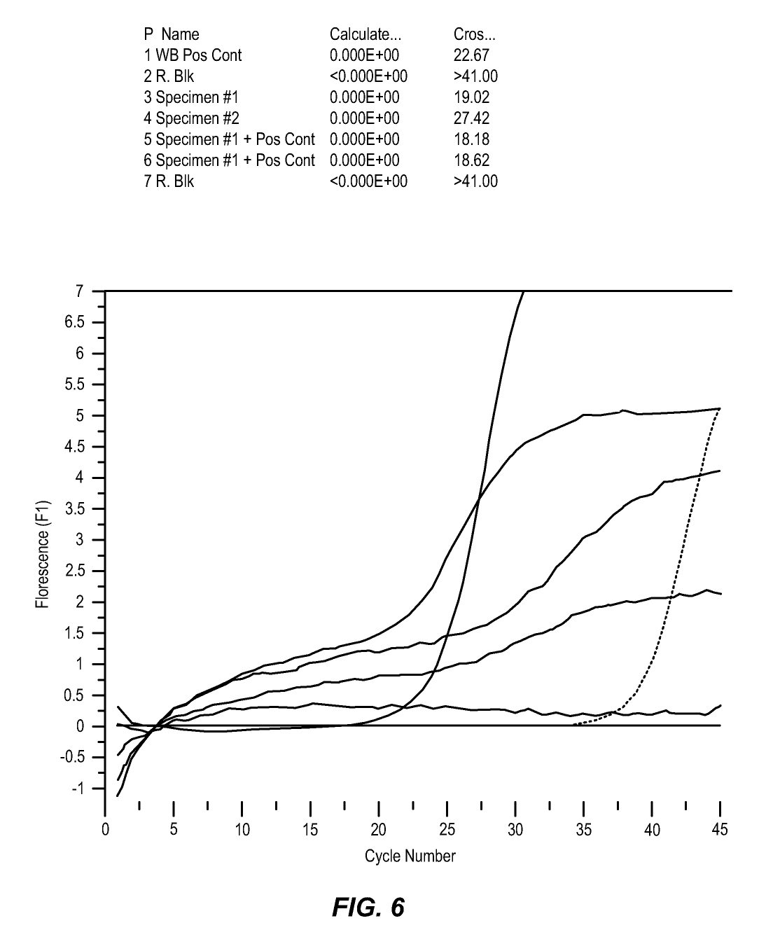

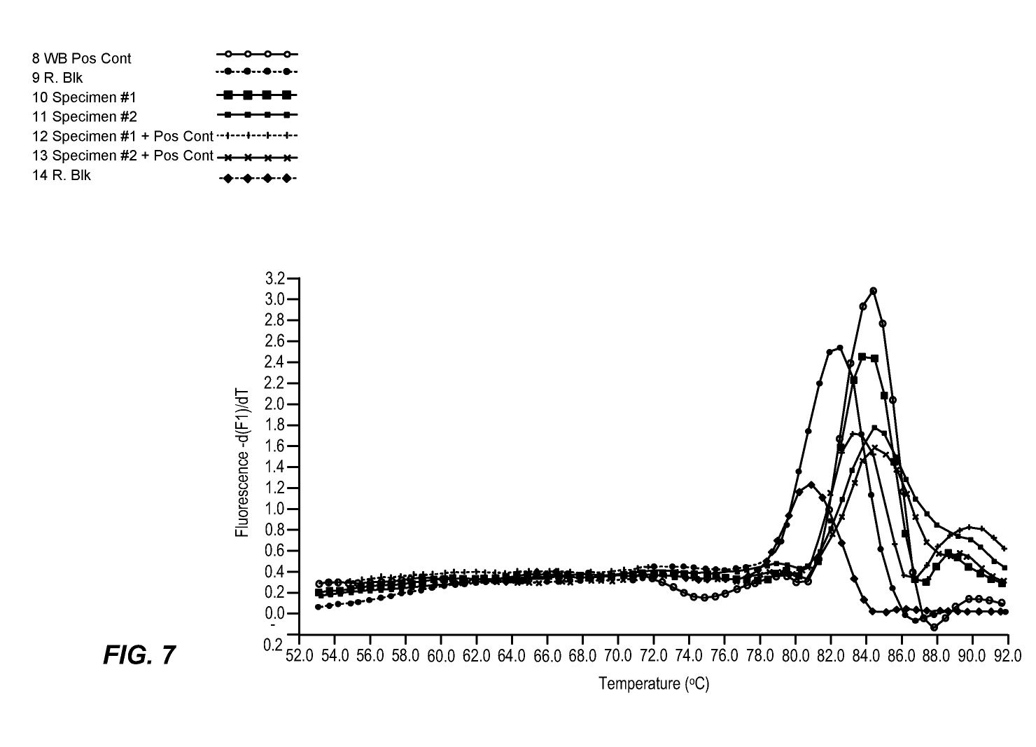

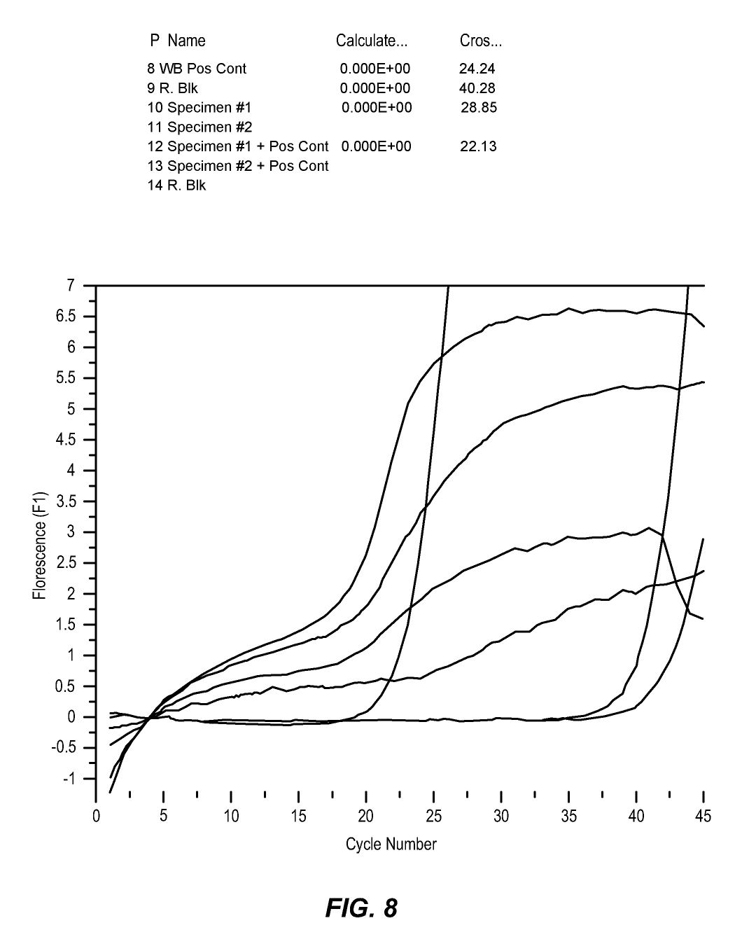

### DETAILED DESCRIPTION OF THE DISCLOSURE

The following description and examples illustrate invention embodiments. It is to be understood that this invention is not limited to the particular embodiments described herein and as such can vary. Those of skill in the art will recognize that there are numerous variations and modifications of this invention, which are encompassed within its scope.

### Definitions

The term “about” in relation to a reference numerical value and its grammatical equivalents as used herein can include a range of values plus or minus 10% from that value. For example the amount “about 10” can include amounts from 9 to 11. In other embodiments, the term “about” in relation to a reference numerical value can include a range of values plus or minus 10%, 9%, 8%, 7%, 6%, 5%, 4%, 3%, 2%, or 1% from that value. In some embodiments, the term “about” can include a range of values plus or minus 10% from that value. In some embodiments, the term “about” can include a range of values plus or minus 10% from a reference value. In some embodiments, the term “about” can include a range of values plus or minus 9% from a reference value. In some embodiments, the term “about” can include a range of values plus or minus 8% from a reference value. In some embodiments, the term “about” can include a range of values plus or minus 7% from a reference value. In some embodiments, the term “about” can include a range of values plus or minus 6% from a reference value. In some embodiments, the term “about” can include a range of values plus or minus 5% from a reference value. In some embodiments, the term “about” can include a range of values plus or minus 4% from a reference value. In some embodiments, the term “about” can include a range of values plus or minus 3% from a reference value. In some embodiments, the term “about” can include a range of values plus or minus 2% from a reference value. In some embodiments, the term “about” can include a range of values plus or minus 1% from a reference value.

The term “diagnosis” and its grammatical equivalents as used herein, can mean the testing of subjects to determine if they have a particular trait for use, e.g., in a clinical decision. Diagnosis can include testing of subjects at risk of developing a particular disease resulting from infection by an infectious organism or a non-infectious disease, such as cancer or a metabolic disease or a genetic disease. Diagnosis can also include testing of subjects who have developed particular symptoms to determine the cause of the symptoms. Diagnosis can also include prognosis, monitoring progress of a disease, and monitoring the efficacy of therapeutic regimens. The result of a diagnosis can be used to classify patients into groups for performance of clinical trials for administration of certain therapies.

The term “drug” and its grammatical equivalents as used herein, can mean any compounds of any degree of complexity that can perturb a biological state, whether by known or unknown mechanisms and whether or not they are used therapeutically. Drugs thus can include: typical small molecules of research or therapeutic interest; naturally-occurring factors, such as endocrine, paracrine, or autocrine factors or factors interacting with cell receptors of all types; intracellular factors, such as elements of intracellular signaling pathways; factors isolated from other natural sources; pesticides; herbicides; and insecticides. The term “drug” can also refer to a hydrate, solution, and/or polymorph. The term “drug” can also refer to and/or include its free-base, acid, salts, esters, and mixtures thereof. If a drug is a salt, it can refer to a pharmaceutically acceptable salt, including but not limited to the salts found in the “Handbook of Pharmaceutical Salts: Properties, Selection, and Use,” R. Heinrich Stahl and Camile G. Wermuth, eds., Wiley-VCH, 2^{nd} Edition (2011). For example, the drugs can be formulated into, but not limited to, hydrochloride salts, hydrobromide salts, hydroiodide salts, fumaric acid salts, maleic acid salts, amino acid salts, mineral acid salts, addition salts, nitrate salts, phosphate salts, succinate salts, maleate salts, fumarate salts, citrate salts, tartrate salts, gluconate salts, lactate salts, lactobionate salts, lauryl sulfate salts, glutamate salts, acetamidobenzoate salts, potassium salts, sodium salts, calcium salts, tromethamine salts, 2-aminoethanol salts, lysine salts, besylate salts, and/or arginine salts.

The term “treating” and its grammatical equivalents as used herein can include achieving a therapeutic benefit and/or a prophylactic benefit. Therapeutic benefit can be amelioration of the underlying disorder being treated. Also, a therapeutic benefit can be achieved with the amelioration of one or more of the physiological symptoms associated with the underlying disorder such that an improvement can be observed in the patient, notwithstanding that the patient may still be afflicted with the underlying disorder. For prophylactic benefit, the compositions can be administered to a patient at risk of developing a particular disease, or to a patient reporting one or more of the physiological symptoms of a disease, even though a diagnosis of this disease may not have been made.

The term “ameliorate” and its grammatical equivalents as used herein can include lessening of the severity (including eradication) of an underlying disease and/or disorder and/or symptom. The lessening of severity (including eradication) can be determined either objectively or subjectively. For example, when referring to symptoms of disease, the term “ameliorate” can mean reducing, lessening, and/or curing the symptoms of a disease.

“Therapeutically effective amount” and its grammatical equivalents as used herein can refer to the amount of an active ingredient, with or without additional active ingredients, which can be effective to achieve its intended purpose. While individual patient needs may vary, determination of optimal ranges for effective amounts of the compounds and compositions is within the skill of an ordinary practitioner of the art. Generally, the dosage required to provide an effective amount of the composition, and which can be adjusted by one of ordinary skill in the art, can vary depending on the age, health, physical condition, sex, weight, extent of the dysfunction of the recipient, frequency of treatment and the nature and scope of the dysfunction.

“Patient” or “subject” and its grammatical equivalents as used herein can include mammals, such as humans, including those in need of treatment thereof. For example, the terms “patient” or “subject” can include males, females, adults, young adults, teenagers, children, infants, and/or fetuses. The terms “patient” and “subject” can sometimes be used interchangeably.

The term “average” and its grammatical equivalents as used herein can refer to the mathematical mean. Typically the mean can be calculated by the adding a defined group of numbers divided by the number of members in the group. The term “average” can also be referred to as the mathematical mean.

The term “activator” and its grammatical equivalents as used herein to describe a substance that leads to an increase in the level of another e.g., measured substance. For example, a cyclic adenosine monophosphate (cAMP) activator can lead to an increase the level of cAMP; a cyclic guanosine monophosphate (cGMP) activator can lead to an increase the level of cGMP; or a sonic hedgehog (SHH) activator can lead to an increase the level of SHH.

The term “level” and its grammatical equivalents as used herein, when used in context with measuring, can refer to e.g., the level of: a nucleic acid, a protein, cells, etc. For example, SHH levels can mean SHH protein or SHH nucleic acid levels. In some cases, e.g., when the term “level” refers to proteins, the term “level” can also refer to enzymatic activity. In some cases, e.g., when the term “level” refers to concentration, the term “level” can also refer to the concentration of substance (e.g., expressed as per protein) or the amount of substance (e.g., expressed as per protein). In some cases, the term “level” can also refer to an amount per unit volume (e.g., mg/ml). In some cases, the term “level” can also refer to an amount per unit area (e.g., mg/cm²).

The terms “dosage” and “dosage amounts” and its grammatical equivalents as used herein can mean that the drugs can be formulated into any type of dosage forms suitable for e.g., oral administration, transmucosal administration, buccal administration, inhalation administration, intranasal administration, parental administration, intravenous administration, subcutaneous administration, intramuscular administration, sublingual administration, transdermal administration, and rectal administration.

The term “communication medium” and its grammatical equivalents as used herein can refer to any means of communicating information. Exemplary types of communication medium can include, but are not limited to written, printed, and electronic types of media. Other types of communication medium will be apparent to those skilled in the relevant arts without departing from the spirit and scope of the present disclosure.

The term “combination thereof” and its grammatical equivalents as used herein can refer to one or more members of the recited group. For example, if the group comprises A, B, or a combination thereof, each of A individually, B individually, and the group A and B are contemplated.

The term “chromosomal abnormalities” and its grammatical equivalents as used herein, e.g., chromosomal anomaly, abnormality, aberration, or mutation, can refer to: a missing, extra, or irregular portion of chromosomal DNA. The term can also refer to an atypical number of chromosomes or a structural abnormality in one or more chromosomes, and can also refer to a chromosomal translocation.

The term “cognitive” and its grammatical equivalents as used herein, can refer to the process by which the sensory input can be transformed, reduced, elaborated, stored, recovered, and/or used. For example, cognition can refer to the mental processing that includes the attention of working memory, comprehending and producing language, calculating, reasoning, problem solving, and/or decision making. The term can also include disease or conditions that affect the brain, such as, neurologic/neurodevelopmental disorders, autism, depression, anxiety, epileptic seizures, and/or dementia.

The term “post-partum” and its grammatical equivalents as used herein, can refer to the period of time beginning immediately after the birth of a child and extending for about six weeks following that time period.

The term “fetus” and its grammatical equivalents as used herein can refer to the time period after conception. The term “embryo” and its grammatical equivalents as used herein can refer to the same being as fetus. Therefore, the term “fetus” and “embryo” and their grammatical equivalents, can refer to a fertilized eggs any time after conception (even in some cases before conception, e.g., within the first 2 weeks of pregnancy).

The term “continuously” and its grammatical equivalents as used herein can refer to an extended period of time. For example, in the context of drug treatment, the term “continuously” and its grammatical equivalents can refer to treatment, e.g., once a day, twice a day, once every week, etc., for an indefinite period of time.

The term “nutrient” and its grammatical equivalents as used herein, can refer to a substance that contains and/or provides nourishment or nutriment. For example, the term “nutrient” can refer to water, carbohydrates, protein (including, e.g., amino acids), fats, vitamins, minerals, and combinations thereof. The term “nutrient” can include breast milk, formula, fruits, vegetables, and anything that contains water, carbohydrates, protein, fats, vitamins, minerals, and combinations thereof. In some cases, the term “nutrient” can be suitable for consumption by a human, e.g., a baby or a pregnant mother.

The term “reproductive system” and its grammatical equivalents as used herein can refer to any tissue or organ that can facilitate the reproduction. For example, a female human's reproductive system can comprise ovaries, fallopian tubes, uterus, vagina, vulva, mammary glands, and/or breasts.

The term “selective” and its grammatical equivalents as used herein can refer to a process or agent that affects some things and not others. In some cases, the term “selective” can also refer to a process or agent that minimally affects one thing, and largely affects another thing. In some cases, the process or agent can affect only a single thing. For example, in the case of phosphodiesterase (PDE) inhibitors, e.g., PDE1 selective inhibitors, this can mean that this drug inhibits PDE1 only, or more than one PDE, wherein the inhibitor of PDE1 can be more selective than for other PDEs.

The term “normalize” and its grammatical equivalents as used herein can refer to bringing or returning something to a normal state or condition. For example, the term “normalize” can refer to making a structure appear closer to and/or similar to a normal structure relative to its beginning structure after e.g., treatment is given. For instance, if a cell(s) was previously elongated but is now spherical in shape, the normalizing of the cell can return the cell into its previously elongated shape. In another example, if a cell proliferates at a rate of 1 cell division per 24 hours, but now divides every 72 hours, the normalizing of the cell can return the cell to a cell division rate of 1 cell division per 24 hours, and/or close to 1 cell division per 24 hours. If referring to a mathematics, the term “normalize” can refer to multiplying (a series, function, or item of data) by a factor that makes the norm or some associated quantity such as an integral equal to a desired value (e.g., 1).

### Method Embodiments

The methods described herein can comprise analyzing one or more biological samples from a subject to determine a level of one or more biological substances. The one or more biological samples can comprise one or more bodily fluids. The one or more bodily fluids can comprise, for example (e.g., as samples), a whole blood sample, a serum sample, a plasma sample, a urine sample, a saliva sample, a mucus sample, a perspiration sample, or a combination thereof. In some instances, the one or more biological samples can comprise the mucus sample. More specifically, the mucus sample can comprise a nasal mucus sample. Use of nasal specimens (e.g., the nasal mucus sample) can provide a minimally invasive manner of obtaining biological samples for analysis. Some patients may prefer to have a blood test. In these instances, the bodily fluids can be whole blood, plasma, and/or serum samples, and can be used separately or in combination with each other. Another minimally invasive way to extract a bodily fluid from a patient can be by collecting a perspiration sample. Methods to collect perspiration samples are within the abilities of a person of skill in the art. The results of this analysis can be suitable for use in diagnosis, prognosis, and determination of suitability of therapeutic interventions and/or the presence or absence of a disease or condition.

The term “one or more members of the hedgehog signaling pathway” and its grammatical equivalents as used herein, can include known or unknown members of the hedgehog signaling pathway. For example, known members of the hedgehog signaling pathway can include the currently known members of the hedgehog signaling pathway, Sonic Hedgehog (SHH) (e.g., SEQ ID Nos. 1-3 (homo sapien)), Desert Hedgehog (DHH) (e.g., SEQ ID Nos. 4-6 (homo sapien)), and Indian hedgehog (IHH) (e.g., SEQ ID Nos. 7-9 (homo sapien)). Unknown members of the hedgehog signaling pathway can be found by comparing the homology of nucleic acid and proteins sequences. For example, by comparing databases with SEQ ID NOs. 1-9. Although, the invention can be directed towards the all of the members of the hedgehog signaling pathway, specific hedgehog members are also contemplated. Therefore, it is contemplated that the invention can focus on SHH, DHH, IHH, or any combination thereof.

The term “biological substance” and its grammatical equivalents as used herein, can include cells and/or their extra-cellular and/or intra-cellular constituent(s). For example, biological substances can include pathogens, metabolites, DNA, RNA, lipids, proteins, carbohydrates, receptors, enzymes, hormones, growth factors, growth inhibitory factors, cells, organs, tissues, portions of cells, tissues, and/or organs, subcellular organelles, chemically reactive molecules like H⁺, superoxides, ATP, citric acid, protein albumin, as well as combinations or aggregate representations of these types of biological variables. In addition, biological substances can include therapeutic agents such as, but not limited to, methotrexate, steroids, non-steroidal anti-inflammatory drugs, soluble TNF-alpha receptor, TNF-alpha antibody, and interleukin-1 receptor activators.

In any embodiment herein, the subject can be a subject in need thereof.

### Sample Collection

One or more biological samples can be collected from a subject for analysis. The one or more biological samples can comprise one or more bodily fluids. For example, the one or more bodily fluids can comprise a whole blood sample, a serum sample, a plasma sample, a urine sample, a saliva sample, a mucus sample, a perspiration sample, or a combination thereof. The one or more bodily fluids can also contain genetic material. A number of biological fluids can be collected that contain genetic materials which can be used in any of the embodiments of this invention. For example, genetic material can be extracted by, e.g., amniocentesis, needles, to obtain for example, blood, serum, saliva, sperm, eggs, etc. One of the most easily accessible bodily fluids can be mucus, which can be a nasal mucus sample; this invention contemplates using nasal mucus samples. Additionally, because blood samples can be sometimes easily accessible as well, the one or more bodily fluids can comprise a plasma sample, a serum sample, a whole blood sample, or a combination thereof. Another easily accessible bodily fluid that can be used in the invention can be a perspiration sample.

If the one or more biological specimens is, for example, from the nasal area (e.g., a nasal mucus sample), the sample of nasal secretions can be collected directly from the nose into a collection tube or device. Alternative collection methods are also contemplated. For example, a sample of nasal secretion can be collected on a sample collection device by passing it into the nostril of a patient. The device may be inserted sequentially into each nostril of the patient and advanced parallel to the hard palate with slow rotation. The device can then be typically transferred to a transport tube, such as a glass or plastic test tube. The transport tube can include a suitable volume of a sterile medium such as ethanol or the like.

Other bodily fluids, such as a saliva sample can be obtained, for example, by draining, spitting, suction, and/or swabbing, to collect saliva, for example, mixed saliva. In order to better promote collection, gustatory or masticatory stimulation can be used to increase the flow of saliva. Another collection method can be by the use of a modified Lashley cup placed over the Stensen's duct, or with lingual stimulation with lemon juice to obtain parotid saliva, for example, pure saliva.

A blood sample can be collected, for example, by venipuncture, or finger sticking. Whole blood samples can be collected, for example, in a tube (e.g., a vacuum tube, a capillary tube), a syringe, and/or a bag. Plasma and serum samples can be derived from blood samples, e.g., by centrifugation.

A urine sample can be collected, e.g., in a cup, or in a 24-hour collection.

A perspiration sample can be collected, e.g., in a tube, and may be further purified for analysis. Collection can occur by any known method. In particular, a sweat sample can be collected using a special sweat stimulation procedure. For example, (a) a sweat-stimulating liquid can be applied to the skin creating a stimulated area; (b) an electrode can be placed on the stimulated area; (c) the stimulated area can be exposed to a weak electrical current; and (d) sweat can be collected from the stimulated area into a plastic coil of tubing or onto a piece of gauze or filter paper.

A nasal sample collection device can be a swab, a wooden spatula, bibulous materials such as a cotton ball, filter, or gauze pad, an absorbent-tipped applicator, capillary tube, or a pipette. A swab can be used as a sample collection device, and the sample processing element can comprise a swab holder or a swab processing insert. The swab holder or swab processing insert can be tapered or angled to allow a single sample processing element to accommodate all types of swabs by allowing swabs with different amounts of fiber, or that can be wound to different levels of tightness, to be held securely within the holder or insert. In certain cases, the swab holder or swab processing insert can securely hold the swab to provide stability. Nasal samples can also be collected from spontaneous discharge from the nasal cavity.

Samples may be collected from individuals repeatedly over a longitudinal period of time (e.g., once a day, once a week, once a month, biannually or annually). Obtaining numerous samples from an individual over a period of time can be used to verify results from earlier detections and/or to identify an alteration as a result of, for example, drug treatment. Samples can be obtained from humans or non-humans.

### Analysis

One or more biological samples can be collected and analyzed using one or more analytical techniques including enzymatic technique, enzyme-linked immunosorbent assay (ELISA), fluorometric technique, mass spectrography, visible spectrophotometric techniques, high-performance liquid chromatography (HPLC), gas-liquid chromatography (GLC), polymerase chain reaction (PCR), protein and nucleic acid sequencing, and/or other similar techniques. The analysis can comprise determining the presence and/or level of one or more biological substance in the one or more biological samples.

Polymerase Chain Reaction (PCR)

The polymerase chain reaction (PCR) is a process for amplifying one or more desired specific nucleic acid sequences found in a nucleic acid. Because large amounts of a specific sequence may be produced by this process, it can be used for improving the efficiency of cloning DNA or messenger RNA and for amplifying a target sequence to facilitate detection thereof.

PCR involves a chain reaction for producing, in exponential quantities relative to the number of reaction steps involved, at least one specific nucleic acid sequence given (a) that the ends of the required sequence are known in sufficient detail that oligonucleotides can be synthesized which will hybridize to them, and (b) that a small amount of the sequence is available to initiate the chain reaction. The product of the chain reaction would be a discrete nucleic acid duplex with termini corresponding to the ends of the specific primers employed.

Any source of nucleic acid, in purified or non-purified form, can be utilized as the starting nucleic acid or acids, provided it contains or is suspected of containing a specific nucleic acid sequence desired. Thus, the process may employ, for example, DNA or RNA, including messenger RNA, which DNA or RNA may be single stranded or double stranded. In addition, a DNA-RNA hybrid which contains one strand of each may be utilized. A mixture of any of these nucleic acids may also be employed, or the nucleic acid produced from a previous amplification reaction herein using the same or different primers may be so utilized. The specific nucleic acid sequence to be amplified may be only a fraction of a larger molecule or can be present initially as a discrete molecule, so that the specific sequence constitutes the entire nucleic acid. It is not necessary that the sequence to be amplified be present initially in a pure form; it may be a minor fraction of a complex mixture, such as a portion of the β-globin gene contained in whole human DNA or a portion of nucleic acid sequence due to a particular microorganism which organism might constitute only a minor fraction of a particular biological sample. The starting nucleic acid may contain more than one desired specific nucleic acid sequence which may be the same or different. Therefore, it can be useful not only for producing large amounts of one specific nucleic acid sequence, but also for amplifying simultaneously more than one different specific nucleic acid sequence located on the same or different nucleic acid molecules.

The nucleic acid or acids may be obtained from any source, for example, from plasmids such as pBR322, from cloned DNA or RNA, or from natural DNA or RNA from any source, including but not limited to, bacteria, yeast, viruses, and higher organisms such as plants or animals. DNA or RNA may be extracted from, including but not limited to, blood (whole blood, plasma, serum), tissue material such as chorionic villi or amniotic cells. The DNA or RNA may be cell-free DNA or RNA.

It will be understood that the word primer as used may refer to more than one primer, particularly in the case where there is some ambiguity in the information regarding the terminal sequence(s) of the fragment to be amplified. For instance, in the case where a nucleic acid sequence is inferred from protein sequence information a collection of primers containing sequences representing all possible codon variations based on degeneracy of the genetic code will be used for each strand. One primer from this collection can be 100% homologous with the end of the desired sequence to be amplified.

An appropriate agent may be added for inducing or catalyzing the primer extension reaction and the reaction can be allowed to occur under conditions known in the art. The inducing agent may be any compound or system which will function to accomplish the synthesis of primer extension products, including, but not limited to, enzymes. Suitable enzymes for this purpose can include, for example, E. coli DNA polymerase I, Klenow fragment of E. coli DNA polymerase I, T4 DNA polymerase, other available DNA polymerases, reverse transcriptase, and other enzymes, including heat-stable enzymes, which will facilitate combination of the nucleotides in the proper manner to form the primer extension products which can be complementary to each nucleic acid strand. Generally, the synthesis can be initiated at the 3′ end of each primer and proceed in the 5′ direction along the template strand, until synthesis terminates, producing molecules of different lengths. There may be inducing agents, however, which initiate synthesis at the 5′ end and proceed in the other direction, using the same process as described above.

The newly synthesized strand and its complementary nucleic acid strand can form a double-stranded molecule which can be used in the succeeding steps of the process. In the next step, the strands of the double-stranded molecule may be separated to provide single-stranded molecules. New nucleic acid may be synthesized on the single-stranded molecules. Additional inducing agent, nucleotides and primers may be added if necessary for the reaction to proceed under the conditions prescribed above. Again, the synthesis can be initiated at one end of the oligonucleotide primers and can proceed along the single strands of the template to produce additional nucleic acid. After this step, half of the extension product can consist of the specific nucleic acid sequence bounded by the two primers. The steps of strand separation and extension product synthesis can be repeated as often as needed to produce the desired quantity of the specific nucleic acid sequence. The amount of the specific nucleic acid sequence produced can accumulate in an exponential fashion. After the appropriate length of time has passed to produce the desired amount of the specific nucleic acid sequence, the reaction may be halted by inactivating the enzymes in any known manner or separating the components of the reaction.

Amplification can be useful when the amount of nucleic acid available for analysis is small, as, for example, in the prenatal diagnosis of sickle cell anemia using DNA obtained from fetal cells or from maternal plasma/serum/blood. Amplification can be particularly useful if such an analysis can be to be done on a small sample using non-radioactive detection techniques that can be inherently insensitive, or where radioactive techniques are employed but where rapid detection can be desirable.

Any known techniques for nucleic acid (e.g., DNA and RNA) amplification can be used with the assays described herein. Some amplification techniques can be the polymerase chain reaction (PCR) methodologies which can include, but are not limited to, solution PCR and in situ PCR.

The invention is not limited to the use of straightforward PCR. A system of nested primers may be used for example. Other suitable amplification methods known in the field can also be applied such as, but not limited to, ligase chain reaction (LCR), strand displacement amplification (SDA), self-sustained sequence replication (3SR), array based test, digital PCR, and TAQMAN.

As used herein “amplification” may refer to any in vitro method for increasing the number of copies of a nucleic acid sequence with the use of a DNA polymerase. Nucleic acid amplification can result in the incorporation of nucleotides into a DNA molecule or primer thereby forming a new DNA molecule complementary to a DNA template. The newly formed DNA molecule and its template can be used as templates to synthesize additional DNA molecules. As used herein, one amplification reaction may consist of many rounds of DNA replication. DNA amplification reactions can include, for example, polymerase chain reactions (PCR). One PCR reaction may consist of 5-100 “cycles” of denaturation, annealing, and synthesis of a DNA molecule.

Nucleic Acid Sequencing

Nucleic acid sequencing can be used for detection of a biological substance in a biological sample. Nucleic acid sequencing enables detection of the presence or absence of nucleic acids, determining the levels of nucleic acids, and also determining the exact nucleotide sequences. The methods can be performed by any known methods, for example, Maxam-Gilbert sequencing, Sanger sequencing, shotgun sequencing, bridge PCR, massively parallel signature sequencing (MPSS), polony sequencing, 454 pyrosequencing, Illumina (Solexa) sequencing, SOLiD sequencing, Ion Torrent semiconductor sequencing, DNA nanoball sequencing, heliscope single molecule sequencing, and/or single molecule real time (SMRT) sequencing. Other sequencing methods can be used such as nanopore DNA sequencing, tunneling currents DNA sequencing, sequencing by hybridization, sequencing with mass spectrometry, microfluidic Sanger sequencing, microscopy-based techniques, RNAP sequencing, and/or in vitro virus high-throughput sequencing.

Fluorescence Microscopy

Fluorescence microscopy can be used for detection of a biological substance in a biological sample. Fluorescence microscopy can enable the molecular composition of the structures being observed to be identified through the use of fluorescently-labeled probes of high chemical specificity such as antibodies. It can be done by directly conjugating a fluorophore to a protein and introducing this back into a cell. Fluorescent analogs may behave like the native protein and can therefore serve to reveal the distribution and behavior of this protein in the cell. Along with NMR, infrared spectroscopy, circular dichroism and other techniques, protein intrinsic fluorescence decay and its associated observation of fluorescence anisotropy, collisional quenching and resonance energy transfer can be techniques for protein detection. Microscopy can also be used to detect and enumerate cells, such as eosinophils.

The naturally fluorescent proteins can be used as fluorescent probes. The jellyfish aequorea victoria produces a naturally fluorescent protein known as green fluorescent protein (GFP). The fusion of these fluorescent probes to a target protein enables visualization by fluorescence microscopy and quantification by flow cytometry. Without limiting the scope of the present invention, some of the probes are as follows:

Labels:

Sensitivity and safety (compared to radioactive methods) of fluorescence has led to an increasing use for specific labeling of nucleic acids, proteins and other biomolecules. Besides fluorescein, other fluorescent labels cover the whole range from 400 to 820 nm. By way of example only, some of the labels can be: fluorescein and its derivatives, carboxyfluoresceins, rhodamines and their derivatives, atto labels, fluorescent red and fluorescent orange: Cy3/Cy5 alternatives, lanthanide complexes with long lifetimes, long wavelength labels—up to 800 nm, DY cyanine labels, and phycobili proteins.

Conjugates:

Antibody conjugates can be generated with specificity for virtually any epitope and can be therefore, applicable to imaging a wide range of biomolecules. By way of example only, some of the conjugates can be: isothiocyanate conjugates, streptavidin conjugates, and/or biotin conjugates.

Enzyme Substrates:

By way of example only, some of the enzyme substrates can be fluorogenic and chromogenic substrates.

Micro- and Nanoparticles:

By way of example only, some of the fluorochromes can be: FITC (green fluorescence, excitation/emission=506/529 nm), rhodamine B (orange fluorescence, excitation/emission=560/584 nm), and nile blue A (red fluorescence, excitation/emission=636/686 nm). Fluorescent nanoparticles can be used for various types of immunoassays. Fluorescent nanoparticles can be based on different materials, such as, polyacrylonitrile, and polystyrene etc.

Molecular Rotors:

Fluorescent molecular rotors are sensors of microenvironmental restriction that become fluorescent when their rotation is constrained. Few examples of molecular constraint can include increased dye (aggregation), binding to antibodies, or being trapped in the polymerization of actin.

IEF-Markers:

IEF (isoelectric focusing) can be an analytical tool for the separation of ampholytes, mainly proteins. An advantage for IEF-Gel electrophoresis with fluorescent IEF-marker can be the possibility to directly observe the formation of gradient. Fluorescent IEF-marker can also be detected by UV-absorption at 280 nm (20° C.).

Any or all of these fluorescent probes can be used for the detection of biological substances in the nasal mucus. A peptide library can be synthesized on solid supports and, by using coloring receptors, subsequent dyed solid supports can be selected one by one. If receptors cannot indicate any color, their binding antibodies can be dyed. The methods can not only be used on protein receptors, but also on screening binding ligands of synthesized artificial receptors and screening new metal binding ligands as well. Automated methods for HTS and FACS (fluorescence activated cell sorter) can also be used. A FACS machine originally runs cells through a capillary tube and separate cells by detecting their fluorescent intensities.

Immunoassays

Immunoassays can be used for detecting a biological substance in a biological sample. In immunoblotting like the western blot of electrophoretically separated proteins a single protein can be identified by its antibody. Immunoassay can be competitive binding immunoassay where analyte competes with a labeled antigen for a limited pool of antibody molecules (e.g., radioimmunoassay, EMIT). Immunoassay is non-competitive where antibody can be present in excess and can be labeled. As analyte antigen complex is increased, the amount of labeled antibody-antigen complex may also increase (e.g., ELISA). Antibodies can be polyclonal if produced by antigen injection into an experimental animal, or monoclonal if produced by cell fusion and cell culture techniques. In immunoassay, the antibody may serve as a specific reagent for the analyte antigen.

Without limiting the scope and content of the present invention, some of the types of immunoassays can be, by way of example only, RIAs (radioimmunoassay), enzyme immunoassays like ELISA (enzyme-linked immunosorbent assay), EMIT (enzyme multiplied immunoassay technique), microparticle enzyme immunoassay (META), LIA (luminescent immunoassay), and FIA (fluorescent immunoassay). These techniques can be used to detect biological substances in the nasal specimen. The antibodies either used as primary or secondary ones can be labeled with radioisotopes (e.g., 125I), fluorescent dyes (e.g., FITC) or enzymes (e.g., HRP or AP) which may catalyze fluorogenic or luminogenic reactions.

EMIT (Enzyme Multiplied Immunoassay Technique):

EMIT is a competitive binding immunoassay that can avoid a separation step. EMIT is a type of immunoassay in which the protein can be labeled with an enzyme, and the enzyme-protein-antibody complex can be enzymatically inactivated, allowing quantitation of unlabeled protein.

ELISA (Enzyme Linked Immunosorbent Assay):

The invention can also use ELISA to detect biological substances in the nasal specimen. ELISA is based on selective antibodies attached to solid supports combined with enzyme reactions to produce systems capable of detecting low levels of proteins. It is also known as enzyme immunoassay or EIA. The protein can be detected by antibodies that have been made against it, that is, for which it is the antigen. Monoclonal antibodies can be often used.

The test may require the antibodies to be fixed to a solid surface, such as the inner surface of a test tube, and a preparation of the same antibodies coupled to an enzyme. The enzyme may be one (e.g., (3-galactosidase) that produces a colored product from a colorless substrate. The test, for example, may be performed by filling the tube with the antigen solution (e.g., protein) to be assayed. Any antigen molecules present may bind to the immobilized antibody molecules. The antibody-enzyme conjugate may be added to the reaction mixture. The antibody part of the conjugate binds to any antigen molecules that were bound previously, creating an antibody-antigen-antibody “sandwich”. After washing away any unbound conjugate, the substrate solution may be added. After a set interval, the reaction can be stopped (e.g., by adding 1 N NaOH) and the concentration of colored product formed can be measured in a spectrophotometer. The intensity of color can be proportional to the concentration of bound antigen.

ELISA can also be adapted to measure the concentration of antibodies, in which case, the wells can be coated with the appropriate antigen. The solution (e.g., serum) containing antibody may be added. After it has had time to bind to the immobilized antigen, an enzyme-conjugated anti-immunoglobulin may be added, consisting of an antibody against the antibodies being tested for. After washing away unreacted reagent, the substrate may be added. The intensity of the color produced can be proportional to the amount of enzyme-labeled antibodies bound (and thus to the concentration of the antibodies being assayed).

Radioimmunoassay:

Some embodiments of the invention can include radioimmunoassays to detect biological substances in the biological samples, e.g., in the nasal specimen. Radioactive isotopes can be used to study in vivo metabolism, distribution, and binding of small amount of compounds. Radioactive isotopes of ¹H, ¹²C, ³¹P, ³²S, and ¹²⁷I in body can be used, such as ³H, ¹⁴C, ³²P, ³⁵S, and ¹²⁵I.

In receptor fixation method in 96-well plates, receptors may be fixed in each well by using antibody or chemical methods and radioactive labeled ligands may be added to each well to induce binding. Unbound ligands may be washed out and then the standard can be determined by quantitative analysis of radioactivity of bound ligands or that of washed-out ligands. Then, addition of screening target compounds may induce competitive binding reaction with receptors. If the compounds show higher affinity to receptors than standard radioactive ligands, most of radioactive ligands would not bind to receptors and may be left in solution. Therefore, by analyzing quantity of bound radioactive ligands (or washed-out ligands), testing compounds' affinity to receptors can be indicated.

The filter membrane method may be needed when receptors cannot be fixed to 96-well plates or when ligand binding needs to be done in solution phase. In other words, after ligand-receptor binding reaction in solution, if the reaction solution can be filtered through nitrocellulose filter paper, small molecules including ligands may go through it and only protein receptors may be left on the paper. Only ligands that strongly bound to receptors may stay on the filter paper and the relative affinity of added compounds can be identified by quantitative analysis of the standard radioactive ligands.

Fluorescence Immunoassays:

The invention can also include fluorescence immunoassays for detecting a biological substance in a biological sample. Fluorescence based immunological methods can be based upon the competitive binding of labeled ligands versus unlabeled ones on highly specific receptor sites. Fluorescence immunoassays can also be used to detect and enumerate cells, such as eosinophils.

The fluorescence technique can be used for immunoassays based on changes in fluorescence lifetime with changing analyte concentration. This technique may work with short lifetime dyes like fluorescein isothiocyanate (FITC) (the donor) whose fluorescence may be quenched by energy transfer to eosin (the acceptor). A number of photoluminescent compounds may be used, such as cyanines, oxazines, thiazines, porphyrins, phthalocyanines, fluorescent infrared-emitting polynuclear aromatic hydrocarbons, phycobiliproteins, squaraines and organo-metallic complexes, hydrocarbons and azo dyes.

Fluorescence based immunological methods can be, for example, heterogenous or homogenous. Heterogenous immunoassays can comprise physical separation of bound from free labeled analyte. The analyte or antibody may be attached to a solid surface. The technique can be competitive (for a higher selectivity) or noncompetitive (for a higher sensitivity). Detection can be direct (only one type of antibody used) or indirect (a second type of antibody can be used). Homogenous immunoassays can comprise no physical separation. Double-antibody fluorophorelabeled antigen can participate in an equilibrium reaction with antibodies directed against both the antigen and the fluorophore. Labeled and unlabeled antigen may compete for a limited number of anti-antigen antibodies.

Some of the fluorescence immunoassay methods can include simple fluorescence labeling method, fluorescence resonance energy transfer (FRET), time resolved fluorescence (TRF), and scanning probe microscopy (SPM). The simple fluorescence labeling method can be used for receptor-ligand binding, enzymatic activity by using pertinent fluorescence, and as a fluorescent indicator of various in vivo physiological changes such as pH, ion concentration, and electric pressure. TRF is a method that can selectively measure fluorescence of the lanthanide series after the emission of other fluorescent molecules is finished. TRF can be used with FRET and the lanthanide series can become donors or acceptors. In scanning probe microscopy, in the capture phase, for example, at least one monoclonal antibody can adhere to a solid phase and a scanning probe microscope can be utilized to detect antigen/antibody complexes which may be present on the surface of the solid phase. The use of scanning tunneling microscopy can eliminate the need for labels which normally can be utilized in many immunoassay systems to detect antigen/antibody complexes.

Nuclear Magnetic Resonance (NMR)

The invention can also include NMR for detecting a biological substance in a biological sample. NMR spectroscopy can determine the structures of biological macromolecules like proteins and nucleic acids at atomic resolution. In addition, it may be possible to study time dependent phenomena with NMR, such as intramolecular dynamics in macromolecules, reaction kinetics, molecular recognition or protein folding. Heteronuclei like ¹⁵N, ¹³C and ²H, can be incorporated in proteins by uniform or selective isotopic labeling. Additionally, some new information about structure and dynamics of macromolecules can be determined with these methods.

X-Ray Crystallography

The invention can also include X-ray crystallography for detecting a biological substance in a biological sample. X-ray crystallography is a technique in which the pattern produced by the diffraction of X-rays through the closely spaced lattice of atoms in a crystal can be recorded and then analyzed to reveal the nature of that lattice. This generally can lead to an understanding of the material and molecular structure of a substance. The spacing in the crystal lattice can be determined using Bragg's law. X-ray diffraction can be commonly carried out using single crystals of a material, but if these are not available, microcrystalline powdered samples may also be used which may require different equipment.

Fluorescence Spectroscopy

The invention can also include fluorescence spectroscopy for detecting a biological substance in a biological sample. By way of example only, conventional fluorometry can be measurement of emission light intensities at defined wavelengths for a certain emission maxima of a fluorophore. Total fluorometry can be a collection of data for a continuum of absorption as well as emission wavelengths. Fluorescence polarization is when polarized light can be used for excitation and binding of fluorochrome-labeled antigens to specific antibodies. Line narrowing spectroscopy is low-temperature solid-state spectroscopy that can derive its selectivity from the narrow-line emission spectra.

Time-dependent fluorescence spectroscopy can comprise time-resolved measurements containing more information than steady-state measurements, since the steady-state values represent the time average of time-resolved determinations. It can be a single photon timing technique where the time between an excitation light pulse and the first photon emitted by the sample can be measured.

Matrix Assisted Laser Desorption Ionization Time-of-Flight Mass Spectrometry (MALDI TOF-MS)

The invention can include MALDI TOF-MS for detecting a biological substance in a biological sample. MALDI TOF-MS provides accurate mass determinations and primary sequence information. Improved mass resolution in MALDI TOF-MS can be obtained by the utilization of a single-stage or a dual-stage reflectron (RETOF-MS). In the reflectron mass spectrum, the isotopic multiplet can be well resolved producing a full width half maximum (FWHM) mass resolution of about 3400. Mass resolutions up to 6000 (FWHM) can be obtained for peptides up to about 3000 Da with RETOF-MS. Enhancing the mass resolution can also increase the mass accuracy when determining the ion's mass.

Both linear and reflectron MALDI-TOF-MS can be utilized for molecular weight determinations of molecular ions and enzymatic digests leading to structural information of proteins. These digests can be typically mass analyzed with or without purification prior to molecular weight determinations. Varieties of methodologies have been developed to obtain primary sequence information for proteins and peptides utilizing MALDI TOF-MS. Two different approaches can be taken. The first method is known as protein ladder sequencing and can be employed to produce structurally informative fragments of the analyte prior to insertion into the TOF mass spectrometer and subsequent analysis. The second approach can utilize the phenomenon of metastable ion decay that occurs inside the TOF mass spectrometer to produce sequence information.

The ladder sequencing with TOF-MS consists of either a time-dependent or concentration-dependent chemical degradation from either the N- or C-terminus of the protein/peptide into fragments, each of which differs by one amino acid residue. The mixture can be mass analyzed in a single MALDI-TOF-MS experiment with mass differences between adjacent mass spectral peaks corresponding to a specific amino acid residue. The order of occurrence in the mass spectrum defines the sequence of amino acids in the original protein/peptide.

Post-source decay with RETOF-MS MALDI is an ionization technique that can produce intact protonated pseudomolecular ion species. A significant degree of metastable ion decay can occur after ion acceleration and prior to detection. The ion fragments produced from the metastable ion decay of peptides and proteins typically can include both neutral molecule losses (such as water, ammonia and portions of the amino acid side chains) and random cleavage at peptide bonds. In-source decay with linear TOF-MS can be an alternative approach to RETOF-MS for studying metastable ion decay of MALDI generated ions. Primary structural information for peptides and proteins can be obtained by this method. Coherent mass spectral peaks can be produced from these metastable decayed ions giving rise to significant structural information for peptides and proteins.

Surface-Enhanced Laser Desorption Ionization-Time Offlight (SELDI-TOF)

The invention can include SELDI TOF-MS for detecting a biological substance in a biological sample. This technique can utilize stainless steel or aluminum-based supports, or chips, engineered with chemical (hydrophilic, hydrophobic, pre-activated, normal-phase, immobilized metal affinity, and cationic or anionic) or biological (antibody, antigen binding fragments (e.g., scFv), DNA, enzyme, or receptor) bait surfaces of 1-2 mm in diameter. These varied chemical and biochemical surfaces can allow differential capture of proteins based on the intrinsic properties of the proteins themselves. Solubilized tissue or body fluids in volumes as small as 0.1 μl can be directly applied to these surfaces, where proteins with affinities to the bait surface may bind. Following a series of washes to remove non-specifically or weakly bound proteins, the bound proteins can be laser desorbed and ionized for MS analysis. Masses of proteins ranging from small peptides of less than 1000 Da up to proteins of greater than 300 kDa can be calculated based on time-of-flight. As mixtures of proteins may be analyzed within different samples, a unique sample fingerprint or signature may result for each sample tested. Consequently, patterns of masses rather than actual protein identifications can be produced by SELDI analysis. These mass spectral patterns can be used to differentiate patient samples from one another, such as diseased from normal.

UV-Vis

The invention can include optical absorption spectroscopy (UV/VIS) for detecting a biological substance in a biological sample. UV/VIS provides light absorption data which helps in the determination of concentration of macromolecules such as, proteins, DNA, nucleotides etc. Organic dyes can be used to enhance the absorption and to shift the absorption into the visible range (e.g., coomassie blue reagents). Resonance raman spectroscopy (RRS) can be used to study molecular structure and dynamics. RRS helps in investigating specific parts of macromolecules by using different excitation wavelengths.

Liquid Chromatography (LC)

The invention can include LC for detecting a biological substance in a biological sample. Examples of LC are but not limited to, affinity chromatography, gel filtration chromatography, anion exchange chromatography, cation exchange chromatography, diode array-LC and high performance liquid chromatography (HPLC).

Gel filtration chromatography can separate proteins, peptides, and oligonucleotides on the basis of size. Molecules may move through a bed of porous beads, diffusing into the beads to greater or lesser degrees. Smaller molecules may diffuse further into the pores of the beads and therefore move through the bed more slowly, while larger molecules may enter less or not at all and thus move through the bed more quickly. Both molecular weight and three dimensional shapes contribute to the degree of retention. Gel Filtration Chromatography may be used for analysis of molecular size, for separations of components in a mixture, or for salt removal or buffer exchange from a preparation of macromolecules.

Affinity chromatography is the process of bioselective adsorption and subsequent recovery of a compound from an immobilized ligand. This process can allow for the specific and efficient purification of many diverse proteins and other compounds. Ion exchange chromatography can separate molecules based on differences between the overall charges of the proteins. It can be used for the purification of protein, oligonucleotides, peptides, or other charged molecules.

HPLC can be used in the separation, purification and detection of biological substances in the nasal mucus. Crude tissue extracts may be loaded directly onto the HPLC system and mobilized by gradient elution. Rechromatography under the identical conditions can be an option if further purification is warranted or necessary. Reversed phase chromatography (RPC) can be utilized in the process of protein structure determination. HPLC may be coupled with MS. The HPLC method described in Henkin et al., New Frontiers in Immunobiology, 2000, pp. 127-152.

The size-exclusion chromatography (SEC) and ion-exchange chromatography (IEC) can be used for separation and purification of biologically active proteins, such as enzymes, hormones, and antibodies. In liquid affinity chromatography (LAC), interaction may be based on binding of the protein due to mimicry of substrate, receptor, etc. The protein may be eluted by introducing a competitive binding agent or altering the protein configuration which may facilitate dissociation. A procedure that can be used in the separation of membrane proteins is the use of nonionic detergents, such as Triton X-100, or protein solubilization by organic solvents with IEC.

Diode array detector-liquid chromatography (DAD-LC) provides complete, multiple spectra for each HPLC peak, which, by comparison, can provide indication of peak purity. These data can also assign presence of tyr, trp, phe, and possibly others (his, met, cys) and can quantitate these amino acids by 2nd derivative or multi-component analysis. By a post-column derivatization, DAD-LC can also identify and quantitate cys, his and arg in individual peptides. Thus, it can be possible to analyze for 6 of the 20 amino acids of each separated peptide in a single LC run, and information can be obtained about presence or absence of these amino acids in a given peptide in a single step. This can be assisted by knowing the number of residues in each peptide.

Electrophoresis

The invention can include electrophoresis for detecting a biological substance in a biological sample. Electrophoresis can be gel electrophoresis or capillary electrophoresis.

Gel Electrophoresis:

Gel electrophoresis is a technique that can be used for the separation of proteins. During electrophoresis, macromolecules can be forced to move through pores when an electrical current can be applied. Their rate of migration through the electric field depends on strength of the field, size and shape of the molecules, relative hydrophobicity of the samples, and on an ionic strength and temperature of a buffer in which the molecules can be moving. After staining, the separated macromolecules in each lane can be seen in a series of bands spread from one end of the gel to the other. Using this technology can be possible to separate and identify protein molecules that differ by as little as a single amino acid. Also, gel electrophoresis can allow determination of crucial properties of a protein such as its isoelectric point and approximate molecular weight. Electrofocusing or isoelectric focusing is a technique for separating different molecules by their electric charge differences (if they have any charge). It is a type of zone electrophoresis that takes advantage of the fact that a molecule's charge changes as the pH of its surroundings changes.

Capillary Electrophoresis:

Capillary electrophoresis is a collection of a range of separation techniques which may involve the application of high voltages across buffer filled capillaries to achieve separations. The variations can include separation based on size and charge differences between analytes (termed capillary zone electrophoresis (CZE) or free solution CE (FSCE)), separation of neutral compounds using surfactant micelles (micellar electrokinetic capillary chromatography (MECC) or sometimes referred to as MEKC) sieving of solutes through a gel network (capillary gel electrophoresis, GCE), separation of cations (or anions) based on electrophoretic mobility (capillary isotachophoresis, CITP), and separation of zwitterionic solutes within a pH gradient (capillary isoelectric focusing, CLEF). Capillary electrochromatography (CEC) can be an associated electrokinetic separation technique which involves applying voltages across capillaries filled with silica gel stationary phases. Separation selectivity in CEC can be a combination of both electrophoretic and chromatographic processes. Many of the CE separation techniques can rely on the presence of an electrically induced flow of solution (electroosmotic flow, EOF) within the capillary to pump solutes towards the detector.

Arrays

The invention can include arrays for detecting a biological substance in a biological sample. Arrays can involve performing parallel analysis of multiple samples against known protein targets. The development of various microarray platforms can enable and accelerate the determination of protein abundance, localization, and interactions in a cell or tissue. Microarrays can provide a platform that allows identification of protein interaction or function against a characterized set of proteins, antibodies, or peptides. Protein-based chips can array proteins on a small surface and can directly measure the levels of proteins in tissues using fluorescence-based imaging. Proteins can be arrayed on either flat solid phases or in capillary systems (microfluidic arrays), and several different proteins can be applied to these arrays. In addition to the use of antibodies as array probes, single-stranded oligonucleotides, whose specificity can be optimized by in vitro elution (aptamers), offer a viable alternative. Nonspecific protein stains can be then used to detect bound proteins.

Arrays can include, but are not limited to, bead arrays, bead based arrays, bioarrays, bioelectronic arrays, cDNA arrays, cell arrays, DNA arrays, gene arrays, gene expression arrays, frozen cell arrays, genome arrays, high density oligonucleotide arrays, hybridization arrays, microcantilever arrays, microelectronic arrays, multiplex DNA hybridization arrays, nanoarrays, oligonucleotide arrays, oligosaccharide arrays, planar arrays, protein arrays, solution arrays, spotted arrays, tissue arrays, exon arrays, filter arrays, macroarrays, small molecule microarrays, suspension arrays, theme arrays, tiling arrays, and transcript arrays.

Sensors

The invention can include sensors for detecting a biological substance in a biological sample. Sensors can be used for both in vivo and in vitro detection. Sensors can be chemical sensors, optical sensors, and biosensors. Chemical sensors can be miniaturized analytical devices which may deliver real-time and online information on the presence of specific compounds or ions in complex samples. Optical sensors can be based on measurement of either intrinsic optical properties of analytes, or of optical properties of indicator dyes or labeled biomolecules attached to solid supports. Biosensors can be affinity biosensor based on capabilities of enzymes to convert substrates into products or catalytic biosensors. Biosensors can detect antibody and analyte complexes using a variety of physical methods. Some biosensors can measure the change in surface charge that occurs when analyte is bound to antibodies or other binding agents, which in turn can be bound to a surface. Other biosensors can use binding agents attached to a surface and measure a change in a physical property of the support, other than surface charge, upon binding of analyte. Some biosensor techniques can use a specific property of a labeled binding agent or antigen to produce a measurable change.

Methods for Identifying Proteins from a Library Screen

Protein identification methods by way of example only can include low-throughput sequencing through Edman degradation, mass spectrometry techniques, peptide mass fingerprinting, de novo sequencing, and antibody-based assays. The protein quantification assays can include fluorescent dye gel staining, tagging or chemical modification methods (i.e., isotope-coded affinity tags (ICATS), combined fractional diagonal chromatography (COFRADIC)). The purified protein may also be used for determination of three-dimensional crystal structure, which can be used for modeling intermolecular interactions. Common methods for determining three-dimensional crystal structure can include x-ray crystallography and NMR spectroscopy. Detailed below can be a few of the methods for identifying proteins in the present invention.

Protein Sequencing:

N-terminal sequencing can aid in the identification of unknown proteins, can confirm recombinant protein identity and fidelity (reading frame, translation start point, etc.), can aid the interpretation of NMR and crystallographic data, can demonstrate degrees of identity between proteins, or can provide data for the design of synthetic peptides for antibody generation, etc. N-terminal sequencing can utilize the Edman degradative chemistry, sequentially removing amino acid residues from the N-terminus of the protein and identifying them by reverse-phase HPLC. Sensitivity can be at the level of 100s femtomoles and long sequence reads (20-40 residues) can often be obtained from a few 10s picomoles of starting material. Pure proteins (>90%) can generate easily interpreted data, but insufficiently purified protein mixtures may also provide useful data, subject to rigorous data interpretation. N-terminally modified (especially acetylated) proteins cannot be sequenced directly, as the absence of a free primary amino-group prevents the Edman chemistry. However, limited proteolysis of the blocked protein (e.g., using cyanogen bromide) may allow a mixture of amino acids to be generated in each cycle of the instrument, which can be subjected to database analysis in order to interpret meaningful sequence information. C-terminal sequencing can be a post-translational modification, affecting the structure and activity of a protein. Various disease situations can be associated with impaired protein processing and C-terminal sequencing provides an additional tool for the investigation of protein structure and processing mechanisms.

Proteome Analyses:

Proteomics can be identified primarily by computer search algorithms that assign sequences to a set of empirically acquired mass/intensity data which can be generated from conducting electrospray ionization (ESI), matrix-assisted laser desorption/ionization (MALDI-TOF), or three-dimensional quadrupole ion traps on the protein of interest.

Methods for Detecting Chromosomal Abnormalities

Invasive Prenatal/in Utero:

Invasive prenatal testing for chromosomal abnormalities can be performed by any number of procedures. For example, methods such as amniocentesis, chorionic villus sampling, embryoscopy, fetoscopy, and/or percutaneous umbilical cord blood sampling, can be used to isolate the DNA and/or chromosomes of fetuses and/or embryos to be tested. Typically, after samples are extracted, the DNA and/or chromosomes can be subjected to genetic testing. Genetic testing can include, e.g., karyotyping, flow cytometry based methods, any method utilizing fluorescence hybridization (e.g., FISH), methods utilizing radioactivity, Comparative Genomic Hybridization (CGH) and/or PCR-based methods.

Non-Invasive Prenatal/in Utero:

Non-invasive prenatal testing for chromosomal abnormalities can be performed by any number of procedures. For example, methods can include analyzing: fetal cells in maternal blood, cell-free fetal DNA in maternal blood, preimplantation genetic diagnosis, external examination, ultrasound detection, fetal heartbeat, non-stress test, transcervical retrieval of trophoblast cells, and maternal serum screening. For example, kits that utilize cell free fetal DNA extracted from the maternal blood can be used. Kits such as Harmony Prenatal Test (Ariosa), MaterniT (Sequenom), MaterniT21 (Sequenom), and Panorama (Natera) can be used.

Post Birth:

Post-birth diagnostics testing for chromosomal abnormalities can be performed by any number of procedures. For example, methods such that described above for invasive prenatal procedures may be used, including but not limited to karyotyping.

### Karyotype

Karyotyping is a process of visualizing chromosomes (or parts thereof). Karyotypes can be performed on, e.g., blood cells, fetal skin cells (from amniotic fluid or the placenta) and/or bone marrow cells.

The following is an example of how a karyotype can be carried out. However, variations in the method may be performed by a person of skill in the art.

Karyotypes can be performed by first collecting a sample. For example, in newborns, a blood sample which contains red bloods cells, white blood cells, serum and other fluids can be collected. A karyotype will be done on the white blood cells which can be actively dividing (a state known as mitosis). If a karyotype is performed on a fetus during pregnancy, the sample can be on, e.g., amniotic fluid collected during an amniocentesis or a piece of the placenta collected during a chorionic villi sampling test (CVS). For example, the amniotic fluid can contain fetal skin cells which can be used to generate a karyotype.

The cells can then be cultured by any known method, in order to have enough cells to analyze. This culturing process can proceed several days, e.g., 1 to 14 days. Cells can then be treated with a chemical to halt the cell cycle and arrest the cells in metaphase. The cells can be then lysed and washed. The chromosomes can then be stained with a dye, for example, Giemsa dye, to visualize the chromosomes. Giemsa dye can stains regions of chromosomes that can be rich in the bases adenine (A) and thymine (T). When stained, the chromosomes can look like strings with light and dark bands. Each chromosome can have a specific pattern of light and dark bands which enables one of skill in the art to distinguish one chromosome from another.

Once chromosomes are stained, the chromosomes (e.g., on a slide) can be put under the microscope and analyzed. A picture can be taken of the chromosomes and at the end of the analysis, the total number of chromosomes will thus be known and there can be a picture of the chromosomes arranged by size.

To detect trisomy, monosomy, and/or polyploidy, the chromosomes can be counted. Any number above or below 46 chromosomes, can indicate chromosomal abnormalities.

Chromosomal translocations and/or chromosomal deletions can be detected by looking at the structure of the specific chromosomes to make sure that there is no missing or additional material (as sometimes additional or missing materials can lead to symptoms associated more typical chromosomal abnormalities, such as, but not limited to, trisomy 18 and 21). This can be done by comparing the size and location of G-bands and/or by comparing the chromosomes themselves.

### Diagnosis

Generally, the compositions and methods of this disclosure can provide for the diagnosis or treatment of one or more symptoms associated with chromosomal abnormalities by detecting one or more members of the hedgehog signaling pathway in one or more biological samples.

### Examples of Biological Substances

Various substances that can be analyzed and/or measured by the methods disclosed herein can include, by way of example only, polynucleotides (e.g., DNA and RNA), proteins, carbohydrates, lipids, hormones (e.g., leptin, ghrelin) in control of appetite, cholesterol and other lipids and lipid carrying proteins in control of lipid metabolism, growth factors (e.g., hepatic growth factor, granulocyte colony growth factor, brain derived neurotrophic factor), and antibodies, liver enzymes (SGOT, SGPT) therapeutic and recreational drugs of abuse, trace metals [either excess as in toxicity (e.g., lead, mercury, arsenic) or in deficiency diseases involving zinc, copper, magnesium] and most other substances found in plasma, erythrocytes, urine, saliva, and perspiration. Each metabolite in nasal mucus may reflect both physiological and pathological changes in human body metabolism specific to each metabolite and may reflect the manner in which nasal mucus provides information both on human body metabolism such as provided by plasma, erythrocytes, urine, saliva, and perspiration or information relatively unique to nasal mucus.

Biological substances can comprise one or more members of the hedgehog signaling pathway, e.g., SHH, DHH, and IHH.

The methods herein can be used to evaluate the efficacy of treatments over time. For example, biological samples can be obtained from a patient over a period of time as the patient is undergoing treatment. The biological substances from the different samples can be compared to each other to determine the efficacy of the treatment. Also, the methods described herein can be used to compare the efficacies of different therapies and/or responses to one or more treatments in different populations (e.g., different age groups, ethnicities, family histories.).

### General Methods for Diagnosis

The members of the hedgehog signaling pathway (SHH, DHH, and/or IHH, and/or a combination thereof) may be decreased or to levels below an assay detectable limit (e.g., as determined by ELISA). In some cases the hedgehog member can be SHH, DHH, IHH, or any combination thereof. The threshold for determining a decrease of the level of SHH in biological fluids can vary. For example, SHH levels can be or about: 0 pg/mL, greater than 0 pg/mL to less than less than 1 pg/mL, 1 pg/mL to 25 pg/mL, 15 pg/mL to 30 pg/mL, 20 pg/mL to 40 pg/mL; 35 pg/mL to 50 pg/mL; 45 pg/mL to 100 pg/mL; 75 pg/mL to 150 pg/mL, 125 pg/mL to 1000 pg/mL, 900 pg/mL to 2500 pg/mL, 2000 pg/mL to 5000 pg/mL, 4000 pg/mL to 7500 pg/mL, 6000 pg/mL to 10,000 pg/mL, greater than 0 pg/mL to less than 25 pg/mL, greater than 0 pg/mL to less than 30 pg/mL, greater than 0 pg/mL to less than 40 pg/mL, greater than 0 pg/mL to less than 50 pg/mL, greater than 0 pg/mL to less than 100 pg/mL, greater than 0 pg/mL to less than 150 pg/mL, greater than 0 pg/mL to less than 1000 pg/mL, greater than 0 pg/mL to less than 2500 pg/mL, greater than 0 pg/mL to less than 5000 pg/mL, greater than 0 pg/mL to less than 7500 pg/mL; greater than 0 pg/mL to less than 10,000 pg/mL, 1 pg/mL to 10,000 pg/mL, 15 pg/mL to 10,000 pg/mL, 20 pg/mL to 10,000 pg/mL, 20 pg/mL to 10,000 pg/mL, 35 pg/mL to 10,000 pg/mL, 45 pg/mL to 10,000 pg/mL, 75 pg/mL to 10,000 pg/mL, 125 pg/mL to 10,000 pg/mL, 900 pg/mL to 10,000 pg/mL, 2000 pg/mL to 10,000 pg/mL, 4000 pg/mL to 10,000 pg/mL, and 5000 pg/mL to 10,000 pg/mL. The threshold for determining a decrease of the level of DHH in biological fluids can vary, for example, DHH can be or about: 0 pg/mL, greater than 0 pg/mL to 0.1 pg/mL, 0.05 pg/mL to 0.15 pg/mL, 0.125 pg/mL to 0.2 pg/mL, 0.15 pg/mL to 0.30 pg/mL, 0.25 pg/mL to 0.5 pg/mL, 0.4 pg/mL to 0.7 pg/mL, 0.6 pg/mL to 0.75 pg/mL, 0.725 pg/mL to 0.9 pg/mL, 0.8 pg/mL to 1.0 pg/mL, 0.9 pg/mL to 1.1 pg/mL, 1.0 pg/mL to 1.3 pg/mL, 1.2 pg/mL to 1.5 pg/mL, 1.4 pg/mL to 2.0 pg/mL, 1.9 pg/mL to 2.5 pg/mL, 2.4 pg/mL to 3.0 pg/mL, 2.9 pg/mL to 3.5 pg/mL, 3.4 pg/mL to 3.8 pg/mL, 3.7 pg/mL to 3.9 pg/mL, 3.85 pg/mL to 5.0 pg/mL, less than 5.0 pg/mL, greater than 0 pg/mL to 0.15 pg/mL, greater than 0 pg/mL to 0.2 pg/mL, greater than 0 pg/mL to 0.3 pg/mL, greater than 0 pg/mL to 0.5 pg/mL, greater than 0 pg/mL to 0.7 pg/mL, greater than 0 pg/mL to 0.75 pg/mL, greater than 0 pg/mL to 0.9 pg/mL, greater than 0 pg/mL to 1.0 pg/mL, greater than 0 pg/mL to 1.1 pg/mL, greater than 0 pg/mL to 1.3 pg/mL, greater than 0 pg/mL to 1.5 pg/mL, greater than 0 pg/mL to 2.0 pg/mL, greater than 0 pg/mL to 2.5 pg/mL, greater than 0 pg/mL to 3.0 pg/mL, greater than 0 pg/mL to 3.5 pg/mL, greater than 0 pg/mL to 3.8 pg/mL, greater than 0 pg/mL to 3.9 pg/mL, greater than 0 pg/mL to 5.0 pg/mL, 0.1 pg/mL to 5.0 pg/mL, 0.05 pg/mL to 5.0 pg/mL, 0.125 pg/mL to 5.0 pg/mL, 0.2 pg/mL to 5.0 pg/mL, 0.15 pg/mL to 5.0 pg/mL, 0.25 pg/mL to 5.0 pg/mL, 0.4 pg/mL to 5.0 pg/mL, 0.6 pg/mL to 5.0 pg/mL, 0.725 pg/mL to 5.0 pg/mL, 0.8 pg/mL to 5.0 pg/mL, 0.9 pg/mL to 5.0 pg/mL, 1.0 pg/mL to 5.0 pg/mL, 1.2 pg/mL to 5.0 pg/mL, 1.4 pg/mL to 5.0 pg/mL, 1.9 pg/mL to 5.0 pg/mL, 2.4 pg/mL to 5.0 pg/mL, 2.9 pg/mL to 5.0 pg/mL, 3.4 pg/mL to 5.0 pg/mL, 3.7 pg/mL to 5.0 pg/mL, 3.85 pg/mL to 5.0 pg/mL, and 4.0 pg/mL to 5.0 pg/mL. The threshold for determining a decrease of the level of IHH in biological fluids can vary, for example, IHH can be or about: 0 pg/mL, greater than 0 pg/mL to 0.1 pg/mL, 0.05 pg/mL to 0.15 pg/mL, 0.125 pg/mL to 0.2 pg/mL, 0.15 pg/mL to 0.30 pg/mL, 0.25 pg/mL to 0.5 pg/mL, 0.4 pg/mL to 0.7 pg/mL, 0.6 pg/mL to 0.75 pg/mL, 0.725 pg/mL to 0.9 pg/mL, 0.8 pg/mL to 1.0 pg/mL, less than 1.0 pg/mL, greater than 0 pg/mL to 0.15 pg/mL, greater than 0 pg/mL to 0.2 pg/mL, greater than 0 pg/mL to 0.3 pg/mL, greater than 0 pg/mL to 0.5 pg/mL, greater than 0 pg/mL to 0.7 pg/mL, greater than 0 pg/mL to 0.75 pg/mL, greater than 0 pg/mL to 0.9 pg/mL, greater than 0 pg/mL to 1.0 pg/mL, 0.05 pg/mL to 1.0 pg/mL, 0.125 pg/mL to 1.0 pg/mL, 0.15 pg/mL to 1.0 pg/mL, 0.25 pg/mL to 1.0 pg/mL, 0.4 pg/mL to 1.0 pg/mL, 0.6 pg/mL to 1.0 pg/mL, 0.725 pg/mL to 1.0 pg/mL, 0.9 pg/mL to 1.0 pg/mL.

Measurements or testing of one or more biological substances may be compared to e.g., thresholds, and/or may be compared to level or amounts of other biological substances. For example, in some cases, the threshold level can be an estimated average of the entire normal population, e.g., having no chromosomal abnormalities or, e.g., in some cases before treatment of a drug. In some cases, the threshold level can be the single measurement or average measurement of a particular individual. For instance, in some cases, the level of one or more biological substances can be measured prior to any treatment. This measurement can be done one or more times. This can be then set as the threshold level.

Methods for Diagnosis, Evaluation, and/or Treatment

The Hedgehog signaling pathway can be a key regulator of animal development, particularly during late stages of embryogenesis and metamorphosis. Mammals are known to have, e.g., three members of the hedgehog signaling pathway, Sonic Hedgehog (SHH) (SEQ ID NOs. 1-3), Desert Hedgehog (DHH) (SEQ ID NO. 4-6), and Indian hedgehog (IHH) (SEQ ID NO. 7-9). Antibodies that bind to SHH, DHH, and IHH, can be commercially purchased or made by conventional methods. For example, SHH antibodies are available through R&D systems or LifeSpan BioSciences, Inc. (SHH: http://www.rndsystems.com/product_results.aspx?m=2109; DHH: http://www.lsbio.com/antibodies/anti-desert-hedgehog-antibody-dhh-antibody-aa194-223-rabbit-anti-human-polyclonal-for-western-blot-ls-c159454/1667741; IHH: http://www.rndsystems.com/product_results.aspx?k=Indian%20Hedgehog%20Antibody). Probes that bind hedgehog nucleic acid sequences can be made using PCR-based methods. However, the role of members of the hedgehog signaling pathway in diagnosing, treating, and/or ameliorating one or more symptoms associated with chromosomal abnormalities is not known.

In one aspect, disclosed herein are methods of diagnosing chromosomal abnormalities in a subject or subject in need thereof, the methods comprising (a) obtaining one or more biological samples from the subject; (b) measuring a level of one or more members of the hedgehog signaling pathway in one or more biological samples from the subject; and (c) diagnosing the subject with chromosomal abnormalities based upon the level of one or more members of the hedgehog signaling pathway that can be lower than a threshold level, wherein threshold level can be the estimated average of the entire normal population, e.g., having no chromosomal abnormalities. Disclosed herein are also methods of evaluating the improvement in, decline in, and/or no change one of more symptoms associated with chromosomal abnormalities in a subject or subject in need thereof, the methods comprising (a) treating the subject with one or more drugs; (b) obtaining one or more biological samples from the subject; (c) measuring a level of one or more members of the hedgehog signaling pathway in one or more biological samples from the subject; and (d) diagnosing the subject with an improvement in, decrease in, and/or no change in, a symptom of chromosomal abnormalities based upon the level of one or more members of the hedgehog signaling pathway that can be above, lower, and/or the same, than a threshold level, wherein said threshold can be the estimated average of the entire a normal population or sample/representative of a normal population, e.g., having no chromosomal abnormalities, or the level of one or more members of the hedgehog signaling pathway prior to treatment with the drug. The threshold level can be an average level for the one or more members of the hedgehog signaling pathway as measured in a control population comprising subjects with no chromosomal abnormalities (e.g., has a normal karyotype). The level of one or more members of the hedgehog signaling pathway can be at least one order of magnitude lower, e.g., 100%, than said threshold level, wherein in some cases, said threshold level can be an estimated average of the entire normal population, e.g., having no chromosomal abnormalities. For example, the drugs that can be used in this method can be theophylline, forskolin, riociguat, selective and non-selective PDE inhibitors (e.g., cilastazol, roflumilast, and/or papaverine), and other drugs mentioned throughout this application. The methods of this invention can further comprise at least one of: (a) treating the subject diagnosed chromosomal abnormalities and/or one or more symptoms associated with chromosomal abnormalities; (b) transferring the diagnosed result via a communication medium; and (c) computer implementing the diagnosis.

In one aspect, disclosed herein are methods of diagnosing chromosomal abnormalities in a subject or subject in need thereof, the methods comprising (a) obtaining one or more biological samples from the subject; (b) performing a test to determine whether the individual has one or more chromosomal abnormalities; and (c) diagnosing the subject with one or more chromosomal abnormalities. For example, the one or more biological sample can be obtained from the subject or subject in need thereof in utero and/or post birth. The test can be any test, for example, karyotypes and/or other nucleic acid based tests. If the tests are positive, then the subject can be diagnosed with one or more chromosomal abnormalities. In some cases, (a) may not be needed. For example, a subject or subject in need thereof, can be diagnosed with one or more chromosomal abnormalities by simply looking for one or more physical features, e.g., stunted growth, umbilical hernia, increased skin on the neck, low muscle tone, narrow roof of mouth, flat head, flexible ligaments, large tongue, abnormal outer ears, flattened nose, separation of first and second toes, abnormal teeth, slanted eyes, shortened hands, short neck, obstructive sleep apnea, bent fifth finger tip, brushfield spots in the iris, cataracts, keratonconus, glaucoma, hearing problems, otitis media with effusion, poor Eustachian tube function, single transverse palmar crease, protruding tongue, congenital heart disease, strabismus, congenital hypothyroidism, diabetes, duodenal atresia, pyloric stenosis, Meckel diverticulum, imperforate anus, celiac disease, gastroesophageal reflux disease, early menopause, infertility, undescended testicles, and combinations thereof. In other examples, a subject or subject in need thereof, can be diagnosed with one or more chromosomal abnormalities by simply looking for one or more cognitive features, e.g., intellectual disability, inability to speak, mental illness, autism, depression, anxiety, epileptic seizures, and dementia. Once these features are identified, the subject or subject in need thereof can be treated with drugs, e.g., any of the drugs presented in this application.

Some patients can be diagnosed with chromosomal abnormalities and/or one or more symptoms associated with chromosomal abnormalities without comparing the levels of members of the hedgehog signaling pathway to a threshold number. Disclosed herein are methods of diagnosing chromosomal abnormalities and/or one or more symptoms associated with chromosomal abnormalities in a subject or subject in need thereof, the methods comprising obtaining one or more biological samples from the subject; measuring a level of one or more members of the hedgehog signaling pathway in one or more biological samples from the subject; and diagnosing the subject with chromosomal abnormalities and/or one or more symptoms associated with chromosomal abnormalities based on one or more of: (i) the level of Sonic Hedgehog (SHH) that can be or can be about greater than 0 pg/mL to 8,500 pg/mL; (ii) the level of Indian hedgehog (IHH) that can be or can be about greater than 0 pg/mL to 1.0 pg/mL; and (iii) the level of Desert Hedgehog (DHH) that can be or can be about greater than 0 pg/mL to 5.0 pg/mL.

In order to evaluative the improvement in, decline in, and/or no change of patients (e.g., patient response) to the treatment of drugs, for example, theophylline (e.g., nasal and/or oral), cGMP activators (e.g., riociguat), cAMP activators (e.g., forskolin), PDE-3 inhibitor (e.g., cilastazol), PDE-4 inhibitor (e.g., roflumilast), PDE-10 inhibitor (e.g., papaverine), any drug described in herein, and/or any combination thereof, the inventor has developed the methods disclosed herein. For example, a patients' and/or subjects' and/or a subjects' in need thereof, response to drugs can be determined by methods of evaluating the improvement in, decline in, and/or no change in one or more symptoms associated with chromosomal abnormalities. The methods can comprise (a) treating the subject with one or more drugs; (b) obtaining one or more biological samples from the subject; (c) measuring a level of one or more members of the hedgehog signaling pathway in one or more biological samples from the subject; and (d) diagnosing the subject with an improvement in, decrease in, and/or no change in, one or more symptoms associated with chromosomal abnormalities based upon the level of one or more members of the hedgehog signaling pathway that can be above, lower, and/or the same, than a threshold level (for example, a subject's earlier baseline levels or a population's threshold levels). In another example, if the subject responds negatively to drug treatment, the method of (a) treating the subject with one or more drugs, (b) obtaining one or more biological samples from the subject; and (c) measuring a level of one or more members of the hedgehog signaling pathway in one or more biological samples from the subject; can lead to (d) diagnosing the subject with an increase or no change in one or more symptoms associated with chromosomal abnormalities based upon the level of one or more members of the hedgehog signaling pathway that can be lower than or the same as a threshold level. In further example, if the subject responds positively to drug treatment, the method of (a) treating the subject with one or more drugs; (b) obtaining one or more biological samples from the subject; and (c) measuring a level of one or more members of the hedgehog signaling pathway in one or more biological samples from the subject; can lead to (d) diagnosing the subject with chromosomal abnormalities and/or one or more symptoms associated with chromosomal abnormalities based upon the level of one or more members of the hedgehog signaling pathway that can be higher than a threshold level. In some embodiments, (b) and (c) can be performed before and/or after (a). For example, the methods described herein can comprise evaluating the improvement in, decline in, and/or no change in one or more symptoms associated with chromosomal abnormalities in a subject or subject in need thereof. The methods can comprise (a) obtaining one or more biological samples from the subject; (b) measuring a level of one or more members of the hedgehog signaling pathway in one or more biological samples from the subject; (c) treating the subject with one or more drugs; (d) obtaining one or more biological samples from the subject; (e) measuring a level of one or more members of the hedgehog signaling pathway in one or more biological samples from the subject; and (f) diagnosing the subject with an improvement in, decrease in, and/or no change in, one or more symptoms associated with chromosomal abnormalities based upon the level of one or more members of the hedgehog signaling pathway that can be above, lower, and/or the same, than a threshold level. In another example, the methods described herein can comprise evaluating the improvement in, decline in, and/or no change in one or more symptoms associated with chromosomal abnormalities in a subject or subject in need thereof, the methods comprising (a) obtaining one or more biological samples from the subject; (b) measuring a level of one or more members of the hedgehog signaling pathway in one or more biological samples from the subject; (c) treating the subject with one or more drugs; and (d) diagnosing the subject with an improvement in, decrease in, and/or no change in, one or more symptoms associated with chromosomal abnormalities based upon the level of one or more members of the hedgehog signaling pathway that can be above, lower, and/or the same, than a threshold level. In additional embodiments, the methods can further comprise one or more of the following: (a) patients described herein can be treated with more drug(s) (e.g., increase in dosage), can be treated with less drug(s) (e.g., decrease in dosage), maintained on the same drug (e.g., same dosage), switched to a different drug (e.g., from a cAMP to a cGMP activator), and/or combinations thereof; (b) the measuring a level of one or more members of the hedgehog signaling pathway can be performed by, e.g., an antibody-based assay, for example, ELISA; (c) the diagnosis can be, e.g., computer implemented; and (d) any combination thereof.

In order to effectively measure levels of members of the hedgehog signaling pathway in subjects, one or more biological samples may be needed. As described in detail above, various methods for retrieving and preparing biological samples are known and can be used to extract and prepare biological samples for testing. Also as described above, the one or more biological samples can comprise one or more bodily fluids. The one or more bodily fluids can also comprise a whole blood sample, a serum sample, a plasma sample, a urine sample, a saliva sample, a mucus sample, a perspiration sample, or a combination thereof. The one or more bodily fluids can also comprise a mucus sample (e.g., a nasal mucus sample), a plasma sample, a serum sample, a whole blood sample, and/or a perspiration sample.

The one or more members of the hedgehog signaling pathway can be selected from a group consisting of: Sonic Hedgehog (SHH), Desert Hedgehog (DHH), and/or Indian hedgehog (IHH). Although a mammalian (e.g., human) hedgehog can be measured, it is also contemplated that a non-mammalian hedgehog can be measured.

The measuring of the level of members of the hedgehog signaling pathway can be performed by using methods in the art. Methods incorporating the use of antibodies can be useful. However, this is not to be construed as limiting the methods of measuring based on antibody tests. The measuring of the level of one or more members of the hedgehog signaling pathway can comprise using one or more antibodies that bind one or more members of the hedgehog signaling pathway. The measuring can further comprise one or more antibodies that bind one or more members of the hedgehog signaling pathway wherein the one or more antibodies can be used in an immunostain, an immunoprecipitation, an immunoelectrophoresis, an immunoblot, a western blot, zestern analysis, and/or a spectrophotometry assay. It is contemplated that the methods can also further comprise one or more antibodies that bind one or more members of the hedgehog signaling pathway wherein the one or more antibodies can be used in the spectrophotometry assay that can be an EMIT (Enzyme Multiplied Immunoassay Technique) assay or an ELISA (Enzyme Linked Immunosorbent Assay). Some examples of measuring techniques are described throughout the specification. For example, the methods can comprise using one or more techniques that can be fluorescence microscopy, a radioimmunoassay, a fluorescence immunoassay, mass spectrometry, liquid chromatography, electrophoresis, or a combination thereof. Antibodies that bind to SHH, DHH, and IHH, can be commercially purchased or made by conventional methods. For example, SHH antibodies are available through R&D systems or LifeSpan BioSciences, Inc. (SHH: http://www.rndsystems.com/product_results.aspx?m=2109; DHH: http://www.lsbio.com/antibodies/anti-desert-hedgehog-antibody-dhh-antibody-aa194-223-rabbit-anti-human-polyclonal-for-western-blot-ls-c159454/1667741; IHH: http://www.rndsystems.com/product_results.aspx?k=Indian%20Hedgehog%20Antibody).

To assess if a subject has chromosomal abnormalities and/or one or more symptoms associated with chromosomal abnormalities based on levels of one or more members of the hedgehog signaling pathway, a threshold comparison, e.g., a basal level can be used. Thus, the threshold level can be an average level for one or more members of the hedgehog signaling pathway as measured in a control population comprising subjects with no chromosomal abnormalities and/or one or more symptoms associated with chromosomal abnormalities. The threshold level can also be the level of the same individual that was previously measured. The level of one or more members of the hedgehog signaling pathway can be at least one order of magnitude lower than said threshold level.

The inventors have found that decreased levels of members of the hedgehog signaling pathway can be used to diagnose and recommend treating subjects with chromosomal abnormalities and/or one or more symptoms associated with chromosomal abnormalities. For example, the subject can be diagnosed with chromosomal abnormalities and/or one or more symptoms associated with chromosomal abnormalities based on one or more of: (a) the level of SHH that can be or about: 0 pg/mL, greater than 0 pg/mL to less than less than 1 pg/mL, 1 pg/mL to 25 pg/mL, 15 pg/mL to 30 pg/mL, 20 pg/mL to 40 pg/mL; 35 pg/mL to 50 pg/mL; 45 pg/mL to 100 pg/mL; 75 pg/mL to 150 pg/mL, 125 pg/mL to 1000 pg/mL, 900 pg/mL to 2500 pg/mL, 2000 pg/mL to 5000 pg/mL, 4000 pg/mL to 7500 pg/mL, 6000 pg/mL to 10,000 pg/mL, greater than 0 pg/mL to less than 25 pg/mL, greater than 0 pg/mL to less than 30 pg/mL, greater than 0 pg/mL to less than 40 pg/mL, greater than 0 pg/mL to less than 50 pg/mL, greater than 0 pg/mL to less than 100 pg/mL, greater than 0 pg/mL to less than 150 pg/mL, greater than 0 pg/mL to less than 1000 pg/mL, greater than 0 pg/mL to less than 2500 pg/mL, greater than 0 pg/mL to less than 5000 pg/mL, greater than 0 pg/mL to less than 7500 pg/mL; greater than 0 pg/mL to less than 10,000 pg/mL, 1 pg/mL to 10,000 pg/mL, 15 pg/mL to 10,000 pg/mL, 20 pg/mL to 10,000 pg/mL, 20 pg/mL to 10,000 pg/mL, 35 pg/mL to 10,000 pg/mL, 45 pg/mL to 10,000 pg/mL, 75 pg/mL to 10,000 pg/mL, 125 pg/mL to 10,000 pg/mL, 900 pg/mL to 10,000 pg/mL, 2000 pg/mL to 10,000 pg/mL, 4000 pg/mL to 10,000 pg/mL, and 5000 pg/mL to 10,000 pg/mL; (b) the level of IHH that can be or about: 0 pg/mL, greater than 0 pg/mL to 0.1 pg/mL, 0.05 pg/mL to 0.15 pg/mL, 0.125 pg/mL to 0.2 pg/mL, 0.15 pg/mL to 0.30 pg/mL, 0.25 pg/mL to 0.5 pg/mL, 0.4 pg/mL to 0.7 pg/mL, 0.6 pg/mL to 0.75 pg/mL, 0.725 pg/mL to 0.9 pg/mL, 0.8 pg/mL to 1.0 pg/mL, less than 1.0 pg/mL, greater than 0 pg/mL to 0.15 pg/mL, greater than 0 pg/mL to 0.2 pg/mL, greater than 0 pg/mL to 0.3 pg/mL, greater than 0 pg/mL to 0.5 pg/mL, greater than 0 pg/mL to 0.7 pg/mL, greater than 0 pg/mL to 0.75 pg/mL, greater than 0 pg/mL to 0.9 pg/mL, greater than 0 pg/mL to 1.0 pg/mL, 0.05 pg/mL to 1.0 pg/mL, 0.125 pg/mL to 1.0 pg/mL, 0.15 pg/mL to 1.0 pg/mL, 0.25 pg/mL to 1.0 pg/mL, 0.4 pg/mL to 1.0 pg/mL, 0.6 pg/mL to 1.0 pg/mL, 0.725 pg/mL to 1.0 pg/mL, 0.9 pg/mL to 1.0 pg/mL; (c) the level of DHH that can be or about: 0 pg/mL, greater than 0 pg/mL to 0.1 pg/mL, 0.05 pg/mL to 0.15 pg/mL, 0.125 pg/mL to 0.2 pg/mL, 0.15 pg/mL to 0.30 pg/mL, 0.25 pg/mL to 0.5 pg/mL, 0.4 pg/mL to 0.7 pg/mL, 0.6 pg/mL to 0.75 pg/mL, 0.725 pg/mL to 0.9 pg/mL, 0.8 pg/mL to 1.0 pg/mL, 0.9 pg/mL to 1.1 pg/mL, 1.0 pg/mL to 1.3 pg/mL, 1.2 pg/mL to 1.5 pg/mL, 1.4 pg/mL to 2.0 pg/mL, 1.9 pg/mL to 2.5 pg/mL, 2.4 pg/mL to 3.0 pg/mL, 2.9 pg/mL to 3.5 pg/mL, 3.4 pg/mL to 3.8 pg/mL, 3.7 pg/mL to 3.9 pg/mL, 3.85 pg/mL to 5.0 pg/mL, less than 5.0 pg/mL, greater than 0 pg/mL to 0.15 pg/mL, greater than 0 pg/mL to 0.2 pg/mL, greater than 0 pg/mL to 0.3 pg/mL, greater than 0 pg/mL to 0.5 pg/mL, greater than 0 pg/mL to 0.7 pg/mL, greater than 0 pg/mL to 0.75 pg/mL, greater than 0 pg/mL to 0.9 pg/mL, greater than 0 pg/mL to 1.0 pg/mL, greater than 0 pg/mL to 1.1 pg/mL, greater than 0 pg/mL to 1.3 pg/mL, greater than 0 pg/mL to 1.5 pg/mL, greater than 0 pg/mL to 2.0 pg/mL, greater than 0 pg/mL to 2.5 pg/mL, greater than 0 pg/mL to 3.0 pg/mL, greater than 0 pg/mL to 3.5 pg/mL, greater than 0 pg/mL to 3.8 pg/mL, greater than 0 pg/mL to 3.9 pg/mL, greater than 0 pg/mL to 5.0 pg/mL, 0.1 pg/mL to 5.0 pg/mL, 0.05 pg/mL to 5.0 pg/mL, 0.125 pg/mL to 5.0 pg/mL, 0.2 pg/mL to 5.0 pg/mL, 0.15 pg/mL to 5.0 pg/mL, 0.25 pg/mL to 5.0 pg/mL, 0.4 pg/mL to 5.0 pg/mL, 0.6 pg/mL to 5.0 pg/mL, 0.725 pg/mL to 5.0 pg/mL, 0.8 pg/mL to 5.0 pg/mL, 0.9 pg/mL to 5.0 pg/mL, 1.0 pg/mL to 5.0 pg/mL, 1.2 pg/mL to 5.0 pg/mL, 1.4 pg/mL to 5.0 pg/mL, 1.9 pg/mL to 5.0 pg/mL, 2.4 pg/mL to 5.0 pg/mL, 2.9 pg/mL to 5.0 pg/mL, 3.4 pg/mL to 5.0 pg/mL, 3.7 pg/mL to 5.0 pg/mL, 3.85 pg/mL to 5.0 pg/mL, and 4.0 pg/mL to 5.0 pg/mL.

The methods can be based upon 2, 3, 4, 5, 6, 7, 8, 9, 10, 11, or 12 of one or more measurements.

After diagnosing a subject with one or more symptoms associated with chromosomal abnormalities, based on one or more of the previously described diagnostics methods, subjects can be treated to ameliorate and/or cure their one or more symptoms associated with chromosomal abnormalities. The diagnostic methods can be supplemented with treatment of one or more symptoms associated with chromosomal abnormalities. The diagnostic methods can further comprise treating the one or more symptoms associated with chromosomal abnormalities in the subject diagnosed with one or more symptoms associated with chromosomal abnormalities can be a subject in need thereof.

The methods herein can further comprise at least one of: (a) treating the subject diagnosed with one or more symptoms associated with chromosomal abnormalities; (b) transferring the e.g., diagnosed, result via a communication medium; and/or (c) computer implementing the diagnosis.

The treating can comprise administering to the subject e.g., in need thereof, at least one therapeutic agent (e.g., drugs).

The at least one therapeutic agent can be a PDE inhibitor. “Phosphodiesterase inhibitor” or “PDE inhibitor” can refer to any compound that inhibits a phosphodiesterase enzyme, isozyme or allozyme. The term can refer to selective or non-selective inhibitors of cyclic guanosine 3′,5′-monophosphate phosphodiesterases (cGMP-PDE) and/or cyclic adenosine 3′,5′-monophosphate phosphodiesterases (cAMP-PDE).

Theophylline is a representative member of non-specific PDE inhibitors. See e.g., U.S. Patent Application Nos. 61/941,199 and 61/983,341, incorporated herein by reference in their entirety.

Theophylline is a methylxanthine derivative; other non-selective phosphodiesterase inhibitors in this class can include caffeine, IBMX (3-isobutyl-1-methylxanthine), aminophylline, doxophylline, cipamphylline, theobromine, pentoxifylline (oxpentifylline) and diprophylline.

Phosphodiesterase 1 (PDE1) selective inhibitors, also known as, calcium- and calmodulin-dependent phosphodiesterase inhibitors, can include eburnamenine-14-carboxylic acid ethyl ester (vinpocetine).

Phosphodiesterase 2 (PDE2) can decrease aldosterone secretion and can play an important role in the regulation of elevated intracellular concentrations of cAMP and cGMP in platelets. Several regions of the brain can express PDE2 and rat experiments indicate that inhibition of PDE2 enhances memory. PDE2 may play a role in regulation of fluid and cell extravasation during inflammatory conditions as PDE2 can be localized to microvessels, especially venous capillary and endothelial cells, but apparently not to larger vessels. PDE2 can also be a pharmacological target for pathological states such as sepsis or in more localized inflammatory responses such as thrombin-induced edema formation in the lung. PDE-2 selective inhibitors can include EHNA (erythro-9-(2-hydroxy-3-nonyl) adenine), 9-(6-phenyl-2-oxohex-3-yl)-2-(3,4-dimethoxybenzyl)-purin-6-one (PDP), and BAY 60-7750.

The phosphodiesterase 3 (PDE3) family can hydrolyze cAMP and cGMP, but in a manner suggesting that in vivo, the hydrolysis of cAMP can be inhibited by cGMP. They can also be distinguished by their ability to be activated by several phosphorylation pathways including the PKA and PI3K/PKB pathways. PDE3A can be relatively highly expressed in platelets, as well as in cardiac myocytes and oocytes. PDE3B can be a major PDE in adipose tissue, liver, and pancreas, as well as in several cardiovascular tissues. Both PDE3A and PDE3B can be highly expressed in vascular smooth muscle cells and may likely modulate contraction.

PDE3-selective inhibitors can include enoximone, milrinone (Primacor), amrinone, cilostamide, cilostazol (Pletal) and trequinsin.

Phosphodiesterase 4 (PDE4) inhibitors can effectively suppress release of inflammatory mediators (e.g., cytokines) and can inhibit the production of reactive oxygen species and immune cell infiltration. PDE4-selective inhibitors can include mesembrine; rolipram; Ibudilast; and roflumilast (Daxas) and cilomilast (Airflo).

Phosphodiesterase 5 (PDE5) can regulate vascular smooth muscle contraction and can be the molecular target for drugs that can be used to treat erectile dysfunction and/or pulmonary hypertension. In the lung, inhibition of PDE5 can oppose smooth muscle vasoconstriction. PDE5 inhibitors can be used to treat pulmonary hypertension.

Phosphodiesterase 5 (PDE5)-selective inhibitors can include sildenafil, tadalafil, vardenafil, udenafil and avanafil.

Phosphodiesterase inhibitors can include, for example, filaminast, piclamilast, rolipram, Org 20241, MCI-154, roflumilast, toborinone, posicar, lixazinone, zaprinast, sildenafil, pyrazolopyrimidinones (such as those disclosed in WO 98/49166), motapizone, pimobendan, zardaverine, siguazodan, CI-930, EMD 53998, imazodan, saterinone, loprinone hydrochloride, 3-pyridinecarbonitrile derivatives, denbufyllene, albifylline, torbafylline, doxofylline, theophylline, pentoxofylline, nanterinone, cilostazol, cilostamide, MS 857, piroximone, milrinone, aminone, tolafentrine, dipyridamole, papaverine, E4021, thienopyrimidine derivatives (such as those disclosed in WO 98/17668), triflusal, ICOS-351, tetrahydropiperazino[1,2-b]beta-carboline-1,4-dione derivatives (such as those disclosed in U.S. Pat. No. 5,859,006, WO 97/03985 and WO 97/03675), carboline derivatives, (such as those disclosed in WO 97/43287), 2-pyrazolin-5-one derivatives (such as those disclosed in U.S. Pat. No. 5,869,516), fused pyridazine derivatives (such as those disclosed in U.S. Pat. No. 5,849,741), quinazoline derivatives (such as those disclosed in U.S. Pat. No. 5,614,627), anthranilic acid derivatives (such as those disclosed in U.S. Pat. No. 5,714,993), imidazoquinazoline derivatives (such as those disclosed in WO 96/26940), and the like. Also included can be those phosphodiesterase inhibitors disclosed in WO 99/21562 and WO 99/30697. It is contemplated that at certain times, the intranasal composition does not comprise at least one or more of the following: a non-selective PDE inhibitor, a PDE-1 selective inhibitor, a PDE-2 selective inhibitor, a PDE-3 selective inhibitor, a PDE-4 selective inhibitor, a PDE-5 selective inhibitor, a PDE-10 selective inhibitor, or a combination thereof. In some embodiments, the intranasal composition does not comprise at least a non-selective PDE inhibitor. In some embodiments, the intranasal composition does not comprise at least a PDE-1 selective inhibitor. In some embodiments, the intranasal composition does not comprise at least PDE-2 selective inhibitor. In some embodiments, the intranasal composition does not comprise at least a PDE-3 selective inhibitor. In some embodiments, the intranasal composition does not comprise at least a PDE-5 selective inhibitor. In some embodiments, the intranasal composition does not comprise at least a PDE-10 selective inhibitor.

Theophylline is an exemplary phosphodiesterase inhibitor that can be administered according to the methods disclosed herein. For example, 20 μg/naris of theophylline can be administered twice daily. 40 μg/naris of theophylline can also be administered once daily. 40 μg/naris of theophylline can also be administered twice daily. 80 μg/naris of theophylline can also be administered once daily. 80 μg/naris of theophylline can also be administered twice daily.

The administration of an effective amount of a phosphodiesterase inhibitor such as theophylline by intranasal administration may not produce a detectable blood level of the PDE inhibitor by using such methods as a fluorescence polarization assay (Abbott Axsym systems). Other methods for detection can include reverse phase HPLC with tandem mass spectrometric detection. See, e.g., Fox et al. (US2008/0318913). The overall level of PDE inhibition can be measured by methods known in the art. For example, methods that can be used to determine the level of PDE, measure the downstream targets of PDE. Commercial tests can also be used. For example, a phosphodiesterase assay can be used as described in Lu et al., Cell Physiology, 2012, V302:C59-C66. The administration of an effective amount of a PDE inhibitor by intranasal administration can produce blood concentrations of the PDE inhibitor that can be less than 5 mg/dl, 2 mg/dl, 1 mg/dl, 500 μg/dl, 250 μg/dl, 100 μg/dl, 50 μg/dl, 25 μg/dl, 10 μg/dl, 5 μg/dl, or 1 μg/dl.

Intranasal administration of an effective amount of a PDE inhibitor such as theophylline can ameliorate one or more symptoms associated with chromosomal abnormalities. The increase in improvement can be at least 5%, 10%, 20%, 30%, 40%, 50%, 75%, or 100% compared to the untreated state. The one or more symptoms can be increased to at least 5%, 10%, 20%, 30%, 40%, 50%, 75%, or 100% of the normal individuals. The one or more symptoms can be measured objectively, while in other embodiments the one or more symptoms can be measured subjectively. According to the NIH (www.nlm.nih.gov/medlinepluse/druginfo/meds/a681006.html) the use of PDE inhibitors such as theophylline can be associated with side effects such as upset stomach, stomach pain, diarrhea, headache, restlessness, insomnia, irritability, vomiting, increased or rapid heart rate, irregular heartbeat, seizures, and/or skin rash. Intranasal administration of PDE inhibitors such as theophylline can cause fewer side effects than other routes of administration. Intranasal administration of PDE inhibitors such as theophylline can cause less severe or no side effects when compared to other routes of administration.

Phosphodiesterase inhibitors such as theophylline can be administered alone or in combination with one or more other active ingredients; for example, 1, 2, 3, 4, 5, 6, 7, 8, 9, 10 or more other active ingredients, such as any drug disclosed herein.

The at least one PDE inhibitor can be a non-selective PDE inhibitor, a PDE-1 selective inhibitor, a PDE-2 selective inhibitor, a PDE-3 selective inhibitor, a PDE-4 selective inhibitor, a PDE-5 selective inhibitor, a PDE-10 selective inhibitor, or a combination thereof. The at least one PDE inhibitor can be a non-selective PDE inhibitor that can be a methylxanthine derivative. The methylxanthine derivative can be caffeine, theophylline, doxophylline, cipamphylline, neuphylline, pentoxiphylline, or diprophylline. The methylxanthine derivative can be theophylline. The PDE 1 inhibitor can be vinpocetine. The PDE 2 inhibitor can be EHNA. The PDE 3 inhibitor can be inamrinone, anagrelide, cilostazol, or cilostamide. The PDE 4 inhibitor can be mesembrine, rolipram, ibudilast, piclamilast, luteolin, drotaverine, or roflumilast. The PDE 5 inhibitor can be sildenafil, tadalafil, vardenafil, udenafil, avanafil, or dipyridamole. The PDE 10 inhibitor can be papaverine, OMS824 (from Omeros Corporation), and/or PF-2545920 (from Pfizer). A combination of one or more PDE inhibitors, as described herein, may be used.

Forskolin is a labdane diterpene that can be produced by the plant Coleus forskohlii. Forskolin can be used to raise levels of cAMP levels. The mechanism can comprise activating adenylyl cyclase.

Riociguat, also known as BAY 63-2521, can be used as a guanylate cyclase (sGC) activator.

The methods described herein can further comprise treating the subject, i.e., in need thereof, with at least one therapeutic agent, wherein the at least one therapeutic agent can be a non-specific PDE inhibitor, forskolin, riociguat, and/or combinations thereof. For example, the at least one therapeutic agent can be theophylline. Various combinations are contemplated. By way of example, several methods are disclosed herein.

The methods can further comprise treating the subject in need thereof with at least one therapeutic agent, wherein the at least one therapeutic agent can be a specific PDE inhibitor, forskolin, and/or riociguat. For example, the specific PDE inhibitor can be cilastazol, roflumilast, papaverine, or combinations thereof.

The methods can further comprise treating the subject in need thereof with at least one therapeutic agent, wherein the at least one therapeutic agent can be a non-specific PDE inhibitor, theophylline, and/or riociguat.

The methods can further comprise treating the subject in need thereof with at least one therapeutic agent, wherein the at least one therapeutic agent can comprise a specific PDE inhibitor, theophylline, and/or riociguat. For example, the specific PDE inhibitor can be cilastazol, roflumilast, papaverine, or combinations thereof.

The methods can further comprise treating the subject in need thereof with at least one therapeutic agent, wherein the at least one therapeutic agent can comprise a non-specific PDE inhibitor and/or riociguat.

The methods can further comprise treating the subject in need thereof with at least one therapeutic agent, wherein the at least one therapeutic agent can comprise a specific PDE inhibitor and/or riociguat. For example, the specific PDE inhibitor can be cilastazol, roflumilast, papaverine, or combinations thereof.

The methods can further comprise treating the subject in need thereof with at least one therapeutic agent, wherein the at least one therapeutic agent can comprise theophylline and/or riociguat.

The methods can further comprise at least one therapeutic agent, wherein the at least one therapeutic agent can comprise forskolin and/or riociguat.

The methods can further comprise at least one therapeutic agent, wherein the at least one therapeutic agent can comprise theophylline, forskolin, and/or riociguat.

The methods can further comprise at least one therapeutic agent, wherein the at least one therapeutic agent can comprise riociguat.

The methods can further comprise at least one therapeutic agent, wherein the at least one therapeutic agent can comprise theophylline.

The methods can further comprise at least one therapeutic agent, wherein the at least one therapeutic agent can comprise forskolin.

The methods can further comprise at least one therapeutic agent, wherein the at least one therapeutic agent can comprise a non-specific PDE inhibitor.

The methods can further comprise at least one therapeutic agent, wherein the at least one therapeutic agent can comprise a specific PDE inhibitor. For example, the specific PDE inhibitor can be cilastazol, roflumilast, papaverine, or combinations thereof.

Representative daily intranasal, lingual or pulmonary dosages can be, for example, from about 1.0 μg and 2000 mg per day, from about 1.0 μg and 500.0 mg per day, from about 10 μg and 100.0 mg per day, from about 10 μg and about 10 mg per day, from about 10 μg and 1.0 mg per day, from about 10 μg and 500 μg per day or from about 1 μg and 50 μg per day of the active ingredient comprising a preferred compound. These ranges of dosage amounts represent total dosage amounts of the active ingredient per day for a given patient. In some embodiments, the daily administered dose can be less than 2000 mg per day, 1000 mg per day, 500 mg per day, 100 mg per day, 10 mg per day, 1.0 mg per day, 500 μg per day, 300 μg per day, 200 μg per day, 100 μg per day or 50 μg per day. In other embodiments, the daily administered dose can be at least 2000 mg per day, 1000 mg per day, 500 mg per day, 100 mg per day, 10 mg per day, 1.0 mg per day, 500 μg per day, 300 μg per day, 200 μg per day, 100 μg per day or 50 μg per day. In some embodiments, on a per kilo basis, suitable dosage levels of the compounds will be from about 0.001 μg/kg and about 10.0 mg/kg of body weight per day, from about 0.5 μg/kg and about 0.5 mg/kg of body weight per day, from about 1.0 μg/kg and about 100 μg/kg of body weight per day, and from about 2.0 μg/kg and about 50 μg/kg of body weight per day of the active ingredient. In other embodiments, the suitable dosage level on a per kilo basis can be less than 10.0 mg/kg of body weight per day, 1 mg/kg of body weight per day, 500 μg/kg of body weight per day, 100 μg/kg of body weight per day, 10 μg/kg of body weight per day of the active ingredient, or 1.0 μg/kg of body weight per day of active ingredient. In further embodiments, the suitable dosage level on a per kilo basis can be at least 10.0 mg/kg of body weight per day, 1 mg/kg of body weight per day, 500 μg/kg of body weight per day, 100 μg/kg of body weight per day, 10 μg/kg of body weight per day of the active ingredient, or 1.0 μg/kg of body weight per day of active ingredient.

In some embodiments, the individual or single intranasal, lingual and/or pulmonary dose of the PDE inhibitors can be less than 10 mg, less than 2 mg, less than 1 mg, less than 500 μg, less than 200 μg, less than 100 μg, or less than 50 μg per dosage unit or application. In other embodiments, the individual or single intranasal, lingual and/or pulmonary dose of the PDE inhibitors can be at least 10 mg, 1 mg, 500 μg, 200 μg, 100 μg, 50 μg per dosage unit or application. In further embodiments, the individual or single intranasal, lingual and/or pulmonary dose of the PDE inhibitors ranges from 1 μg to 10 mg, 10μ to 1 mg, 10 μg to 500 μg, 10 μg to 250 μg, 10 μg to 200 μg, 10 μg to 100 μg, 10 μg to 50 μg, 25 μg to 100 μg, 25 μg to 250 μg, 50 μg to 500 μg, or 100 μg to 1.0 mg

The number of times per day that a dose can be administered will depend upon such pharmacological and pharmacokinetic factors as the half-life of the active ingredient, which reflects its rate of catabolism and clearance, as well as the minimal and optimal blood plasma or other body fluid levels of said active ingredient attained in the patient which can be required for therapeutic efficacy. Typically, the PDE inhibitors can be given once, twice, trice, or four times daily. PDE inhibitors may also be administered on a less frequent basis, such as every other day, every three, four, five, six or seven days.

Other factors may also be considered in deciding upon the number of doses per day and the amount of active ingredient per dose to be administered. Not the least important of such other factors can be the individual response of the patient being treated. Thus, for example, where the active ingredient is used to treat or prevent asthma, and is administered loco-regionally via aerosol inhalation into the lungs, from one to four doses consisting of actuations of a dispensing device, e.g., “puffs” of an inhaler, may be administered each day, with each dose containing from about 10.0 μg to about 10.0 mg of active ingredient.

Effective doses can be extrapolated from dose-response curves derived from in vitro or animal model test systems and can be in the same ranges or less than as described for the commercially available compounds in the Physician's Desk Reference (49th Ed.).

Riociguat can be used to effectively treat one or more symptoms associated with chromosomal abnormalities. In particular, effective dosages of riociguat can differ from high to low levels. Riociguat can be given, e.g., intranasally, and/or present in a positive amount selected from a group consisting of: greater than 0.0 μg to 1 μg, 0.5 μg to 2 μg, 1.5 μg to 3.0 μg, 2.5 μg to 10 μg, 5 μg to 15 μg, 12.5 μg to 30 μg, 25 μg to 50 μg, 40 μg to 80 μg, 60 μg to 100 μg, 90 μg to 120 μg, 110 μg to 130 μg, 125 μg to 150 μg, 140 μg to 180 μg, 170 μg to 200 μg, 200 μg to 230 μg, 215 μg to 240 μg, 235 μg to less than 250 μg, and less than 250 μg, and greater than about 0.0 μg to about 1 μg, about 0.5 μg to about 2 μg, about 1.5 μg to about 3.0 μg, about 2.5 μg to about 10 μg, about 5 μg to about 15 μg, about 12.5 μg to about 30 μg, about 25 μg to about 50 μg, about 40 μg to about 80 μg, about 60 μg to about 100 μg, about 90 μg to about 120 μg, about 110 μg to about 130 μg, about 125 μg to about 150 μg, about 140 μg to about 180 μg, about 170 μg to about 200 μg, about 200 μg to about 230 μg, about 215 μg to about 240 μg, and about 235 μg to less than 250 μg greater than 0.0 μg to 2 μg, greater than 0.0 μg to 3 μg, greater than 0.0 μg to 10 μg, greater than 0.0 μg to 15 μg, greater than 0.0 μg to 30 μg, greater than 0.0 μg to 50 μg, greater than 0.0 μg to 80 μg, greater than 0.0 μg to 100 μg, greater than 0.0 μg to 120 μg, greater than 0.0 μg to 130 μg, greater than 0.0 μg to 150 μg, greater than 0.0 μg to 180 μg, greater than 0.0 μg to 200 μg, greater than 0.0 μg to 230 μg, greater than 0.0 μg to 240 μg, greater than 0.0 μg to 250 μg, greater than 0.0 μg to about 2 μg, greater than 0.0 μg to about 3 μg, greater than 0.0 μg to about 10 μg, greater than 0.0 μg to about 15 μg, greater than 0.0 μg to about 30 μg, greater than 0.0 μg to about 50 μg, greater than 0.0 μg to about 80 μg, greater than 0.0 μg to about 100 μg, greater than 0.0 μg to about 120 μg, greater than 0.0 μg to about 130 μg, greater than 0.0 μg to about 150 μg, greater than 0.0 μg to about 180 μg, greater than 0.0 μg to about 200 μg, greater than 0.0 μg to about 230 μg, greater than 0.0 μg to about 240 μg, greater than 0.0 μg to about 250 μg, 0 μg to less than 250 μg, 0.5 μg to less than 250 μg, 1.5 μg to less than 250 μg, 2.5 μg to less than 250 μg, 5 μg to less than 250 μg, 12.5 μg to less than 250 μg, 25 μg to less than 250 μg, 40 μg to less than 250 μg, 60 μg to less than 250 μg, 90 μg to less than 250 μg, 110 μg to less than 250 μg, 125 μg to less than 250 μg, 140 μg to less than 250 μg, 170 μg to less than 250 μg, 200 μg to less than 250 μg, 215 μg to less than 250 μg, 0 μg to less than about 250 μg, about 0.5 μg to less than about 250 μg, about 1.5 μg to less than about 250 μg, about 2.5 μg to less than about 250 μg, about 5 μg to less than about 250 μg, about 12.5 μg to less than about 250 μg, about 25 μg to less than about 250 μg, about 40 μg to less than about 250 μg, about 60 μg to less than about 250 μg, about 90 μg to less than about 250 μg, about 110 μg to less than about 250 μg, about 125 μg to less than about 250 μg, about 140 μg to less than about 250 μg, about 170 μg to less than about 250 μg, about 200 μg to less than about 250 μg, and/or about 215 μg to less about than 250 μg.

Theophylline can be used to effectively treat one or more symptoms associated with chromosomal abnormalities. In particular, effective dosages of theophylline can differ from high to low levels. Theophylline can be given, e.g., intranasally, and/or present in a positive amount selected from a group consisting of: less than 45 mg, 30 mg, 15 mg, 10 mg, 5 mg, 1 mg, 500 μg, 250 μg, 120 μg, 80 μg, 40 μg, or 20 μg and less than about 45 mg, about 30 mg, about 15 mg, about 10 mg, about 5 mg, about 1 mg, about 500 μg, about 250 μg, about 120 μg, about 80 μg, about 40 μg, or about 20 μg. Theophylline can be also given, e.g., intranasally, and/or present in a positive amount selected from a group consisting of: greater than 0 μg to 20 μg, 10 μg to 40 μg, 30 μg to 80 μg, 70 μg to 120 μg, 100 μg to 250 μg, 200 μg to 500 μg, 400 μg to 1 mg, 900 μg to 5 mg, 4 mg to 10 mg, 9 mg to 15 mg, 14 mg to 30 mg, 25 mg to 45 mg, greater than 0 μg to about 20 μg, about 10 μg to about 40 μg, about 30 μg to about 80 μg, about 70 μg to about 120 μg, about 100 μg to about 250 μg, about 200 μg to about 500 μg, about 400 μg to about 1 mg, about 900 μg to about 5 mg, about 4 mg to about 10 mg, about 9 mg to about 15 mg, about 14 mg to about 30 mg, about 25 mg to about 45 mg, greater than 0 μg to 40 μg, greater than 0 μg to 80 μg, greater than 0 μg to 120 μg, greater than 0 μg to 250 μg, greater than 0 μg to 500 μg, greater than 0 μg to 1 mg, greater than 0 μg to 5 mg, greater than 0 μg to 10 mg, greater than 0 μg to 15 mg, greater than 0 μg to 30 mg, greater than 0 μg to 45 mg, greater than 0 μg to about 40 μg, greater than 0 μg to about 80 μg, greater than 0 μg to about 120 μg, greater than 0 μg to about 250 μg, greater than 0 μg to about 500 μg, greater than 0 μg to about 1 mg, greater than 0 μg to about 5 mg, greater than 0 μg to about 10 mg, greater than 0 μg to about 15 mg, greater than 0 μg to about 30 mg, greater than 0 μg to about 45 mg, greater than 0 μg to 45 mg, 10 μg to 45 mg, 30 μg to 45 mg, 70 μg to 45 mg, 100 μg to 45 mg, 200 μg to 45 mg, 400 μg to 45 mg, 900 μg to 45 mg, 4 mg to 45 mg, 9 mg to 45 mg, 14 mg to 45 mg, 35 mg to 45 mg, greater than 0 μg to about 45 mg, about 10 μg to about 45 mg, about 30 μg to about 45 mg, about 70 μg to about 45 mg, about 100 μg to about 45 mg, about 200 μg to about 45 mg, about 400 μg to about 45 mg, about 900 μg to about 45 mg, about 4 mg to about 5 mg, about 9 mg to about 45 mg, about 14 mg to about 45 mg, and/or about 35 mg to about 45 mg.

Cilastazol can be used to effectively treat one or more symptoms associated with chromosomal abnormalities. In particular, effective dosages of cilastazol can differ from high to low levels. Cilastazol can be given, e.g., intranasally, and/or present in a positive amount selected from a group consisting of: less than 500 mg to 450 mg, 475 mg to 425 mg, 435 mg to 400 mg, 415 mg to 300 mg, 325 mg to 250 mg, 275 mg to 150 mg, 200 mg to 100 mg, 135 mg to 80 mg, 95 mg to 65 mg, 75 mg to 50 mg, 60 mg to 40 mg, 45 mg to 25 mg, 30 mg to 20 mg, 15 mg to 5 mg, 10 mg to 2.5 mg, 3.5 mg to 1 mg, 2 mg to greater than 0 mg, less than about 500 mg to about 450 mg, about 475 mg to about 425 mg, about 435 mg to about 400 mg, about 415 mg to about 300 mg, about 325 mg to about 250 mg, about 275 mg to about 150 mg, about 200 mg to about 100 mg, about 135 mg to about 80 mg, about 95 mg to about 65 mg, about 75 mg to about 50 mg, about 60 mg to about 40 mg, about 45 mg to about 25 mg, about 30 mg to about 20 mg, about 15 mg to about 5 mg, about 10 mg to about 2.5 mg, about 3.5 mg to about 1 mg, about 2 mg to greater than 0 mg, 500 mg, less than 500 mg to 450 mg, less than 500 mg to 425 mg, less than 500 mg to 400 mg, less than 500 mg to 300 mg, less than 500 mg to 250 mg, less than 500 mg to 150 mg, less than 500 mg to 100 mg, less than 500 mg to 80 mg, less than 500 mg to 65 mg, less than 500 mg to 50 mg, less than 500 mg to 40 mg, less than 500 mg to 25 mg, less than 500 mg to 20 mg, less than 500 mg to 5 mg, less than 500 mg to 2.5 mg, less than 500 mg to 1 mg, less than 500 mg to greater than 0 mg, about 500 mg, less than about 500 mg to about 450 mg, less than about 500 mg to about 425 mg, less than about 500 mg to about 400 mg, less than about 500 mg to about 300 mg, less than about 500 mg to about 250 mg, less than about 500 mg to about 150 mg, less than about 500 mg to about 100 mg, less than about 500 mg to about 80 mg, less than about 500 mg to about 65 mg, less than about 500 mg to about 50 mg, less than about 500 mg to about 40 mg, less than about 500 mg to about 25 mg, less than about 500 mg to about 20 mg, less than about 500 mg to about 5 mg, less than about 500 mg to about 2.5 mg, less than about 500 mg to about 1 mg, less than about 500 mg to greater than about 0 mg, greater than 0 mg to 450 mg, greater than 0 mg to 425 mg, greater than 0 mg to 400 mg, greater than 0 mg to 300 mg, greater than 0 mg to 250 mg, greater than 0 mg to 150 mg, greater than 0 mg to 100 mg, greater than 0 mg to 80 mg, greater than 0 mg to 65 mg, greater than 0 mg to 50 mg, greater than 0 mg to 40 mg, greater than 0 mg to 25 mg, greater than 0 mg to 20 mg, greater than 0 mg to 5 mg, greater than 0 mg to 2.5 mg, greater than 0 mg to 1 mg, greater than 0 mg to about 450 mg, greater than 0 mg to about 425 mg, greater than 0 mg to about 400 mg, greater than 0 mg to about 300 mg, greater than 0 mg to about 250 mg, greater than 0 mg to about 150 mg, greater than 0 mg to about 100 mg, greater than 0 mg to about 80 mg, greater than 0 mg to about 65 mg, greater than 0 mg to about 50 mg, greater than 0 mg to about 40 mg, greater than 0 mg to about 25 mg, greater than 0 mg to about 20 mg, greater than 0 mg to about 5 mg, greater than 0 mg to about 2.5 mg, and/or greater than 0 mg to about 1 mg.

Roflumilast can be used to effectively treat one or more symptoms associated with chromosomal abnormalities. In particular, effective dosages of roflumilast can differ from high to low levels. Roflumilast can be given, e.g., intranasally, and/or present in a positive amount selected from a group consisting of: less than 10 mg, 5 mg, 1 mg, 500 μg, 250 μg, 120 μg, 80 μg, 40 μg, or 20 μg and less than about 10 mg, about 5 mg, about 1 mg, about 500 μg, about 250 μg, about 120 μg, about 80 μg, about 40 μg, or about 20 μg. Theophylline can be also given, e.g., intranasally, and/or present in a positive amount selected from a group consisting of: greater than 0 μg to 20 μg, 10 μg to 40 μg, 30 μg to 80 μg, 70 μg to 120 μg, 100 μg to 250 μg, 200 μg to 500 μg, 400 μg to 1 mg, 900 μg to 5 mg, 4 mg to 10 mg, 9 mg to 15 mg, 14 mg to 30 mg, 25 mg to 45 mg, greater than 0 μg to about 20 μg, about 10 μg to about 40 μg, about 30 μg to about 80 μg, about 70 μg to about 120 μg, about 100 μg to about 250 μg, about 200 μg to about 500 μg, about 400 μg to about 1 mg, about 900 μg to about 5 mg, about 4 mg to about 10 mg, greater than 0 μg to 40 μg, greater than 0 μg to 80 μg, greater than 0 μg to 120 μg, greater than 0 μg to 250 μg, greater than 0 μg to 500 μg, greater than 0 μg to 1 mg, greater than 0 μg to 5 mg, greater than 0 μg to 10 mg, greater than 0 μg to 15 mg, greater than 0 μg to 30 mg, greater than 0 μg to 45 mg, greater than 0 μg to about 40 μg, greater than 0 μg to about 80 μg, greater than 0 μg to about 120 μg, greater than 0 μg to about 250 μg, greater than 0 μg to about 500 μg, greater than 0 μg to about 1 mg, greater than 0 μg to about 5 mg, greater than 0 μg to about 10 mg, greater than 0 μg to 10 mg, 10 μg to 10 mg, 30 μg to 10 mg, 70 μg to 10 mg, 100 μg to 10 mg, 200 μg to 10 mg, 400 μg to 10 mg, 900 μg to 10 mg, 4 mg to 10 mg, 9 mg to 10 mg, greater than 0 μg to about 10 mg, about 10 μg to about 10 mg, about 30 μg to about 10 mg, about 70 μg to about 10 mg, about 100 μg to about 10 mg, about 200 μg to about 10 mg, about 400 μg to about 10 mg, about 900 μg to about 10 mg, about 4 mg to about 10 mg, and/or about 9 mg to about 10.

Papaverine can be used to effectively treat one or more symptoms associated with chromosomal abnormalities. In particular, effective dosages of papaverine can differ from high to low levels. Papaverine can be given, e.g., intranasally, and/or present in a positive amount selected from a group consisting of: less than 500 mg to 450 mg, 475 mg to 425 mg, 435 mg to 400 mg, 415 mg to 300 mg, 325 mg to 250 mg, 275 mg to 150 mg, 200 mg to 100 mg, 135 mg to 80 mg, 95 mg to 65 mg, 75 mg to 50 mg, 60 mg to 40 mg, 45 mg to 25 mg, 30 mg to 20 mg, 15 mg to 5 mg, 10 mg to 2.5 mg, 3.5 mg to 1 mg, 2 mg to greater than 0 mg, less than about 500 mg to about 450 mg, about 475 mg to about 425 mg, about 435 mg to about 400 mg, about 415 mg to about 300 mg, about 325 mg to about 250 mg, about 275 mg to about 150 mg, about 200 mg to about 100 mg, about 135 mg to about 80 mg, about 95 mg to about 65 mg, about 75 mg to about 50 mg, about 60 mg to about 40 mg, about 45 mg to about 25 mg, about 30 mg to about 20 mg, about 15 mg to about 5 mg, about 10 mg to about 2.5 mg, about 3.5 mg to about 1 mg, about 2 mg to greater than 0 mg, 500 mg, less than 500 mg to 450 mg, less than 500 mg to 425 mg, less than 500 mg to 400 mg, less than 500 mg to 300 mg, less than 500 mg to 250 mg, less than 500 mg to 150 mg, less than 500 mg to 100 mg, less than 500 mg to 80 mg, less than 500 mg to 65 mg, less than 500 mg to 50 mg, less than 500 mg to 40 mg, less than 500 mg to 25 mg, less than 500 mg to 20 mg, less than 500 mg to 5 mg, less than 500 mg to 2.5 mg, less than 500 mg to 1 mg, less than 500 mg to greater than 0 mg, about 500 mg, less than about 500 mg to about 450 mg, less than about 500 mg to about 425 mg, less than about 500 mg to about 400 mg, less than about 500 mg to about 300 mg, less than about 500 mg to about 250 mg, less than about 500 mg to about 150 mg, less than about 500 mg to about 100 mg, less than about 500 mg to about 80 mg, less than about 500 mg to about 65 mg, less than about 500 mg to about 50 mg, less than about 500 mg to about 40 mg, less than about 500 mg to about 25 mg, less than about 500 mg to about 20 mg, less than about 500 mg to about 5 mg, less than about 500 mg to about 2.5 mg, less than about 500 mg to about 1 mg, less than about 500 mg to greater than about 0 mg, greater than 0 mg to 450 mg, greater than 0 mg to 425 mg, greater than 0 mg to 400 mg, greater than 0 mg to 300 mg, greater than 0 mg to 250 mg, greater than 0 mg to 150 mg, greater than 0 mg to 100 mg, greater than 0 mg to 80 mg, greater than 0 mg to 65 mg, greater than 0 mg to 50 mg, greater than 0 mg to 40 mg, greater than 0 mg to 25 mg, greater than 0 mg to 20 mg, greater than 0 mg to 5 mg, greater than 0 mg to 2.5 mg, greater than 0 mg to 1 mg, greater than 0 mg to about 450 mg, greater than 0 mg to about 425 mg, greater than 0 mg to about 400 mg, greater than 0 mg to about 300 mg, greater than 0 mg to about 250 mg, greater than 0 mg to about 150 mg, greater than 0 mg to about 100 mg, greater than 0 mg to about 80 mg, greater than 0 mg to about 65 mg, greater than 0 mg to about 50 mg, greater than 0 mg to about 40 mg, greater than 0 mg to about 25 mg, greater than 0 mg to about 20 mg, greater than 0 mg to about 5 mg, greater than 0 mg to about 2.5 mg, and/or greater than 0 mg to about 1 mg.

Forskolin can be used to effectively treat one or more symptoms associated with chromosomal abnormalities. In particular, effective dosages of forskolin can differ from high to low levels. Forskolin can be given, e.g., intranasally, and/or present in a positive amount selected from a group consisting of: less than 500 mg to 450 mg, 475 mg to 425 mg, 435 mg to 400 mg, 415 mg to 300 mg, 325 mg to 250 mg, 275 mg to 150 mg, 200 mg to 100 mg, 135 mg to 80 mg, 95 mg to 65 mg, 75 mg to 50 mg, 60 mg to 40 mg, 45 mg to 25 mg, 30 mg to 20 mg, 15 mg to 5 mg, 10 mg to 2.5 mg, 3.5 mg to 1 mg, 2 mg to greater than 0 mg, less than about 500 mg to about 450 mg, about 475 mg to about 425 mg, about 435 mg to about 400 mg, about 415 mg to about 300 mg, about 325 mg to about 250 mg, about 275 mg to about 150 mg, about 200 mg to about 100 mg, about 135 mg to about 80 mg, about 95 mg to about 65 mg, about 75 mg to about 50 mg, about 60 mg to about 40 mg, about 45 mg to about 25 mg, about 30 mg to about 20 mg, about 15 mg to about 5 mg, about 10 mg to about 2.5 mg, about 3.5 mg to about 1 mg, about 2 mg to greater than 0 mg, 500 mg, less than 500 mg to 450 mg, less than 500 mg to 425 mg, less than 500 mg to 400 mg, less than 500 mg to 300 mg, less than 500 mg to 250 mg, less than 500 mg to 150 mg, less than 500 mg to 100 mg, less than 500 mg to 80 mg, less than 500 mg to 65 mg, less than 500 mg to 50 mg, less than 500 mg to 40 mg, less than 500 mg to 25 mg, less than 500 mg to 20 mg, less than 500 mg to 5 mg, less than 500 mg to 2.5 mg, less than 500 mg to 1 mg, less than 500 mg to greater than 0 mg, about 500 mg, less than about 500 mg to about 450 mg, less than about 500 mg to about 425 mg, less than about 500 mg to about 400 mg, less than about 500 mg to about 300 mg, less than about 500 mg to about 250 mg, less than about 500 mg to about 150 mg, less than about 500 mg to about 100 mg, less than about 500 mg to about 80 mg, less than about 500 mg to about 65 mg, less than about 500 mg to about 50 mg, less than about 500 mg to about 40 mg, less than about 500 mg to about 25 mg, less than about 500 mg to about 20 mg, less than about 500 mg to about 5 mg, less than about 500 mg to about 2.5 mg, less than about 500 mg to about 1 mg, less than about 500 mg to greater than about 0 mg, greater than 0 mg to 450 mg, greater than 0 mg to 425 mg, greater than 0 mg to 400 mg, greater than 0 mg to 300 mg, greater than 0 mg to 250 mg, greater than 0 mg to 150 mg, greater than 0 mg to 100 mg, greater than 0 mg to 80 mg, greater than 0 mg to 65 mg, greater than 0 mg to 50 mg, greater than 0 mg to 40 mg, greater than 0 mg to 25 mg, greater than 0 mg to 20 mg, greater than 0 mg to 5 mg, greater than 0 mg to 2.5 mg, greater than 0 mg to 1 mg, greater than 0 mg to about 450 mg, greater than 0 mg to about 425 mg, greater than 0 mg to about 400 mg, greater than 0 mg to about 300 mg, greater than 0 mg to about 250 mg, greater than 0 mg to about 150 mg, greater than 0 mg to about 100 mg, greater than 0 mg to about 80 mg, greater than 0 mg to about 65 mg, greater than 0 mg to about 50 mg, greater than 0 mg to about 40 mg, greater than 0 mg to about 25 mg, greater than 0 mg to about 20 mg, greater than 0 mg to about 5 mg, greater than 0 mg to about 2.5 mg, and/or greater than 0 mg to about 1 mg.

The cytochrome P450 superfamily (CYP) can be a large and diverse group of enzymes that catalyze the oxidation of organic substances. CYPs are enzymes involved in drug metabolism and bioactivation. The inventors have found that by inhibiting CYPs, the effect of the therapeutic agents herein can be prolonged and have a more profound effect. This can allow for e.g., lower dosing and delivery via a multitude of different routes of administration. The inventors have also found that different routes of administration may, e.g., circumvent drug resistance.

The methods herein can further comprise treating a subject in need thereof, wherein the treating can comprise administering to a subject, an effective amount of cytochrome p450 inhibitors. The methods can further comprise administering to a subject, an effective amount of cytochrome p450 inhibitors, wherein the cytochrome p450 inhibitors can fully or partially inhibit a cytochrome selected from a group consisting of: CYP1, CYP1A1, CYP1A2, CYP1B1, CYP2, CYP2A6, CYP2A7, CYP2A13, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3, CYP3A4, CYP3A5, CYP3A7, CYP3A43, CYP4, CYP4A11, CYP4A22, CYP4B1, CYP4F2, CYP4F3, CYP4F8, CYP4F11, CYP4F12, CYP4F22, CYP4V2, CYP4X1, CYP4Z1, CYP5, CYP5A1, CYP7, CYP7A1, CYP7B1, CYP8, CYP8A1, CYP8B1, CYP11, CYP11A1, CYP11B1, CYP11B2, CYP17, CYP17A1, CYP19, CYP19A1, CYP20, CYP20A1, CYP21, CYP21A2, CYP24, CYP24A1, CYP26, CYP26A1, CYP26B1, CYP26C1, CYP27, CYP27A1, CYP27B1, CYP27C1, CYP39, CYP39A1, CYP46, CYP46A1, CYP51, and CYP51A1. The methods can also further comprise administering to a subject, an effective amount of a cytochrome p450 inhibitor, wherein the cytochrome p450 inhibitor can fully or partially inhibit CYP1. The methods can also further comprise administering to a subject, an effective amount of a cytochrome p450 inhibitor, wherein the cytochrome p450 inhibitor can fully or partially inhibit CYP1A2. The CYP1A2 inhibitor can be selected from a group consisting of: fluoroquinolone, selective serotonin reuptake inhibitor (SSRI), calcium channel blocker, herbal tea, naringenin, H2-receptor activator, antiarrhythmic agent, interferon, xanthotoxin, mibefradil, cumin, turmeric, and isoniazid. The one or more CYP1A2 inhibitors can be grapefruit juice, or a component thereof. The one or more CYP1A2 inhibitor can be naringenin.

The methods can further comprise administration of a composition or dosage unit that can be steroid-free.

Another aspect of this invention can be to restore the levels of members of the hedgehog signaling pathway to a therapeutically effective level. This can be achieved by various methods. The treatment can comprise increasing the level of one or more members of the hedgehog signaling pathway by administration of an effective amount of one or more members of the hedgehog signaling pathway. The increasing the level of one or more members of the hedgehog signaling pathway can also comprise administration of an effective amount of the one or more exogenous members of the hedgehog signaling pathway. The increasing the level of one or more members of the hedgehog signaling pathway can also comprise activating expression of an effective amount of one or more members of the hedgehog signaling pathway. The activating expression of an effective amount of one or more members of the hedgehog signaling pathway can be done by genetic manipulation of genes responsible for the expression of one or more members of the hedgehog signaling pathway. The activating expression of an effective amount of one or more members of the hedgehog signaling pathway can also be effectuated through a therapeutic agent. The treatment can directly or indirectly affect levels of one or more members of the hedgehog signaling pathway.

Some embodiments can include increasing the level of one or more hedgehog proteins by treating the patient with an isolated SHH. In some cases, the isolated SHH can be a purified natural or purified recombinant SHH. The isolated SHH can be a purified recombinant SHH, which can have at least about: 70, 75, 80, 85, 90, 91, 92, 93, 94, 95, 96, 97, 98, 99, and/or 100% homology and/or identity to SEQ ID. No. 1, 2, or 3. For example, the purified recombinant SHH can have at least about 70% homology to SEQ ID. No. 1. The purified recombinant SHH can also have at least about 70% homology to SEQ ID. No. 2. The purified recombinant SHH can also have at least about 70% homology to SEQ ID. No. 3. The purified recombinant SHH can have at least about 75% homology to SEQ ID. No. 1. The purified recombinant SHH can also have at least about 75% homology to SEQ ID. No. 2. The purified recombinant SHH can also have at least about 75% homology to SEQ ID. No. 3. The purified recombinant SHH can have at least about 80% homology to SEQ ID. No. 1. The purified recombinant SHH can also have at least about 80% homology to SEQ ID. No. 2. The purified recombinant SHH can also have at least about 80% homology to SEQ ID. No. 3. The purified recombinant SHH can have at least about 85% homology to SEQ ID. No. 1. The purified recombinant SHH can also have at least about 85% homology to SEQ ID. No. 2. The purified recombinant SHH can also have at least about 85% homology to SEQ ID. No. 3. The purified recombinant SHH can have at least about 90% homology to SEQ ID. No. 1. The purified recombinant SHH can also have at least about 90% homology to SEQ ID. No. 2. The purified recombinant SHH can also have at least about 90% homology to SEQ ID. No. 3. The purified recombinant SHH can have at least about 95% homology to SEQ ID. No. 1. The purified recombinant SHH can also have at least about 95% homology to SEQ ID. No. 2. The purified recombinant SHH can also have at least about 95% homology to SEQ ID. No. 3. The purified recombinant SHH can have 100% homology to SEQ ID. No. 1. The purified recombinant SHH can also have 100% homology to SEQ ID. No. 2. The purified recombinant SHH can also have 100% homology to SEQ ID. No. 3. The purified recombinant SHH can have at least about 70% identity to SEQ ID. No. 1. The purified recombinant SHH can also have at least about 70% identity to SEQ ID. No. 2. The purified recombinant SHH can also have at least about 70% identity to SEQ ID. No. 3. The purified recombinant SHH can have at least about 75% identity to SEQ ID. No. 1. The purified recombinant SHH can also have at least about 75% identity to SEQ ID. No. 2. The purified recombinant SHH can also have at least about 75% identity to SEQ ID. No. 3. The purified recombinant SHH can have at least about 80% identity to SEQ ID. No. 1. The purified recombinant SHH can also have at least about 80% identity to SEQ ID. No. 2. The purified recombinant SHH can also have at least about 80% identity to SEQ ID. No. 3. The purified recombinant SHH can have at least about 85% identity to SEQ ID. No. 1. The purified recombinant SHH can also have at least about 85% identity to SEQ ID. No. 2. The purified recombinant SHH can also have at least about 85% identity to SEQ ID. No. 3. The purified recombinant SHH can have at least about 90% identity to SEQ ID. No. 1. The purified recombinant SHH can also have at least about 90% identity to SEQ ID. No. 2. The purified recombinant SHH can also have at least about 90% identity to SEQ ID. No. 3. The purified recombinant SHH can have at least about 95% identity to SEQ ID. No. 1. The purified recombinant SHH can also have at least about 95% identity to SEQ ID. No. 2. The purified recombinant SHH can also have at least about 95% identity to SEQ ID. No. 3. The purified recombinant SHH can have 100% identity to SEQ ID. No. 1. The purified recombinant SHH can also have 100% identity to SEQ ID. No. 2. The purified recombinant SHH can also have 100% identity to SEQ ID. No. 3.

The purified recombinant SHH can also be a partial sequence of SEQ ID. No. 1, 2, or 3. For example, the purified recombinant SHH can be at least 5 amino acids of SEQ ID. No. 1, 2, or 3. The purified recombinant SHH can be at least 6 amino acids of SEQ ID. No. 1, 2, or 3. The purified recombinant SHH can be at least 7 amino acids of SEQ ID. No. 1, 2, or 3. The purified recombinant SHH can be at least 8 amino acids of SEQ ID. No. 1, 2, or 3. The purified recombinant SHH can be at least 9 amino acids of SEQ ID. No. 1, 2, or 3. The purified recombinant SHH can be at least 10 amino acids of SEQ ID. No. 1, 2, or 3. The purified recombinant SHH can be at least 15 amino acids of SEQ ID. No. 1, 2, or 3. The purified recombinant SHH can be at least 20 amino acids of SEQ ID. No. 1, 2, or 3. The purified recombinant SHH can be at least 25 amino acids of SEQ ID. No. 1, 2, or 3. The purified recombinant SHH can be at least 30 amino acids of SEQ ID. No. 1, 2, or 3. The purified recombinant SHH can be at least 35 amino acids of SEQ ID. No. 1, 2, or 3. The purified recombinant SHH can be at least 40 amino acids of SEQ ID. No. 1, 2, or 3. The purified recombinant SHH can be at least 45 amino acids of SEQ ID. No. 1, 2, or 3. The purified recombinant SHH can be at least 50 amino acids of SEQ ID. No. 1, 2, or 3. The purified recombinant SHH can be at least 60 amino acids of SEQ ID. No. 1, 2, or 3. The purified recombinant SHH can be at least 70 amino acids of SEQ ID. No. 1, 2, or 3. The purified recombinant SHH can be at least 80 amino acids of SEQ ID. No. 1, 2, or 3. The purified recombinant SHH can be at least 90 amino acids of SEQ ID. No. 1, 2, or 3. The purified recombinant SHH can be at least 100 amino acids of SEQ ID. No. 1, 2, or 3. The purified recombinant SHH can be 10 amino acids or more of SEQ ID. No. 1, 2, or 3.

Some embodiments can include increasing the level of one or more hedgehog proteins by treating the patient with an isolated DHH. In some cases, the isolated DHH can be a purified natural or purified recombinant DHH. The isolated DHH can be a purified recombinant DHH, which can have at least about: 70, 75, 80, 85, 90, 91, 92, 93, 94, 95, 96, 97, 98, 99, and/or 100% homology and/or identity to SEQ ID. No. 4, 5, or 6. For example, the purified recombinant DHH can have at least about 70% homology to SEQ ID. No. 4. The purified recombinant DHH can also have at least about 70% homology to SEQ ID. No. 5. The purified recombinant DHH can also have at least about 70% homology to SEQ ID. No. 6. The purified recombinant DHH can have at least about 75% homology to SEQ ID. No. 4. The purified recombinant DHH can also have at least about 75% homology to SEQ ID. No. 5. The purified recombinant DHH can also have at least about 75% homology to SEQ ID. No. 6. The purified recombinant DHH can have at least about 80% homology to SEQ ID. No. 4. The purified recombinant DHH can also have at least about 80% homology to SEQ ID. No. 5. The purified recombinant DHH can also have at least about 80% homology to SEQ ID. No. 6. The purified recombinant DHH can have at least about 85% homology to SEQ ID. No. 4. The purified recombinant DHH can also have at least about 85% homology to SEQ ID. No. 5. The purified recombinant DHH can also have at least about 85% homology to SEQ ID. No. 6. The purified recombinant DHH can have at least about 90% homology to SEQ ID. No. 4. The purified recombinant DHH can also have at least about 90% homology to SEQ ID. No. 5. The purified recombinant DHH can also have at least about 90% homology to SEQ ID. No. 6. The purified recombinant DHH can have at least about 95% homology to SEQ ID. No. 4. The purified recombinant DHH can also have at least about 95% homology to SEQ ID. No. 5. The purified recombinant DHH can also have at least about 95% homology to SEQ ID. No. 6. The purified recombinant DHH can have 100% homology to SEQ ID. No. 4. The purified recombinant DHH can also have 100% homology to SEQ ID. No. 5. The purified recombinant DHH can also have 100% homology to SEQ ID. No. 6. The purified recombinant DHH can have at least about 70% identity to SEQ ID. No. 4. The purified recombinant DHH can also have at least about 70% identity to SEQ ID. No. 5. The purified recombinant DHH can also have at least about 70% identity to SEQ ID. No. 6. The purified recombinant DHH can have at least about 75% identity to SEQ ID. No. 4. The purified recombinant DHH can also have at least about 75% identity to SEQ ID. No. 5. The purified recombinant DHH can also have at least about 75% identity to SEQ ID. No. 6. The purified recombinant DHH can have at least about 80% identity to SEQ ID. No. 4. The purified recombinant DHH can also have at least about 80% identity to SEQ ID. No. 5. The purified recombinant DHH can also have at least about 80% identity to SEQ ID. No. 6. The purified recombinant DHH can have at least about 85% identity to SEQ ID. No. 4. The purified recombinant DHH can also have at least about 85% identity to SEQ ID. No. 5. The purified recombinant DHH can also have at least about 85% identity to SEQ ID. No. 6. The purified recombinant DHH can have at least about 90% identity to SEQ ID. No. 4. The purified recombinant DHH can also have at least about 90% identity to SEQ ID. No. 5. The purified recombinant DHH can also have at least about 90% identity to SEQ ID. No. 6. The purified recombinant DHH can have at least about 95% identity to SEQ ID. No. 4. The purified recombinant DHH can also have at least about 95% identity to SEQ ID. No. 5. The purified recombinant DHH can also have at least about 95% identity to SEQ ID. No. 6. The purified recombinant DHH can have 100% identity to SEQ ID. No. 4. The purified recombinant DHH can also have 100% identity to SEQ ID. No. 5. The purified recombinant DHH can also have 100% identity to SEQ ID. No. 6.

The purified recombinant DHH can also be a partial sequence of SEQ ID. No. 4, 5, or 6. For example, the purified recombinant DHH can be at least 5 amino acids of SEQ ID. No. 4, 5, or 6. The purified recombinant DHH can be at least 6 amino acids of SEQ ID. No. 4, 5, or 6. The purified recombinant DHH can be at least 7 amino acids of SEQ ID. No. 4, 5, or 6. The purified recombinant DHH can be at least 8 amino acids of SEQ ID. No. 4, 5, or 6. The purified recombinant DHH can be at least 9 amino acids of SEQ ID. No. 4, 5, or 6. The purified recombinant DHH can be at least 10 amino acids of SEQ ID. No. 4, 5, or 6. The purified recombinant DHH can be at least 15 amino acids of SEQ ID. No. 4, 5, or 6. The purified recombinant DHH can be at least 20 amino acids of SEQ ID. No. 4, 5, or 6. The purified recombinant DHH can be at least 25 amino acids of SEQ ID. No. 4, 5, or 6. The purified recombinant DHH can be at least 30 amino acids of SEQ ID. No. 4, 5, or 6. The purified recombinant DHH can be at least 35 amino acids of SEQ ID. No. 4, 5, or 6. The purified recombinant DHH can be at least 40 amino acids of SEQ ID. No. 4, 5, or 6. The purified recombinant DHH can be at least 45 amino acids of SEQ ID. No. 4, 5, or 6. The purified recombinant DHH can be at least 50 amino acids of SEQ ID. No. 4, 5, or 6. The purified recombinant DHH can be at least 60 amino acids of SEQ ID. No. 4, 5, or 6. The purified recombinant DHH can be at least 70 amino acids of SEQ ID. No. 4, 5, or 6. The purified recombinant DHH can be at least 80 amino acids of SEQ ID. No. 4, 5, or 6. The purified recombinant DHH can be at least 90 amino acids of SEQ ID. No. 4, 5, or 6. The purified recombinant DHH can be at least 100 amino acids of SEQ ID. No. 4, 5, or 6. The purified recombinant DHH can be 10 amino acids or more of SEQ ID. No. 4, 5, or 6.

Some embodiments can include increasing the level of one or more hedgehog proteins by treating the patient with an isolated IHH. In some cases, the isolated IHH can be a purified natural or purified recombinant IHH. The isolated IHH can be a purified recombinant IHH which can have at least about: 70, 75, 80, 85, 90, 91, 92, 93, 94, 95, 96, 97, 98, 99, and/or 100% homology and/or identity to SEQ ID. No. 7, 8, or 9. For example, the purified recombinant IHH can have at least about 70% homology to SEQ ID. No. 7. The purified recombinant IHH can also have at least about 70% homology to SEQ ID. No. 8. The purified recombinant IHH can also have at least about 70% homology to SEQ ID. No. 9. The purified recombinant IHH can have at least about 75% homology to SEQ ID. No. 7. The purified recombinant IHH can also have at least about 75% homology to SEQ ID. No. 8. The purified recombinant IHH can also have at least about 75% homology to SEQ ID. No. 9. The purified recombinant IHH can have at least about 80% homology to SEQ ID. No. 7. The purified recombinant IHH can also have at least about 80% homology to SEQ ID. No. 8. The purified recombinant IHH can also have at least about 80% homology to SEQ ID. No. 9. The purified recombinant IHH can have at least about 85% homology to SEQ ID. No. 7. The purified recombinant IHH can also have at least about 85% homology to SEQ ID. No. 8. The purified recombinant IHH can also have at least about 85% homology to SEQ ID. No. 9. The purified recombinant IHH can have at least about 90% homology to SEQ ID. No. 7. The purified recombinant IHH can also have at least about 90% homology to SEQ ID. No. 8. The purified recombinant IHH can also have at least about 90% homology to SEQ ID. No. 9. The purified recombinant IHH can have at least about 95% homology to SEQ ID. No. 7. The purified recombinant IHH can also have at least about 95% homology to SEQ ID. No. 8. The purified recombinant IHH can also have at least about 95% homology to SEQ ID. No. 9. The purified recombinant IHH can have 100% homology to SEQ ID. No. 7. The purified recombinant IHH can also have 100% homology to SEQ ID. No. 8. The purified recombinant IHH can also have 100% homology to SEQ ID. No. 9. The purified recombinant IHH can have at least about 70% identity to SEQ ID. No. 7. The purified recombinant IHH can also have at least about 70% identity to SEQ ID. No. 8. The purified recombinant IHH can also have at least about 70% identity to SEQ ID. No. 9. The purified recombinant IHH can have at least about 75% identity to SEQ ID. No. 7. The purified recombinant IHH can also have at least about 75% identity to SEQ ID. No. 8. The purified recombinant IHH can also have at least about 75% identity to SEQ ID. No. 9. The purified recombinant IHH can have at least about 80% identity to SEQ ID. No. 7. The purified recombinant IHH can also have at least about 80% identity to SEQ ID. No. 8. The purified recombinant IHH can also have at least about 80% identity to SEQ ID. No. 9. The purified recombinant IHH can have at least about 85% identity to SEQ ID. No. 7. The purified recombinant IHH can also have at least about 85% identity to SEQ ID. No. 8. The purified recombinant IHH can also have at least about 85% identity to SEQ ID. No. 9. The purified recombinant IHH can have at least about 90% identity to SEQ ID. No. 7. The purified recombinant IHH can also have at least about 90% identity to SEQ ID. No. 8. The purified recombinant IHH can also have at least about 90% identity to SEQ ID. No. 9. The purified recombinant IHH can have at least about 95% identity to SEQ ID. No. 7. The purified recombinant IHH can also have at least about 95% identity to SEQ ID. No. 8. The purified recombinant IHH can also have at least about 95% identity to SEQ ID. No. 9. The purified recombinant IHH can have 100% identity to SEQ ID. No. 7. The purified recombinant IHH can also have 100% identity to SEQ ID. No. 8. The purified recombinant IHH can also have 100% identity to SEQ ID. No. 9.

The purified recombinant IHH can also be a partial sequence of SEQ ID. No. 7, 8, or 9. For example, the purified recombinant IHH can be at least 5 amino acids of SEQ ID. No. 7, 8, or 9. The purified recombinant IHH can be at least 6 amino acids of SEQ ID. No. 7, 8, or 9. The purified recombinant IHH can be at least 7 amino acids of SEQ ID. No. 7, 8, or 9. The purified recombinant IHH can be at least 8 amino acids of SEQ ID. No. 7, 8, or 9. The purified recombinant IHH can be at least 9 amino acids of SEQ ID. No. 7, 8, or 9. The purified recombinant IHH can be at least 10 amino acids of SEQ ID. No. 7, 8, or 9. The purified recombinant IHH can be at least 15 amino acids of SEQ ID. No. 7, 8, or 9. The purified recombinant IHH can be at least 20 amino acids of SEQ ID. No. 7, 8, or 9. The purified recombinant IHH can be at least 25 amino acids of SEQ ID. No. 7, 8, or 9. The purified recombinant IHH can be at least 30 amino acids of SEQ ID. No. 7, 8, or 9. The purified recombinant IHH can be at least 35 amino acids of SEQ ID. No. 7, 8, or 9. The purified recombinant IHH can be at least 40 amino acids of SEQ ID. No. 7, 8, or 9. The purified recombinant IHH can be at least 45 amino acids of SEQ ID. No. 7, 8, or 9. The purified recombinant IHH can be at least 50 amino acids of SEQ ID. No. 7, 8, or 9. The purified recombinant IHH can be at least 60 amino acids of SEQ ID. No. 7, 8, or 9. The purified recombinant IHH can be at least 70 amino acids of SEQ ID. No. 7, 8, or 9. The purified recombinant IHH can be at least 80 amino acids of SEQ ID. No. 7, 8, or 9. The purified recombinant IHH can be at least 90 amino acids of SEQ ID. No. 7, 8, or 9. The purified recombinant IHH can be at least 100 amino acids of SEQ ID. No. 7, 8, or 9. The purified recombinant IHH can be 10 amino acids or more of SEQ ID. No. 7, 8, or 9.

The methods of this invention can include different routes of administration for the one or more therapeutic agents. Known methods in the art can be used to make different formulations. For example, the one or more therapeutic agents or composition can comprise one or more therapeutic agents can be suitable for administration by a methods selected from a group consisting of: oral administration, transmucosal administration, buccal administration, inhalation administration, intranasal administration, parental administration, intravenous administration, subcutaneous administration, intramuscular administration, sublingual administration, transdermal administration, and rectal administration. Because of the ease of use, the one or more therapeutic agents or composition comprising one or more therapeutic agents can be suitable for oral administration, inhalational administration, intranasal administration, or a combination thereof. In some embodiments, the route of administration can penetrate the placental barrier and/or the blood brain barrier. For example, in some embodiments, intranasal drug administration, e.g., theophylline, can be delivered into the brain (1) directly by absorption through the cribriform plate along the olfactory bulb, (2) indirectly by absorption through blood-brain barrier receptors, or (3) through combinations of both methods. In another example, in some embodiments, pregnant mothers can be given a dose of a drug, which will then reach the fetus by crossing the placental barrier. The drug can then enter the fetus and find its way to target sites, e.g., the brain. If the drug reaches the brain, it can cross the blood/brain barrier. In some other embodiments, a breast feeding mother can be given a dose of drug, which will then reach the baby by flowing to the mother's breast milk. The breast milk containing the drug can be then fed to the baby. In some embodiments, the drug can be used to fortify baby's formula and/or milk and then fed to the baby. The drug will eventually find its way to the brain by crossing the blood/brain barrier. In some embodiments, the drug may or may not cross the blood/brain barrier.

The methods of this invention can include treating a woman when she is sexually active, is attempting to conceive, and/or actually conceives. The treating can be performed 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 weeks after conception. The treating can be performed during labor. The treating can be performed immediately after birth. In some embodiments, the treating can be performed 1 week after conception. In some embodiments, the treating can be performed 2 weeks after conception. In some embodiments, the treating can be performed 3 weeks after conception. In some embodiments, the treating can be performed 4 weeks after conception. In some embodiments, the treating can be performed 5 weeks after conception. In some embodiments, the treating can be performed 6 weeks after conception. In some embodiments, the treating can be performed 7 weeks after conception. In some embodiments, the treating can be performed 8 weeks after conception. In some embodiments, the treating can be performed 9 weeks after conception. In some embodiments, the treating can be performed 10 weeks after conception. In some embodiments, the treating can be performed 11 weeks after conception. In some embodiments, the treating can be performed 12 weeks after conception. In some embodiments, the treating can be performed 13 weeks after conception. In some embodiments, the treating can be performed 14 weeks after conception. In some embodiments, the treating can be performed 15 weeks after conception. In some embodiments, the treating can be performed 16 weeks after conception. In some embodiments, the treating can be performed 17 weeks after conception. In some embodiments, the treating can be performed 18 weeks after conception. In some embodiments, the treating can be performed 19 weeks after conception. In some embodiments, the treating can be performed 20 weeks after conception. In some embodiments, the treating can be performed 21 weeks after conception. In some embodiments, the treating can be performed 22 weeks after conception. In some embodiments, the treating can be performed 23 weeks after conception. In some embodiments, the treating can be performed 24 weeks after conception. In some embodiments, the treating can be performed 25 weeks after conception. In some embodiments, the treating can be performed 26 weeks after conception. In some embodiments, the treating can be performed 27 weeks after conception. In some embodiments, the treating can be performed 28 weeks after conception. In some embodiments, the treating can be performed 29 weeks after conception. In some embodiments, the treating can be performed 30 weeks after conception. In some embodiments, the treating can be performed 31 weeks after conception. In some embodiments, the treating can be performed 32 weeks after conception. In some embodiments, the treating can be performed 33 weeks after conception. In some embodiments, the treating can be performed 34 weeks after conception. In some embodiments, the treating can be performed 35 weeks after conception. In some embodiments, the treating can be performed 36 weeks after conception. In some embodiments, the treating can be performed 37 weeks after conception. In some embodiments, the treating can be performed 38 weeks after conception. In some embodiments, the treating can be performed 39 weeks after conception. In some embodiments, the treating can be performed 40 weeks after conception.

The subject of this invention can be a mammal. For example, the subject can be a human. The subject can also be a subject in need thereof.

### Methods for Treating

Previous studies in mice have shown that injecting sonic hedgehog agonists into the cerebellum of newborn mice exhibiting some down syndrome-like symptoms (a down syndrome mouse model), resulted in the brain developing into a normal size and at a normal pace. Underwood et al., “Can Down Syndrome Be Treated,” Science, Vol. 343, pp. 964-967 (2014); Ishita Das et al., “Hedgehog Agonist Therapy Corrects Structural and Cognitive Deficits in a Down Syndrome Mouse Model,” Science Translational Medicine, Vol. 5, Issue 201; p. 201ra120 (2013). When the mice aged, some of their learning deficits were also lessened. Id.

Here, the inventors have found that one or more symptoms associated with chromosomal abnormalities can be treated by altering levels of one or more members of the hedgehog signaling pathway.

Disclosed herein are methods of ameliorating one or more symptoms associated with one or more chromosomal abnormalities in a subject or subject in need thereof, the methods comprising increasing and/or maintaining the level of one or more members of the hedgehog signaling pathway.

The one or more members of the hedgehog signaling pathway can be selected from a group consisting of: SHH, DHH, and IHH. The one or more members of the hedgehog signaling pathway can be SHH, DHH, IHH, or a combination thereof.

The one or more members of the hedgehog signaling pathway can be increased and/or maintained by increasing cGMP levels. The increasing and/or maintaining the level of one or more members of the hedgehog signaling pathway can comprise giving the subject one or more cGMP activators. The one or more cGMP activators can be given in combination with one or more additional therapeutic agents. The cGMP activator can be riociguat. The one or more additional therapeutic agents can comprise one or more non-specific PDE inhibitors and/or forskolin, or combinations thereof. The one or more additional therapeutic agents can comprise one or more specific PDE inhibitors and/or forskolin, or combinations thereof.

The methods can further comprise administering to the subject one or more additional therapeutic agents, wherein the one or more additional therapeutic agents can, e.g., comprise riociguat given or present in a positive amount selected from a group consisting of: greater than 0.0 μg to 1 μg, 0.5 μg to 2 μg, 1.5 μg to 3.0 μg, 2.5 μg to 10 μg, 5 μg to 15 μg, 12.5 μg to 30 μg, 25 μg to 50 μg, 40 μg to 80 μg, 60 μg to 100 μg, 90 μg to 120 μg, 110 μg to 130 μg, 125 μg to 150 μg, 140 μg to 180 μg, 170 μg to 200 μg, 200 μg to 230 μg, 215 μg to 240 μg, 235 μg to less than 250 μg, less than 250 μg, greater than about 0.0 μg to about 1 μg, about 0.5 μg to about 2 μg, about 1.5 μg to about 3.0 μg, about 2.5 μg to about 10 μg, about 5 μg to about 15 μg, about 12.5 μg to about 30 μg, about 25 μg to about 50 μg, about 40 μg to about 80 μg, about 60 μg to about 100 μg, about 90 μg to about 120 μg, about 110 μg to about 130 μg, about 125 μg to about 150 μg, about 140 μg to about 180 μg, about 170 μg to about 200 μg, about 200 μg to about 230 μg, about 215 μg to about 240 μg, about 235 μg to less than 250 μg greater than 0.0 μg to 2 μg, greater than 0.0 μg to 3 μg, greater than 0.0 μg to 10 μg, greater than 0.0 μg to 15 μg, greater than 0.0 μg to 30 μg, greater than 0.0 μg to 50 μg, greater than 0.0 μg to 80 μg, greater than 0.0 μg to 100 μg, greater than 0.0 μg to 120 μg, greater than 0.0 μg to 130 μg, greater than 0.0 μg to 150 μg, greater than 0.0 μg to 180 μg, greater than 0.0 μg to 200 μg, greater than 0.0 μg to 230 μg, greater than 0.0 μg to 240 μg, greater than 0.0 μg to 250 μg, greater than 0.0 μg to about 2 μg, greater than 0.0 μg to about 3 μg, greater than 0.0 μg to about 10 μg, greater than 0.0 μg to about 15 μg, greater than 0.0 μg to about 30 μg, greater than 0.0 μg to about 50 μg, greater than 0.0 μg to about 80 μg, greater than 0.0 μg to about 100 μg, greater than 0.0 μg to about 120 μg, greater than 0.0 μg to about 130 μg, greater than 0.0 μg to about 150 μg, greater than 0.0 μg to about 180 μg, greater than 0.0 μg to about 200 μg, greater than 0.0 μg to about 230 μg, greater than 0.0 μg to about 240 μg, greater than 0.0 μg to about 250 μg, 0 μg to less than 250 μg, 0.5 μg to less than 250 μg, 1.5 μg to less than 250 μg, 2.5 μg to less than 250 μg, 5 μg to less than 250 μg, 12.5 μg to less than 250 μg, 25 μg to less than 250 μg, 40 μg to less than 250 μg, 60 μg to less than 250 μg, 90 μg to less than 250 μg, 110 μg to less than 250 μg, 125 μg to less than 250 μg, 140 μg to less than 250 μg, 170 μg to less than 250 μg, 200 μg to less than 250 μg, 215 μg to less than 250 μg, 0 μg to less than about 250 μg, about 0.5 μg to less than about 250 μg, about 1.5 μg to less than about 250 μg, about 2.5 μg to less than about 250 μg, about 5 μg to less than about 250 μg, about 12.5 μg to less than about 250 μg, about 25 μg to less than about 250 μg, about 40 μg to less than about 250 μg, about 60 μg to less than about 250 μg, about 90 μg to less than about 250 μg, about 110 μg to less than about 250 μg, about 125 μg to less than about 250 μg, about 140 μg to less than about 250 μg, about 170 μg to less than about 250 μg, about 200 μg to less than about 250 μg, and/or about 215 μg to less about than 250 μg.

The methods can further comprise administering to the subject one or more additional therapeutic agents, wherein the one or more additional therapeutic agents can comprise theophylline given or present in a positive amount selected from a group consisting of: less than 45 mg, 30 mg, 15 mg, 10 mg, 5 mg, 1 mg, 500 μg, 250 μg, 120 μg, 80 μg, 40 μg, or 20 μg and less than about 45 mg, about 30 mg, about 15 mg, about 10 mg, about 5 mg, about 1 mg, about 500 μg, about 250 μg, about 120 μg, about 80 μg, about 40 μg, or about 20 μg, greater than 0 μg to 20 μg, 10 μg to 40 μg, 30 μg to 80 μg, 70 μg to 120 μg, 100 μg to 250 μg, 200 μg to 500 μg, 400 μg to 1 mg, 900 μg to 5 mg, 4 mg to 10 mg, 9 mg to 15 mg, 14 mg to 30 mg, 25 mg to 45 mg, greater than 0 μg to about 20 μg, about 10 μg to about 40 μg, about 30 μg to about 80 μg, about 70 μg to about 120 μg, about 100 μg to about 250 μg, about 200 μg to about 500 μg, about 400 μg to about 1 mg, about 900 μg to about 5 mg, about 4 mg to about 10 mg, about 9 mg to about 15 mg, about 14 mg to about 30 mg, about 25 mg to about 45 mg, greater than 0 μg to 40 μg, greater than 0 μg to 80 μg, greater than 0 μg to 120 μg, greater than 0 μg to 250 μg, greater than 0 μg to 500 μg, greater than 0 μg to 1 mg, greater than 0 μg to 5 mg, greater than 0 μg to 10 mg, greater than 0 μg to 15 mg, greater than 0 μg to 30 mg, greater than 0 μg to 45 mg, greater than 0 μg to about 40 μg, greater than 0 μg to about 80 μg, greater than 0 μg to about 120 μg, greater than 0 μg to about 250 μg, greater than 0 μg to about 500 μg, greater than 0 μg to about 1 mg, greater than 0 μg to about 5 mg, greater than 0 μg to about 10 mg, greater than 0 μg to about 15 mg, greater than 0 μg to about 30 mg, greater than 0 μg to about 45 mg, greater than 0 μg to 45 mg, 10 μg to 45 mg, 30 μg to 45 mg, 70 μg to 45 mg, 100 μg to 45 mg, 200 μg to 45 mg, 400 μg to 45 mg, 900 μg to 45 mg, 4 mg to 45 mg, 9 mg to 45 mg, 14 mg to 45 mg, 35 mg to 45 mg, greater than 0 μg to about 45 mg, about 10 μg to about 45 mg, about 30 μg to about 45 mg, about 70 μg to about 45 mg, about 100 μg to about 45 mg, about 200 μg to about 45 mg, about 400 μg to about 45 mg, about 900 μg to about 45 mg, about 4 mg to about 5 mg, about 9 mg to about 45 mg, about 14 mg to about 45 mg, and/or about 35 mg to about 45 mg.

The methods can further comprise administering to the subject one or more additional therapeutic agents, wherein the one or more additional therapeutic agents can comprise forskolin given or present in a positive amount selected from a group consisting of: less than 500 mg to 450 mg, 475 mg to 425 mg, 435 mg to 400 mg, 415 mg to 300 mg, 325 mg to 250 mg, 275 mg to 150 mg, 200 mg to 100 mg, 135 mg to 80 mg, 95 mg to 65 mg, 75 mg to 50 mg, 60 mg to 40 mg, 45 mg to 25 mg, 30 mg to 20 mg, 15 mg to 5 mg, 10 mg to 2.5 mg, 3.5 mg to 1 mg, 2 mg to greater than 0 mg, less than about 500 mg to about 450 mg, about 475 mg to about 425 mg, about 435 mg to about 400 mg, about 415 mg to about 300 mg, about 325 mg to about 250 mg, about 275 mg to about 150 mg, about 200 mg to about 100 mg, about 135 mg to about 80 mg, about 95 mg to about 65 mg, about 75 mg to about 50 mg, about 60 mg to about 40 mg, about 45 mg to about 25 mg, about 30 mg to about 20 mg, about 15 mg to about 5 mg, about 10 mg to about 2.5 mg, about 3.5 mg to about 1 mg, about 2 mg to greater than 0 mg, 500 mg, less than 500 mg to 450 mg, less than 500 mg to 425 mg, less than 500 mg to 400 mg, less than 500 mg to 300 mg, less than 500 mg to 250 mg, less than 500 mg to 150 mg, less than 500 mg to 100 mg, less than 500 mg to 80 mg, less than 500 mg to 65 mg, less than 500 mg to 50 mg, less than 500 mg to 40 mg, less than 500 mg to 25 mg, less than 500 mg to 20 mg, less than 500 mg to 5 mg, less than 500 mg to 2.5 mg, less than 500 mg to 1 mg, less than 500 mg to greater than 0 mg, about 500 mg, less than about 500 mg to about 450 mg, less than about 500 mg to about 425 mg, less than about 500 mg to about 400 mg, less than about 500 mg to about 300 mg, less than about 500 mg to about 250 mg, less than about 500 mg to about 150 mg, less than about 500 mg to about 100 mg, less than about 500 mg to about 80 mg, less than about 500 mg to about 65 mg, less than about 500 mg to about 50 mg, less than about 500 mg to about 40 mg, less than about 500 mg to about 25 mg, less than about 500 mg to about 20 mg, less than about 500 mg to about 5 mg, less than about 500 mg to about 2.5 mg, less than about 500 mg to about 1 mg, less than about 500 mg to greater than about 0 mg, greater than 0 mg to 450 mg, greater than 0 mg to 425 mg, greater than 0 mg to 400 mg, greater than 0 mg to 300 mg, greater than 0 mg to 250 mg, greater than 0 mg to 150 mg, greater than 0 mg to 100 mg, greater than 0 mg to 80 mg, greater than 0 mg to 65 mg, greater than 0 mg to 50 mg, greater than 0 mg to 40 mg, greater than 0 mg to 25 mg, greater than 0 mg to 20 mg, greater than 0 mg to 5 mg, greater than 0 mg to 2.5 mg, greater than 0 mg to 1 mg, greater than 0 mg to about 450 mg, greater than 0 mg to about 425 mg, greater than 0 mg to about 400 mg, greater than 0 mg to about 300 mg, greater than 0 mg to about 250 mg, greater than 0 mg to about 150 mg, greater than 0 mg to about 100 mg, greater than 0 mg to about 80 mg, greater than 0 mg to about 65 mg, greater than 0 mg to about 50 mg, greater than 0 mg to about 40 mg, greater than 0 mg to about 25 mg, greater than 0 mg to about 20 mg, greater than 0 mg to about 5 mg, greater than 0 mg to about 2.5 mg, and/or greater than 0 mg to about 1 mg.

The methods can further comprise administering to the subject one or more additional therapeutic agents, wherein the one or more additional therapeutic agents can comprise cilastazol given or present in a positive amount selected from a group consisting of: less than 500 mg to 450 mg, 475 mg to 425 mg, 435 mg to 400 mg, 415 mg to 300 mg, 325 mg to 250 mg, 275 mg to 150 mg, 200 mg to 100 mg, 135 mg to 80 mg, 95 mg to 65 mg, 75 mg to 50 mg, 60 mg to 40 mg, 45 mg to 25 mg, 30 mg to 20 mg, 15 mg to 5 mg, 10 mg to 2.5 mg, 3.5 mg to 1 mg, 2 mg to greater than 0 mg, less than about 500 mg to about 450 mg, about 475 mg to about 425 mg, about 435 mg to about 400 mg, about 415 mg to about 300 mg, about 325 mg to about 250 mg, about 275 mg to about 150 mg, about 200 mg to about 100 mg, about 135 mg to about 80 mg, about 95 mg to about 65 mg, about 75 mg to about 50 mg, about 60 mg to about 40 mg, about 45 mg to about 25 mg, about 30 mg to about 20 mg, about 15 mg to about 5 mg, about 10 mg to about 2.5 mg, about 3.5 mg to about 1 mg, about 2 mg to greater than 0 mg, 500 mg, less than 500 mg to 450 mg, less than 500 mg to 425 mg, less than 500 mg to 400 mg, less than 500 mg to 300 mg, less than 500 mg to 250 mg, less than 500 mg to 150 mg, less than 500 mg to 100 mg, less than 500 mg to 80 mg, less than 500 mg to 65 mg, less than 500 mg to 50 mg, less than 500 mg to 40 mg, less than 500 mg to 25 mg, less than 500 mg to 20 mg, less than 500 mg to 5 mg, less than 500 mg to 2.5 mg, less than 500 mg to 1 mg, less than 500 mg to greater than 0 mg, about 500 mg, less than about 500 mg to about 450 mg, less than about 500 mg to about 425 mg, less than about 500 mg to about 400 mg, less than about 500 mg to about 300 mg, less than about 500 mg to about 250 mg, less than about 500 mg to about 150 mg, less than about 500 mg to about 100 mg, less than about 500 mg to about 80 mg, less than about 500 mg to about 65 mg, less than about 500 mg to about 50 mg, less than about 500 mg to about 40 mg, less than about 500 mg to about 25 mg, less than about 500 mg to about 20 mg, less than about 500 mg to about 5 mg, less than about 500 mg to about 2.5 mg, less than about 500 mg to about 1 mg, less than about 500 mg to greater than about 0 mg, greater than 0 mg to 450 mg, greater than 0 mg to 425 mg, greater than 0 mg to 400 mg, greater than 0 mg to 300 mg, greater than 0 mg to 250 mg, greater than 0 mg to 150 mg, greater than 0 mg to 100 mg, greater than 0 mg to 80 mg, greater than 0 mg to 65 mg, greater than 0 mg to 50 mg, greater than 0 mg to 40 mg, greater than 0 mg to 25 mg, greater than 0 mg to 20 mg, greater than 0 mg to 5 mg, greater than 0 mg to 2.5 mg, greater than 0 mg to 1 mg, greater than 0 mg to about 450 mg, greater than 0 mg to about 425 mg, greater than 0 mg to about 400 mg, greater than 0 mg to about 300 mg, greater than 0 mg to about 250 mg, greater than 0 mg to about 150 mg, greater than 0 mg to about 100 mg, greater than 0 mg to about 80 mg, greater than 0 mg to about 65 mg, greater than 0 mg to about 50 mg, greater than 0 mg to about 40 mg, greater than 0 mg to about 25 mg, greater than 0 mg to about 20 mg, greater than 0 mg to about 5 mg, greater than 0 mg to about 2.5 mg, and/or greater than 0 mg to about 1 mg.

The methods can further comprise administering to the subject one or more additional therapeutic agents, wherein the one or more additional therapeutic agents can comprise roflumilast given or present in a positive amount selected from a group consisting of: less than 10 mg, 5 mg, 1 mg, 500 μg, 250 μg, 120 μg, 80 μg, 40 μg, or 20 μg and less than about 10 mg, about 5 mg, about 1 mg, about 500 μg, about 250 μg, about 120 μg, about 80 μg, about 40 μg, or about 20 μg. Theophylline can be also given, e.g., intranasally, and/or present in a positive amount selected from a group consisting of: greater than 0 μg to 20 μg, 10 μg to 40 μg, 30 μg to 80 μg, 70 μg to 120 μg, 100 μg to 250 μg, 200 μg to 500 μg, 400 μg to 1 mg, 900 μg to 5 mg, 4 mg to 10 mg, 9 mg to 15 mg, 14 mg to 30 mg, 25 mg to 45 mg, greater than 0 μg to about 20 μg, about 10 μg to about 40 μg, about 30 μg to about 80 μg, about 70 μg to about 120 μg, about 100 μg to about 250 μg, about 200 μg to about 500 μg, about 400 μg to about 1 mg, about 900 μg to about 5 mg, about 4 mg to about 10 mg, greater than 0 μg to 40 μg, greater than 0 μg to 80 μg, greater than 0 μg to 120 μg, greater than 0 μg to 250 μg, greater than 0 μg to 500 μg, greater than 0 μg to 1 mg, greater than 0 μg to 5 mg, greater than 0 μg to 10 mg, greater than 0 μg to 15 mg, greater than 0 μg to 30 mg, greater than 0 μg to 45 mg, greater than 0 μg to about 40 μg, greater than 0 μg to about 80 μg, greater than 0 μg to about 120 μg, greater than 0 μg to about 250 μg, greater than 0 μg to about 500 μg, greater than 0 μg to about 1 mg, greater than 0 μg to about 5 mg, greater than 0 μg to about 10 mg, greater than 0 μg to 10 mg, 10 μg to 10 mg, 30 μg to 10 mg, 70 μg to 10 mg, 100 μg to 10 mg, 200 μg to 10 mg, 400 μg to 10 mg, 900 μg to 10 mg, 4 mg to 10 mg, 9 mg to 10 mg, greater than 0 μg to about 10 mg, about 10 μg to about 10 mg, about 30 μg to about 10 mg, about 70 μg to about 10 mg, about 100 μg to about 10 mg, about 200 μg to about 10 mg, about 400 μg to about 10 mg, about 900 μg to about 10 mg, about 4 mg to about 10 mg, and/or about 9 mg to about 10.

The methods can further comprise administering to the subject one or more additional therapeutic agents, wherein the one or more additional therapeutic agents can comprise papaverine given or present in a positive amount selected from a group consisting of: less than 500 mg to 450 mg, 475 mg to 425 mg, 435 mg to 400 mg, 415 mg to 300 mg, 325 mg to 250 mg, 275 mg to 150 mg, 200 mg to 100 mg, 135 mg to 80 mg, 95 mg to 65 mg, 75 mg to 50 mg, 60 mg to 40 mg, 45 mg to 25 mg, 30 mg to 20 mg, 15 mg to 5 mg, 10 mg to 2.5 mg, 3.5 mg to 1 mg, 2 mg to greater than 0 mg, less than about 500 mg to about 450 mg, about 475 mg to about 425 mg, about 435 mg to about 400 mg, about 415 mg to about 300 mg, about 325 mg to about 250 mg, about 275 mg to about 150 mg, about 200 mg to about 100 mg, about 135 mg to about 80 mg, about 95 mg to about 65 mg, about 75 mg to about 50 mg, about 60 mg to about 40 mg, about 45 mg to about 25 mg, about 30 mg to about 20 mg, about 15 mg to about 5 mg, about 10 mg to about 2.5 mg, about 3.5 mg to about 1 mg, about 2 mg to greater than 0 mg, 500 mg, less than 500 mg to 450 mg, less than 500 mg to 425 mg, less than 500 mg to 400 mg, less than 500 mg to 300 mg, less than 500 mg to 250 mg, less than 500 mg to 150 mg, less than 500 mg to 100 mg, less than 500 mg to 80 mg, less than 500 mg to 65 mg, less than 500 mg to 50 mg, less than 500 mg to 40 mg, less than 500 mg to 25 mg, less than 500 mg to 20 mg, less than 500 mg to 5 mg, less than 500 mg to 2.5 mg, less than 500 mg to 1 mg, less than 500 mg to greater than 0 mg, about 500 mg, less than about 500 mg to about 450 mg, less than about 500 mg to about 425 mg, less than about 500 mg to about 400 mg, less than about 500 mg to about 300 mg, less than about 500 mg to about 250 mg, less than about 500 mg to about 150 mg, less than about 500 mg to about 100 mg, less than about 500 mg to about 80 mg, less than about 500 mg to about 65 mg, less than about 500 mg to about 50 mg, less than about 500 mg to about 40 mg, less than about 500 mg to about 25 mg, less than about 500 mg to about 20 mg, less than about 500 mg to about 5 mg, less than about 500 mg to about 2.5 mg, less than about 500 mg to about 1 mg, less than about 500 mg to greater than about 0 mg, greater than 0 mg to 450 mg, greater than 0 mg to 425 mg, greater than 0 mg to 400 mg, greater than 0 mg to 300 mg, greater than 0 mg to 250 mg, greater than 0 mg to 150 mg, greater than 0 mg to 100 mg, greater than 0 mg to 80 mg, greater than 0 mg to 65 mg, greater than 0 mg to 50 mg, greater than 0 mg to 40 mg, greater than 0 mg to 25 mg, greater than 0 mg to 20 mg, greater than 0 mg to 5 mg, greater than 0 mg to 2.5 mg, greater than 0 mg to 1 mg, greater than 0 mg to about 450 mg, greater than 0 mg to about 425 mg, greater than 0 mg to about 400 mg, greater than 0 mg to about 300 mg, greater than 0 mg to about 250 mg, greater than 0 mg to about 150 mg, greater than 0 mg to about 100 mg, greater than 0 mg to about 80 mg, greater than 0 mg to about 65 mg, greater than 0 mg to about 50 mg, greater than 0 mg to about 40 mg, greater than 0 mg to about 25 mg, greater than 0 mg to about 20 mg, greater than 0 mg to about 5 mg, greater than 0 mg to about 2.5 mg, and/or greater than 0 mg to about 1 mg.

The methods can further comprise one or more non-specific PDE inhibitors wherein the one or more non-specific PDE inhibitors can comprise theophylline. The methods can further comprise one or more non-selective PDE inhibitors, wherein the one or more selective PDE inhibitors can be selected from a group consisting of: a PDE-1 selective inhibitor, a PDE-2 selective inhibitor, a PDE-3 selective inhibitor, a PDE-4 selective inhibitor, a PDE-5 selective inhibitor, or a combination thereof.

The methods herein can comprise maintaining and/or increasing the level of one or more members of the hedgehog signaling pathway can comprise administration of an effective amount of one or more members of the hedgehog signaling pathway. The maintaining and/or increasing the level of one or more members of the hedgehog signaling pathway can also comprise administration of an effective amount of the one or more exogenous members of the hedgehog signaling pathway (e.g., SHH, DHH, and/or IHH). The maintaining and/or increasing the level of one or more members of the hedgehog signaling pathway can also comprise activating expression of an effective amount of one or more members of the hedgehog signaling pathway. The activating expression of an effective amount of one or more members of the hedgehog signaling pathway can be effectuated by genetic manipulation of one or more genes responsible for the expression of one or more members of the hedgehog signaling pathway. The activating expression of an effective amount of one or more members of the hedgehog signaling pathway can also be effectuated through a therapeutic agent. The therapeutic agent can directly affect the levels of one or more members of the hedgehog signaling pathway. The therapeutic agent can indirectly affect the levels of one or more members of the hedgehog signaling pathway.

The one or more therapeutic agents or compositions comprising one or more therapeutic agents can be suitable for administration by a methods selected from a group consisting of: oral administration, transmucosal administration, buccal administration, inhalation administration, intranasal administration, parental administration, intravenous administration, subcutaneous administration, intramuscular administration, sublingual administration, transdermal administration, and rectal administration. Because of the ease of use, the one or more therapeutic agents or composition comprising one or more therapeutic agents can be suitable for oral administration, inhalational administration, intranasal administration, or a combination thereof. In some embodiments, the route of administration can penetrate the placental barrier and/or the blood brain barrier. For example, in some embodiments, intranasal drug administration, e.g., theophylline, can be delivered into the brain (1) directly by absorption through the cribriform plate along the olfactory bulb, (2) indirectly by absorption through blood-brain barrier receptors, or (3) through combinations of both methods. In another example, in some embodiments, pregnant mothers can be given a dose of a drug, which will then reach the fetus by crossing the placental barrier. The drug can then enter the fetus and find its way to target sites, e.g., the brain. If the drug reaches the brain, it can cross the blood/brain barrier. In some other embodiments, a breast feeding mother can be given a dose of drug, which will then reach the baby by flowing to the mother's breast milk. The breast milk containing the drug can be then fed to the baby. In some embodiments, the drug can be used to fortify baby's formula and/or milk and then fed to the baby. The drug will eventually find its way to the brain by crossing the blood/brain barrier. In some embodiments, the drug may or may not cross the blood/brain barrier.

The methods herein can comprise treating a woman when she is sexually active, is attempting to conceive, and/or actually conceives. The treating can be performed 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 weeks after conception. The treating can be performed during labor. The treating can be performed immediately after birth. In some embodiments, the treating can be performed 1 week after conception. In some embodiments, the treating can be performed 2 weeks after conception. In some embodiments, the treating can be performed 3 weeks after conception. In some embodiments, the treating can be performed 4 weeks after conception. In some embodiments, the treating can be performed 5 weeks after conception. In some embodiments, the treating can be performed 6 weeks after conception. In some embodiments, the treating can be performed 7 weeks after conception. In some embodiments, the treating can be performed 8 weeks after conception. In some embodiments, the treating can be performed 9 weeks after conception. In some embodiments, the treating can be performed 10 weeks after conception. In some embodiments, the treating can be performed 11 weeks after conception. In some embodiments, the treating can be performed 12 weeks after conception. In some embodiments, the treating can be performed 13 weeks after conception. In some embodiments, the treating can be performed 14 weeks after conception. In some embodiments, the treating can be performed 15 weeks after conception. In some embodiments, the treating can be performed 16 weeks after conception. In some embodiments, the treating can be performed 17 weeks after conception. In some embodiments, the treating can be performed 18 weeks after conception. In some embodiments, the treating can be performed 19 weeks after conception. In some embodiments, the treating can be performed 20 weeks after conception. In some embodiments, the treating can be performed 21 weeks after conception. In some embodiments, the treating can be performed 22 weeks after conception. In some embodiments, the treating can be performed 23 weeks after conception. In some embodiments, the treating can be performed 24 weeks after conception. In some embodiments, the treating can be performed 25 weeks after conception. In some embodiments, the treating can be performed 26 weeks after conception. In some embodiments, the treating can be performed 27 weeks after conception. In some embodiments, the treating can be performed 28 weeks after conception. In some embodiments, the treating can be performed 29 weeks after conception. In some embodiments, the treating can be performed 30 weeks after conception. In some embodiments, the treating can be performed 31 weeks after conception. In some embodiments, the treating can be performed 32 weeks after conception. In some embodiments, the treating can be performed 33 weeks after conception. In some embodiments, the treating can be performed 34 weeks after conception. In some embodiments, the treating can be performed 35 weeks after conception. In some embodiments, the treating can be performed 36 weeks after conception. In some embodiments, the treating can be performed 37 weeks after conception. In some embodiments, the treating can be performed 38 weeks after conception. In some embodiments, the treating can be performed 39 weeks after conception. In some embodiments, the treating can be performed 40 weeks after conception.

The one or more therapeutic agents can comprise one or more cGMP activators, one or more cAMP activators, or any combination thereof.

The methods can further comprise one or more cGMP activators, wherein the one or more cGMP activators can be selected from a group consisting of 3-(5′-hydroxymethyl-2′-furyl)-1-benzylindazole (YC-1), YC-1 derivatives, anthranilic acids derivatives, ataciguat (HMR1766), benzydamine analogs, CFM1517, A-350619, nitrovasodilators, molsidomine, nitroxyl (HNO), BAY 41-2272, BAY 41-8543, BAY 58-2667, cinaciguat (BAY 58-2667), and riociguat (BAY 63-2521). The one or more cGMP activators can be riociguat.

The methods herein can further comprise administering one or more cAMP activators wherein the one or more cAMP activators can be selected from a group consisting of 3-(5′-hydroxymethyl-2′-furyl)-1-benzylindazole (YC-1), glucagon, PDE inhibitors, prostaglandin E1 (PGE1; pharmaceutically known as alprostadil), forskolin, and β-adrenergic activators. The methods can further comprise one or more cAMP activators wherein the one or more cAMP activators can comprise one or more PDE inhibitors and/or forskolin. The methods can further comprise administering one or more cAMP activators wherein the one or more cAMP activators can be forskolin.

The methods can further comprise administering one or more cAMP activators. The one or more cAMP activators can be one or more PDE inhibitors. The one or more PDE inhibitors can comprise a non-selective PDE inhibitor, a PDE-1 selective inhibitor, a PDE-2 selective inhibitor, a PDE-3 selective inhibitor, a PDE-4 selective inhibitor, a PDE-5 selective inhibitor, a PDE-10 selective inhibitor, or a combination thereof. The one or more selective PDE inhibitors can be a specific PDE inhibitor. The one or more PDE inhibitors can comprise a non-selective PDE inhibitor that can be a methylxanthine derivative. The methylxanthine derivative can be caffeine, theophylline, doxophylline, cipamphylline, neuphylline, pentoxiphylline, or diprophylline. The methylxanthine derivative can be theophylline. The PDE 1 inhibitor can be vinpocetine. The PDE 2 inhibitor that can be EHNA. The PDE 3 inhibitor can be inamrinone, anagrelide, or cilostazol. The PDE 4 inhibitor can be mesembrine, rolipram, ibudilast, piclamilast, luteolin, drotaverine, or roflumilast. The PDE 5 inhibitor can be sildenafil, tadalafil, vardenafil, udenafil, avanafil, or dipyridamole. The PDE 10 inhibitor can be papaverine, OMS824 (from Omeros Corporation), and/or PF-2545920 (from Pfizer).

The one or more therapeutic agents can comprise a non-specific PDE inhibitor, forskolin, and riociguat. The one or more therapeutic agents can comprise a specific PDE inhibitor, forskolin, and riociguat. Various combinations are also contemplated. For example, the one or more therapeutic agents can comprise a non-specific PDE inhibitor, theophylline, and riociguat. The one or more therapeutic agents can comprise a specific PDE inhibitor, theophylline, and riociguat. The one or more therapeutic agents can comprise a non-specific PDE inhibitor and riociguat. The one or more therapeutic agents can comprise a specific PDE inhibitor and riociguat. The one or more therapeutic agents can comprise theophylline and riociguat. The one or more therapeutic agents can comprise forskolin and riociguat. The one or more therapeutic agents can comprise theophylline, forskolin, and riociguat.

The methods of this invention can comprise one or more therapeutic agents, wherein the one or more therapeutic agents can be steroid-free.

Riociguat can be given, e.g., intranasally, and/or present in a positive amount selected from a group consisting of: greater than greater than 0.0 μg to 1 μg, 0.5 μg to 2 μg, 1.5 μg to 3.0 μg, 2.5 μg to 10 μg, 5 μg to 15 μg, 12.5 μg to 30 μg, 25 μg to 50 μg, 40 μg to 80 μg, 60 μg to 100 μg, 90 μg to 120 μg, 110 μg to 130 μg, 125 μg to 150 μg, 140 μg to 180 μg, 170 μg to 200 μg, 200 μg to 230 μg, 215 μg to 240 μg, 235 μg to less than 250 μg, less than 250 μg, greater than about 0.0 μg to about 1 μg, about 0.5 μg to about 2 μg, about 1.5 μg to about 3.0 μg, about 2.5 μg to about 10 μg, about 5 μg to about 15 μg, about 12.5 μg to about 30 μg, about 25 μg to about 50 μg, about 40 μg to about 80 μg, about 60 μg to about 100 μg, about 90 μg to about 120 μg, about 110 μg to about 130 μg, about 125 μg to about 150 μg, about 140 μg to about 180 μg, about 170 μg to about 200 μg, about 200 μg to about 230 μg, about 215 μg to about 240 μg, about 235 μg to less than 250 μg greater than 0.0 μg to 2 μg, greater than 0.0 μg to 3 μg, greater than 0.0 μg to 10 μg, greater than 0.0 μg to 15 μg, greater than 0.0 μg to 30 μg, greater than 0.0 μg to 50 μg, greater than 0.0 μg to 80 μg, greater than 0.0 μg to 100 μg, greater than 0.0 μg to 120 μg, greater than 0.0 μg to 130 μg, greater than 0.0 μg to 150 μg, greater than 0.0 μg to 180 μg, greater than 0.0 μg to 200 μg, greater than 0.0 μg to 230 μg, greater than 0.0 μg to 240 μg, greater than 0.0 μg to 250 μg, greater than 0.0 μg to about 2 μg, greater than 0.0 μg to about 3 μg, greater than 0.0 μg to about 10 μg, greater than 0.0 μg to about 15 μg, greater than 0.0 μg to about 30 μg, greater than 0.0 μg to about 50 μg, greater than 0.0 μg to about 80 μg, greater than 0.0 μg to about 100 μg, greater than 0.0 μg to about 120 μg, greater than 0.0 μg to about 130 μg, greater than 0.0 μg to about 150 μg, greater than 0.0 μg to about 180 μg, greater than 0.0 μg to about 200 μg, greater than 0.0 μg to about 230 μg, greater than 0.0 μg to about 240 μg, greater than 0.0 μg to about 250 μg, 0 μg to less than 250 μg, 0.5 μg to less than 250 μg, 1.5 μg to less than 250 μg, 2.5 μg to less than 250 μg, 5 μg to less than 250 μg, 12.5 μg to less than 250 μg, 25 μg to less than 250 μg, 40 μg to less than 250 μg, 60 μg to less than 250 μg, 90 μg to less than 250 μg, 110 μg to less than 250 μg, 125 μg to less than 250 μg, 140 μg to less than 250 μg, 170 μg to less than 250 μg, 200 μg to less than 250 μg, 215 μg to less than 250 μg, 0 μg to less than about 250 μg, about 0.5 μg to less than about 250 μg, about 1.5 μg to less than about 250 μg, about 2.5 μg to less than about 250 μg, about 5 μg to less than about 250 μg, about 12.5 μg to less than about 250 μg, about 25 μg to less than about 250 μg, about 40 μg to less than about 250 μg, about 60 μg to less than about 250 μg, about 90 μg to less than about 250 μg, about 110 μg to less than about 250 μg, about 125 μg to less than about 250 μg, about 140 μg to less than about 250 μg, about 170 μg to less than about 250 μg, about 200 μg to less than about 250 μg, and/or about 215 μg to less about than 250 μg.

Theophylline can be given, e.g., intranasally, and/or present in a positive amount selected from a group consisting of: less than 45 mg, 30 mg, 15 mg, 10 mg, 5 mg, 1 mg, 500 μg, 250 μg, 120 μg, 80 μg, 40 μg, or 20 μg and less than about 45 mg, about 30 mg, about 15 mg, about 10 mg, about 5 mg, about 1 mg, about 500 μg, about 250 μg, about 120 μg, about 80 μg, about 40 μg, or about 20 μg, greater than 0 μg to 20 μg, 10 μg to 40 μg, 30 μg to 80 μg, 70 μg to 120 μg, 100 μg to 250 μg, 200 μg to 500 μg, 400 μg to 1 mg, 900 μg to 5 mg, 4 mg to 10 mg, 9 mg to 15 mg, 14 mg to 30 mg, 25 mg to 45 mg, greater than 0 μg to about 20 μg, about 10 μg to about 40 μg, about 30 μg to about 80 μg, about 70 μg to about 120 μg, about 100 μg to about 250 μg, about 200 μg to about 500 μg, about 400 μg to about 1 mg, about 900 μg to about 5 mg, about 4 mg to about 10 mg, about 9 mg to about 15 mg, about 14 mg to about 30 mg, about 25 mg to about 45 mg, greater than 0 μg to 40 μg, greater than 0 μg to 80 μg, greater than 0 μg to 120 μg, greater than 0 μg to 250 μg, greater than 0 μg to 500 μg, greater than 0 μg to 1 mg, greater than 0 μg to 5 mg, greater than 0 μg to 10 mg, greater than 0 μg to 15 mg, greater than 0 μg to 30 mg, greater than 0 μg to 45 mg, greater than 0 μg to about 40 μg, greater than 0 μg to about 80 μg, greater than 0 μg to about 120 μg, greater than 0 μg to about 250 μg, greater than 0 μg to about 500 μg, greater than 0 μg to about 1 mg, greater than 0 μg to about 5 mg, greater than 0 μg to about 10 mg, greater than 0 μg to about 15 mg, greater than 0 μg to about 30 mg, greater than 0 μg to about 45 mg, greater than 0 μg to 45 mg, 10 μg to 45 mg, 30 μg to 45 mg, 70 μg to 45 mg, 100 μg to 45 mg, 200 μg to 45 mg, 400 μg to 45 mg, 900 μg to 45 mg, 4 mg to 45 mg, 9 mg to 45 mg, 14 mg to 45 mg, 35 mg to 45 mg, greater than 0 μg to about 45 mg, about 10 μg to about 45 mg, about 30 μg to about 45 mg, about 70 μg to about 45 mg, about 100 μg to about 45 mg, about 200 μg to about 45 mg, about 400 μg to about 45 mg, about 900 μg to about 45 mg, about 4 mg to about 5 mg, about 9 mg to about 45 mg, about 14 mg to about 45 mg, and/or about 35 mg to about 45 mg.

Forskolin can be given, e.g., intranasally, and/or present in a positive amount selected from a group consisting of: less than 500 mg to 450 mg, 475 mg to 425 mg, 435 mg to 400 mg, 415 mg to 300 mg, 325 mg to 250 mg, 275 mg to 150 mg, 200 mg to 100 mg, 135 mg to 80 mg, 95 mg to 65 mg, 75 mg to 50 mg, 60 mg to 40 mg, 45 mg to 25 mg, 30 mg to 20 mg, 15 mg to 5 mg, 10 mg to 2.5 mg, 3.5 mg to 1 mg, 2 mg to greater than 0 mg, less than about 500 mg to about 450 mg, about 475 mg to about 425 mg, about 435 mg to about 400 mg, about 415 mg to about 300 mg, about 325 mg to about 250 mg, about 275 mg to about 150 mg, about 200 mg to about 100 mg, about 135 mg to about 80 mg, about 95 mg to about 65 mg, about 75 mg to about 50 mg, about 60 mg to about 40 mg, about 45 mg to about 25 mg, about 30 mg to about 20 mg, about 15 mg to about 5 mg, about 10 mg to about 2.5 mg, about 3.5 mg to about 1 mg, about 2 mg to greater than 0 mg, 500 mg, less than 500 mg to 450 mg, less than 500 mg to 425 mg, less than 500 mg to 400 mg, less than 500 mg to 300 mg, less than 500 mg to 250 mg, less than 500 mg to 150 mg, less than 500 mg to 100 mg, less than 500 mg to 80 mg, less than 500 mg to 65 mg, less than 500 mg to 50 mg, less than 500 mg to 40 mg, less than 500 mg to 25 mg, less than 500 mg to 20 mg, less than 500 mg to 5 mg, less than 500 mg to 2.5 mg, less than 500 mg to 1 mg, less than 500 mg to greater than 0 mg, about 500 mg, less than about 500 mg to about 450 mg, less than about 500 mg to about 425 mg, less than about 500 mg to about 400 mg, less than about 500 mg to about 300 mg, less than about 500 mg to about 250 mg, less than about 500 mg to about 150 mg, less than about 500 mg to about 100 mg, less than about 500 mg to about 80 mg, less than about 500 mg to about 65 mg, less than about 500 mg to about 50 mg, less than about 500 mg to about 40 mg, less than about 500 mg to about 25 mg, less than about 500 mg to about 20 mg, less than about 500 mg to about 5 mg, less than about 500 mg to about 2.5 mg, less than about 500 mg to about 1 mg, less than about 500 mg to greater than about 0 mg, greater than 0 mg to 450 mg, greater than 0 mg to 425 mg, greater than 0 mg to 400 mg, greater than 0 mg to 300 mg, greater than 0 mg to 250 mg, greater than 0 mg to 150 mg, greater than 0 mg to 100 mg, greater than 0 mg to 80 mg, greater than 0 mg to 65 mg, greater than 0 mg to 50 mg, greater than 0 mg to 40 mg, greater than 0 mg to 25 mg, greater than 0 mg to 20 mg, greater than 0 mg to 5 mg, greater than 0 mg to 2.5 mg, greater than 0 mg to 1 mg, greater than 0 mg to about 450 mg, greater than 0 mg to about 425 mg, greater than 0 mg to about 400 mg, greater than 0 mg to about 300 mg, greater than 0 mg to about 250 mg, greater than 0 mg to about 150 mg, greater than 0 mg to about 100 mg, greater than 0 mg to about 80 mg, greater than 0 mg to about 65 mg, greater than 0 mg to about 50 mg, greater than 0 mg to about 40 mg, greater than 0 mg to about 25 mg, greater than 0 mg to about 20 mg, greater than 0 mg to about 5 mg, greater than 0 mg to about 2.5 mg, and/or greater than 0 mg to about 1 mg.

Cilastazol can be given, e.g., intranasally, and/or present in a positive amount selected from a group consisting of: less than 500 mg to 450 mg, 475 mg to 425 mg, 435 mg to 400 mg, 415 mg to 300 mg, 325 mg to 250 mg, 275 mg to 150 mg, 200 mg to 100 mg, 135 mg to 80 mg, 95 mg to 65 mg, 75 mg to 50 mg, 60 mg to 40 mg, 45 mg to 25 mg, 30 mg to 20 mg, 15 mg to 5 mg, 10 mg to 2.5 mg, 3.5 mg to 1 mg, 2 mg to greater than 0 mg, less than about 500 mg to about 450 mg, about 475 mg to about 425 mg, about 435 mg to about 400 mg, about 415 mg to about 300 mg, about 325 mg to about 250 mg, about 275 mg to about 150 mg, about 200 mg to about 100 mg, about 135 mg to about 80 mg, about 95 mg to about 65 mg, about 75 mg to about 50 mg, about 60 mg to about 40 mg, about 45 mg to about 25 mg, about 30 mg to about 20 mg, about 15 mg to about 5 mg, about 10 mg to about 2.5 mg, about 3.5 mg to about 1 mg, about 2 mg to greater than 0 mg, 500 mg, less than 500 mg to 450 mg, less than 500 mg to 425 mg, less than 500 mg to 400 mg, less than 500 mg to 300 mg, less than 500 mg to 250 mg, less than 500 mg to 150 mg, less than 500 mg to 100 mg, less than 500 mg to 80 mg, less than 500 mg to 65 mg, less than 500 mg to 50 mg, less than 500 mg to 40 mg, less than 500 mg to 25 mg, less than 500 mg to 20 mg, less than 500 mg to 5 mg, less than 500 mg to 2.5 mg, less than 500 mg to 1 mg, less than 500 mg to greater than 0 mg, about 500 mg, less than about 500 mg to about 450 mg, less than about 500 mg to about 425 mg, less than about 500 mg to about 400 mg, less than about 500 mg to about 300 mg, less than about 500 mg to about 250 mg, less than about 500 mg to about 150 mg, less than about 500 mg to about 100 mg, less than about 500 mg to about 80 mg, less than about 500 mg to about 65 mg, less than about 500 mg to about 50 mg, less than about 500 mg to about 40 mg, less than about 500 mg to about 25 mg, less than about 500 mg to about 20 mg, less than about 500 mg to about 5 mg, less than about 500 mg to about 2.5 mg, less than about 500 mg to about 1 mg, less than about 500 mg to greater than about 0 mg, greater than 0 mg to 450 mg, greater than 0 mg to 425 mg, greater than 0 mg to 400 mg, greater than 0 mg to 300 mg, greater than 0 mg to 250 mg, greater than 0 mg to 150 mg, greater than 0 mg to 100 mg, greater than 0 mg to 80 mg, greater than 0 mg to 65 mg, greater than 0 mg to 50 mg, greater than 0 mg to 40 mg, greater than 0 mg to 25 mg, greater than 0 mg to 20 mg, greater than 0 mg to 5 mg, greater than 0 mg to 2.5 mg, greater than 0 mg to 1 mg, greater than 0 mg to about 450 mg, greater than 0 mg to about 425 mg, greater than 0 mg to about 400 mg, greater than 0 mg to about 300 mg, greater than 0 mg to about 250 mg, greater than 0 mg to about 150 mg, greater than 0 mg to about 100 mg, greater than 0 mg to about 80 mg, greater than 0 mg to about 65 mg, greater than 0 mg to about 50 mg, greater than 0 mg to about 40 mg, greater than 0 mg to about 25 mg, greater than 0 mg to about 20 mg, greater than 0 mg to about 5 mg, greater than 0 mg to about 2.5 mg, and/or greater than 0 mg to about 1 mg.

Roflumilast can be given, e.g., intranasally, and/or present in a positive amount selected from a group consisting of: less than 10 mg, 5 mg, 1 mg, 500 μg, 250 μg, 120 μg, 80 μg, 40 μg, or 20 μg and less than about 10 mg, about 5 mg, about 1 mg, about 500 μg, about 250 μg, about 120 μg, about 80 μg, about 40 μg, or about 20 μg. Theophylline can be also given, e.g., intranasally, and/or present in a positive amount selected from a group consisting of: greater than 0 μg to 20 μg, 10 μg to 40 μg, 30 μg to 80 μg, 70 μg to 120 μg, 100 μg to 250 μg, 200 μg to 500 μg, 400 μg to 1 mg, 900 μg to 5 mg, 4 mg to 10 mg, 9 mg to 15 mg, 14 mg to 30 mg, 25 mg to 45 mg, greater than 0 μg to about 20 μg, about 10 μg to about 40 μg, about 30 μg to about 80 μg, about 70 μg to about 120 μg, about 100 μg to about 250 μg, about 200 μg to about 500 μg, about 400 μg to about 1 mg, about 900 μg to about 5 mg, about 4 mg to about 10 mg, greater than 0 μg to 40 μg, greater than 0 μg to 80 μg, greater than 0 μg to 120 μg, greater than 0 μg to 250 μg, greater than 0 μg to 500 μg, greater than 0 μg to 1 mg, greater than 0 μg to 5 mg, greater than 0 μg to 10 mg, greater than 0 μg to 15 mg, greater than 0 μg to 30 mg, greater than 0 μg to 45 mg, greater than 0 μg to about 40 μg, greater than 0 μg to about 80 μg, greater than 0 μg to about 120 μg, greater than 0 μg to about 250 μg, greater than 0 μg to about 500 μg, greater than 0 μg to about 1 mg, greater than 0 μg to about 5 mg, greater than 0 μg to about 10 mg, greater than 0 μg to 10 mg, 10 μg to 10 mg, 30 μg to 10 mg, 70 μg to 10 mg, 100 μg to 10 mg, 200 μg to 10 mg, 400 μg to 10 mg, 900 μg to 10 mg, 4 mg to 10 mg, 9 mg to 10 mg, greater than 0 μg to about 10 mg, about 10 μg to about 10 mg, about 30 μg to about 10 mg, about 70 μg to about 10 mg, about 100 μg to about 10 mg, about 200 μg to about 10 mg, about 400 μg to about 10 mg, about 900 μg to about 10 mg, about 4 mg to about 10 mg, and/or about 9 mg to about 10.

Papaverine can be given, e.g., intranasally, and/or present in a positive amount selected from a group consisting of: less than 500 mg to 450 mg, 475 mg to 425 mg, 435 mg to 400 mg, 415 mg to 300 mg, 325 mg to 250 mg, 275 mg to 150 mg, 200 mg to 100 mg, 135 mg to 80 mg, 95 mg to 65 mg, 75 mg to 50 mg, 60 mg to 40 mg, 45 mg to 25 mg, 30 mg to 20 mg, 15 mg to 5 mg, 10 mg to 2.5 mg, 3.5 mg to 1 mg, 2 mg to greater than 0 mg, less than about 500 mg to about 450 mg, about 475 mg to about 425 mg, about 435 mg to about 400 mg, about 415 mg to about 300 mg, about 325 mg to about 250 mg, about 275 mg to about 150 mg, about 200 mg to about 100 mg, about 135 mg to about 80 mg, about 95 mg to about 65 mg, about 75 mg to about 50 mg, about 60 mg to about 40 mg, about 45 mg to about 25 mg, about 30 mg to about 20 mg, about 15 mg to about 5 mg, about 10 mg to about 2.5 mg, about 3.5 mg to about 1 mg, about 2 mg to greater than 0 mg, 500 mg, less than 500 mg to 450 mg, less than 500 mg to 425 mg, less than 500 mg to 400 mg, less than 500 mg to 300 mg, less than 500 mg to 250 mg, less than 500 mg to 150 mg, less than 500 mg to 100 mg, less than 500 mg to 80 mg, less than 500 mg to 65 mg, less than 500 mg to 50 mg, less than 500 mg to 40 mg, less than 500 mg to 25 mg, less than 500 mg to 20 mg, less than 500 mg to 5 mg, less than 500 mg to 2.5 mg, less than 500 mg to 1 mg, less than 500 mg to greater than 0 mg, about 500 mg, less than about 500 mg to about 450 mg, less than about 500 mg to about 425 mg, less than about 500 mg to about 400 mg, less than about 500 mg to about 300 mg, less than about 500 mg to about 250 mg, less than about 500 mg to about 150 mg, less than about 500 mg to about 100 mg, less than about 500 mg to about 80 mg, less than about 500 mg to about 65 mg, less than about 500 mg to about 50 mg, less than about 500 mg to about 40 mg, less than about 500 mg to about 25 mg, less than about 500 mg to about 20 mg, less than about 500 mg to about 5 mg, less than about 500 mg to about 2.5 mg, less than about 500 mg to about 1 mg, less than about 500 mg to greater than about 0 mg, greater than 0 mg to 450 mg, greater than 0 mg to 425 mg, greater than 0 mg to 400 mg, greater than 0 mg to 300 mg, greater than 0 mg to 250 mg, greater than 0 mg to 150 mg, greater than 0 mg to 100 mg, greater than 0 mg to 80 mg, greater than 0 mg to 65 mg, greater than 0 mg to 50 mg, greater than 0 mg to 40 mg, greater than 0 mg to 25 mg, greater than 0 mg to 20 mg, greater than 0 mg to 5 mg, greater than 0 mg to 2.5 mg, greater than 0 mg to 1 mg, greater than 0 mg to about 450 mg, greater than 0 mg to about 425 mg, greater than 0 mg to about 400 mg, greater than 0 mg to about 300 mg, greater than 0 mg to about 250 mg, greater than 0 mg to about 150 mg, greater than 0 mg to about 100 mg, greater than 0 mg to about 80 mg, greater than 0 mg to about 65 mg, greater than 0 mg to about 50 mg, greater than 0 mg to about 40 mg, greater than 0 mg to about 25 mg, greater than 0 mg to about 20 mg, greater than 0 mg to about 5 mg, greater than 0 mg to about 2.5 mg, and/or greater than 0 mg to about 1 mg.

The methods herein can comprise increasing and/or maintaining the level of one or more members of the hedgehog signaling pathway can comprise administering an effective amount of one or more members of the hedgehog signaling pathway. The increasing and/or maintaining the level of one or more members of the hedgehog signaling pathway can comprise administering an effective amount of one or more exogenous members of the hedgehog signaling pathway. The increasing and/or maintaining the level of one or more members of the hedgehog signaling pathway can comprise activating expression of an effective amount of one or more members of the hedgehog signaling pathway. The activating expression of an effective amount of one or more members of the hedgehog signaling pathway can be effectuated by genetic manipulation of one or more genes responsible for the expression of one or more members of the hedgehog signaling pathway. The activating expression of an effective amount of one or more members of the hedgehog signaling pathway can be effectuated through a therapeutic agent. The therapeutic agent can directly affect the levels of one or more members of the hedgehog signaling pathway. The therapeutic agent can indirectly affect the levels of one or more members of the hedgehog signaling pathway.

The one or more therapeutic agents or composition comprising one or more therapeutic agents can be a liquid. The dosage unit or composition comprising the dosage unit can have a pH of 7.0 or less than 7.0. For example, the dosage unit or composition comprising the dosage unit can have a pH of 7.0. The dosage unit or composition comprising the dosage unit can have a pH of less than 7.0. The dosage unit or composition comprising the dosage unit can have a pH of less than 6.9. The dosage unit or composition comprising the dosage unit can have a pH of less than 6.5. The dosage unit or composition comprising the dosage unit can have a pH of greater than 7.0. For example, the dosage unit or composition comprising the dosage unit can have a pH of greater than 7.1. The dosage unit or composition comprising the dosage unit can have a pH of greater than 7.5. The dosage unit or composition comprising the dosage unit can have a pH of greater than 8.0. The dosage unit or composition comprising the dosage unit can have a pH selected from the group consisting of: 5.0 to 5.5; 5.1 to 5.6; 5.2 to 5.7; 5.3 to 5.8; 5.4 to 5.9; 5.5 to 6.0; 5.6 to 6.1; 5.7 to 6.2; 5.8 to 6.3; 5.9 to 6.4; 6.0 to 6.5; 6.1 to 6.6; 6.2 to 6.7; 6.3 to 6.8; 6.4 to 6.9; 6.5 to 7.0; 6.6 to 7.1; 6.7 to 7.2; 6.8 to 7.3; 6.9 to 7.4; 7.0 to 7.5; 7.1 to 7.6; 7.2 to 7.7; 7.3 to 7.8; 7.4 to 7.9; 7.5 to 8.0; 7.6 to 8.1; 7.7 to 8.2; 7.8 to 8.3; 7.9 to 8.4; 8.0 to 8.5; 8.1 to 8.6; 8.2 to 8.7; 8.3 to 8.8; 8.4 to 8.9; 8.5 to 9.0; 8.6 to 9.1; 8.7 to 9.2; 8.8 to 9.3; 8.9 to 9.4; 9.0 to 9.5; about 5.0 to about 5.5; about 5.1 to about 5.6; about 5.2 to about 5.7; about 5.3 to about 5.8; about 5.4 to about 5.9; about 5.5 to about 6.0; about 5.6 to about 6.1; about 5.7 to about 6.2; about 5.8 to about 6.3; about 5.9 to about 6.4; about 6.0 to about 6.5; about 6.1 to about 6.6; about 6.2 to about 6.7; about 6.3 to about 6.8; about 6.4 to about 6.9; about 6.5 to about 7.0; about 6.6 to about 7.1; about 6.7 to about 7.2; about 6.8 to about 7.3; about 6.9 to about 7.4; about 7.0 to about 7.5; about 7.1 to about 7.6; about 7.2 to about 7.7; about 7.3 to about 7.8; about 7.4 to about 7.9; about 7.5 to about 8.0; about 7.6 to about 8.1; about 7.7 to about 8.2; about 7.8 to about 8.3; about 7.9 to about 8.4; about 8.0 to about 8.5; about 8.1 to about 8.6; about 8.2 to about 8.7; about 8.3 to about 8.8; about 8.4 to about 8.9; about 8.5 to about 9.0; about 8.6 to about 9.1; about 8.7 to about 9.2; about 8.8 to about 9.3; about 8.9 to about 9.4; and/or about 9.0 to about 9.5.

Excipients can be added to one or more therapeutic agents or compositions. The excipients can include those found in the Handbook of Pharmaceutical Excipients, Sixth Edition (2009), Eds. R. C. Rowe, P. J. Shesky, and M. E. Quinn. For example, it is contemplated that the following excipients can be added separately or in any combination, to one or more therapeutic agents or compositions: Acacia, Acesulfame Potassium, Acetic Acid—Glacial, Acetone, Acetyltributyl Citrate, Acetyltriethyl Citrate, Adipic Acid, Agar, Albumin, Alcohol, Alginic Acid, Aliphatic Polyesters, Alitame, Almond Oil, Alpha Tocopherol, Aluminum Hydroxide Adjuvant, Aluminum Monostearate, Aluminum Oxide, Aluminum Phosphate Adjuvant, Ammonia Solution, Ammonium Alginate, Ammonium Chloride, Ascorbic Acid, Ascorbyl Palmitate, Aspartame, Attapulgite, Bentonite, Benzalkonium Chloride, Benzethonium Chloride, Benzoic Acid, Benzyl Alcohol, Benzyl Benzoate, Boric Acid, Bronopol, Butylated Hydroxyanisole, Butylated Hydroxytoluene, Butylene Glycol, Butylparaben, Calcium Acetate, Calcium Alginate, Calcium Carbonate, Calcium Chloride, Calcium Hydroxide, Calcium Lactate, Calcium Phosphate—Dibasic Anhydrous, Calcium Phosphate—Dibasic Dihydrate, Calcium Phosphate—Tribasic, Calcium Silicate, Calcium Stearate, Calcium Sulfate, Canola Oil, Carbomer, Carbon Dioxide, Carboxymethylcellulose Calcium, Carboxymethylcellulose Sodium, Carrageenan, Castor Oil, Castor Oil—Hydrogenated, Cellulose—Microcrystalline, Cellulose—Microcrystalline and Carboxymethylcellulose Sodium, Cellulose—Powdered, Cellulose—Silicified Microcrystalline, Cellulose Acetate, Cellulose Acetate Phthalate, Ceratonia, Ceresin, Cetostearyl Alcohol, Cetrimide, Cetyl Alcohol, Cetylpyridinium Chloride, Chitosan, Chlorhexidine, Chlorobutanol, Chlorocresol, Chlorodifluoroethane (HCFC), Chlorofluorocarbons (CFC), Chloroxylenol, Cholesterol, Citric Acid Monohydrate, Coconut Oil, Colloidal Silicon Dioxide, Coloring Agents, Copovidone, Corn Oil, Corn Starchand Pregelatinized Starch, Cottonseed Oil, Cresol, Croscarmellose Sodium, Crospovidone, Cyclodextrins, Cyclomethicone, Denatonium Benzoate, Dextrates, Dextrin, Dextrose, Dibutyl Phthalate, Dibutyl Sebacate, Diethanolamine, Diethyl Phthalate, Difluoroethane (HFC), Dimethicone, Dimethyl Ether, Dimethyl Phthalate, Dimethyl Sulfoxide, Dimethylacetamide, Disodium Edetate, Docusate Sodium, Edetic Acid, Erythorbic Acid, Erythritol, Ethyl Acetate, Ethyl Lactate, Ethyl Maltol, Ethyl Oleate, Ethyl Vanillin, Ethylcellulose, Ethylene Glycol Stearates, Ethylene Vinyl Acetate, Ethylparaben, Fructose, Fumaric Acid, Gelatin, Glucose—Liquid, Glycerin, Glyceryl Behenate, Glyceryl Monooleate, Glyceryl Monostearate, Glyceryl Palmitostearate, Glycine, Glycofurol, Guar Gum, Hectorite, Heptafluoropropane (HFC), Hexetidine, Hydrocarbons (HC), Hydrochloric Acid, Hydrophobic Colloidal Silica, Hydroxyethyl Cellulose, Hydroxyethylmethyl Cellulose, Hydroxypropyl Betadex, Hydroxypropyl Cellulose, Hydroxypropyl Cellulose—Low-substituted, Hydroxypropyl Starch, Hypromellose, Hypromellose Acetate Succinate, Hypromellose Phthalate, Imidurea, Inulin, Iron Oxides, Isomalt, Isopropyl Alcohol, Isopropyl Myristate, Isopropyl Palmitate, Kaolin, Lactic Acid, Lactitol, Lactose—Anhydrous, Lactose—Inhalation, Lactose—Monohydrate, Lactose—Monohydrate and Corn Starch, Lactose—Monohydrate and Microcrystalline Cellulose, Lactose—Monohydrate and Povidone, Lactose—Monohydrate and Powdered Cellulose, Lactose—Spray-Dried, Lanolin, Lanolin—Hydrous, Lanolin Alcohols, Laurie Acid, Lecithin, Leucine, Linoleic Acid, Macrogol 15 Hydroxystearate, Magnesium Aluminum Silicate, Magnesium Carbonate, Magnesium Oxide, Magnesium Silicate, Magnesium Stearate, Magnesium Trisilicate, Maleic Acid, Malic Acid, Maltitol, Maltitol Solution, Maltodextrin, Maltol, Maltose, Mannitol, Medium-chain Triglycerides, Meglumine, Menthol, Methionine, Methylcellulose, Methylparaben, Mineral Oil, Mineral Oil—Light, Mineral Oil and Lanolin Alcohols, Monoethanolamine, Monosodium Glutamate, Monothioglycerol, Myristic Acid, Myristyl Alcohol, Neohesperidin Dihydrochalcone, Neotame, Nitrogen, Nitrous Oxide, Octyldodecanol, Oleic Acid, Oleyl Alcohol, Olive Oil, Palmitic Acid, Paraffin, Peanut Oil, Pectin, Pentetic Acid, Petrolatum, Petrolatum and Lanolin Alcohols, Phenol, Phenoxyethanol, Phenylethyl Alcohol, Phenylmercuric Acetate, Phenylmercuric Borate, Phenylmercuric Nitrate, Phospholipids, Phosphoric Acid, Polacrilin Potassium, Poloxamer, Polycarbophil, Polydextrose, Poly (DL-Lactic Acid), Polyethylene Glycol, Polyethylene Oxide, Polymethacrylates, Poly(methyl vinylether/maleic anhydride), Polyoxyethylene Alkyl Ethers, Polyoxyethylene Castor Oil Derivatives, Polyoxyethylene Sorbitan Fatty Acid Esters, Polyoxyethylene Stearates, Polyoxylglycerides, Polyvinyl Acetate Phthalate, Polyvinyl Alcohol, Potassium Alginate, Potassium Alum, Potassium Benzoate, Potassium Bicarbonate, Potassium Chloride, Potassium Citrate, Potassium Hydroxide, Potassium Metabisulfite, Potassium Sorbate, Povidone, Propionic Acid, Propyl Gallate, Propylene Carbonate, Propylene Glycol, Propylene Glycol Alginate, Propylparaben, Propylparaben Sodium, Pyrrolidone, Raffinose, Saccharin, Saccharin Sodium, Safflower Oil, Saponite, Sesame Oil, Shellac, Simethicone, Sodium Acetate, Sodium Alginate, Sodium Ascorbate, Sodium Benzoate, Sodium Bicarbonate, Sodium Borate, Sodium Carbonate, Sodium Chloride, Sodium Citrate Dihydrate, Sodium Cyclamate, Sodium Formaldehyde Sulfoxylate, Sodium Hyaluronate, Sodium Hydroxide, Sodium Lactate, Sodium Lauryl Sulfate, Sodium Metabisulfite, Sodium Phosphate—Dibasic, Sodium Phosphate—Monobasic, Sodium Propionate, Sodium Starch Glycolate, Sodium Stearyl Fumarate, Sodium Sulfite, Sodium Thiosulfate, Sorbic Acid, Sorbitan Esters (Sorbitan Fatty Acid Esters), Sorbitol, Soybean Oil, Starch, Starch—Pregelatinized, Starch—Sterilizable Maize, Stearic Acid, Stearyl Alcohol, Sucralose, Sucrose, Sucrose Octaacetate, Sugar—Compressible, Sugar—Confectioner's, Sugar Spheres, Sulfobutylether b-Cyclodextrin, Sulfur Dioxide, Sulfuric Acid, Sunflower Oil, Suppository Bases—Hard Fat, Tagatose, Talc, Tartaric Acid, Tetrafluoroethane (HFC), Thaumatin, Thimerosal, Thymol, Titanium Dioxide, Tragacanth, Trehalose, Triacetin, Tributyl Citrate, Tricaprylin, Triethanolamine, Triethyl Citrate, Triolein, Vanillin, Vegetable Oil—Hydrogenated, Vitamin E Polyethylene Glycol Succinate, Water, Wax—Anionic Emulsifying, Wax—Carnauba, Wax—Cetyl Esters, Wax—Microcrystalline, Wax—Nonionic Emulsifying, Wax—White, Wax—Yellow, Xanthan Gum, Xylitol, Zein, Zinc Acetate, and/or Zinc Stearate.

The one or more therapeutic agents or compositions comprising one or more therapeutic agents can further comprise one or more excipients. The one or more therapeutic agents or composition comprising one or more therapeutic agents can further comprise one or more excipients wherein the one or more excipients can be selected from a group consisting of: detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

The one or more therapeutic agents can comprise one or more cytochrome p450 inhibitors. The one or more therapeutic agents or composition comprising one or more therapeutic agents can further comprise one or more cytochrome p450 inhibitors wherein the one or more cytochrome p450 inhibitors can fully or partially inhibit a cytochrome p450 selected from a group consisting of: CYP1, CYP1A1, CYP1A2, CYP1B1, CYP2, CYP2A6, CYP2A7, CYP2A13, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3, CYP3A4, CYP3A5, CYP3A7, CYP3A43, CYP4, CYP4A11, CYP4A22, CYP4B1, CYP4F2, CYP4F3, CYP4F8, CYP4F11, CYP4F12, CYP4F22, CYP4V2, CYP4X1, CYP4Z1, CYP5, CYP5A1, CYP7, CYP7A1, CYP7B1, CYP8, CYP8A1, CYP8B1, CYP11, CYP11A1, CYP11B1, CYP11B2, CYP17, CYP17A1, CYP19, CYP19A1, CYP20, CYP20A1, CYP21, CYP21A2, CYP24, CYP24A1, CYP26, CYP26A1, CYP26B1, CYP26C1, CYP27, CYP27A1, CYP27B1, CYP27C1, CYP39, CYP39A1, CYP46, CYP46A1, CYP51, and CYP51A1. The one or more cytochrome p450 inhibitors can fully or partially inhibit CYP1. The one or more cytochrome p450 inhibitors can fully or partially inhibit CYP1A2. The one or more CYP1A2 inhibitor can be selected from a group consisting of: fluoroquinolone, selective serotonin reuptake inhibitor (SSRI), calcium channel blocker, herbal tea, naringenin, H2-receptor activators, antiarrhythmic agent, interferon, xanthotoxin, mibefradil, cumin, turmeric, and isoniazid. The one or more CYP1A2 inhibitor can be grapefruit juice or a component thereof. The one or more CYP1A2 inhibitor can be naringenin. The one or more CYP3A4 inhibitor can be aminodarone, anastrozole, azithromzcin, cannabinoids, cimetidine, clarithromycin, clotrimazole, cyclosporine, danazol, delavirdine, dexamethasone, diethyldithiocarbamate, diltiazem, dirithyromycin, disulfiram, entacapone, erythromycin, ethinyl estradiol, fluconazole, fluoxetine, fluvoaxamine, gestodene, grapefruit juice, indinavir, isoniazid, ketoconazole, metronidazole, mibefradil, miconazole, nefazodone, nelfinavir, nevirapine, norfloxacin, norfluoxetine, omeprazole, oxiconazole, paroxetine, propoxyphene, quinidine, quinine, quinupristine, dalfopristin, ranitidine, ritonavir, saquinavir, sertindole, sertraline, troglitazone, troleandomycin, valproic acid, and/or any combination thereof.

The one or more therapeutic agents or compositions comprising one or more therapeutic agents can further comprise one or more β-adrenergic activators. The one or more 3-adrenergic activators can be a β₁-adrenergic activators and/or β₂-adrenergic activators. The one or more therapeutic agents or composition can comprise one or more 3-adrenergic activators wherein the one or more β-adrenergic activators can be a β₁-adrenergic activators. The one or more therapeutic agents or composition can comprise one or more β-adrenergic activators wherein the one or more β-adrenergic activators can be a β₁-adrenergic activators selected from a group consisting of: dobutamine, isoproterenol, xamoterol and epinephrine. The one or more therapeutic agents or composition can comprise one or more β-adrenergic activators wherein the one or more β-adrenergic activators can be a β₂-adrenergic activators. The one or more therapeutic agents or composition can comprise one or more β-adrenergic activators wherein the one or more β-adrenergic activators can be a β₂-adrenergic activators selected from a group consisting of: albuterol, levalbuterol, fenoterol, formoterol, isoproterenol (β₁ and β₂), metaproterenol, salmeterol, terbutaline, clenbuterol, isoetarine, pirbuterol, procaterol, ritodrine, and epinephrine. The one or more therapeutic agents or composition can comprise one or more β-adrenergic activators wherein the one or more β-adrenergic activators can be selected from a group consisting of: arbutamine, befunolol, bromoacetylalprenololmenthane, broxaterol, cimaterol, cirazoline, denopamine, dopexamine, etilefrine, hexoprenaline, higenamine, isoxsuprine, mabuterol, methoxyphenamine, nylidrin, oxyfedrine, prenalterol, ractopamine, reproterol, rimiterol, tretoquinol, tulobuterol, zilpaterol, and zinterol.

It is also contemplated that the drugs and/or methods disclosed herein can be used to prophylactically preempt one or more symptoms of chromosomal abnormalities. For example, in some embodiments, disclosed is a method of prophylactically preempting one or more symptoms associated with one or more chromosomal abnormalities in a subject or subject in need thereof comprising, (a) accessing if a human is or will become pregnant; (b) ascertaining the age of said human; and (c) administering to said human one or more hedgehog activators if said human is between 15 and 45 years of age at conception. In some embodiments, the human can be greater than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 years of age at conception. In some embodiments, the human can be greater than 45 years of age at conception. In some embodiments, the human can be greater than 40 years of age at conception. In some embodiments, the human can be greater than 39 years of age at conception. In some embodiments, the human can be greater than 38 years of age at conception. In some embodiments, the human can be greater than 37 years of age at conception. In some embodiments, the human can be greater than 36 years of age at conception. In some embodiments, the human can be greater than 35 years of age at conception. In some embodiments, the human can be greater than 40 years of age at conception. In some embodiments, the human can be greater than 34 years of age at conception. In some embodiments, the human can be greater than 33 years of age at conception. In some embodiments, the human can be greater than 32 years of age at conception. In some embodiments, the human can be greater than 31 years of age at conception. In some embodiments, the human can be greater than 30 years of age at conception. In some embodiments, the human can be greater than 29 years of age at conception. In some embodiments, the human can be greater than 28 years of age at conception. In some embodiments, the human can be greater than 27 years of age at conception. In some embodiments, the human can be greater than 26 years of age at conception. In some embodiments, the human can be greater than 25 years of age at conception. In some embodiments, the human can be greater than 24 years of age at conception. In some embodiments, the human can be greater than 23 years of age at conception. In some embodiments, the human can be greater than 22 years of age at conception. In some embodiments, the human can be greater than 21 years of age at conception. In some embodiments, the human can be greater than 20 years of age at conception. In some embodiments, the human can be greater than 19 years of age at conception. In some embodiments, the human can be greater than 18 years of age at conception. In some embodiments, the human can be greater than 17 years of age at conception. In some embodiments, the human can be greater than 16 years of age at conception. In some embodiments, the human can be greater than 15 years of age at conception. In some embodiments, the human can be greater than 14 years of age at conception. In some embodiments, the human can be greater than 13 years of age at conception.

In some embodiments, accessing if a human is or will become pregnant can be performed by ascertaining whether by examination if the human is a male or female and/or sexually active. In other embodiments, accessing if a human is or will become pregnant can be performed by ascertaining whether by inquire if the human is a male or female and/or sexually active.

In other embodiments, ascertaining the age of said human can be performed by inquiry, e.g., asking the human or examining records.

It is also contemplated that the invention can include a method of prophylactically preempting one or more symptoms associated with one or more chromosomal abnormalities in a subject or subject in need thereof comprising administering to said human one or more activators of the hedgehog signaling pathway. In some embodiments, the method can further comprise assessing if a human is or will become pregnant prior to administering to said human one or more activators of the hedgehog signaling pathway. The method can also comprise ascertaining the age of said human. For example, the human can be greater than 35 years of age at conception. In other embodiments, the human can be between 15 and 45 years of age at conception.

It is also contemplated within this invention, a method of normalizing cerebellar structure of a subject or a subject in need thereof comprising administering to said subject or said subject in need thereof a drug. In some embodiments, the drug can be selected from the group consisting of: theophylline, riociguat, forskolin, selective PDE inhibitor, non-selective PDE inhibitor, and any combination thereof. It is also contemplated that any of the drugs mentioned throughout this application can be used.

It is also contemplated within this invention, a method of normalizing hippocampal function of a subject or a subject in need thereof comprising administering to said subject or said subject in need thereof a drug. In some embodiments, the drug can be selected from the group consisting of: theophylline, riociguat, forskolin, selective PDE inhibitor, non-selective PDE inhibitor, and any combination thereof. It is also contemplated that any of the drugs mentioned throughout this application can be used.

It is also contemplated within this invention, a method of treating one or more chromosomal abnormalities in a subject or a subject in need thereof comprising treating said subject or said subject in need thereof with one or more drugs selected from the group consisting of: theophylline, riociguat, forskolin, selective PDE inhibitor, non-selective PDE inhibitor, and any combination thereof and wherein said treating results in at least one of the following phenotypes selected from the group consisting of: normalized cerebellar structure, normalized hippocampal function, normalized cerebellar area, normalized hippocampal area, increased cellular proliferation within the cerebellum, increased cellular proliferation within the hippocampus, increased number of cells within the cerebellum, increased number of cells within the cerebellum, increased cerebellar volume, increased hippocampal volume, increased cerebellar area, increased hippocampal area and any combination thereof.

Any method disclosed in this application wherein drugs and/or one or more activators of the hedgehog signaling pathway can be administered to the subject or subject in need thereof, the drugs and/or one or more activators of the hedgehog signaling pathway can be directly administered into the brain.

### Drug Compositions for Treatment

The inventors have found that one or more symptoms associated with chromosomal abnormalities can be effectively ameliorated by using a pharmaceutical dosage unit comprising theophylline, one or more cGMP activators, one or more cAMP activators, one or more PDE inhibitors (specific and non-specific) and/or combinations thereof.

In an additional aspect of the invention, disclosed herein is a pharmaceutical dosage unit comprising theophylline, one or more cGMP activators, one or more cAMP activators, one or more PDE inhibitors (specific and non-specific) and/or any combination thereof.

The pharmaceutical dosage unit can comprise one or more cGMP activators wherein the one or more cGMP activators can be selected from a group consisting of 3-(5′-hydroxymethyl-2′-furyl)-1-benzylindazole (YC-1), YC-1 derivatives, anthranilic acids derivatives, ataciguat (HMR1766), benzydamine analogs, CFM1517, A-350619, nitrovasodilators, molsidomine, nitroxyl (HNO), BAY 41-2272, BAY 41-8543, BAY 58-2667, cinaciguat (BAY 58-2667), and riociguat (BAY 63-2521). The pharmaceutical dosage unit can also comprise one or more cGMP activators wherein the one or more cGMP activators can be riociguat.

The pharmaceutical dosage unit can comprise one or more cAMP activators wherein the one or more cAMP activators can be selected from a group consisting of: 3-(5′-hydroxymethyl-2′-furyl)-1-benzylindazole (YC-1), glucagon, PDE inhibitors, prostaglandin E1 (PGE1; pharmaceutically known as alprostadil), forskolin, and β-adrenergic activators. The pharmaceutical dosage unit can also comprise one or more cAMP activators wherein the one or more cAMP activators can comprise one or more PDE inhibitors and/or forskolin. The pharmaceutical dosage unit can also comprise one or more cAMP activators wherein the one or more cAMP activators can comprise forskolin.

The pharmaceutical dosage unit can further comprise one or more PDE inhibitors. The one or more PDE inhibitors can comprise a non-selective PDE inhibitor, a PDE-1 selective inhibitor, a PDE-2 selective inhibitor, a PDE-3 selective inhibitor, a PDE-4 selective inhibitor, a PDE-5 selective inhibitor, a PDE-10 selective inhibitor, or a combination thereof. The one or more PDE inhibitors can comprise a specific PDE inhibitor. The one or more PDE inhibitor can be a non-selective PDE inhibitor that can be a methylxanthine derivative. The methylxanthine derivative can be caffeine, theophylline, doxophylline, cipamphylline, neuphylline, pentoxiphylline, or diprophylline. The methylxanthine derivative can be theophylline. The PDE 1 inhibitor can be vinpocetine. The PDE 2 inhibitor can be EHNA. The PDE 3 inhibitor can be inamrinone, anagrelide, or cilostazol. The PDE 4 inhibitor can be mesembrine, rolipram, ibudilast, piclamilast, luteolin, drotaverine, or roflumilast. The PDE 5 inhibitor can be sildenafil, tadalafil, vardenafil, udenafil, avanafil, or dipyridamole. The PDE 10 inhibitor can be papaverine, OMS824 (from Omeros Corporation), and/or PF-2545920 (from Pfizer).

The pharmaceutical dosage unit can comprise a non-specific PDE inhibitor, forskolin, and riociguat. The dosage unit can comprise a specific PDE inhibitor, forskolin, and riociguat. Various combination can be used. For example, the dosage unit can comprise a non-specific PDE inhibitor, theophylline, and riociguat. The dosage unit can comprise a specific PDE inhibitor, theophylline, and riociguat. The dosage unit can comprise a non-specific PDE inhibitor and riociguat. The dosage unit can comprise a specific PDE inhibitor and riociguat. The dosage unit can comprise theophylline, forskolin, and riociguat. The dosage unit can comprise theophylline and riociguat. The dosage unit can comprise forskolin and riociguat. The dosage unit can comprise riociguat.

The invention can include a dosage unit, wherein the dosage unit can be steroid-free.

The pharmaceutical dosage unit can comprise riociguat can in a positive amount selected from a group consisting of: greater than 0.0 μg to 1 μg, 0.5 μg to 2 μg, 1.5 μg to 3.0 μg, 2.5 μg to 10 μg, 5 μg to 15 μg, 12.5 μg to 30 μg, 25 μg to 50 μg, 40 μg to 80 μg, 60 μg to 100 μg, 90 μg to 120 μg, 110 μg to 130 μg, 125 μg to 150 μg, 140 μg to 180 μg, 170 μg to 200 μg, 200 μg to 230 μg, 215 μg to 240 μg, 235 μg to less than 250 μg, less than 250 μg, greater than about 0.0 μg to about 1 μg, about 0.5 μg to about 2 μg, about 1.5 μg to about 3.0 μg, about 2.5 μg to about 10 μg, about 5 μg to about 15 μg, about 12.5 μg to about 30 μg, about 25 μg to about 50 μg, about 40 μg to about 80 μg, about 60 μg to about 100 μg, about 90 μg to about 120 μg, about 110 μg to about 130 μg, about 125 μg to about 150 μg, about 140 μg to about 180 μg, about 170 μg to about 200 μg, about 200 μg to about 230 μg, about 215 μg to about 240 μg, about 235 μg to less than 250 μg greater than 0.0 μg to 2 μg, greater than 0.0 μg to 3 μg, greater than 0.0 μg to 10 μg, greater than 0.0 μg to 15 μg, greater than 0.0 μg to 30 μg, greater than 0.0 μg to 50 μg, greater than 0.0 μg to 80 μg, greater than 0.0 μg to 100 μg, greater than 0.0 μg to 120 μg, greater than 0.0 μg to 130 μg, greater than 0.0 μg to 150 μg, greater than 0.0 μg to 180 μg, greater than 0.0 μg to 200 μg, greater than 0.0 μg to 230 μg, greater than 0.0 μg to 240 μg, greater than 0.0 μg to 250 μg, greater than 0.0 μg to about 2 μg, greater than 0.0 μg to about 3 μg, greater than 0.0 μg to about 10 μg, greater than 0.0 μg to about 15 μg, greater than 0.0 μg to about 30 μg, greater than 0.0 μg to about 50 μg, greater than 0.0 μg to about 80 μg, greater than 0.0 μg to about 100 μg, greater than 0.0 μg to about 120 μg, greater than 0.0 μg to about 130 μg, greater than 0.0 μg to about 150 μg, greater than 0.0 μg to about 180 μg, greater than 0.0 μg to about 200 μg, greater than 0.0 μg to about 230 μg, greater than 0.0 μg to about 240 μg, greater than 0.0 μg to about 250 μg, 0 μg to less than 250 μg, 0.5 μg to less than 250 μg, 1.5 μg to less than 250 μg, 2.5 μg to less than 250 μg, 5 μg to less than 250 μg, 12.5 μg to less than 250 μg, 25 μg to less than 250 μg, 40 μg to less than 250 μg, 60 μg to less than 250 μg, 90 μg to less than 250 μg, 110 μg to less than 250 μg, 125 μg to less than 250 μg, 140 μg to less than 250 μg, 170 μg to less than 250 μg, 200 μg to less than 250 μg, 215 μg to less than 250 μg, 0 μg to less than about 250 μg, about 0.5 μg to less than about 250 μg, about 1.5 μg to less than about 250 μg, about 2.5 μg to less than about 250 μg, about 5 μg to less than about 250 μg, about 12.5 μg to less than about 250 μg, about 25 μg to less than about 250 μg, about 40 μg to less than about 250 μg, about 60 μg to less than about 250 μg, about 90 μg to less than about 250 μg, about 110 μg to less than about 250 μg, about 125 μg to less than about 250 μg, about 140 μg to less than about 250 μg, about 170 μg to less than about 250 μg, about 200 μg to less than about 250 μg, and/or about 215 μg to less about than 250 μg.

The pharmaceutical dosage unit can comprise theophylline in a positive amount selected from a group consisting of: less than 45 mg, 30 mg, 15 mg, 10 mg, 5 mg, 1 mg, 500 μg, 250 μg, 120 μg, 80 μg, 40 μg, or 20 μg and less than about 45 mg, about 30 mg, about 15 mg, about 10 mg, about 5 mg, about 1 mg, about 500 μg, about 250 μg, about 120 μg, about 80 μg, about 40 μg, or about 20 μg, greater than 0 μg to 20 μg, 10 μg to 40 μg, 30 μg to 80 μg, 70 μg to 120 μg, 100 μg to 250 μg, 200 μg to 500 μg, 400 μg to 1 mg, 900 μg to 5 mg, 4 mg to 10 mg, 9 mg to 15 mg, 14 mg to 30 mg, 25 mg to 45 mg, greater than 0 μg to about 20 μg, about 10 μg to about 40 μg, about 30 μg to about 80 μg, about 70 μg to about 120 μg, about 100 μg to about 250 μg, about 200 μg to about 500 μg, about 400 μg to about 1 mg, about 900 μg to about 5 mg, about 4 mg to about 10 mg, about 9 mg to about 15 mg, about 14 mg to about 30 mg, about 25 mg to about 45 mg, greater than 0 μg to 40 μg, greater than 0 μg to 80 μg, greater than 0 μg to 120 μg, greater than 0 μg to 250 μg, greater than 0 μg to 500 μg, greater than 0 μg to 1 mg, greater than 0 μg to 5 mg, greater than 0 μg to 10 mg, greater than 0 μg to 15 mg, greater than 0 μg to 30 mg, greater than 0 μg to 45 mg, greater than 0 μg to about 40 μg, greater than 0 μg to about 80 μg, greater than 0 μg to about 120 μg, greater than 0 μg to about 250 μg, greater than 0 μg to about 500 μg, greater than 0 μg to about 1 mg, greater than 0 μg to about 5 mg, greater than 0 μg to about 10 mg, greater than 0 μg to about 15 mg, greater than 0 μg to about 30 mg, greater than 0 μg to about 45 mg, greater than 0 μg to 45 mg, 10 μg to 45 mg, 30 μg to 45 mg, 70 μg to 45 mg, 100 μg to 45 mg, 200 μg to 45 mg, 400 μg to 45 mg, 900 μg to 45 mg, 4 mg to 45 mg, 9 mg to 45 mg, 14 mg to 45 mg, 35 mg to 45 mg, greater than 0 μg to about 45 mg, about 10 μg to about 45 mg, about 30 μg to about 45 mg, about 70 μg to about 45 mg, about 100 μg to about 45 mg, about 200 μg to about 45 mg, about 400 μg to about 45 mg, about 900 μg to about 45 mg, about 4 mg to about 5 mg, about 9 mg to about 45 mg, about 14 mg to about 45 mg, and/or about 35 mg to about 45 mg.

The pharmaceutical dosage unit can comprise forskolin in a positive amount selected from a group consisting of: less than 500 mg to 450 mg, 475 mg to 425 mg, 435 mg to 400 mg, 415 mg to 300 mg, 325 mg to 250 mg, 275 mg to 150 mg, 200 mg to 100 mg, 135 mg to 80 mg, 95 mg to 65 mg, 75 mg to 50 mg, 60 mg to 40 mg, 45 mg to 25 mg, 30 mg to 20 mg, 15 mg to 5 mg, 10 mg to 2.5 mg, 3.5 mg to 1 mg, 2 mg to greater than 0 mg, less than about 500 mg to about 450 mg, about 475 mg to about 425 mg, about 435 mg to about 400 mg, about 415 mg to about 300 mg, about 325 mg to about 250 mg, about 275 mg to about 150 mg, about 200 mg to about 100 mg, about 135 mg to about 80 mg, about 95 mg to about 65 mg, about 75 mg to about 50 mg, about 60 mg to about 40 mg, about 45 mg to about 25 mg, about 30 mg to about 20 mg, about 15 mg to about 5 mg, about 10 mg to about 2.5 mg, about 3.5 mg to about 1 mg, about 2 mg to greater than 0 mg, 500 mg, less than 500 mg to 450 mg, less than 500 mg to 425 mg, less than 500 mg to 400 mg, less than 500 mg to 300 mg, less than 500 mg to 250 mg, less than 500 mg to 150 mg, less than 500 mg to 100 mg, less than 500 mg to 80 mg, less than 500 mg to 65 mg, less than 500 mg to 50 mg, less than 500 mg to 40 mg, less than 500 mg to 25 mg, less than 500 mg to 20 mg, less than 500 mg to 5 mg, less than 500 mg to 2.5 mg, less than 500 mg to 1 mg, less than 500 mg to greater than 0 mg, about 500 mg, less than about 500 mg to about 450 mg, less than about 500 mg to about 425 mg, less than about 500 mg to about 400 mg, less than about 500 mg to about 300 mg, less than about 500 mg to about 250 mg, less than about 500 mg to about 150 mg, less than about 500 mg to about 100 mg, less than about 500 mg to about 80 mg, less than about 500 mg to about 65 mg, less than about 500 mg to about 50 mg, less than about 500 mg to about 40 mg, less than about 500 mg to about 25 mg, less than about 500 mg to about 20 mg, less than about 500 mg to about 5 mg, less than about 500 mg to about 2.5 mg, less than about 500 mg to about 1 mg, less than about 500 mg to greater than about 0 mg, greater than 0 mg to 450 mg, greater than 0 mg to 425 mg, greater than 0 mg to 400 mg, greater than 0 mg to 300 mg, greater than 0 mg to 250 mg, greater than 0 mg to 150 mg, greater than 0 mg to 100 mg, greater than 0 mg to 80 mg, greater than 0 mg to 65 mg, greater than 0 mg to 50 mg, greater than 0 mg to 40 mg, greater than 0 mg to 25 mg, greater than 0 mg to 20 mg, greater than 0 mg to 5 mg, greater than 0 mg to 2.5 mg, greater than 0 mg to 1 mg, greater than 0 mg to about 450 mg, greater than 0 mg to about 425 mg, greater than 0 mg to about 400 mg, greater than 0 mg to about 300 mg, greater than 0 mg to about 250 mg, greater than 0 mg to about 150 mg, greater than 0 mg to about 100 mg, greater than 0 mg to about 80 mg, greater than 0 mg to about 65 mg, greater than 0 mg to about 50 mg, greater than 0 mg to about 40 mg, greater than 0 mg to about 25 mg, greater than 0 mg to about 20 mg, greater than 0 mg to about 5 mg, greater than 0 mg to about 2.5 mg, and/or greater than 0 mg to about 1 mg.

The pharmaceutical dosage unit can comprise cilastazol in a positive amount selected from a group consisting of: less than 500 mg to 450 mg, 475 mg to 425 mg, 435 mg to 400 mg, 415 mg to 300 mg, 325 mg to 250 mg, 275 mg to 150 mg, 200 mg to 100 mg, 135 mg to 80 mg, 95 mg to 65 mg, 75 mg to 50 mg, 60 mg to 40 mg, 45 mg to 25 mg, 30 mg to 20 mg, 15 mg to 5 mg, 10 mg to 2.5 mg, 3.5 mg to 1 mg, 2 mg to greater than 0 mg, less than about 500 mg to about 450 mg, about 475 mg to about 425 mg, about 435 mg to about 400 mg, about 415 mg to about 300 mg, about 325 mg to about 250 mg, about 275 mg to about 150 mg, about 200 mg to about 100 mg, about 135 mg to about 80 mg, about 95 mg to about 65 mg, about 75 mg to about 50 mg, about 60 mg to about 40 mg, about 45 mg to about 25 mg, about 30 mg to about 20 mg, about 15 mg to about 5 mg, about 10 mg to about 2.5 mg, about 3.5 mg to about 1 mg, about 2 mg to greater than 0 mg, 500 mg, less than 500 mg to 450 mg, less than 500 mg to 425 mg, less than 500 mg to 400 mg, less than 500 mg to 300 mg, less than 500 mg to 250 mg, less than 500 mg to 150 mg, less than 500 mg to 100 mg, less than 500 mg to 80 mg, less than 500 mg to 65 mg, less than 500 mg to 50 mg, less than 500 mg to 40 mg, less than 500 mg to 25 mg, less than 500 mg to 20 mg, less than 500 mg to 5 mg, less than 500 mg to 2.5 mg, less than 500 mg to 1 mg, less than 500 mg to greater than 0 mg, about 500 mg, less than about 500 mg to about 450 mg, less than about 500 mg to about 425 mg, less than about 500 mg to about 400 mg, less than about 500 mg to about 300 mg, less than about 500 mg to about 250 mg, less than about 500 mg to about 150 mg, less than about 500 mg to about 100 mg, less than about 500 mg to about 80 mg, less than about 500 mg to about 65 mg, less than about 500 mg to about 50 mg, less than about 500 mg to about 40 mg, less than about 500 mg to about 25 mg, less than about 500 mg to about 20 mg, less than about 500 mg to about 5 mg, less than about 500 mg to about 2.5 mg, less than about 500 mg to about 1 mg, less than about 500 mg to greater than about 0 mg, greater than 0 mg to 450 mg, greater than 0 mg to 425 mg, greater than 0 mg to 400 mg, greater than 0 mg to 300 mg, greater than 0 mg to 250 mg, greater than 0 mg to 150 mg, greater than 0 mg to 100 mg, greater than 0 mg to 80 mg, greater than 0 mg to 65 mg, greater than 0 mg to 50 mg, greater than 0 mg to 40 mg, greater than 0 mg to 25 mg, greater than 0 mg to 20 mg, greater than 0 mg to 5 mg, greater than 0 mg to 2.5 mg, greater than 0 mg to 1 mg, greater than 0 mg to about 450 mg, greater than 0 mg to about 425 mg, greater than 0 mg to about 400 mg, greater than 0 mg to about 300 mg, greater than 0 mg to about 250 mg, greater than 0 mg to about 150 mg, greater than 0 mg to about 100 mg, greater than 0 mg to about 80 mg, greater than 0 mg to about 65 mg, greater than 0 mg to about 50 mg, greater than 0 mg to about 40 mg, greater than 0 mg to about 25 mg, greater than 0 mg to about 20 mg, greater than 0 mg to about 5 mg, greater than 0 mg to about 2.5 mg, and/or greater than 0 mg to about 1 mg.

The pharmaceutical dosage unit can comprise roflumilast in a positive amount selected from a group consisting of: less than 10 mg, 5 mg, 1 mg, 500 μg, 250 μg, 120 μg, 80 μg, 40 μg, or 20 μg and less than about 10 mg, about 5 mg, about 1 mg, about 500 μg, about 250 μg, about 120 μg, about 80 μg, about 40 μg, or about 20 μg. Roflumilast can be also given, e.g., intranasally, and/or present in an amount selected from a group consisting of: greater than 0 μg to 20 μg, 10 μg to 40 μg, 30 μg to 80 μg, 70 μg to 120 μg, 100 μg to 250 μg, 200 μg to 500 μg, 400 μg to 1 mg, 900 μg to 5 mg, 4 mg to 10 mg, 9 mg to 15 mg, 14 mg to 30 mg, 25 mg to 45 mg, greater than 0 μg to about 20 μg, about 10 μg to about 40 μg, about 30 μg to about 80 μg, about 70 μg to about 120 μg, about 100 μg to about 250 μg, about 200 μg to about 500 μg, about 400 μg to about 1 mg, about 900 μg to about 5 mg, about 4 mg to about 10 mg, greater than 0 μg to 40 μg, greater than 0 μg to 80 μg, greater than 0 μg to 120 μg, greater than 0 μg to 250 μg, greater than 0 μg to 500 μg, greater than 0 μg to 1 mg, greater than 0 μg to 5 mg, greater than 0 ng to 10 mg, greater than 0 μg to 15 mg, greater than 0 μg to 30 mg, greater than 0 μg to 45 mg, greater than 0 μg to about 40 μg, greater than 0 μg to about 80 μg, greater than 0 μg to about 120 μg, greater than 0 μg to about 250 μg, greater than 0 μg to about 500 μg, greater than 0 μg to about 1 mg, greater than 0 μg to about 5 mg, greater than 0 μg to about 10 mg, greater than 0 μg to 10 mg, 10 μg to 10 mg, 30 μg to 10 mg, 70 μg to 10 mg, 100 μg to 10 mg, 200 μg to 10 mg, 400 μg to 10 mg, 900 μg to 10 mg, 4 mg to 10 mg, 9 mg to 10 mg, greater than 0 μg to about 10 mg, about 10 μg to about 10 mg, about 30 μg to about 10 mg, about 70 μg to about 10 mg, about 100 μg to about 10 mg, about 200 μg to about 10 mg, about 400 μg to about 10 mg, about 900 ng to about 10 mg, about 4 mg to about 10 mg, and/or about 9 mg to about 10.

The pharmaceutical dosage unit can comprise papaverine in a positive amount selected from a group consisting of: less than 500 mg to 450 mg, 475 mg to 425 mg, 435 mg to 400 mg, 415 mg to 300 mg, 325 mg to 250 mg, 275 mg to 150 mg, 200 mg to 100 mg, 135 mg to 80 mg, 95 mg to 65 mg, 75 mg to 50 mg, 60 mg to 40 mg, 45 mg to 25 mg, 30 mg to 20 mg, 15 mg to 5 mg, 10 mg to 2.5 mg, 3.5 mg to 1 mg, 2 mg to greater than 0 mg, less than about 500 mg to about 450 mg, about 475 mg to about 425 mg, about 435 mg to about 400 mg, about 415 mg to about 300 mg, about 325 mg to about 250 mg, about 275 mg to about 150 mg, about 200 mg to about 100 mg, about 135 mg to about 80 mg, about 95 mg to about 65 mg, about 75 mg to about 50 mg, about 60 mg to about 40 mg, about 45 mg to about 25 mg, about 30 mg to about 20 mg, about 15 mg to about 5 mg, about 10 mg to about 2.5 mg, about 3.5 mg to about 1 mg, about 2 mg to greater than 0 mg, 500 mg, less than 500 mg to 450 mg, less than 500 mg to 425 mg, less than 500 mg to 400 mg, less than 500 mg to 300 mg, less than 500 mg to 250 mg, less than 500 mg to 150 mg, less than 500 mg to 100 mg, less than 500 mg to 80 mg, less than 500 mg to 65 mg, less than 500 mg to 50 mg, less than 500 mg to 40 mg, less than 500 mg to 25 mg, less than 500 mg to 20 mg, less than 500 mg to 5 mg, less than 500 mg to 2.5 mg, less than 500 mg to 1 mg, less than 500 mg to greater than 0 mg, about 500 mg, less than about 500 mg to about 450 mg, less than about 500 mg to about 425 mg, less than about 500 mg to about 400 mg, less than about 500 mg to about 300 mg, less than about 500 mg to about 250 mg, less than about 500 mg to about 150 mg, less than about 500 mg to about 100 mg, less than about 500 mg to about 80 mg, less than about 500 mg to about 65 mg, less than about 500 mg to about 50 mg, less than about 500 mg to about 40 mg, less than about 500 mg to about 25 mg, less than about 500 mg to about 20 mg, less than about 500 mg to about 5 mg, less than about 500 mg to about 2.5 mg, less than about 500 mg to about 1 mg, less than about 500 mg to greater than about 0 mg, greater than 0 mg to 450 mg, greater than 0 mg to 425 mg, greater than 0 mg to 400 mg, greater than 0 mg to 300 mg, greater than 0 mg to 250 mg, greater than 0 mg to 150 mg, greater than 0 mg to 100 mg, greater than 0 mg to 80 mg, greater than 0 mg to 65 mg, greater than 0 mg to 50 mg, greater than 0 mg to 40 mg, greater than 0 mg to 25 mg, greater than 0 mg to 20 mg, greater than 0 mg to 5 mg, greater than 0 mg to 2.5 mg, greater than 0 mg to 1 mg, greater than 0 mg to about 450 mg, greater than 0 mg to about 425 mg, greater than 0 mg to about 400 mg, greater than 0 mg to about 300 mg, greater than 0 mg to about 250 mg, greater than 0 mg to about 150 mg, greater than 0 mg to about 100 mg, greater than 0 mg to about 80 mg, greater than 0 mg to about 65 mg, greater than 0 mg to about 50 mg, greater than 0 mg to about 40 mg, greater than 0 mg to about 25 mg, greater than 0 mg to about 20 mg, greater than 0 mg to about 5 mg, greater than 0 mg to about 2.5 mg, and/or greater than 0 mg to about 1 mg.

The dosage unit or composition comprising the dosage unit can be suitable for administration by a method selected from a group consisting of: oral administration, transmucosal administration, buccal administration, inhalation administration, intranasal administration, parental administration, intravenous administration, subcutaneous administration, intramuscular administration, sublingual administration, transdermal administration, and rectal administration. For ease of use, the dosage unit or composition comprising the dosage unit can be suitable for oral administration, inhalational administration, intranasal administration, or a combination thereof. The dosage unit or composition comprising the dosage unit can also be a liquid. For example, the drug can be breast milk containing the drug. In some embodiments, the drug can be fortified baby's formula and/or milk.

The dosage unit or composition can be affected by pH. In some embodiments, the dosage unit or composition comprising the dosage unit can have a pH of 7.0 or less than 7.0. For example, the dosage unit or composition comprising the dosage unit can have a pH of 7.0. The dosage unit or composition comprising the dosage unit can have a pH of less than 7.0. The dosage unit or composition comprising the dosage unit can have a pH of less than 6.9. The dosage unit or composition comprising the dosage unit can have a pH of less than 6.5. The dosage unit or composition comprising the dosage unit can have a pH of greater than 7.0. For example, the dosage unit or composition comprising the dosage unit can have a pH of greater than 7.1. The dosage unit or composition comprising the dosage unit can have a pH of greater than 7.5. The dosage unit or composition comprising the dosage unit can have a pH of greater than 8.0. The dosage unit or composition comprising the dosage unit can have a pH of greater than 9.0. The dosage unit or composition comprising the dosage unit can have a pH selected from the group consisting of: 5.0 to 5.5; 5.1 to 5.6; 5.2 to 5.7; 5.3 to 5.8; 5.4 to 5.9; 5.5 to 6.0; 5.6 to 6.1; 5.7 to 6.2; 5.8 to 6.3; 5.9 to 6.4; 6.0 to 6.5; 6.1 to 6.6; 6.2 to 6.7; 6.3 to 6.8; 6.4 to 6.9; 6.5 to 7.0; 6.6 to 7.1; 6.7 to 7.2; 6.8 to 7.3; 6.9 to 7.4; 7.0 to 7.5; 7.1 to 7.6; 7.2 to 7.7; 7.3 to 7.8; 7.4 to 7.9; 7.5 to 8.0; 7.6 to 8.1; 7.7 to 8.2; 7.8 to 8.3; 7.9 to 8.4; 8.0 to 8.5; 8.1 to 8.6; 8.2 to 8.7; 8.3 to 8.8; 8.4 to 8.9; 8.5 to 9.0; 8.6 to 9.1; 8.7 to 9.2; 8.8 to 9.3; 8.9 to 9.4; 9.0 to 9.5; about 5.0 to about 5.5; about 5.1 to about 5.6; about 5.2 to about 5.7; about 5.3 to about 5.8; about 5.4 to about 5.9; about 5.5 to about 6.0; about 5.6 to about 6.1; about 5.7 to about 6.2; about 5.8 to about 6.3; about 5.9 to about 6.4; about 6.0 to about 6.5; about 6.1 to about 6.6; about 6.2 to about 6.7; about 6.3 to about 6.8; about 6.4 to about 6.9; about 6.5 to about 7.0; about 6.6 to about 7.1; about 6.7 to about 7.2; about 6.8 to about 7.3; about 6.9 to about 7.4; about 7.0 to about 7.5; about 7.1 to about 7.6; about 7.2 to about 7.7; about 7.3 to about 7.8; about 7.4 to about 7.9; about 7.5 to about 8.0; about 7.6 to about 8.1; about 7.7 to about 8.2; about 7.8 to about 8.3; about 7.9 to about 8.4; about 8.0 to about 8.5; about 8.1 to about 8.6; about 8.2 to about 8.7; about 8.3 to about 8.8; about 8.4 to about 8.9; about 8.5 to about 9.0; about 8.6 to about 9.1; about 8.7 to about 9.2; about 8.8 to about 9.3; about 8.9 to about 9.4; and/or about 9.0 to about 9.5.

Excipients can be added to one or more therapeutic agents or compositions. The excipients that can be used in the invention can include those found in the Handbook of Pharmaceutical Excipients, Sixth Edition (2009), Eds. R. C. Rowe, P. J. Shesky, and M. E. Quinn. For example, it is contemplated that the following excipients can be added separately or in any combination, to one or more therapeutic agents or composition: Acacia, Acesulfame Potassium, Acetic Acid—Glacial, Acetone, Acetyltributyl Citrate, Acetyltriethyl Citrate, Adipic Acid, Agar, Albumin, Alcohol, Alginic Acid, Aliphatic Polyesters, Alitame, Almond Oil, Alpha Tocopherol, Aluminum Hydroxide Adjuvant, Aluminum Monostearate, Aluminum Oxide, Aluminum Phosphate Adjuvant, Ammonia Solution, Ammonium Alginate, Ammonium Chloride, Ascorbic Acid, Ascorbyl Palmitate, Aspartame, Attapulgite, Bentonite, Benzalkonium Chloride, Benzethonium Chloride, Benzoic Acid, Benzyl Alcohol, Benzyl Benzoate, Boric Acid, Bronopol, Butylated Hydroxyanisole, Butylated Hydroxytoluene, Butylene Glycol, Butylparaben, Calcium Acetate, Calcium Alginate, Calcium Carbonate, Calcium Chloride, Calcium Hydroxide, Calcium Lactate, Calcium Phosphate—Dibasic Anhydrous, Calcium Phosphate—Dibasic Dihydrate, Calcium Phosphate—Tribasic, Calcium Silicate, Calcium Stearate, Calcium Sulfate, Canola Oil, Carbomer, Carbon Dioxide, Carboxymethylcellulose Calcium, Carboxymethylcellulose Sodium, Carrageenan, Castor Oil, Castor Oil—Hydrogenated, Cellulose—Microcrystalline, Cellulose—Microcrystalline and Carboxymethylcellulose Sodium, Cellulose—Powdered, Cellulose—Silicified Microcrystalline, Cellulose Acetate, Cellulose Acetate Phthalate, Ceratonia, Ceresin, Cetostearyl Alcohol, Cetrimide, Cetyl Alcohol, Cetylpyridinium Chloride, Chitosan, Chlorhexidine, Chlorobutanol, Chlorocresol, Chlorodifluoroethane (HCFC), Chlorofluorocarbons (CFC), Chloroxylenol, Cholesterol, Citric Acid Monohydrate, Coconut Oil, Colloidal Silicon Dioxide, Coloring Agents, Copovidone, Corn Oil, Corn Starchand Pregelatinized Starch, Cottonseed Oil, Cresol, Croscarmellose Sodium, Crospovidone, Cyclodextrins, Cyclomethicone, Denatonium Benzoate, Dextrates, Dextrin, Dextrose, Dibutyl Phthalate, Dibutyl Sebacate, Diethanolamine, Diethyl Phthalate, Difluoroethane (HFC), Dimethicone, Dimethyl Ether, Dimethyl Phthalate, Dimethyl Sulfoxide, Dimethylacetamide, Disodium Edetate, Docusate Sodium, Edetic Acid, Erythorbic Acid, Erythritol, Ethyl Acetate, Ethyl Lactate, Ethyl Maltol, Ethyl Oleate, Ethyl Vanillin, Ethylcellulose, Ethylene Glycol Stearates, Ethylene Vinyl Acetate, Ethylparaben, Fructose, Fumaric Acid, Gelatin, Glucose—Liquid, Glycerin, Glyceryl Behenate, Glyceryl Monooleate, Glyceryl Monostearate, Glyceryl Palmitostearate, Glycine, Glycofurol, Guar Gum, Hectorite, Heptafluoropropane (HFC), Hexetidine, Hydrocarbons (HC), Hydrochloric Acid, Hydrophobic Colloidal Silica, Hydroxyethyl Cellulose, Hydroxyethylmethyl Cellulose, Hydroxypropyl Betadex, Hydroxypropyl Cellulose, Hydroxypropyl Cellulose—Low-substituted, Hydroxypropyl Starch, Hypromellose, Hypromellose Acetate Succinate, Hypromellose Phthalate, Imidurea, Inulin, Iron Oxides, Isomalt, Isopropyl Alcohol, Isopropyl Myristate, Isopropyl Palmitate, Kaolin, Lactic Acid, Lactitol, Lactose—Anhydrous, Lactose—Inhalation, Lactose—Monohydrate, Lactose—Monohydrate and Corn Starch, Lactose—Monohydrate and Microcrystalline Cellulose, Lactose—Monohydrate and Povidone, Lactose—Monohydrate and Powdered Cellulose, Lactose—Spray-Dried, Lanolin, Lanolin—Hydrous, Lanolin Alcohols, Laurie Acid, Lecithin, Leucine, Linoleic Acid, Macrogol 15 Hydroxystearate, Magnesium Aluminum Silicate, Magnesium Carbonate, Magnesium Oxide, Magnesium Silicate, Magnesium Stearate, Magnesium Trisilicate, Maleic Acid, Malic Acid, Maltitol, Maltitol Solution, Maltodextrin, Maltol, Maltose, Mannitol, Medium-chain Triglycerides, Meglumine, Menthol, Methionine, Methylcellulose, Methylparaben, Mineral Oil, Mineral Oil—Light, Mineral Oil and Lanolin Alcohols, Monoethanolamine, Monosodium Glutamate, Monothioglycerol, Myristic Acid, Myristyl Alcohol, Neohesperidin Dihydrochalcone, Neotame, Nitrogen, Nitrous Oxide, Octyldodecanol, Oleic Acid, Oleyl Alcohol, Olive Oil, Palmitic Acid, Paraffin, Peanut Oil, Pectin, Pentetic Acid, Petrolatum, Petrolatum and Lanolin Alcohols, Phenol, Phenoxyethanol, Phenylethyl Alcohol, Phenylmercuric Acetate, Phenylmercuric Borate, Phenylmercuric Nitrate, Phospholipids, Phosphoric Acid, Polacrilin Potassium, Poloxamer, Polycarbophil, Polydextrose, Poly (DL-Lactic Acid), Polyethylene Glycol, Polyethylene Oxide, Polymethacrylates, Poly(methyl vinylether/maleic anhydride), Polyoxyethylene Alkyl Ethers, Polyoxyethylene Castor Oil Derivatives, Polyoxyethylene Sorbitan Fatty Acid Esters, Polyoxyethylene Stearates, Polyoxylglycerides, Polyvinyl Acetate Phthalate, Polyvinyl Alcohol, Potassium Alginate, Potassium Alum, Potassium Benzoate, Potassium Bicarbonate, Potassium Chloride, Potassium Citrate, Potassium Hydroxide, Potassium Metabisulfite, Potassium Sorbate, Povidone, Propionic Acid, Propyl Gallate, Propylene Carbonate, Propylene Glycol, Propylene Glycol Alginate, Propylparaben, Propylparaben Sodium, Pyrrolidone, Raffinose, Saccharin, Saccharin Sodium, Safflower Oil, Saponite, Sesame Oil, Shellac, Simethicone, Sodium Acetate, Sodium Alginate, Sodium Ascorbate, Sodium Benzoate, Sodium Bicarbonate, Sodium Borate, Sodium Carbonate, Sodium Chloride, Sodium Citrate Dihydrate, Sodium Cyclamate, Sodium Formaldehyde Sulfoxylate, Sodium Hyaluronate, Sodium Hydroxide, Sodium Lactate, Sodium Lauryl Sulfate, Sodium Metabisulfite, Sodium Phosphate—Dibasic, Sodium Phosphate—Monobasic, Sodium Propionate, Sodium Starch Glycolate, Sodium Stearyl Fumarate, Sodium Sulfite, Sodium Thiosulfate, Sorbic Acid, Sorbitan Esters (Sorbitan Fatty Acid Esters), Sorbitol, Soybean Oil, Starch, Starch—Pregelatinized, Starch—Sterilizable Maize, Stearic Acid, Stearyl Alcohol, Sucralose, Sucrose, Sucrose Octaacetate, Sugar—Compressible, Sugar—Confectioner's, Sugar Spheres, Sulfobutylether b-Cyclodextrin, Sulfur Dioxide, Sulfuric Acid, Sunflower Oil, Suppository Bases—Hard Fat, Tagatose, Talc, Tartaric Acid, Tetrafluoroethane (HFC), Thaumatin, Thimerosal, Thymol, Titanium Dioxide, Tragacanth, Trehalose, Triacetin, Tributyl Citrate, Tricaprylin, Triethanolamine, Triethyl Citrate, Triolein, Vanillin, Vegetable Oil—Hydrogenated, Vitamin E Polyethylene Glycol Succinate, Water, Wax—Anionic Emulsifying, Wax—Carnauba, Wax—Cetyl Esters, Wax—Microcrystalline, Wax—Nonionic Emulsifying, Wax—White, Wax—Yellow, Xanthan Gum, Xylitol, Zein, Zinc Acetate, and/or Zinc Stearate.

The dosage unit or composition comprising the dosage unit can further comprise one or more excipients. The dosage unit or composition can further comprise one or more excipients, wherein the one or more excipients can be selected from a group consisting of: detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

The dosage unit or composition can further comprise one or more cytochrome p450 inhibitors. The dosage unit or composition can further comprise one or more cytochrome p450 inhibitors wherein the one or more cytochrome p450 inhibitors can fully or partially inhibit a cytochrome p450 selected from a group consisting of: CYP1, CYP1A1, CYP1A2, CYP1B1, CYP2, CYP2A6, CYP2A7, CYP2A13, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3, CYP3A4, CYP3A5, CYP3A7, CYP3A43, CYP4, CYP4A11, CYP4A22, CYP4B1, CYP4F2, CYP4F3, CYP4F8, CYP4F11, CYP4F12, CYP4F22, CYP4V2, CYP4X1, CYP4Z1, CYP5, CYP5A1, CYP7, CYP7A1, CYP7B1, CYP8, CYP8A1, CYP8B1, CYP11, CYP11A1, CYP11B1, CYP11B2, CYP17, CYP17A1, CYP19, CYP19A1, CYP20, CYP20A1, CYP21, CYP21A2, CYP24, CYP24A1, CYP26, CYP26A1, CYP26B1, CYP26C1, CYP27, CYP27A1, CYP27B1, CYP27C1, CYP39, CYP39A1, CYP46, CYP46A1, CYP51, and CYP51A1. The dosage unit or composition can also comprise one or more cytochrome p450 inhibitors wherein the one or more cytochrome p450 inhibitors can fully or partially inhibit CYP1. The dosage unit or composition can also comprise one or more cytochrome p450 inhibitors wherein the one or more cytochrome p450 inhibitors can fully or partially inhibit CYP1A2. The dosage unit or composition can also comprise one or more CYP1A2 inhibitors wherein the one or more CYP1A2 inhibitors can be selected from a group consisting of: fluoroquinolone, selective serotonin reuptake inhibitor (SSRI), calcium channel blocker, herbal tea, naringenin, H2-receptor activators, antiarrhythmic agent, interferon, xanthotoxin, mibefradil, cumin, turmeric, and isoniazid. The dosage unit or composition can also further comprise one or more CYP1A2 inhibitors wherein the one or more CYP1A2 inhibitors can be grapefruit juice. The dosage unit or composition can also further comprise one or more CYP1A2 inhibitors wherein the one or more CYP1A2 inhibitors can be naringenin.

The dosage unit or composition can further comprise one or more β-adrenergic activators. The dosage unit or composition can also further comprise one or more β-adrenergic activators wherein the one or more β-adrenergic activators can be a β₁-adrenergic activators and/or β2-adrenergic activators. The dosage unit or composition can also further comprise one or more β-adrenergic activators wherein the one or more β-adrenergic activators can be a β₁-adrenergic activators. The dosage unit or composition can also further comprise one or more 3-adrenergic activators wherein the one or more β-adrenergic activators can be a β₁-adrenergic activators selected from a group consisting of: dobutamine, isoproterenol, xamoterol and epinephrine. The dosage unit or composition can also further comprise one or more β-adrenergic activators wherein the one or more β-adrenergic activators can be a β₂-adrenergic activators. The dosage unit or composition can also further comprise one or more β-adrenergic activators wherein the one or more β-adrenergic activators can be a β₂-adrenergic activators selected from a group consisting of: albuterol, levalbuterol, fenoterol, formoterol, isoproterenol (β₁ and β₂), metaproterenol, salmeterol, terbutaline, clenbuterol, isoetarine, pirbuterol, procaterol, ritodrine, and epinephrine. The dosage unit or composition can also further comprise one or more β-adrenergic activators wherein the one or more β-adrenergic activators can be selected from a group consisting of: arbutamine, befunolol, bromoacetylalprenololmenthane, broxaterol, cimaterol, cirazoline, denopamine, dopexamine, etilefrine, hexoprenaline, higenamine, isoxsuprine, mabuterol, methoxyphenamine, nylidrin, oxyfedrine, prenalterol, ractopamine, reproterol, rimiterol, tretoquinol, tulobuterol, zilpaterol, and zinterol.

Diagnosing and Treating Chromosomal Abnormalities and/or One or More Symptoms Associated with Chromosomal Abnormalities

Also disclosed are methods of diagnosing chromosomal abnormalities and/or one or more symptoms associated with chromosomal abnormalities, the methods comprising (a) obtaining one or more biological samples from the subject; (b) measuring a level of one or more members of the hedgehog signaling pathway in the one or more biological samples from the subject; (c) diagnosing the subject with chromosomal abnormalities and/or one or more symptoms associated with chromosomal abnormalities based upon the level of one or more members of the hedgehog signaling pathway that can be lower than a threshold level; and (d) administering to the subject a treatment for one or more symptoms associated with chromosomal abnormalities. Any of the methods of diagnosing chromosomal abnormalities and/or one or more symptoms associated with chromosomal abnormalities disclosed herein can be used in combination with any of the pharmaceutical dosage units or composition disclosed herein.

### Making Drug Compositions

In an additional aspect of the invention, disclosed herein are methods of making a pharmaceutical dosage unit comprising combining one or more cGMP activators, one or more cAMP activators, one or more PDE inhibitors (specific and/or non-specific) in any combination thereof.

The methods can comprise combining one or more cGMP activators wherein the one or more cGMP activators can be selected from a group consisting of: 3-(5′-hydroxymethyl-2′-furyl)-1-benzylindazole (YC-1), YC-1 derivatives, anthranilic acids derivatives, ataciguat (HMR1766), benzydamine analogs, CFM1517, A-350619, nitrovasodilators, molsidomine, nitroxyl (HNO), BAY 41-2272, BAY 41-8543, BAY 58-2667, cinaciguat (BAY 58-2667), and riociguat (BAY 63-2521). The methods can also comprise combining one or more cGMP activators wherein the one or more cGMP activators can comprise riociguat. The methods can comprise combining one or more cAMP activators wherein the one or more cAMP activators can be selected from a group consisting of: 3-(5′-hydroxymethyl-2′-furyl)-1-benzylindazole (YC-1), glucagon, PDE inhibitors, prostaglandin E1 (PGE1; pharmaceutically known as alprostadil), forskolin, and β-adrenergic activators.

The methods can comprise combining one or more cAMP activators wherein the one or more cAMP activators can further comprise combining one or more PDE inhibitors and/or forskolin.

The methods can comprise combining one or more cAMP activators wherein the one or more cAMP activators can be forskolin.

The one or more PDE inhibitors can be a non-selective PDE inhibitor, a PDE-1 selective inhibitor, a PDE-2 selective inhibitor, a PDE-3 selective inhibitor, a PDE-4 selective inhibitor, a PDE-5 selective inhibitor, a PDE-10 selective inhibitor, or a combination thereof. The methods can also comprise combining one or more PDE inhibitors wherein the one or more one or more PDE inhibitors can be a specific PDE inhibitor. The methods can also comprise combining one or more PDE inhibitors wherein the one or more PDE inhibitors can be a non-selective PDE inhibitor that can be a methylxanthine derivative. The methods can also comprise combining methylxanthine derivative that can be caffeine, theophylline, doxophylline, cipamphylline, neuphylline, pentoxiphylline, or diprophylline. The methods can also comprise combining the methylxanthine derivative that can be theophylline. The methods can also comprise combining a PDE 1 inhibitor can be vinpocetine. The methods can also comprise combining a PDE 2 inhibitor that can be EHNA. The methods can also comprise combining a PDE 3 inhibitor that can be inamrinone, anagrelide, or cilostazol. The methods can also comprise combining a PDE 4 inhibitor that can be mesembrine, rolipram, ibudilast, piclamilast, luteolin, drotaverine, or roflumilast. The methods can also comprise combining a PDE 5 inhibitor that can be sildenafil, tadalafil, vardenafil, udenafil, avanafil, or dipyridamole. The methods can also comprise combining a PDE 10 inhibitor that can be papaverine, OMS824 (from Omeros Corporation), and/or PF-2545920 (from Pfizer).

The dosage unit can be formed by combining a non-specific PDE inhibitor, forskolin, and riociguat. The dosage unit can be formed by combining a specific PDE inhibitor, forskolin, and riociguat. The dosage unit can be formed by combining a non-specific PDE inhibitor, theophylline, and riociguat. The dosage unit can be formed by combining a specific PDE inhibitor, theophylline, and riociguat. The dosage unit can be formed by combining a non-specific PDE inhibitor and riociguat. The dosage unit can be formed by combining a specific PDE inhibitor and riociguat. The dosage unit can be formed by combining theophylline and riociguat. The dosage unit can be formed by combining forskolin and riociguat. The dosage unit can be formed by combining theophylline, forskolin, and riociguat.

Riociguat can be combined or present in a positive amount selected from a group consisting of: greater than 0.0 μg to 1 μg, 0.5 μg to 2 μg, 1.5 μg to 3.0 μg, 2.5 μg to 10 μg, 5 μg to 15 μg, 12.5 μg to 30 μg, 25 μg to 50 μg, 40 μg to 80 μg, 60 μg to 100 μg, 90 μg to 120 μg, 110 μg to 130 μg, 125 μg to 150 μg, 140 μg to 180 μg, 170 μg to 200 μg, 200 μg to 230 μg, 215 μg to 240 μg, 235 μg to less than 250 μg, less than 250 μg, greater than about 0.0 μg to about 1 μg, about 0.5 μg to about 2 μg, about 1.5 μg to about 3.0 μg, about 2.5 μg to about 10 μg, about 5 μg to about 15 μg, about 12.5 μg to about 30 μg, about 25 μg to about 50 μg, about 40 μg to about 80 μg, about 60 μg to about 100 μg, about 90 μg to about 120 μg, about 110 μg to about 130 μg, about 125 μg to about 150 μg, about 140 μg to about 180 μg, about 170 μg to about 200 μg, about 200 μg to about 230 μg, about 215 μg to about 240 μg, about 235 μg to less than 250 μg greater than 0.0 μg to 2 μg, greater than 0.0 μg to 3 μg, greater than 0.0 μg to 10 μg, greater than 0.0 μg to 15 μg, greater than 0.0 μg to 30 μg, greater than 0.0 μg to 50 μg, greater than 0.0 μg to 80 μg, greater than 0.0 μg to 100 μg, greater than 0.0 μg to 120 μg, greater than 0.0 μg to 130 μg, greater than 0.0 μg to 150 μg, greater than 0.0 μg to 180 μg, greater than 0.0 μg to 200 μg, greater than 0.0 μg to 230 μg, greater than 0.0 μg to 240 μg, greater than 0.0 μg to 250 μg, greater than 0.0 μg to about 2 μg, greater than 0.0 μg to about 3 μg, greater than 0.0 μg to about 10 μg, greater than 0.0 μg to about 15 μg, greater than 0.0 μg to about 30 μg, greater than 0.0 μg to about 50 μg, greater than 0.0 μg to about 80 μg, greater than 0.0 μg to about 100 μg, greater than 0.0 μg to about 120 μg, greater than 0.0 μg to about 130 μg, greater than 0.0 μg to about 150 μg, greater than 0.0 μg to about 180 μg, greater than 0.0 μg to about 200 μg, greater than 0.0 μg to about 230 μg, greater than 0.0 μg to about 240 μg, greater than 0.0 μg to about 250 μg, 0 μg to less than 250 μg, 0.5 μg to less than 250 μg, 1.5 μg to less than 250 μg, 2.5 μg to less than 250 μg, 5 μg to less than 250 μg, 12.5 μg to less than 250 μg, 25 μg to less than 250 μg, 40 μg to less than 250 μg, 60 μg to less than 250 μg, 90 μg to less than 250 μg, 110 μg to less than 250 μg, 125 μg to less than 250 μg, 140 μg to less than 250 μg, 170 μg to less than 250 μg, 200 μg to less than 250 μg, 215 μg to less than 250 μg, 0 μg to less than about 250 μg, about 0.5 μg to less than about 250 μg, about 1.5 μg to less than about 250 μg, about 2.5 μg to less than about 250 μg, about 5 μg to less than about 250 μg, about 12.5 μg to less than about 250 μg, about 25 μg to less than about 250 μg, about 40 μg to less than about 250 μg, about 60 μg to less than about 250 μg, about 90 μg to less than about 250 μg, about 110 μg to less than about 250 μg, about 125 μg to less than about 250 μg, about 140 μg to less than about 250 μg, about 170 μg to less than about 250 μg, about 200 μg to less than about 250 μg, and/or about 215 μg to less about than 250 μg.

Theophylline can be combined or present in a positive amount selected from a group consisting of: less than 45 mg, 30 mg, 15 mg, 10 mg, 5 mg, 1 mg, 500 μg, 250 μg, 120 μg, 80 μg, 40 μg, or 20 μg and less than about 45 mg, about 30 mg, about 15 mg, about 10 mg, about 5 mg, about 1 mg, about 500 μg, about 250 μg, about 120 μg, about 80 μg, about 40 μg, or about 20 μg, greater than 0 μg to 20 μg, 10 μg to 40 μg, 30 μg to 80 μg, 70 μg to 120 μg, 100 μg to 250 μg, 200 μg to 500 μg, 400 μg to 1 mg, 900 μg to 5 mg, 4 mg to 10 mg, 9 mg to 15 mg, 14 mg to 30 mg, 25 mg to 45 mg, greater than 0 μg to about 20 μg, about 10 μg to about 40 μg, about 30 μg to about 80 μg, about 70 μg to about 120 μg, about 100 μg to about 250 μg, about 200 μg to about 500 μg, about 400 μg to about 1 mg, about 900 μg to about 5 mg, about 4 mg to about 10 mg, about 9 mg to about 15 mg, about 14 mg to about 30 mg, about 25 mg to about 45 mg, greater than 0 μg to 40 μg, greater than 0 μg to 80 μg, greater than 0 μg to 120 μg, greater than 0 μg to 250 μg, greater than 0 μg to 500 μg, greater than 0 μg to 1 mg, greater than 0 μg to 5 mg, greater than 0 μg to 10 mg, greater than 0 μg to 15 mg, greater than 0 μg to 30 mg, greater than 0 μg to 45 mg, greater than 0 μg to about 40 μg, greater than 0 μg to about 80 μg, greater than 0 μg to about 120 μg, greater than 0 μg to about 250 μg, greater than 0 μg to about 500 μg, greater than 0 μg to about 1 mg, greater than 0 μg to about 5 mg, greater than 0 μg to about 10 mg, greater than 0 μg to about 15 mg, greater than 0 μg to about 30 mg, greater than 0 μg to about 45 mg, greater than 0 μg to 45 mg, 10 μg to 45 mg, 30 μg to 45 mg, 70 μg to 45 mg, 100 ng to 45 mg, 200 μg to 45 mg, 400 μg to 45 mg, 900 μg to 45 mg, 4 mg to 45 mg, 9 mg to 45 mg, 14 mg to 45 mg, 35 mg to 45 mg, greater than 0 μg to about 45 mg, about 10 μg to about 45 mg, about 30 μg to about 45 mg, about 70 μg to about 45 mg, about 100 μg to about 45 mg, about 200 μg to about 45 mg, about 400 μg to about 45 mg, about 900 μg to about 45 mg, about 4 mg to about 5 mg, about 9 mg to about 45 mg, about 14 mg to about 45 mg, and/or about 35 mg to about 45 mg.

Forskolin can be combined or present in a positive amount selected from a group consisting of: less than 500 mg to 450 mg, 475 mg to 425 mg, 435 mg to 400 mg, 415 mg to 300 mg, 325 mg to 250 mg, 275 mg to 150 mg, 200 mg to 100 mg, 135 mg to 80 mg, 95 mg to 65 mg, 75 mg to 50 mg, 60 mg to 40 mg, 45 mg to 25 mg, 30 mg to 20 mg, 15 mg to 5 mg, 10 mg to 2.5 mg, 3.5 mg to 1 mg, 2 mg to greater than 0 mg, less than about 500 mg to about 450 mg, about 475 mg to about 425 mg, about 435 mg to about 400 mg, about 415 mg to about 300 mg, about 325 mg to about 250 mg, about 275 mg to about 150 mg, about 200 mg to about 100 mg, about 135 mg to about 80 mg, about 95 mg to about 65 mg, about 75 mg to about 50 mg, about 60 mg to about 40 mg, about 45 mg to about 25 mg, about 30 mg to about 20 mg, about 15 mg to about 5 mg, about 10 mg to about 2.5 mg, about 3.5 mg to about 1 mg, about 2 mg to greater than 0 mg, 500 mg, less than 500 mg to 450 mg, less than 500 mg to 425 mg, less than 500 mg to 400 mg, less than 500 mg to 300 mg, less than 500 mg to 250 mg, less than 500 mg to 150 mg, less than 500 mg to 100 mg, less than 500 mg to 80 mg, less than 500 mg to 65 mg, less than 500 mg to 50 mg, less than 500 mg to 40 mg, less than 500 mg to 25 mg, less than 500 mg to 20 mg, less than 500 mg to 5 mg, less than 500 mg to 2.5 mg, less than 500 mg to 1 mg, less than 500 mg to greater than 0 mg, about 500 mg, less than about 500 mg to about 450 mg, less than about 500 mg to about 425 mg, less than about 500 mg to about 400 mg, less than about 500 mg to about 300 mg, less than about 500 mg to about 250 mg, less than about 500 mg to about 150 mg, less than about 500 mg to about 100 mg, less than about 500 mg to about 80 mg, less than about 500 mg to about 65 mg, less than about 500 mg to about 50 mg, less than about 500 mg to about 40 mg, less than about 500 mg to about 25 mg, less than about 500 mg to about 20 mg, less than about 500 mg to about 5 mg, less than about 500 mg to about 2.5 mg, less than about 500 mg to about 1 mg, less than about 500 mg to greater than about 0 mg, greater than 0 mg to 450 mg, greater than 0 mg to 425 mg, greater than 0 mg to 400 mg, greater than 0 mg to 300 mg, greater than 0 mg to 250 mg, greater than 0 mg to 150 mg, greater than 0 mg to 100 mg, greater than 0 mg to 80 mg, greater than 0 mg to 65 mg, greater than 0 mg to 50 mg, greater than 0 mg to 40 mg, greater than 0 mg to 25 mg, greater than 0 mg to 20 mg, greater than 0 mg to 5 mg, greater than 0 mg to 2.5 mg, greater than 0 mg to 1 mg, greater than 0 mg to about 450 mg, greater than 0 mg to about 425 mg, greater than 0 mg to about 400 mg, greater than 0 mg to about 300 mg, greater than 0 mg to about 250 mg, greater than 0 mg to about 150 mg, greater than 0 mg to about 100 mg, greater than 0 mg to about 80 mg, greater than 0 mg to about 65 mg, greater than 0 mg to about 50 mg, greater than 0 mg to about 40 mg, greater than 0 mg to about 25 mg, greater than 0 mg to about 20 mg, greater than 0 mg to about 5 mg, greater than 0 mg to about 2.5 mg, and/or greater than 0 mg to about 1 mg.

Cilastazol can be combined or present in a positive amount selected from a group consisting of: less than 500 mg to 450 mg, 475 mg to 425 mg, 435 mg to 400 mg, 415 mg to 300 mg, 325 mg to 250 mg, 275 mg to 150 mg, 200 mg to 100 mg, 135 mg to 80 mg, 95 mg to 65 mg, 75 mg to 50 mg, 60 mg to 40 mg, 45 mg to 25 mg, 30 mg to 20 mg, 15 mg to 5 mg, 10 mg to 2.5 mg, 3.5 mg to 1 mg, 2 mg to greater than 0 mg, less than about 500 mg to about 450 mg, about 475 mg to about 425 mg, about 435 mg to about 400 mg, about 415 mg to about 300 mg, about 325 mg to about 250 mg, about 275 mg to about 150 mg, about 200 mg to about 100 mg, about 135 mg to about 80 mg, about 95 mg to about 65 mg, about 75 mg to about 50 mg, about 60 mg to about 40 mg, about 45 mg to about 25 mg, about 30 mg to about 20 mg, about 15 mg to about 5 mg, about 10 mg to about 2.5 mg, about 3.5 mg to about 1 mg, about 2 mg to greater than 0 mg, 500 mg, less than 500 mg to 450 mg, less than 500 mg to 425 mg, less than 500 mg to 400 mg, less than 500 mg to 300 mg, less than 500 mg to 250 mg, less than 500 mg to 150 mg, less than 500 mg to 100 mg, less than 500 mg to 80 mg, less than 500 mg to 65 mg, less than 500 mg to 50 mg, less than 500 mg to 40 mg, less than 500 mg to 25 mg, less than 500 mg to 20 mg, less than 500 mg to 5 mg, less than 500 mg to 2.5 mg, less than 500 mg to 1 mg, less than 500 mg to greater than 0 mg, about 500 mg, less than about 500 mg to about 450 mg, less than about 500 mg to about 425 mg, less than about 500 mg to about 400 mg, less than about 500 mg to about 300 mg, less than about 500 mg to about 250 mg, less than about 500 mg to about 150 mg, less than about 500 mg to about 100 mg, less than about 500 mg to about 80 mg, less than about 500 mg to about 65 mg, less than about 500 mg to about 50 mg, less than about 500 mg to about 40 mg, less than about 500 mg to about 25 mg, less than about 500 mg to about 20 mg, less than about 500 mg to about 5 mg, less than about 500 mg to about 2.5 mg, less than about 500 mg to about 1 mg, less than about 500 mg to greater than about 0 mg, greater than 0 mg to 450 mg, greater than 0 mg to 425 mg, greater than 0 mg to 400 mg, greater than 0 mg to 300 mg, greater than 0 mg to 250 mg, greater than 0 mg to 150 mg, greater than 0 mg to 100 mg, greater than 0 mg to 80 mg, greater than 0 mg to 65 mg, greater than 0 mg to 50 mg, greater than 0 mg to 40 mg, greater than 0 mg to 25 mg, greater than 0 mg to 20 mg, greater than 0 mg to 5 mg, greater than 0 mg to 2.5 mg, greater than 0 mg to 1 mg, greater than 0 mg to about 450 mg, greater than 0 mg to about 425 mg, greater than 0 mg to about 400 mg, greater than 0 mg to about 300 mg, greater than 0 mg to about 250 mg, greater than 0 mg to about 150 mg, greater than 0 mg to about 100 mg, greater than 0 mg to about 80 mg, greater than 0 mg to about 65 mg, greater than 0 mg to about 50 mg, greater than 0 mg to about 40 mg, greater than 0 mg to about 25 mg, greater than 0 mg to about 20 mg, greater than 0 mg to about 5 mg, greater than 0 mg to about 2.5 mg, and/or greater than 0 mg to about 1 mg.

Roflumilast can be combined or present in a positive amount selected from a group consisting of: less than 10 mg, 5 mg, 1 mg, 500 μg, 250 μg, 120 μg, 80 μg, 40 μg, or 20 μg and less than about 10 mg, about 5 mg, about 1 mg, about 500 μg, about 250 μg, about 120 μg, about 80 μg, about 40 μg, or about 20 μg. Roflumilast can be also given, e.g., intranasally, and/or present in an amount selected from a group consisting of: greater than 0 μg to 20 μg, 10 μg to 40 μg, 30 μg to 80 μg, 70 μg to 120 μg, 100 μg to 250 μg, 200 μg to 500 μg, 400 μg to 1 mg, 900 ng to 5 mg, 4 mg to 10 mg, 9 mg to 15 mg, 14 mg to 30 mg, 25 mg to 45 mg, greater than 0 μg to about 20 μg, about 10 μg to about 40 μg, about 30 μg to about 80 μg, about 70 μg to about 120 μg, about 100 μg to about 250 μg, about 200 μg to about 500 μg, about 400 μg to about 1 mg, about 900 μg to about 5 mg, about 4 mg to about 10 mg, greater than 0 μg to 40 μg, greater than 0 μg to 80 μg, greater than 0 μg to 120 μg, greater than 0 μg to 250 μg, greater than 0 μg to 500 μg, greater than 0 μg to 1 mg, greater than 0 μg to 5 mg, greater than 0 μg to 10 mg, greater than 0 μg to 15 mg, greater than 0 μg to 30 mg, greater than 0 μg to 45 mg, greater than 0 μg to about 40 μg, greater than 0 μg to about 80 μg, greater than 0 μg to about 120 μg, greater than 0 ng to about 250 μg, greater than 0 μg to about 500 μg, greater than 0 μg to about 1 mg, greater than 0 μg to about 5 mg, greater than 0 μg to about 10 mg, greater than 0 μg to 10 mg, 10 μg to 10 mg, 30 μg to 10 mg, 70 μg to 10 mg, 100 μg to 10 mg, 200 μg to 10 mg, 400 μg to 10 mg, 900 μg to 10 mg, 4 mg to 10 mg, 9 mg to 10 mg, greater than 0 μg to about 10 mg, about 10 μg to about 10 mg, about 30 μg to about 10 mg, about 70 μg to about 10 mg, about 100 μg to about 10 mg, about 200 μg to about 10 mg, about 400 μg to about 10 mg, about 900 μg to about 10 mg, about 4 mg to about 10 mg, and/or about 9 mg to about 10.

Papaverine can be combined or present in a positive amount selected from a group consisting of: less than 500 mg to 450 mg, 475 mg to 425 mg, 435 mg to 400 mg, 415 mg to 300 mg, 325 mg to 250 mg, 275 mg to 150 mg, 200 mg to 100 mg, 135 mg to 80 mg, 95 mg to 65 mg, 75 mg to 50 mg, 60 mg to 40 mg, 45 mg to 25 mg, 30 mg to 20 mg, 15 mg to 5 mg, 10 mg to 2.5 mg, 3.5 mg to 1 mg, 2 mg to greater than 0 mg, less than about 500 mg to about 450 mg, about 475 mg to about 425 mg, about 435 mg to about 400 mg, about 415 mg to about 300 mg, about 325 mg to about 250 mg, about 275 mg to about 150 mg, about 200 mg to about 100 mg, about 135 mg to about 80 mg, about 95 mg to about 65 mg, about 75 mg to about 50 mg, about 60 mg to about 40 mg, about 45 mg to about 25 mg, about 30 mg to about 20 mg, about 15 mg to about 5 mg, about 10 mg to about 2.5 mg, about 3.5 mg to about 1 mg, about 2 mg to greater than 0 mg, 500 mg, less than 500 mg to 450 mg, less than 500 mg to 425 mg, less than 500 mg to 400 mg, less than 500 mg to 300 mg, less than 500 mg to 250 mg, less than 500 mg to 150 mg, less than 500 mg to 100 mg, less than 500 mg to 80 mg, less than 500 mg to 65 mg, less than 500 mg to 50 mg, less than 500 mg to 40 mg, less than 500 mg to 25 mg, less than 500 mg to 20 mg, less than 500 mg to 5 mg, less than 500 mg to 2.5 mg, less than 500 mg to 1 mg, less than 500 mg to greater than 0 mg, about 500 mg, less than about 500 mg to about 450 mg, less than about 500 mg to about 425 mg, less than about 500 mg to about 400 mg, less than about 500 mg to about 300 mg, less than about 500 mg to about 250 mg, less than about 500 mg to about 150 mg, less than about 500 mg to about 100 mg, less than about 500 mg to about 80 mg, less than about 500 mg to about 65 mg, less than about 500 mg to about 50 mg, less than about 500 mg to about 40 mg, less than about 500 mg to about 25 mg, less than about 500 mg to about 20 mg, less than about 500 mg to about 5 mg, less than about 500 mg to about 2.5 mg, less than about 500 mg to about 1 mg, less than about 500 mg to greater than about 0 mg, greater than 0 mg to 450 mg, greater than 0 mg to 425 mg, greater than 0 mg to 400 mg, greater than 0 mg to 300 mg, greater than 0 mg to 250 mg, greater than 0 mg to 150 mg, greater than 0 mg to 100 mg, greater than 0 mg to 80 mg, greater than 0 mg to 65 mg, greater than 0 mg to 50 mg, greater than 0 mg to 40 mg, greater than 0 mg to 25 mg, greater than 0 mg to 20 mg, greater than 0 mg to 5 mg, greater than 0 mg to 2.5 mg, greater than 0 mg to 1 mg, greater than 0 mg to about 450 mg, greater than 0 mg to about 425 mg, greater than 0 mg to about 400 mg, greater than 0 mg to about 300 mg, greater than 0 mg to about 250 mg, greater than 0 mg to about 150 mg, greater than 0 mg to about 100 mg, greater than 0 mg to about 80 mg, greater than 0 mg to about 65 mg, greater than 0 mg to about 50 mg, greater than 0 mg to about 40 mg, greater than 0 mg to about 25 mg, greater than 0 mg to about 20 mg, greater than 0 mg to about 5 mg, greater than 0 mg to about 2.5 mg, and/or greater than 0 mg to about 1 mg.

In some aspects of the invention, riociguat, theophylline, forskolin, cilastazol, roflumilast, papaverine, and/or combinations thereof can be combined. For example, (a) riociguat can be combined or present in a positive amount selected from a group consisting of: greater than 0.0 μg to 1 μg, 0.5 μg to 2 μg, 1.5 μg to 3.0 μg, 2.5 μg to 10 μg, 5 μg to 15 μg, 12.5 ng to 30 μg, 25 μg to 50 μg, 40 μg to 80 μg, 60 μg to 100 μg, 90 μg to 120 μg, 110 μg to 130 μg, 125 μg to 150 μg, 140 μg to 180 μg, 170 μg to 200 μg, 200 μg to 230 μg, 215 μg to 240 μg, 235 μg to less than 250 μg, less than 250 μg, greater than about 0.0 μg to about 1 μg, about 0.5 ng to about 2 μg, about 1.5 μg to about 3.0 μg, about 2.5 μg to about 10 μg, about 5 μg to about 15 μg, about 12.5 μg to about 30 μg, about 25 μg to about 50 μg, about 40 μg to about 80 μg, about 60 μg to about 100 μg, about 90 μg to about 120 μg, about 110 μg to about 130 μg, about 125 μg to about 150 μg, about 140 μg to about 180 μg, about 170 μg to about 200 μg, about 200 ng to about 230 μg, about 215 μg to about 240 μg, about 235 μg to less than 250 μg greater than 0.0 μg to 2 μg, greater than 0.0 μg to 3 μg, greater than 0.0 μg to 10 μg, greater than 0.0 μg to 15 μg, greater than 0.0 μg to 30 μg, greater than 0.0 μg to 50 μg, greater than 0.0 μg to 80 μg, greater than 0.0 μg to 100 μg, greater than 0.0 μg to 120 μg, greater than 0.0 μg to 130 μg, greater than 0.0 μg to 150 μg, greater than 0.0 μg to 180 μg, greater than 0.0 μg to 200 μg, greater than 0.0 μg to 230 μg, greater than 0.0 μg to 240 μg, greater than 0.0 μg to 250 μg, greater than 0.0 μg to about 2 μg, greater than 0.0 μg to about 3 μg, greater than 0.0 μg to about 10 μg, greater than 0.0 μg to about 15 μg, greater than 0.0 μg to about 30 μg, greater than 0.0 μg to about 50 μg, greater than 0.0 μg to about 80 μg, greater than 0.0 μg to about 100 μg, greater than 0.0 μg to about 120 μg, greater than 0.0 μg to about 130 μg, greater than 0.0 μg to about 150 μg, greater than 0.0 μg to about 180 μg, greater than 0.0 μg to about 200 μg, greater than 0.0 ng to about 230 μg, greater than 0.0 μg to about 240 μg, greater than 0.0 μg to about 250 μg, 0 ng to less than 250 μg, 0.5 μg to less than 250 μg, 1.5 μg to less than 250 μg, 2.5 μg to less than 250 μg, 5 μg to less than 250 μg, 12.5 μg to less than 250 μg, 25 μg to less than 250 μg, 40 μg to less than 250 μg, 60 μg to less than 250 μg, 90 μg to less than 250 μg, 110 μg to less than 250 μg, 125 μg to less than 250 μg, 140 μg to less than 250 μg, 170 μg to less than 250 μg, 200 μg to less than 250 μg, 215 μg to less than 250 μg, 0 μg to less than about 250 μg, about 0.5 μg to less than about 250 μg, about 1.5 μg to less than about 250 μg, about 2.5 μg to less than about 250 μg, about 5 μg to less than about 250 μg, about 12.5 μg to less than about 250 μg, about 25 μg to less than about 250 μg, about 40 μg to less than about 250 μg, about 60 μg to less than about 250 μg, about 90 μg to less than about 250 μg, about 110 μg to less than about 250 μg, about 125 μg to less than about 250 μg, about 140 μg to less than about 250 μg, about 170 μg to less than about 250 μg, about 200 μg to less than about 250 μg, and/or about 215 μg to less about than 250 ng; (b) theophylline can be combined or present in a positive amount selected from a group consisting of: less than 45 mg, 30 mg, 15 mg, 10 mg, 5 mg, 1 mg, 500 μg, 250 μg, 120 μg, 80 μg, 40 μg, or 20 μg and less than about 45 mg, about 30 mg, about 15 mg, about 10 mg, about 5 mg, about 1 mg, about 500 μg, about 250 μg, about 120 μg, about 80 μg, about 40 μg, or about 20 μg, greater than 0 μg to 20 μg, 10 μg to 40 μg, 30 μg to 80 μg, 70 μg to 120 μg, 100 μg to 250 μg, 200 μg to 500 μg, 400 μg to 1 mg, 900 μg to 5 mg, 4 mg to 10 mg, 9 mg to 15 mg, 14 mg to 30 mg, 25 mg to 45 mg, greater than 0 μg to about 20 μg, about 10 μg to about 40 μg, about 30 μg to about 80 μg, about 70 μg to about 120 μg, about 100 μg to about 250 μg, about 200 μg to about 500 μg, about 400 μg to about 1 mg, about 900 μg to about 5 mg, about 4 mg to about 10 mg, about 9 mg to about 15 mg, about 14 mg to about 30 mg, about 25 mg to about 45 mg, greater than 0 μg to 40 μg, greater than 0 μg to 80 μg, greater than 0 μg to 120 μg, greater than 0 μg to 250 μg, greater than 0 μg to 500 μg, greater than 0 μg to 1 mg, greater than 0 μg to 5 mg, greater than 0 μg to 10 mg, greater than 0 μg to 15 mg, greater than 0 μg to 30 mg, greater than 0 μg to 45 mg, greater than 0 μg to about 40 μg, greater than 0 μg to about 80 μg, greater than 0 μg to about 120 μg, greater than 0 μg to about 250 μg, greater than 0 μg to about 500 μg, greater than 0 μg to about 1 mg, greater than 0 μg to about 5 mg, greater than 0 μg to about 10 mg, greater than 0 μg to about 15 mg, greater than 0 μg to about 30 mg, greater than 0 μg to about 45 mg, greater than 0 μg to 45 mg, 10 μg to 45 mg, 30 μg to 45 mg, 70 μg to 45 mg, 100 ng to 45 mg, 200 μg to 45 mg, 400 μg to 45 mg, 900 μg to 45 mg, 4 mg to 45 mg, 9 mg to 45 mg, 14 mg to 45 mg, 35 mg to 45 mg, greater than 0 μg to about 45 mg, about 10 μg to about 45 mg, about 30 μg to about 45 mg, about 70 μg to about 45 mg, about 100 μg to about 45 mg, about 200 μg to about 45 mg, about 400 μg to about 45 mg, about 900 μg to about 45 mg, about 4 mg to about 5 mg, about 9 mg to about 45 mg, about 14 mg to about 45 mg, and/or about 35 mg to about 45 mg; (c) forskolin can be combined or present in a positive amount selected from a group consisting of: less than 500 mg to 450 mg, 475 mg to 425 mg, 435 mg to 400 mg, 415 mg to 300 mg, 325 mg to 250 mg, 275 mg to 150 mg, 200 mg to 100 mg, 135 mg to 80 mg, 95 mg to 65 mg, 75 mg to 50 mg, 60 mg to 40 mg, 45 mg to 25 mg, 30 mg to 20 mg, 15 mg to 5 mg, 10 mg to 2.5 mg, 3.5 mg to 1 mg, 2 mg to greater than 0 mg, less than about 500 mg to about 450 mg, about 475 mg to about 425 mg, about 435 mg to about 400 mg, about 415 mg to about 300 mg, about 325 mg to about 250 mg, about 275 mg to about 150 mg, about 200 mg to about 100 mg, about 135 mg to about 80 mg, about 95 mg to about 65 mg, about 75 mg to about 50 mg, about 60 mg to about 40 mg, about 45 mg to about 25 mg, about 30 mg to about 20 mg, about 15 mg to about 5 mg, about 10 mg to about 2.5 mg, about 3.5 mg to about 1 mg, about 2 mg to greater than 0 mg, 500 mg, less than 500 mg to 450 mg, less than 500 mg to 425 mg, less than 500 mg to 400 mg, less than 500 mg to 300 mg, less than 500 mg to 250 mg, less than 500 mg to 150 mg, less than 500 mg to 100 mg, less than 500 mg to 80 mg, less than 500 mg to 65 mg, less than 500 mg to 50 mg, less than 500 mg to 40 mg, less than 500 mg to 25 mg, less than 500 mg to 20 mg, less than 500 mg to 5 mg, less than 500 mg to 2.5 mg, less than 500 mg to 1 mg, less than 500 mg to greater than 0 mg, about 500 mg, less than about 500 mg to about 450 mg, less than about 500 mg to about 425 mg, less than about 500 mg to about 400 mg, less than about 500 mg to about 300 mg, less than about 500 mg to about 250 mg, less than about 500 mg to about 150 mg, less than about 500 mg to about 100 mg, less than about 500 mg to about 80 mg, less than about 500 mg to about 65 mg, less than about 500 mg to about 50 mg, less than about 500 mg to about 40 mg, less than about 500 mg to about 25 mg, less than about 500 mg to about 20 mg, less than about 500 mg to about 5 mg, less than about 500 mg to about 2.5 mg, less than about 500 mg to about 1 mg, less than about 500 mg to greater than about 0 mg, greater than 0 mg to 450 mg, greater than 0 mg to 425 mg, greater than 0 mg to 400 mg, greater than 0 mg to 300 mg, greater than 0 mg to 250 mg, greater than 0 mg to 150 mg, greater than 0 mg to 100 mg, greater than 0 mg to 80 mg, greater than 0 mg to 65 mg, greater than 0 mg to 50 mg, greater than 0 mg to 40 mg, greater than 0 mg to 25 mg, greater than 0 mg to 20 mg, greater than 0 mg to 5 mg, greater than 0 mg to 2.5 mg, greater than 0 mg to 1 mg, greater than 0 mg to about 450 mg, greater than 0 mg to about 425 mg, greater than 0 mg to about 400 mg, greater than 0 mg to about 300 mg, greater than 0 mg to about 250 mg, greater than 0 mg to about 150 mg, greater than 0 mg to about 100 mg, greater than 0 mg to about 80 mg, greater than 0 mg to about 65 mg, greater than 0 mg to about 50 mg, greater than 0 mg to about 40 mg, greater than 0 mg to about 25 mg, greater than 0 mg to about 20 mg, greater than 0 mg to about 5 mg, greater than 0 mg to about 2.5 mg, and/or greater than 0 mg to about 1 mg; (d) cilastazol can be combined or present in a positive amount selected from a group consisting of: less than 500 mg to 450 mg, 475 mg to 425 mg, 435 mg to 400 mg, 415 mg to 300 mg, 325 mg to 250 mg, 275 mg to 150 mg, 200 mg to 100 mg, 135 mg to 80 mg, 95 mg to 65 mg, 75 mg to 50 mg, 60 mg to 40 mg, 45 mg to 25 mg, 30 mg to 20 mg, 15 mg to 5 mg, 10 mg to 2.5 mg, 3.5 mg to 1 mg, 2 mg to greater than 0 mg, less than about 500 mg to about 450 mg, about 475 mg to about 425 mg, about 435 mg to about 400 mg, about 415 mg to about 300 mg, about 325 mg to about 250 mg, about 275 mg to about 150 mg, about 200 mg to about 100 mg, about 135 mg to about 80 mg, about 95 mg to about 65 mg, about 75 mg to about 50 mg, about 60 mg to about 40 mg, about 45 mg to about 25 mg, about 30 mg to about 20 mg, about 15 mg to about 5 mg, about 10 mg to about 2.5 mg, about 3.5 mg to about 1 mg, about 2 mg to greater than 0 mg, 500 mg, less than 500 mg to 450 mg, less than 500 mg to 425 mg, less than 500 mg to 400 mg, less than 500 mg to 300 mg, less than 500 mg to 250 mg, less than 500 mg to 150 mg, less than 500 mg to 100 mg, less than 500 mg to 80 mg, less than 500 mg to 65 mg, less than 500 mg to 50 mg, less than 500 mg to 40 mg, less than 500 mg to 25 mg, less than 500 mg to 20 mg, less than 500 mg to 5 mg, less than 500 mg to 2.5 mg, less than 500 mg to 1 mg, less than 500 mg to greater than 0 mg, about 500 mg, less than about 500 mg to about 450 mg, less than about 500 mg to about 425 mg, less than about 500 mg to about 400 mg, less than about 500 mg to about 300 mg, less than about 500 mg to about 250 mg, less than about 500 mg to about 150 mg, less than about 500 mg to about 100 mg, less than about 500 mg to about 80 mg, less than about 500 mg to about 65 mg, less than about 500 mg to about 50 mg, less than about 500 mg to about 40 mg, less than about 500 mg to about 25 mg, less than about 500 mg to about 20 mg, less than about 500 mg to about 5 mg, less than about 500 mg to about 2.5 mg, less than about 500 mg to about 1 mg, less than about 500 mg to greater than about 0 mg, greater than 0 mg to 450 mg, greater than 0 mg to 425 mg, greater than 0 mg to 400 mg, greater than 0 mg to 300 mg, greater than 0 mg to 250 mg, greater than 0 mg to 150 mg, greater than 0 mg to 100 mg, greater than 0 mg to 80 mg, greater than 0 mg to 65 mg, greater than 0 mg to 50 mg, greater than 0 mg to 40 mg, greater than 0 mg to 25 mg, greater than 0 mg to 20 mg, greater than 0 mg to 5 mg, greater than 0 mg to 2.5 mg, greater than 0 mg to 1 mg, greater than 0 mg to about 450 mg, greater than 0 mg to about 425 mg, greater than 0 mg to about 400 mg, greater than 0 mg to about 300 mg, greater than 0 mg to about 250 mg, greater than 0 mg to about 150 mg, greater than 0 mg to about 100 mg, greater than 0 mg to about 80 mg, greater than 0 mg to about 65 mg, greater than 0 mg to about 50 mg, greater than 0 mg to about 40 mg, greater than 0 mg to about 25 mg, greater than 0 mg to about 20 mg, greater than 0 mg to about 5 mg, greater than 0 mg to about 2.5 mg, and/or greater than 0 mg to about 1 mg; (e) roflumilast can be combined or present in a positive amount selected from a group consisting of: greater than 0 μg to 20 μg, 10 μg to 40 μg, 30 μg to 80 μg, 70 μg to 120 μg, 100 μg to 250 μg, 200 μg to 500 μg, 400 μg to 1 mg, 900 μg to 5 mg, 4 mg to 10 mg, 9 mg to 15 mg, 14 mg to 30 mg, 25 mg to 45 mg, greater than 0 μg to about 20 μg, about 10 μg to about 40 μg, about 30 μg to about 80 μg, about 70 μg to about 120 μg, about 100 μg to about 250 μg, about 200 μg to about 500 μg, about 400 μg to about 1 mg, about 900 μg to about 5 mg, about 4 mg to about 10 mg, greater than 0 μg to 40 μg, greater than 0 μg to 80 μg, greater than 0 μg to 120 μg, greater than 0 μg to 250 μg, greater than 0 μg to 500 μg, greater than 0 μg to 1 mg, greater than 0 μg to 5 mg, greater than 0 μg to 10 mg, greater than 0 μg to 15 mg, greater than 0 μg to 30 mg, greater than 0 μg to 45 mg, greater than 0 μg to about 40 μg, greater than 0 μg to about 80 μg, greater than 0 μg to about 120 μg, greater than 0 μg to about 250 μg, greater than 0 μg to about 500 μg, greater than 0 μg to about 1 mg, greater than 0 μg to about 5 mg, greater than 0 μg to about 10 mg, greater than 0 μg to 10 mg, 10 μg to 10 mg, 30 μg to 10 mg, 70 μg to 10 mg, 100 μg to 10 mg, 200 μg to 10 mg, 400 μg to 10 mg, 900 μg to 10 mg, 4 mg to 10 mg, 9 mg to 10 mg, greater than 0 μg to about 10 mg, about 10 μg to about 10 mg, about 30 μg to about 10 mg, about 70 μg to about 10 mg, about 100 μg to about 10 mg, about 200 μg to about 10 mg, about 400 μg to about 10 mg, about 900 μg to about 10 mg, about 4 mg to about 10 mg, and/or about 9 mg to about 10; and/or (f) papaverine can be combined or present in a positive amount selected from a group consisting of: less than 500 mg to 450 mg, 475 mg to 425 mg, 435 mg to 400 mg, 415 mg to 300 mg, 325 mg to 250 mg, 275 mg to 150 mg, 200 mg to 100 mg, 135 mg to 80 mg, 95 mg to 65 mg, 75 mg to 50 mg, 60 mg to 40 mg, 45 mg to 25 mg, 30 mg to 20 mg, 15 mg to 5 mg, 10 mg to 2.5 mg, 3.5 mg to 1 mg, 2 mg to greater than 0 mg, less than about 500 mg to about 450 mg, about 475 mg to about 425 mg, about 435 mg to about 400 mg, about 415 mg to about 300 mg, about 325 mg to about 250 mg, about 275 mg to about 150 mg, about 200 mg to about 100 mg, about 135 mg to about 80 mg, about 95 mg to about 65 mg, about 75 mg to about 50 mg, about 60 mg to about 40 mg, about 45 mg to about 25 mg, about 30 mg to about 20 mg, about 15 mg to about 5 mg, about 10 mg to about 2.5 mg, about 3.5 mg to about 1 mg, about 2 mg to greater than 0 mg, 500 mg, less than 500 mg to 450 mg, less than 500 mg to 425 mg, less than 500 mg to 400 mg, less than 500 mg to 300 mg, less than 500 mg to 250 mg, less than 500 mg to 150 mg, less than 500 mg to 100 mg, less than 500 mg to 80 mg, less than 500 mg to 65 mg, less than 500 mg to 50 mg, less than 500 mg to 40 mg, less than 500 mg to 25 mg, less than 500 mg to 20 mg, less than 500 mg to 5 mg, less than 500 mg to 2.5 mg, less than 500 mg to 1 mg, less than 500 mg to greater than 0 mg, about 500 mg, less than about 500 mg to about 450 mg, less than about 500 mg to about 425 mg, less than about 500 mg to about 400 mg, less than about 500 mg to about 300 mg, less than about 500 mg to about 250 mg, less than about 500 mg to about 150 mg, less than about 500 mg to about 100 mg, less than about 500 mg to about 80 mg, less than about 500 mg to about 65 mg, less than about 500 mg to about 50 mg, less than about 500 mg to about 40 mg, less than about 500 mg to about 25 mg, less than about 500 mg to about 20 mg, less than about 500 mg to about 5 mg, less than about 500 mg to about 2.5 mg, less than about 500 mg to about 1 mg, less than about 500 mg to greater than about 0 mg, greater than 0 mg to 450 mg, greater than 0 mg to 425 mg, greater than 0 mg to 400 mg, greater than 0 mg to 300 mg, greater than 0 mg to 250 mg, greater than 0 mg to 150 mg, greater than 0 mg to 100 mg, greater than 0 mg to 80 mg, greater than 0 mg to 65 mg, greater than 0 mg to 50 mg, greater than 0 mg to 40 mg, greater than 0 mg to 25 mg, greater than 0 mg to 20 mg, greater than 0 mg to 5 mg, greater than 0 mg to 2.5 mg, greater than 0 mg to 1 mg, greater than 0 mg to about 450 mg, greater than 0 mg to about 425 mg, greater than 0 mg to about 400 mg, greater than 0 mg to about 300 mg, greater than 0 mg to about 250 mg, greater than 0 mg to about 150 mg, greater than 0 mg to about 100 mg, greater than 0 mg to about 80 mg, greater than 0 mg to about 65 mg, greater than 0 mg to about 50 mg, greater than 0 mg to about 40 mg, greater than 0 mg to about 25 mg, greater than 0 mg to about 20 mg, greater than 0 mg to about 5 mg, greater than 0 mg to about 2.5 mg, and/or greater than 0 mg to about 1 mg.

The dosage unit or composition comprising the dosage unit can be formed into a dosage unit suitable for administration by a method selected from a group consisting of: oral administration, transmucosal administration, buccal administration, inhalation administration, intranasal administration, parental administration, intravenous administration, subcutaneous administration, intramuscular administration, sublingual administration, transdermal administration, and rectal administration. For ease of use, the dosage unit or composition comprising the dosage unit can be formed into a dosage unit suitable for oral administration, inhalational administration, nasal administration, or a combination thereof. The dosage unit or composition comprising the dosage unit can be a liquid. In some embodiments, the route of administration can penetrate the placental barrier and/or the blood brain barrier. For example, in some embodiments, intranasal drug administration, e.g., theophylline, can be delivered into the brain (1) directly by absorption through the cribriform plate along the olfactory bulb, (2) indirectly by absorption through blood-brain barrier receptors, or (3) through combinations of both methods. In another example, in some embodiments, pregnant mothers can be given a dose of a drug, which will then reach the fetus by crossing the placental barrier. The drug can then enter the fetus and find its way to target sites, e.g., the brain. If the drug reaches the brain, it can cross the blood/brain barrier. In some other embodiments, a breast feeding mother can be given a dose of drug, which will then reach the baby by flowing to the mother's breast milk. The breast milk containing the drug can be then fed to the baby. In some embodiments, the drug can be used to fortify baby's formula and/or milk and then fed to the baby. The drug will eventually find its way to the brain by crossing the blood/brain barrier. In some embodiments, the drug may or may not cross the blood/brain barrier.

The dosage unit or composition comprising the dosage unit can have a pH of 7.0 or less than 7.0. For example, the dosage unit or composition comprising the dosage unit can have a pH of 7.0. The dosage unit or composition comprising the dosage unit can have a pH of less than 7.0. The dosage unit or composition comprising the dosage unit can have a pH of less than 6.9. The dosage unit or composition comprising the dosage unit can have a pH of less than 6.5. The dosage unit or composition comprising the dosage unit can have a pH of greater than 7.0. For example, the dosage unit or composition comprising the dosage unit can have a pH of greater than 7.1. The dosage unit or composition comprising the dosage unit can have a pH of greater than 7.5. The dosage unit or composition comprising the dosage unit can have a pH of greater than 8.0. The dosage unit or composition comprising the dosage unit can have a pH of greater than 9.0. The dosage unit or composition comprising the dosage unit can have a pH selected from the group consisting of: 5.0 to 5.5; 5.1 to 5.6; 5.2 to 5.7; 5.3 to 5.8; 5.4 to 5.9; 5.5 to 6.0; 5.6 to 6.1; 5.7 to 6.2; 5.8 to 6.3; 5.9 to 6.4; 6.0 to 6.5; 6.1 to 6.6; 6.2 to 6.7; 6.3 to 6.8; 6.4 to 6.9; 6.5 to 7.0; 6.6 to 7.1; 6.7 to 7.2; 6.8 to 7.3; 6.9 to 7.4; 7.0 to 7.5; 7.1 to 7.6; 7.2 to 7.7; 7.3 to 7.8; 7.4 to 7.9; 7.5 to 8.0; 7.6 to 8.1; 7.7 to 8.2; 7.8 to 8.3; 7.9 to 8.4; 8.0 to 8.5; 8.1 to 8.6; 8.2 to 8.7; 8.3 to 8.8; 8.4 to 8.9; 8.5 to 9.0; 8.6 to 9.1; 8.7 to 9.2; 8.8 to 9.3; 8.9 to 9.4; 9.0 to 9.5; about 5.0 to about 5.5; about 5.1 to about 5.6; about 5.2 to about 5.7; about 5.3 to about 5.8; about 5.4 to about 5.9; about 5.5 to about 6.0; about 5.6 to about 6.1; about 5.7 to about 6.2; about 5.8 to about 6.3; about 5.9 to about 6.4; about 6.0 to about 6.5; about 6.1 to about 6.6; about 6.2 to about 6.7; about 6.3 to about 6.8; about 6.4 to about 6.9; about 6.5 to about 7.0; about 6.6 to about 7.1; about 6.7 to about 7.2; about 6.8 to about 7.3; about 6.9 to about 7.4; about 7.0 to about 7.5; about 7.1 to about 7.6; about 7.2 to about 7.7; about 7.3 to about 7.8; about 7.4 to about 7.9; about 7.5 to about 8.0; about 7.6 to about 8.1; about 7.7 to about 8.2; about 7.8 to about 8.3; about 7.9 to about 8.4; about 8.0 to about 8.5; about 8.1 to about 8.6; about 8.2 to about 8.7; about 8.3 to about 8.8; about 8.4 to about 8.9; about 8.5 to about 9.0; about 8.6 to about 9.1; about 8.7 to about 9.2; about 8.8 to about 9.3; about 8.9 to about 9.4; and/or about 9.0 to about 9.5.

The dosage unit or composition comprising the dosage unit can further comprise one or more excipients. The dosage unit can be formed to comprise one or more excipients, wherein the one or more excipients can be selected from a group consisting of: detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

The dosage unit or composition comprising the dosage unit can further comprise one or more cytochrome p450 inhibitors. The dosage unit or composition comprising the dosage unit can also comprise one or more cytochrome p450 inhibitors wherein the cytochrome p450 inhibitors can fully or partially inhibit a cytochrome selected from a group consisting of: CYP1, CYP1A1, CYP1A2, CYP1B1, CYP2, CYP2A6, CYP2A7, CYP2A13, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3, CYP3A4, CYP3A5, CYP3A7, CYP3A43, CYP4, CYP4A11, CYP4A22, CYP4B1, CYP4F2, CYP4F3, CYP4F8, CYP4F11, CYP4F12, CYP4F22, CYP4V2, CYP4X1, CYP4Z1, CYP5, CYP5A1, CYP7, CYP7A1, CYP7B1, CYP8, CYP8A1, CYP8B1, CYP11, CYP11A1, CYP11B1, CYP11B2, CYP17, CYP17A1, CYP19, CYP19A1, CYP20, CYP20A1, CYP21, CYP21A2, CYP24, CYP24A1, CYP26, CYP26A1, CYP26B1, CYP26C1, CYP27, CYP27A1, CYP27B1, CYP27C1, CYP39, CYP39A1, CYP46, CYP46A1, CYP51, and CYP51A1. The dosage unit or composition comprising the dosage unit can also comprise one or more cytochrome p450 inhibitors wherein the cytochrome p450 inhibitors can fully or partially inhibit CYP1. The dosage unit or composition comprising the dosage unit can also comprise one or more cytochrome p450 inhibitors wherein the cytochrome p450 inhibitors can fully or partially inhibit CYP1A2. The dosage unit or composition comprising the dosage unit can also comprise one or more CYP1A2 inhibitors wherein the CYP1A2 inhibitors can be selected from a group consisting of: fluoroquinolone, selective serotonin reuptake inhibitor (SSRI), calcium channel blocker, herbal tea, naringenin, H2-receptor activators, antiarrhythmic agent, interferon, xanthotoxin, mibefradil, cumin, turmeric, and isoniazid. The dosage unit or composition comprising the dosage unit can also comprise one or more CYP1A2 inhibitors wherein the CYP1A2 inhibitors can be grapefruit juice. The dosage unit or composition comprising the dosage unit can also comprise one or more CYP1A2 inhibitors wherein the CYP1A2 inhibitors can be naringenin.

The dosage unit or composition comprising the dosage unit can further comprise combining one or more β-adrenergic activators. The dosage unit or composition comprising the dosage unit can also comprise one or more β-adrenergic activators wherein the one or more 3-activators can be a β₁-adrenergic activators and/or β2-adrenergic activators. The dosage unit or composition comprising the dosage unit can also comprise one or more β-adrenergic activators wherein the one or more β-activators can be a β₁-adrenergic activators. The dosage unit or composition comprising the dosage unit can also comprise one or more β-adrenergic activators wherein the one or more β-activators can be a β₁-adrenergic activators selected from a group consisting of dobutamine, isoproterenol, xamoterol and epinephrine. The dosage unit or composition comprising the dosage unit can also comprise one or more β-adrenergic activators wherein the one or more β-activators can be a β₂-adrenergic activators. The dosage unit or composition comprising the dosage unit can also comprise one or more β-adrenergic activators wherein the one or more β-activators can be a β₂-adrenergic activators selected from a group consisting of: albuterol, levalbuterol, fenoterol, formoterol, isoproterenol (ρ₁ and β₂), metaproterenol, salmeterol, terbutaline, clenbuterol, isoetarine, pirbuterol, procaterol, ritodrine, and epinephrine. The dosage unit or composition comprising the dosage unit can also comprise one or more β-adrenergic activators wherein the one or more β-activators can be selected from a group consisting of: arbutamine, befunolol, bromoacetylalprenololmenthane, broxaterol, cimaterol, cirazoline, denopamine, dopexamine, etilefrine, hexoprenaline, higenamine, isoxsuprine, mabuterol, methoxyphenamine, nylidrin, oxyfedrine, prenalterol, ractopamine, reproterol, rimiterol, tretoquinol, tulobuterol, zilpaterol, and zinterol.

Kits for Diagnosis and/or Treatment

A kit that can be used to diagnose chromosomal abnormalities and/or one or more symptoms associated with chromosomal abnormalities is contemplated as part of the invention.

In an additional aspect of the invention, disclosed herein is a kit that can comprise: (a) antibodies that bind one or more members of the hedgehog signaling pathway; and (b) an insert that describes how to diagnose a subject with chromosomal abnormalities and/or one or more symptoms associated with chromosomal abnormalities based upon the level of one or more members of the hedgehog signaling pathway that can be lower than a threshold level.

The antibodies can be SHH specific antibodies. The antibodies can be IHH specific antibodies. The antibodies can be DHH specific antibodies. For examples, antibodies that bind to SHH, DHH, and IHH, can be commercially purchased or made by conventional methods. For example, SHH antibodies are available through R&D systems or LifeSpan BioSciences, Inc. (SHH: http://www.rndsystems.com/product_results.aspx?m=2109; DHH: http://www.lsbio.com/antibodies/anti-desert-hedgehog-antibody-dhh-antibody-aa194-223-rabbit-anti-human-polyclonal-for-western-blot-ls-c159454/1667741; IHH: http://www.rndsystems.com/product_results.aspx?k=Indian%20Hedgehog%20Antibody).

The kit can further comprise an enzyme-linked immunosorbent (ELISA) assay.

The kit can further comprise one or more therapeutic agents capable of maintaining and/or increasing one or more members of the hedgehog signaling pathway.

### Drug Compositions Comprising Riociguat

Riociguat is believed to be helpful in treating two forms of pulmonary hypertension (PH): chronic thromboembolic pulmonary hypertension (CTEPH) and pulmonary arterial hypertension (PAH). However, the dosages can be typically in the milligram range and can be given as an oral dosage. However, if the dosage form is changed, for example, into a form suitable for nasal administration, riociguat may be given at a much lower dosage (in the microgram or lower range). Additionally, when riociguat is presented in other dosage forms, it can be effective in treating other diseases, such as pulmonary hypertension, loss and/or one or more symptoms associated with chromosomal abnormalities.

In an additional aspect of the invention, disclosed herein is a pharmaceutical dosage unit comprising riociguat. A pharmaceutical dosage unit with riociguat in the microgram or lower range, e.g., below 250 micrograms, is also disclosed.

The ricociguat can be suitable for administration by a method selected from a group consisting of: transmucosal administration, inhalation administration, intranasal administration, parental administration, intravenous administration, subcutaneous administration, intramuscular administration, sublingual administration, transdermal administration, and rectal administration. For ease of use, the ricociguat can be suitable for administration by inhalation administration, intranasal administration, intravenous administration, or a combination thereof. In some embodiments, the route of administration can penetrate the placental barrier and/or the blood brain barrier. For example, in some embodiments, intranasal drug administration, e.g., theophylline, can be delivered into the brain (1) directly by absorption through the cribriform plate along the olfactory bulb, (2) indirectly by absorption through blood-brain barrier receptors, or (3) through combinations of both methods. In another example, in some embodiments, pregnant mothers can be given a dose of a drug, which will then reach the fetus by crossing the placental barrier. The drug can then enter the fetus and find its way to target sites, e.g., the brain. If the drug reaches the brain, it can cross the blood/brain barrier. In some other embodiments, a breast feeding mother can be given a dose of drug, which will then reach the baby by flowing to the mother's breast milk. The breast milk containing the drug can be then fed to the baby. In some embodiments, the drug can be used to fortify baby's formula and/or milk and then fed to the baby. The drug will eventually find its way to the brain by crossing the blood/brain barrier. In some embodiments, the drug may or may not cross the blood/brain barrier.

Riociguat can be present in a positive amount selected from a group consisting of: greater than 0.0 μg to 1 μg, 0.5 μg to 2 μg, 1.5 μg to 3.0 μg, 2.5 μg to 10 μg, 5 μg to 15 μg, 12.5 μg to 30 μg, 25 μg to 50 μg, 40 μg to 80 μg, 60 μg to 100 μg, 90 μg to 120 μg, 110 μg to 130 μg, 125 μg to 150 μg, 140 μg to 180 μg, 170 μg to 200 μg, 200 μg to 230 μg, 215 μg to 240 μg, 235 μg to less than 250 μg, less than 250 μg, greater than about 0.0 μg to about 1 μg, about 0.5 μg to about 2 μg, about 1.5 μg to about 3.0 μg, about 2.5 μg to about 10 μg, about 5 μg to about 15 μg, about 12.5 μg to about 30 μg, about 25 μg to about 50 μg, about 40 μg to about 80 μg, about 60 μg to about 100 μg, about 90 μg to about 120 μg, about 110 μg to about 130 μg, about 125 μg to about 150 μg, about 140 μg to about 180 μg, about 170 μg to about 200 μg, about 200 μg to about 230 μg, about 215 μg to about 240 μg, about 235 μg to less than 250 μg greater than 0.0 μg to 2 μg, greater than 0.0 μg to 3 μg, greater than 0.0 μg to 10 μg, greater than 0.0 μg to 15 μg, greater than 0.0 μg to 30 μg, greater than 0.0 μg to 50 μg, greater than 0.0 μg to 80 μg, greater than 0.0 μg to 100 μg, greater than 0.0 μg to 120 μg, greater than 0.0 μg to 130 μg, greater than 0.0 μg to 150 μg, greater than 0.0 μg to 180 μg, greater than 0.0 μg to 200 μg, greater than 0.0 μg to 230 μg, greater than 0.0 μg to 240 μg, greater than 0.0 μg to 250 μg, greater than 0.0 μg to about 2 μg, greater than 0.0 μg to about 3 μg, greater than 0.0 μg to about 10 μg, greater than 0.0 μg to about 15 μg, greater than 0.0 μg to about 30 μg, greater than 0.0 μg to about 50 μg, greater than 0.0 μg to about 80 μg, greater than 0.0 μg to about 100 μg, greater than 0.0 μg to about 120 μg, greater than 0.0 μg to about 130 μg, greater than 0.0 μg to about 150 μg, greater than 0.0 μg to about 180 μg, greater than 0.0 μg to about 200 μg, greater than 0.0 μg to about 230 μg, greater than 0.0 μg to about 240 μg, greater than 0.0 μg to about 250 μg, 0 μg to less than 250 μg, 0.5 μg to less than 250 μg, 1.5 μg to less than 250 μg, 2.5 μg to less than 250 μg, 5 μg to less than 250 μg, 12.5 μg to less than 250 μg, 25 μg to less than 250 μg, 40 μg to less than 250 μg, 60 μg to less than 250 μg, 90 μg to less than 250 μg, 110 μg to less than 250 μg, 125 μg to less than 250 μg, 140 μg to less than 250 μg, 170 μg to less than 250 μg, 200 μg to less than 250 μg, 215 μg to less than 250 μg, 0 μg to less than about 250 μg, about 0.5 μg to less than about 250 μg, about 1.5 μg to less than about 250 μg, about 2.5 μg to less than about 250 μg, about 5 μg to less than about 250 μg, about 12.5 μg to less than about 250 μg, about 25 μg to less than about 250 μg, about 40 μg to less than about 250 μg, about 60 μg to less than about 250 μg, about 90 μg to less than about 250 μg, about 110 μg to less than about 250 μg, about 125 μg to less than about 250 μg, about 140 μg to less than about 250 μg, about 170 μg to less than about 250 μg, about 200 μg to less than about 250 μg, and/or about 215 μg to less about than 250 μg. Riociguat can be present in a positive amount less than 200 μg to greater than 0 or about less than 200 μg to greater than 0. Riociguat can be present in a positive amount less than 150 μg to greater than 0 or about less than 150 μg to greater than 0. Riociguat can be present in a positive amount less than 100 μg to greater than 0 or about less than 100 μg to greater than 0. Riociguat can be present in a positive amount less than 50 μg to greater than 0 or about less than 50 μg to greater than 0.

It is contemplated that the dosage unit can be steroid-free.

### Business Methods

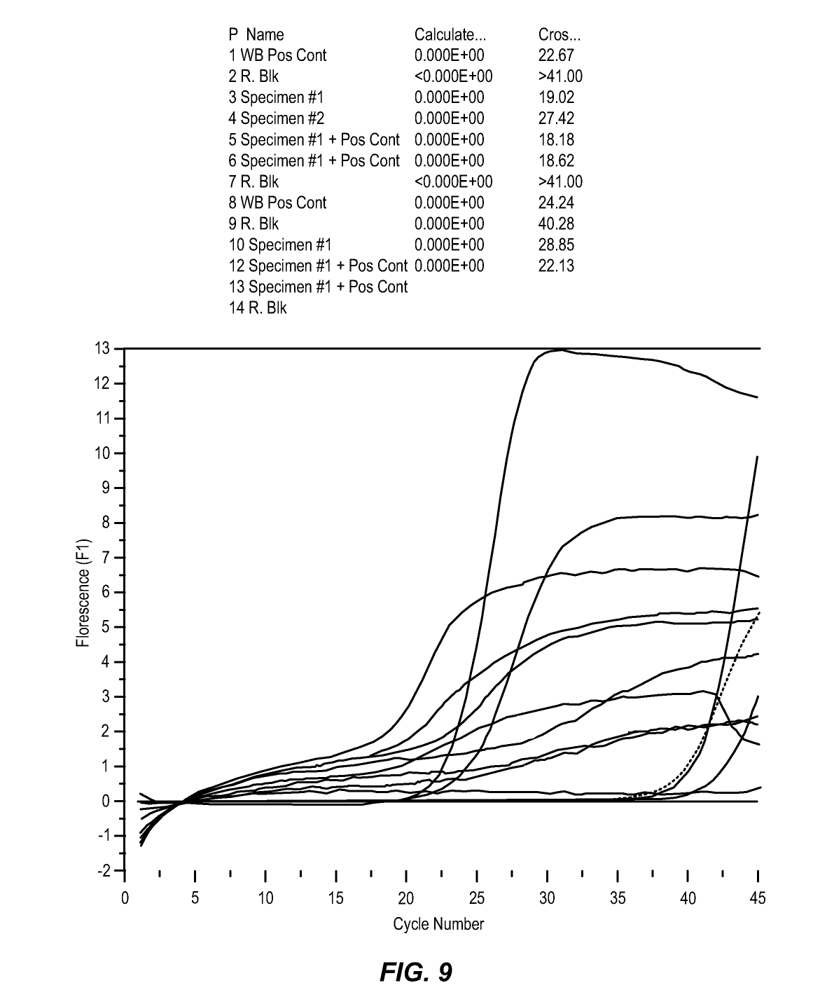

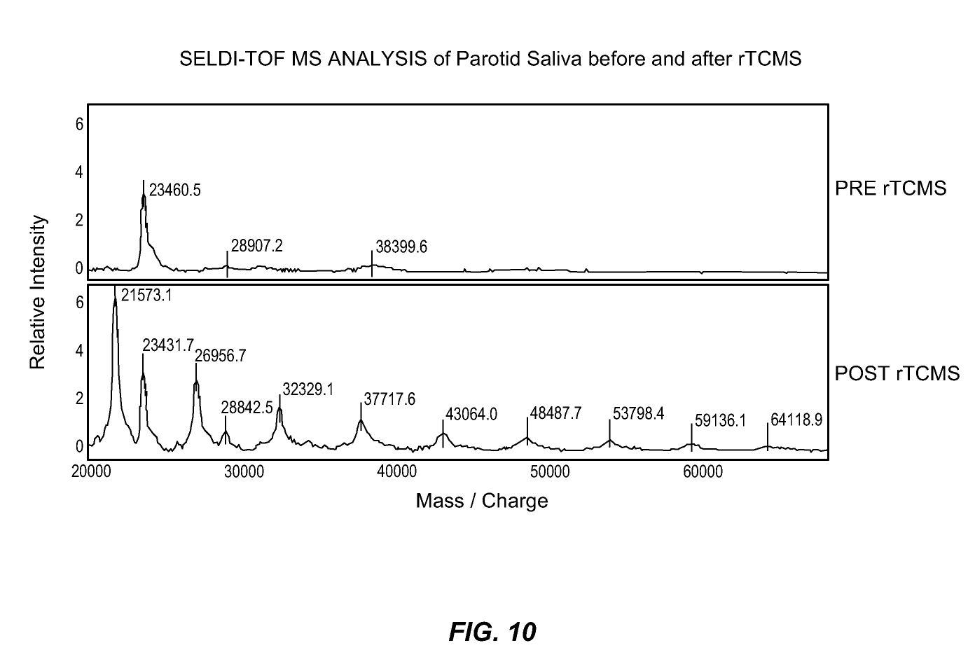

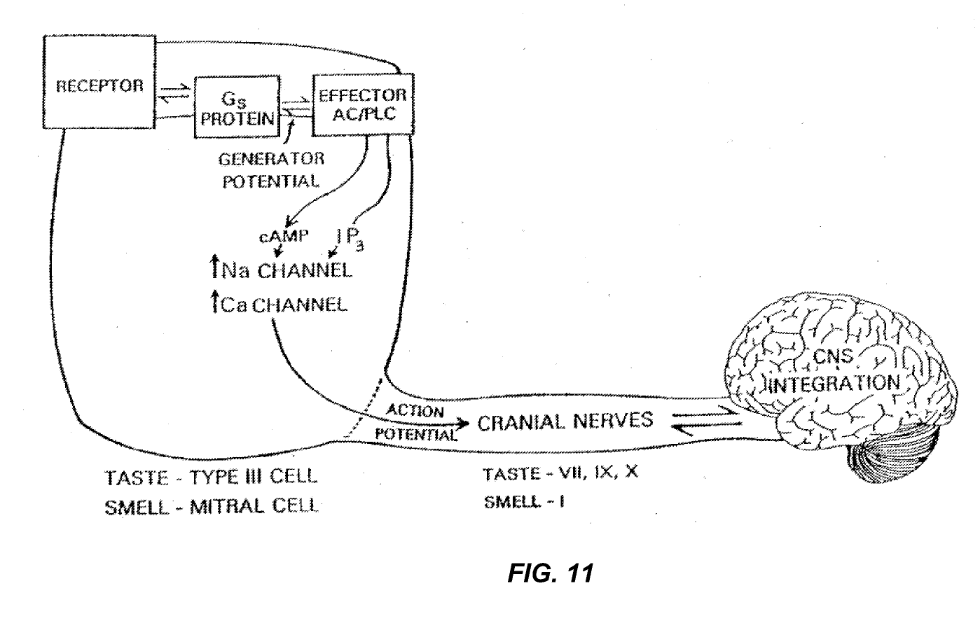

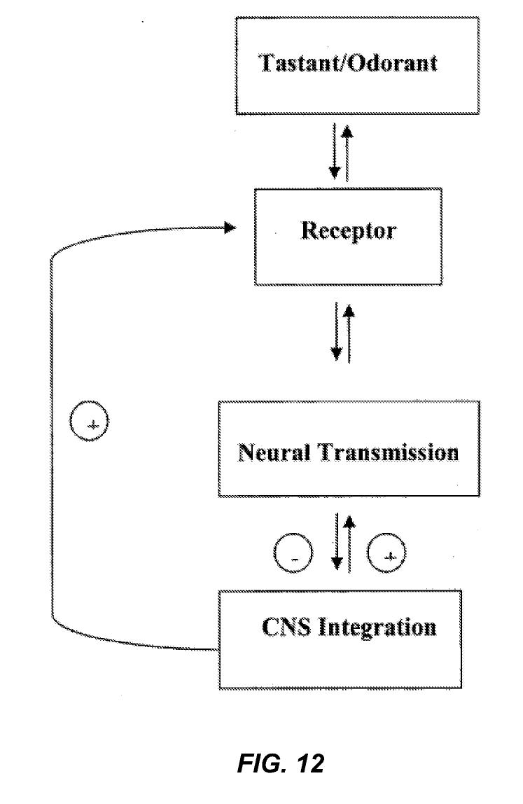

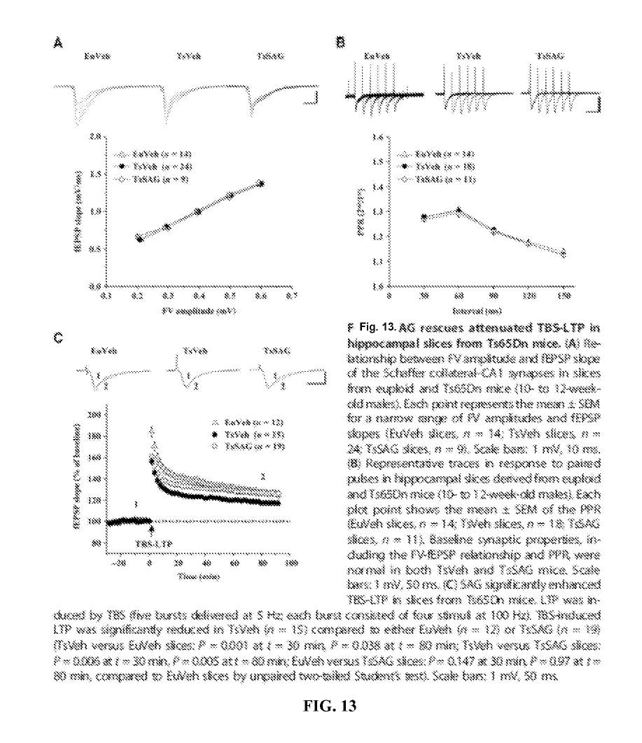

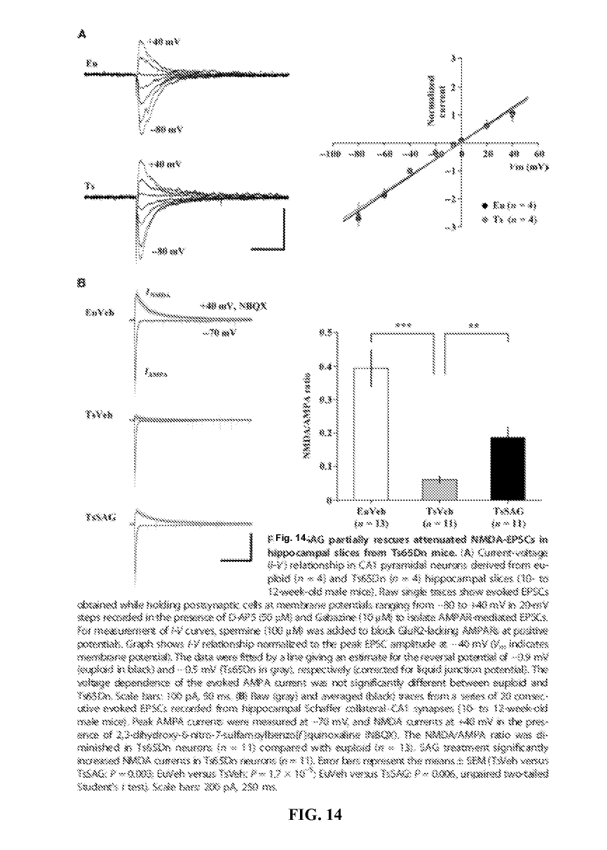

One or more computers may be utilized in the diagnostic methods disclosed herein, such as a computer 800 as illustrated in FIG. 9. It is contemplated that the computer 800 may be uniquely designed for the task at hand, e.g., the computer is not a general computer. The computer 800 may be used for managing subject and sample information such as sample or subject tracking, database management, analyzing biomarker data, analyzing cytological data, storing data, billing, marketing, reporting results, or storing results. The computer may include a monitor 807 or other graphical interface for displaying data, results, billing information, marketing information (e.g. demographics), subject information, or sample information. The computer may also include data or information input 816, 815. The computer may include a processing unit 801 and fixed 803 or removable 811 media or a combination thereof. The computer may be accessed by a user in physical proximity to the computer, for example via a keyboard and/or mouse, or by a user 822 that does not necessarily have access to the physical computer through a communication medium 805 such as a modem, an internet connection, a telephone connection, or a wired or wireless communication signal carrier wave. In some cases, the computer may be connected to a server 809 or other communication device for relaying information from a user to the computer or from the computer to a user. In some cases, the user may store data or information obtained from the computer through a communication medium 805 on media, such as removable media 812. It is envisioned that data or diagnoses can be transmitted over such networks or connections for reception and/or review by a party. The receiving party can be, but is not limited to, an individual, a health care provider, or a health care manager. For example, a computer-readable medium includes a medium suitable for transmission of a result of an analysis of a biological sample, such as a level of one or more biomarker. The medium can include a result regarding a diagnosis of having chromosomal abnormalities and/or one or more symptoms associated with chromosomal abnormalities, wherein such a result can be derived using the methods described herein.

Sample information can be entered into a database for the purpose of one or more of the following: inventory tracking, assay result tracking, order tracking, subject management, subject service, billing, and sales. Sample information may include, but is not limited to: subject name, unique subject identification, subject-associated medical professional, indicated assay or assays, assay results, adequacy status, indicated adequacy tests, medical history of the subject, preliminary diagnosis, suspected diagnosis, sample history, insurance provider, medical provider, third party testing center or any information suitable for storage in a database. Sample history may include but is not limited to: age of the sample, type of sample, method of acquisition, method of storage, or method of transport.

The database may be accessible by a subject, medical professional, insurance provider, third party, or any individual or entity granted access. Database access may take the form of electronic communication such as a computer or telephone. The database may be accessed through an intermediary such as a customer service representative, business representative, consultant, independent testing center, or medical professional. The availability or degree of database access or sample information, such as assay results, may change upon payment of a fee for products and services rendered or to be rendered. The degree of database access or sample information may be restricted to comply with generally accepted or legal requirements for patient or subject confidentiality.

### Diagnosing and Treating Symptoms of Chromosomal Abnormalities

Decreased levels of SHH or other hedgehog family members (e.g., DHH and IHH) may play an important role in the development of symptoms associated with chromosomal abnormalities, e.g., chromosomal translocations, trisomy 13, trisomy 18, and trisomy 21. To ameliorate one or more symptoms associated with chromosomal defects, a subject can be first diagnosed (in utero or post-birth) with having a chromosomal abnormality, e.g., chromosomal translocations, trisomy 13, trisomy 18, and trisomy 21. For example, prenatal genetic testing can be used to detect the presence or absence of a chromosomal abnormality in utero. Any method can be used to diagnosis a subject with carrying a fetus and/or embryo with chromosomal abnormalities, e.g., chromosomal translocations, trisomy 13, trisomy 18, and trisomy 21. If an invasive prenatal test is used, any known method can be used, e.g., amniocentesis, chorionic villus sampling, embryoscopy, fetoscopy, and/or percutaneous umbilical cord blood sampling. If a non-invasive prenatal test is used, any known method can be used, e.g., fetal cells in maternal blood, cell-free fetal DNA in maternal blood, preimplantation genetic diagnosis, external examination, ultrasound detection, fetal heartbeat, non-stress test, transcervical retrieval of trophoblast cells, and maternal serum screening. For example, kits that utilize cell free fetal DNA extracted from the maternal blood can be used. Kits such as Harmony Prenatal Test (Ariosa), MaterniT (Sequenom), MaterniT21 (Sequenom), and Panorama (Natera) can be used. If diagnostic tests are performed post-birth, any known methods such as simply karyotyping, methods using fluorescence hybridization, and/or any other genetic tests can be used.

After a subject is diagnosed with carrying a fetus and/or an embryo with chromosomal abnormalities, the subject can be treated with one or more activators of the hedgehog signaling pathway that agonizes one or more members of the hedgehog signaling pathway selected from a group consisting of: SHH, DHH, and IHH, and combinations thereof. In some cases, the activators of the hedgehog signaling pathway agonizes SHH. The one or more activators of the hedgehog signaling pathway can be selected from the group consisting of: cyclic adenosine monophosphate activator and/or cyclic guanosine monophosphate activator. The one or more activators of the hedgehog signaling pathway can also be selected from the group consisting of: ELND005, a drug that decreases myo-inositol, RG1662, Picrotoxin, GABA blocked drugs, PTZ, Nicotine, Green tea extract, Nerve growth factors, introducing a XIST gene, theophylline, riociguat, forskolin, phosphodiesterase inhibitor, or combinations thereof. Some subjects can be given theophylline.

In some instances, the one or more activators of the hedgehog signaling can be formulated as at least one composition or dosage unit. These formulations can be steroid-free. These formulations can also be formulated for intranasal administration.

Some subjects having one or more chromosomal abnormalities and/or one or more symptoms associated with chromosomal abnormalities can require continuous, indefinite treatment of the one or more activators of the hedgehog signaling pathway. For example, in some cases, the subject having one or more chromosomal abnormalities and/or one or more symptoms associated with chromosomal abnormalities can be treated in utero by giving the pregnant mother one or more activators of the hedgehog signaling pathway. However, the one or more activators of the hedgehog signaling pathway must be able to cross the fetal-placental barrier and the blood/brain barrier. For instance, theophylline, e.g., oral or nasal dosage forms, can be given to pregnant mothers. In other cases, the subject having one or more chromosomal abnormalities and/or one or more symptoms associated with chromosomal abnormalities can be treated at birth by giving the infant one or more activators of the hedgehog signaling pathway. For example, theophylline can be given orally, e.g., by mixing it with milk/formula or by giving theophylline to a breast-feeding mother, wherein the theophylline can be within the breast milk, or theophylline can be injected directly or given as a nasal dosage to the infant. The same can be done during the period of time known as post-partum, e.g., given theophylline to the infant direct or to a breast feeding mother. The subject having one or more chromosomal abnormalities and/or one or more symptoms of chromosomal abnormalities, can be also treated continuously (in utero and/or at birth), or any duration, including for the rest of its life. For example, the subject can be treated about 1, 2, 3, 4, 5, 6, 7, 8, 9, 10, 11, 12, 13, 14, or 15, times a day. The subject can be treated once a day. The subject can be treated two times a day. The subject can be treated three times a day. The subject can be treated four times a day. The subject can be treated five times a day. The subject can be treated six times a day. The subject can be treated seven times a day. The subject can be treated eight times a day. The subject can be treated nine times a day. The subject can be treated ten times a day. The subject can also be treated once about every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days. The subject can also be treated once about every 1, 2, 3, 4, 5, 6, 7, 8, 9, 10, 11, or 12 months. The subject can also be treated once about every 1, 2, 3, 4, 5, 6, 7, 8, 9, or 10 years.

By giving to a subject having one or more chromosomal abnormalities and/or one or more symptoms of chromosomal abnormalities at least one or more activators of the hedgehog signaling pathway, one or more symptoms associated with the chromosomal abnormality can be ameliorated. For example, cognitive and/or physical symptoms can be ameliorated. Some cognitive symptoms that can be ameliorated by this method can be intellectual disability, inability to speak, mental illness, autism, depression, anxiety, epileptic seizures, and dementia. Additionally, one or more physical abnormalities can be ameliorated, e.g., stunted growth, umbilical hernia, increased skin on the neck, low muscle tone, narrow roof of mouth, flat head, flexible ligaments, large tongue, abnormal outer ears, flattened nose, separation of first and second toes, abnormal teeth, slanted eyes, shortened hands, short neck, obstructive sleep apnea, bent fifth finger tip, brushfield spots in the iris, cataracts, keratonconus, glaucoma, hearing problems, otitis media with effusion, poor Eustachian tube function, single transverse palmar crease, protruding tongue, congenital heart disease, strabismus, congenital hypothyroidism, diabetes, duodenal atresia, pyloric stenosis, Meckel diverticulum, imperforate anus, celiac disease, gastroesophageal reflux disease, early menopause, infertility, and undescended testicles.

If a patient has, for example, down syndrome, treatment with one or more activators of the hedgehog signaling pathway can lead to ameliorating some abnormalities that can be common in almost all down syndrome patients, such as facial dysmorphology, a small and hypocellular brain, and/or the histopathology of Alzheimer disease. Other diseases that can be treated by methods disclosed herein, include, but are not limited to, vision problems (such as cataracts, near-sightedness, “crossed” eyes, and rapid, involuntary eye movements), hearing loss, infections, hypothyroidism, blood disorders (such as leukemia, anemia, and polycythemia), hypotonia, problems with upper part of the spine (such as misshapen bones in the upper part of the spine, underneath the base of the skull), disrupted sleep patterns and sleep disorders (such as sleep apnea), gum disease and dental problems (such as slower developing teeth, developing teeth in a different order, developing fewer teeth, or having misaligned teeth, compared to normal counterparts (e.g., persons without down syndrome), epilepsy, digestive problems, celiac disease (such as intestinal problems when down syndrome persons eat gluten), and/or mental health and emotional problems (such as anxiety, depression, and Attention Deficit Hyperactivity Disorder, repetitive movements, aggression, autism, psychosis, and/or social withdrawal).

Furthermore, down syndrome patients treated with one or more activators of the hedgehog signaling pathway can decrease the risk of certain diseases, for example, congenital heart disease (CHD). The decrease in risk can be lowered, e.g., to levels associated with people with no known chromosomal abnormalities. Some diseases risks that can be lowered by the methods herein, can be e.g., CHD, leukemia, and Hirschsprung.

Any of the methods and compositions disclosed herein can be used to treat patients having chromosomal abnormalities, e.g., down syndrome.

In some embodiments, treatment can be performed after birth and not during the fetal/embryonic stage. For example, treatment can be performed when a woman is sexually active, is attempting to conceive, and/or actually conceives. Treatment can be performed 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 weeks after conception. Treatment can be performed during labor. Treatment can be performed immediately after birth.

In some embodiments, treatment can be performed 1 week after conception. In some embodiments, treatment can be performed 2 weeks after conception. In some embodiments, treatment can be performed 3 weeks after conception. In some embodiments, treatment can be performed 4 weeks after conception. In some embodiments, treatment can be performed 5 weeks after conception. In some embodiments, treatment can be performed 6 weeks after conception. In some embodiments, treatment can be performed 7 weeks after conception. In some embodiments, treatment can be performed 8 weeks after conception. In some embodiments, treatment can be performed 9 weeks after conception. In some embodiments, treatment can be performed 10 weeks after conception. In some embodiments, treatment can be performed 11 weeks after conception. In some embodiments, treatment can be performed 12 weeks after conception. In some embodiments, treatment can be performed 13 weeks after conception. In some embodiments, treatment can be performed 14 weeks after conception. In some embodiments, treatment can be performed 15 weeks after conception. In some embodiments, treatment can be performed 16 weeks after conception. In some embodiments, treatment can be performed 17 weeks after conception. In some embodiments, treatment can be performed 18 weeks after conception. In some embodiments, treatment can be performed 19 weeks after conception. In some embodiments, treatment can be performed 20 weeks after conception. In some embodiments, treatment can be performed 21 weeks after conception. In some embodiments, treatment can be performed 22 weeks after conception. In some embodiments, treatment can be performed 23 weeks after conception. In some embodiments, treatment can be performed 24 weeks after conception. In some embodiments, treatment can be performed 25 weeks after conception. In some embodiments, treatment can be performed 26 weeks after conception. In some embodiments, treatment can be performed 27 weeks after conception. In some embodiments, treatment can be performed 28 weeks after conception. In some embodiments, treatment can be performed 29 weeks after conception. In some embodiments, treatment can be performed 30 weeks after conception. In some embodiments, treatment can be performed 31 weeks after conception. In some embodiments, treatment can be performed 32 weeks after conception. In some embodiments, treatment can be performed 33 weeks after conception. In some embodiments, treatment can be performed 34 weeks after conception. In some embodiments, treatment can be performed 35 weeks after conception. In some embodiments, treatment can be performed 36 weeks after conception. In some embodiments, treatment can be performed 37 weeks after conception. In some embodiments, treatment can be performed 38 weeks after conception. In some embodiments, treatment can be performed 39 weeks after conception. In some embodiments, treatment can be performed 40 weeks after conception.

### Hedgehog in Mice

Ishita Das et al., reported that “[d]own syndrome (DS) is among the most frequent genetic causes of intellectual disability, and ameliorating this deficit is a major goal in support of people with trisomy 21. The Ts65Dn mouse recapitulates some major brain structural and behavioral phenotypes of DS, including reduced size and cellularity of the cerebellum and learning deficits associated with the hippocampus. We show that a single treatment of newborn mice with the sonic hedgehog pathway agonist SAG 1.1 (SAG) results in normal cerebellar morphology in adults. Further, SAG treatment at birth rescued phenotypes associated with hippocampal deficits that occur in untreated adult Ts65Dn mice. This treatment resulted in behavioral improvements and normalized performance in the Morris water maze task for learning and memory. SAG treatment also produced physiological effects and partially rescued both N-methyl-D-aspartate (NMDA) receptordependent synaptic plasticity and NMDA/AMPA receptor ratio, physiological measures associated with memory. These outcomes confirm an important role for the hedgehog pathway in cerebellar development and raise the possibility for its direct influence in hippocampal function. The positive results from this approach suggest a possible direction for therapeutic intervention to improve cognitive function for this population.

### Introduction

Trisomy for human chromosome 21 (Hsa21) results in Down syndrome (DS), which is among the most complex genetic conditions compatible with survival past term. Mouse models with segmental trisomy for orthologs of Hsa21 genes show a number of complex outcomes with regard to development and function that are relevant to DS. A phenotype-based approach made possible by these animal models has supported progress in understanding many outcomes of trisomy and has led to the development of therapeutic interventions.

The cerebellum is much smaller and hypocellular in people with DS, and in the Ts65Dn and other mouse models. A hallmark of the Ts65Dn cerebellum, reduced density of granule cell neuron (GC) cell bodies in the internal granule layer, also occurs in people with DS across the entire life span. A critical reason for the reduced number of cerebellar GC in trisomic adults is a substantial reduction in the rate of cell division of trisomic granule cell precursors (GCPs) in the first days after birth. This reduction has been related to a prolonged cell cycle and results at least in part from the attenuated response of trisomic GCP to the mitogenic effects of Sonic hedgehog (Shh) growth factor, the major mitogen for this cell population. When we administered a Shh pathway agonist, known as SAG, subcutaneously to trisomic Ts65Dn mice on the day of birth, we observed increased proliferation of GCPs. This treatment normalized GCP number 6 days later (P6) when Ts65Dn mice normally have a significant deficit in this cell population. Despite the initial delay, the mitotic index of GCPs in untreated trisomic mice reached the same rate as in euploid animals by postnatal day 6 (P6).

SAG 1.1 (SAG) is a derivative of chlorobenzo[b]thiophene, which was identified as a Shh pathway agonist. SAG binds to and activates Smo, thus up-regulating the canonical Shh pathway and reproducing many activities of Shh in vitro. It is a small molecule that crosses the gut, the placenta, and the blood-brain barrier. SAG has been shown to stimulate division of neurons in the subgranular zone of the dentate gyrus (DG) after oral administration to adult mice. SAG has recently been given to newborn mice to stimulate GCP division, thereby counteracting the inhibition of GCP proliferation caused by administration of glucocorticoids.

The cognitive impairment seen in Ts65Dn, the most widely studied mouse model of DS, arises because of structural and functional differences in the trisomic brain compared to euploid. Several potential therapeutic approaches converge on the hippocampus because of its central role in learning and memory, functions that are disrupted in mouse models and also in people with DS. Ts65Dn mice are markedly impaired in learning and memory, as evidenced by their performance in the Morris water maze (MWM), and are quite different from euploid animals in the induction of long-term potentiation (LTP) in the CA1 and DG of the hippocampus.

Here, we asked whether the positive effects on cerebellar development of perinatal treatment with SAG would persist in adult trisomic mice and what this might imply as a model for therapy in DS.

Results

SAG treatment at birth normalizes cerebellar structure in adult mice

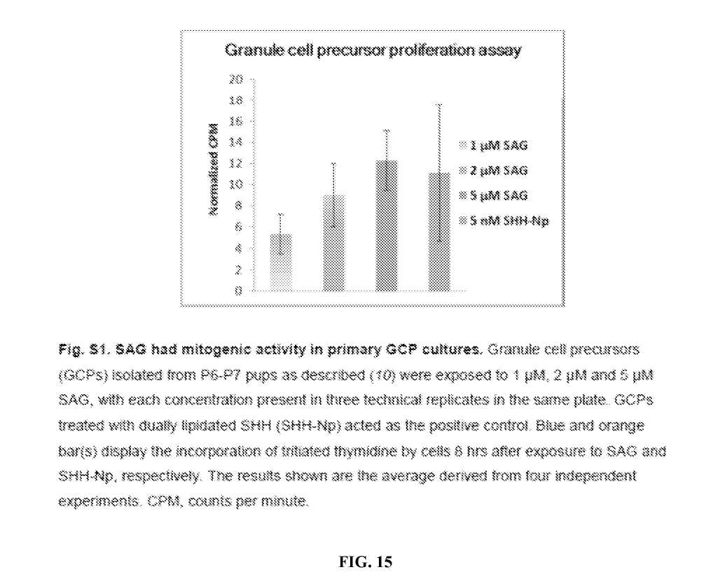

We synthesized SAG as described and compared its ability to stimulate proliferation of GCPs relative to dually lipidated Shh (Shh-Np) ([FIG. 15]). Newborn pups were injected with SAG (20 mg/g). This dose successfully normalizes proliferation of GCP in Ts65Dn mice for the first week of life, stimulates the Shh pathway in utero when given orally to pregnant dams, and stimulates proliferation of cells in the DG in young adult mice. A dose in the same range (14.0 to 25.2 mg/kg) induces maximum expression of a Gli-luciferase reporter of Shh pathway activity in the brain.

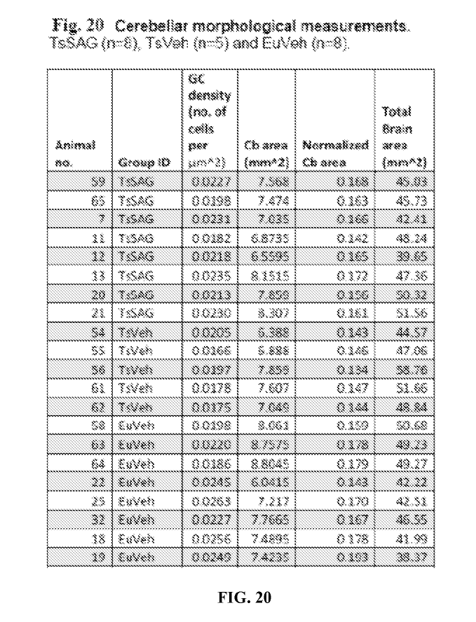

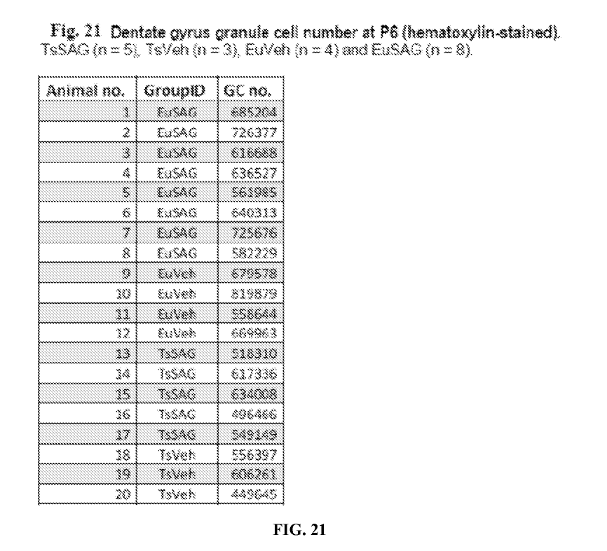

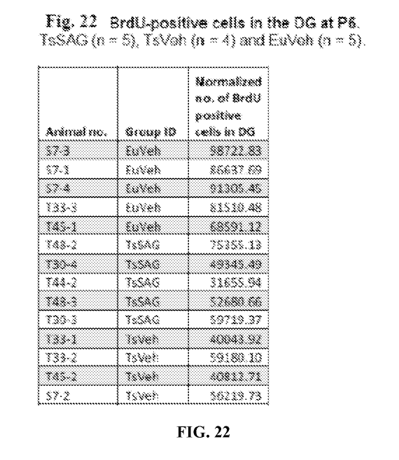

At about 16 weeks of age, we determined cerebellar area at the midline in sagittal sections and cerebellar GC density of SAG-injected Ts65Dn mice (TsSAG), euploid animals injected with vehicle (EuVeh), and vehicle-injected trisomic animals (TsVeh) (FIG. [10]). Adult TsSAG mice that received a single injection of SAG on the day of birth had the same cross-sectional area and GC density as EuVeh, and both were significantly greater than TsVeh (FIG. [10], A to C, and [FIG. 20]). We showed previously that a single dose of SAG given to euploid mice (EuSAG) at P0 did not significantly increase GCP number at P6.

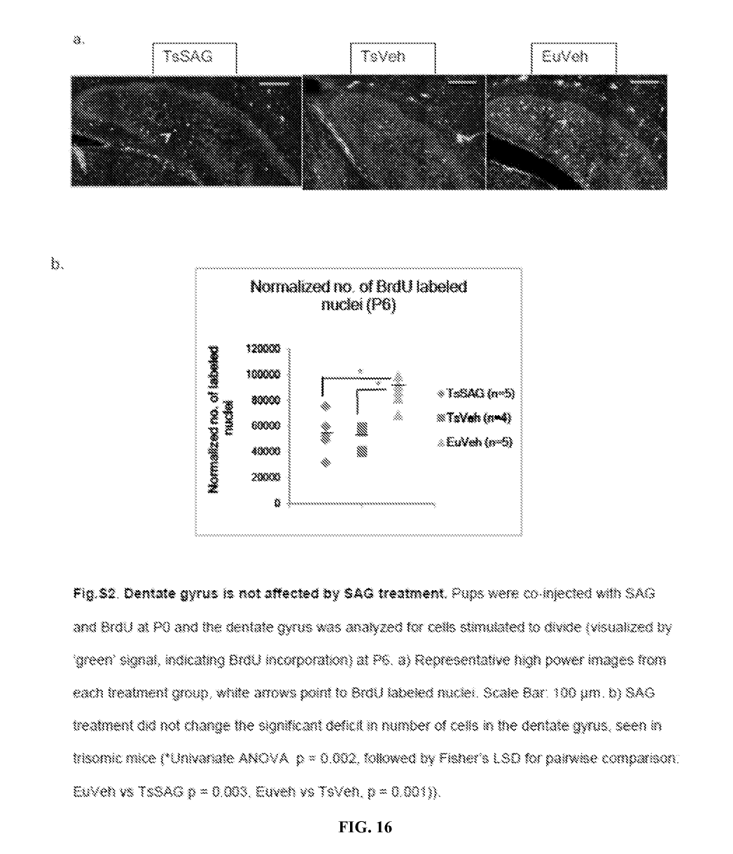

We and others have shown that the number of granule cells in DG is reduced in Ts65Dn mice as early as P6, an effect that persists through the first year of life and presumably beyond. Adult DG cell number is also influenced by external factors, such as activity or nutrition. Accordingly, we looked for acute SAG effects on proliferation in the DG by co-injecting 5-bromo-2′-deoxyuridine (BrdU) and SAG at P0 and analyzing cell number at P6. In contrast to the normalization of cerebellar granule cell number 6 days after injection of SAG, the DG deficit in Ts65Dn mice that received SAG treatment was not ameliorated (FIG. [10]D). TsVeh and TsSAG mice were not different from each other, and both showed a lower rate of proliferation in DG after SAG and BrdU labeling than did EuVeh (FIG. [10]D, [FIG. 16], and [FIGS.] [21] and [22]).

SAG does not normalize long-term depression from cerebellar Purkinje cells

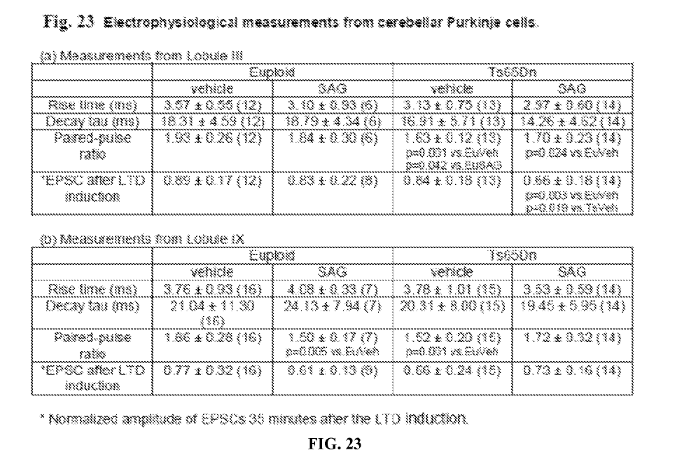

We asked whether normalization of cerebellar morphology would affect the synaptic function of cerebellar circuits measured in brain slice preparations. Excitatory postsynaptic currents (EPSCs) were recorded from Purkinje cells in lobule III and in lobule IX (FIG. [11]), because there are known differences in electrophysiological properties between these areas. Despite the pronounced morphological differences between the Ts65Dn and euploid cerebellum, we did not find differences in EPSC kinetics as indexed by rise time or decay tau in either lobule III or lobule IX ([FIG. 23]).

As an index of release probability at presynaptic terminal of GCs, we measured EPSC paired-pulse ratios (PPRs). PPRs were significantly lower in trisomic mice than in euploid in both lobule III and lobule IX (P=0.0009 and 0.0006, respectively) (FIG. [11]B and [FIG. 23]). SAG treatment did not restore the PPR values in trisomic mice. The lower PPR values in Ts65Dn suggest that release probability is increased at these synapses. This is consistent with a recent report that cerebellar GCs (the cells of origin of the parallel fiber axons) in Ts65Dn mice show increased excitability and larger action potential amplitude. We did not find any significant differences in long-term depression (LTD) expression in either lobule III or lobule IX ([FIG. 23]). SAG treatment of Ts65Dn mice resulted in more depression of EPSC after the induction of LTD (P=0.019), but this was limited to lobule III. Accordingly, it is difficult to relate therapeutic actions of SAG to effects on LTD. SAG treatment of euploid mice significantly decreased the PPR values measured in lobule IX (P=0.005), but did not affect any other parameters.

SAG normalizes performance in hippocampal but not prefrontal tasks

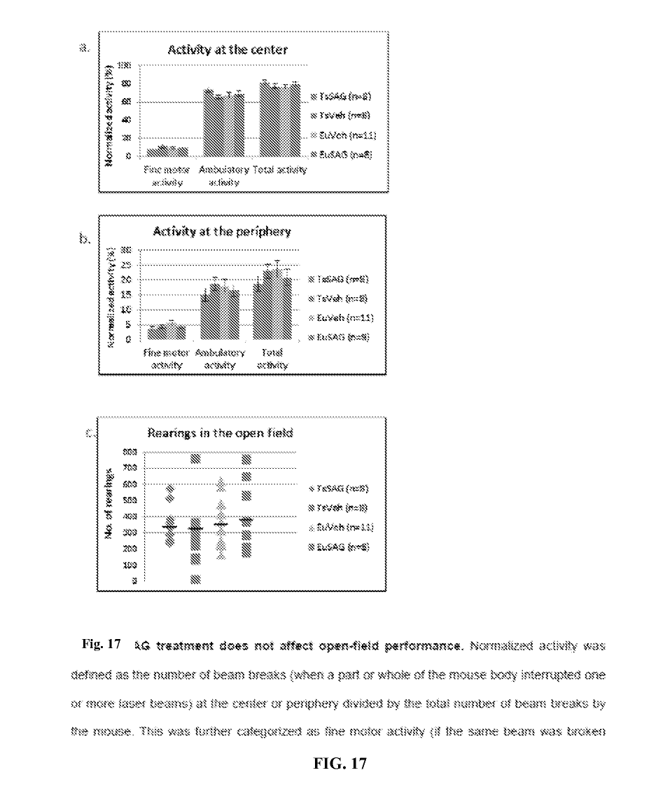

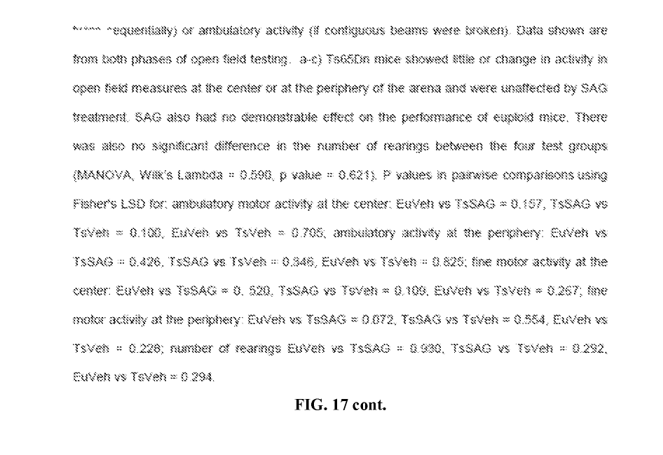

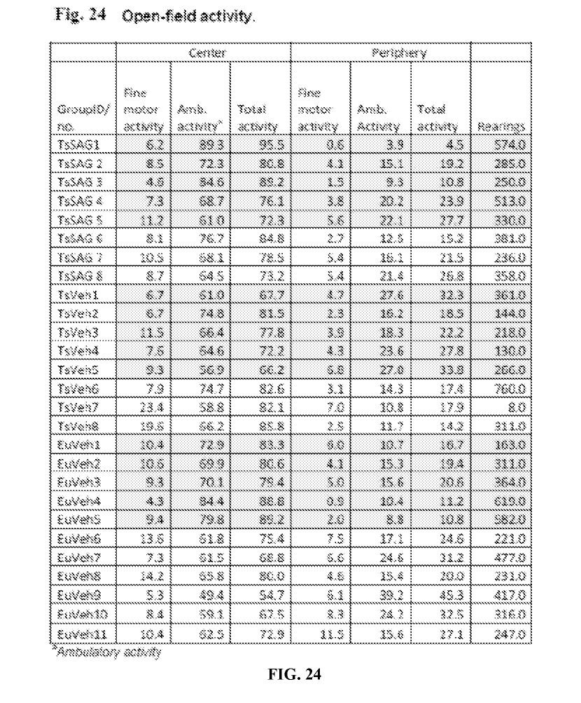

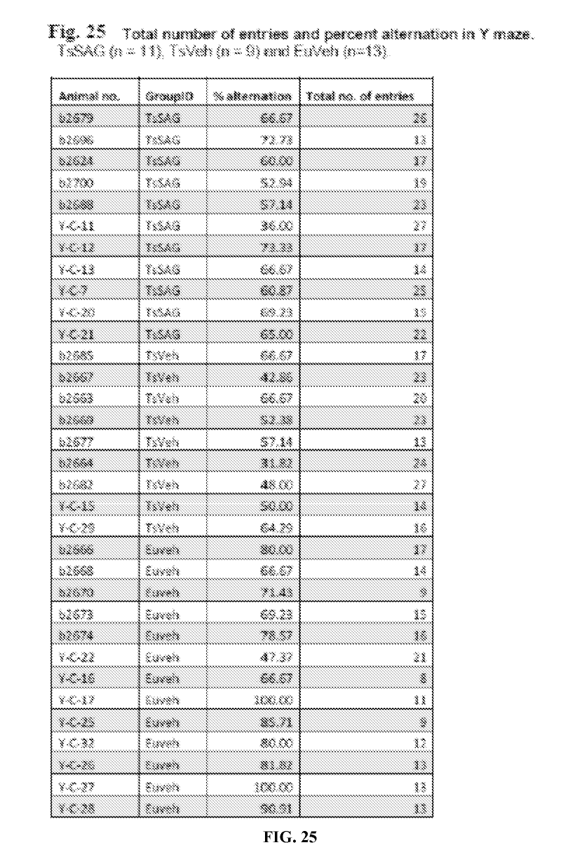

Ts65Dn mice have been tested in numerous open-field paradigms for hyperactivity and anxiety levels, with highly inconsistent results. We used the open field to look for gross effects on general locomotor function while familiarizing the mice to handling. These procedures were not powered to detect small differences consistent with anxiolytic or anxiogenic effects (power is 60%). We found that the time spent in the periphery or center of the maze was similar for EuVeh and TsVeh groups, and no significant differences were observed with TsSAG ([FIG. 17]). Separate measurements were made for the number of rearings and the number of beam breaks at the center or at the periphery, and these were further categorized into fine motor activity or ambulatory activity if the same beam was broken twice or if consecutive beams were broken, respectively ([FIG. 17] and [FIG. 24]).

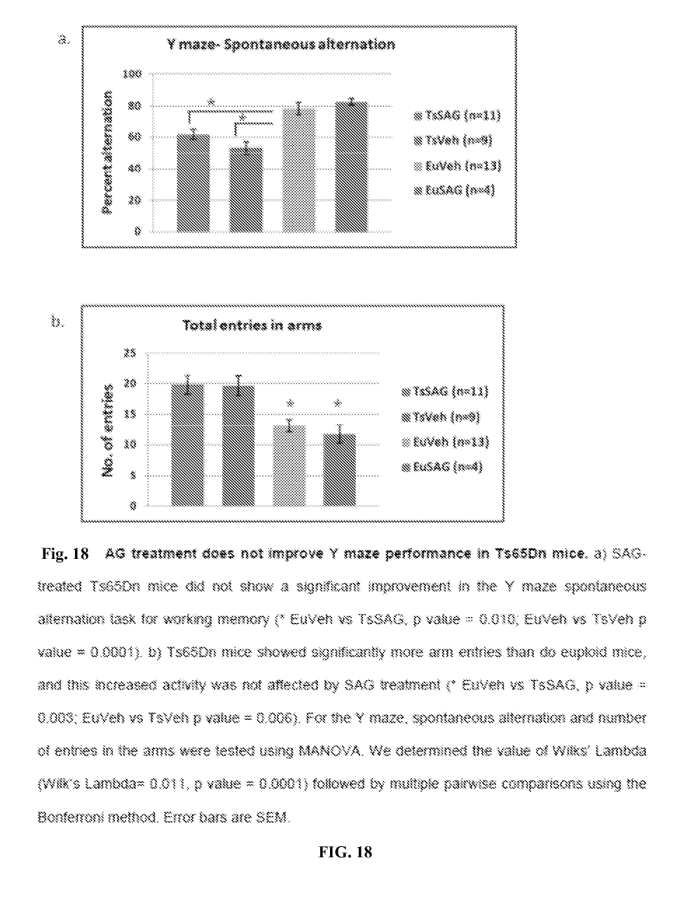

Next, we assessed a previously described deficit of Ts65Dn in the Y maze test of working memory, a non-aversive task that does not involve training or a strong stimulus. EuVeh mice (n=13) showed an average of 78% alternation. TsSAG and TsVeh mice were significantly impaired, with both groups showing only 60% alternation [EuVeh versus TsSAG: P=0.003; EuVeh versus TsVeh: P=0.0001, Fisher's least significant difference (LSD)] ([FIG. 18]A and [FIG. 25]). With more power to detect differences in activity than in open field (about 85%), we observed significantly more activity, measured as number of arm entrances, in trisomic mice with or without SAG than in euploid mice (EuVeh versus TsSAG: P=0.001; EuVeh versus TsVeh: P=0.002, Fisher's LSD) ([FIG. 18]B). SAG treatment did not have an effect on either outcome in Ts65Dn mice, nor did it alter outcomes in EuSAG compared to EuVeh.

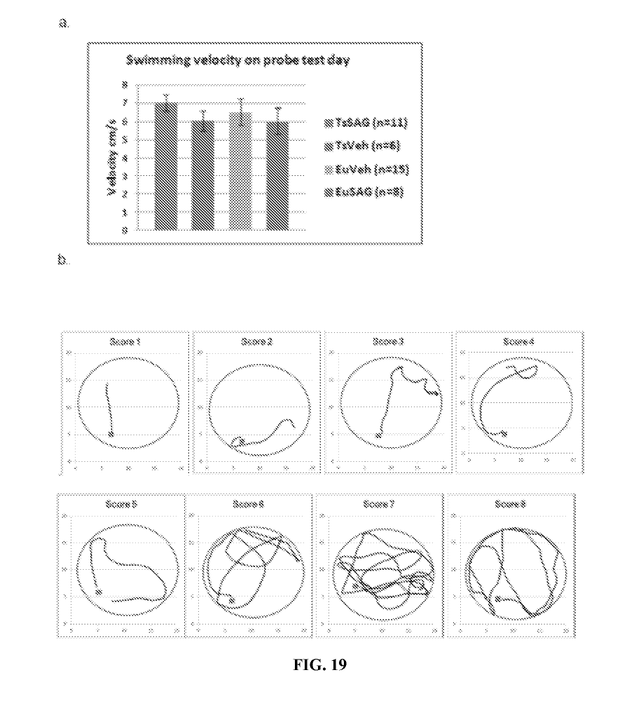

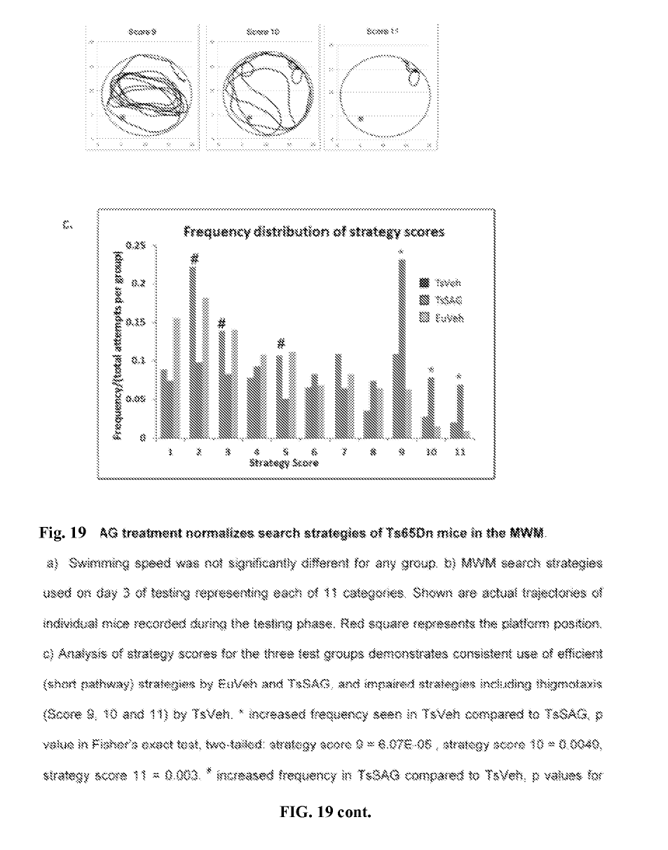

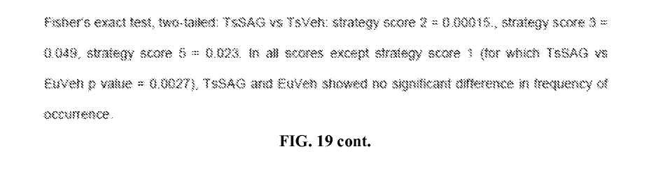

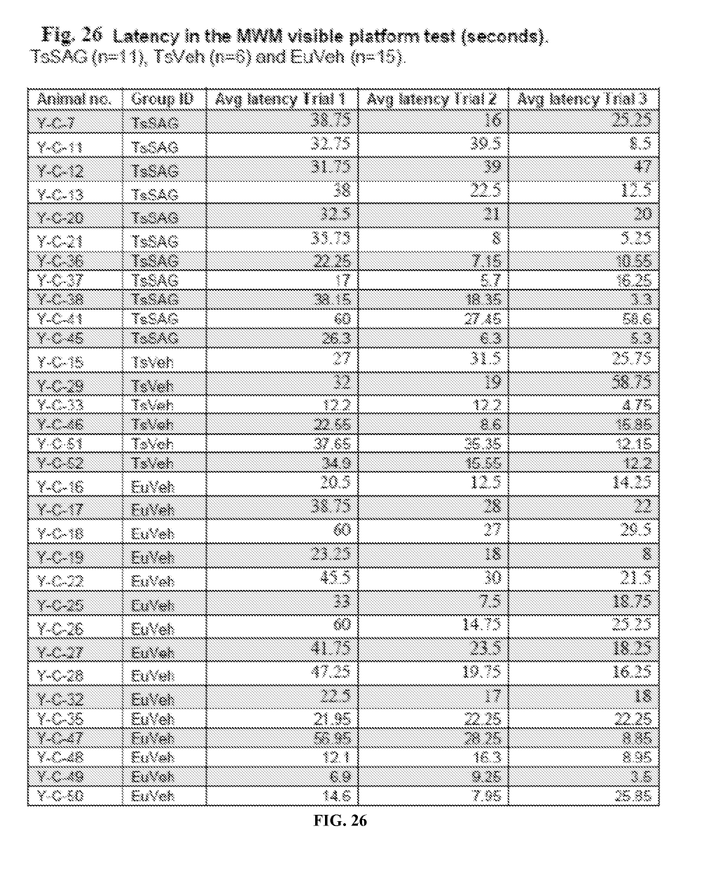

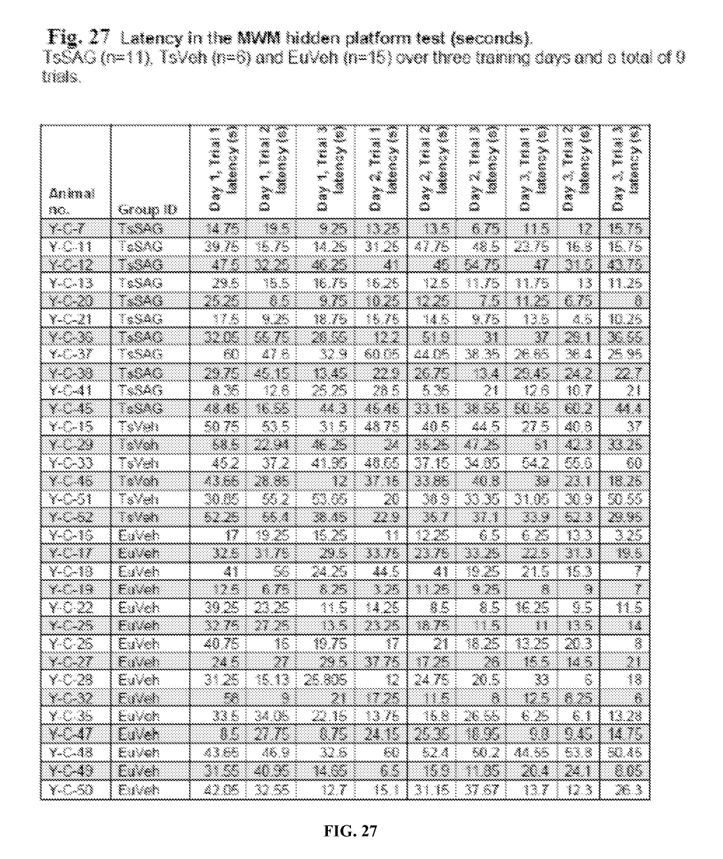

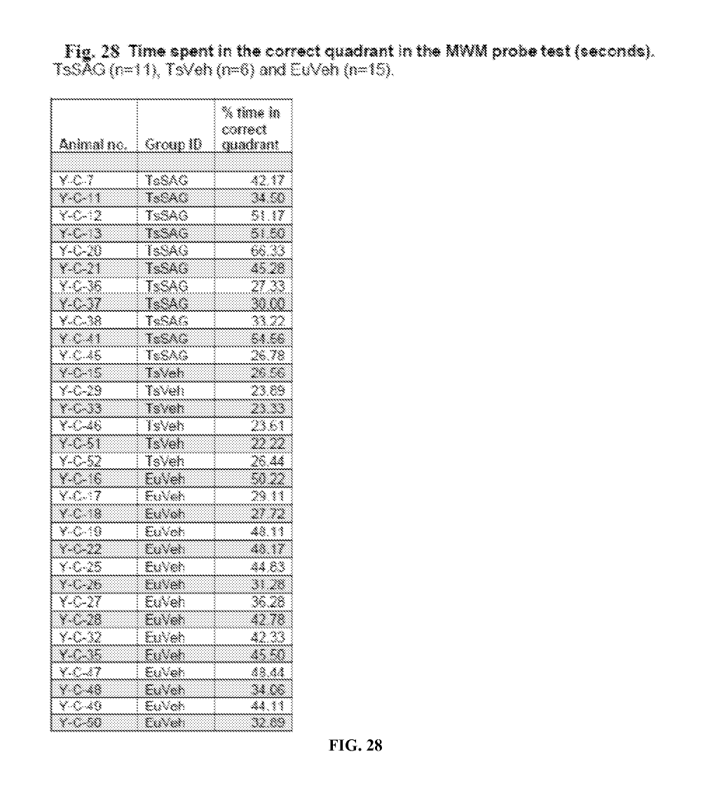

Multiple investigators have reported that Ts65Dn mice display a robust deficit in hidden platform and probe component of the MWM task. All four groups of mice tested here performed similarly in the visible platform component, as expected (FIG. [12]A and [FIG. 26]). Swimming velocities were not different between groups ([FIG. 19]A). In the hidden platform paradigm, mice learn to navigate to the platform using visuospatial cues outside the tank. As expected, TsVeh mice had prolonged escape latencies compared to EuVeh. Bonferroni-corrected P values showed a significant difference between TsVeh and EuVeh in pairwise comparison (corrected P=0.003). The pairwise comparisons were preceded by two-way repeated-measures ANOVA, which indicated a significant difference between the three groups (F2,29=6.5, P=0.005, a=0.05). TsSAG mice had similar escape latencies as EuVeh mice (P=0.91), and latencies for both groups were significantly shorter than those for TsVeh mice (TsSAG versus TsVeh, corrected P=0.042) (FIG. [12]B and [FIG. 27]). The improvement in learning was also evident in the probe test (FIGS. [12], C and D, and [FIG. 28]) (Kruskal-Wallis rank test, P=0.001; Mann-Whitney test for pairwise comparison, TsVeh versus TsSAG and TsVeh versus EuVeh, P=0.001 and 0.0001, respectively) and reflected improved memory in TsSAG mice compared to TsVeh ([FIG. 19]). SAG treatment had no effect on the performance of euploid mice (FIG. [12]).

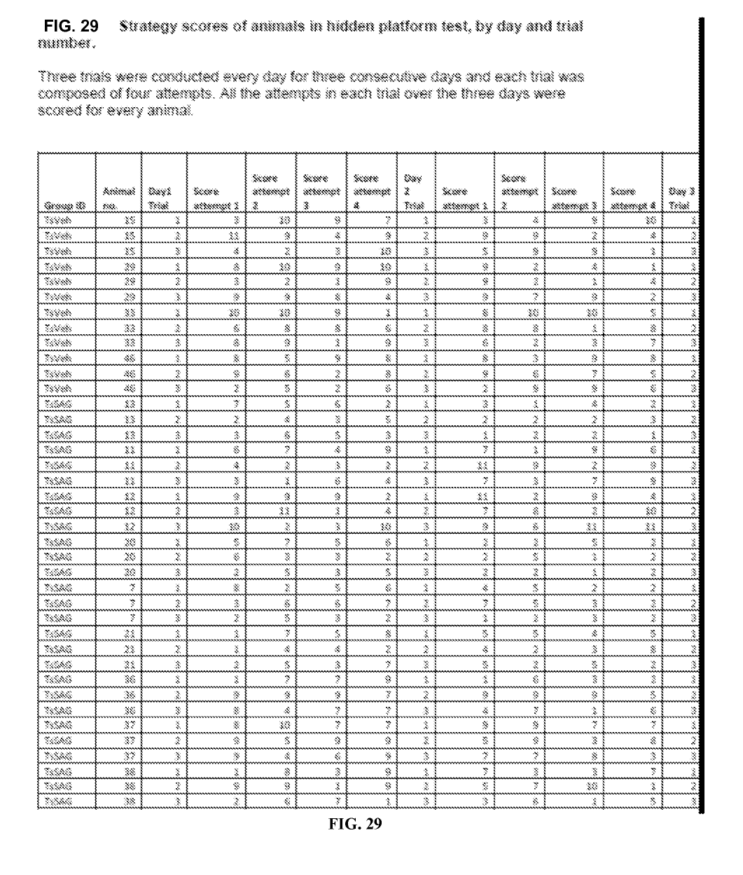

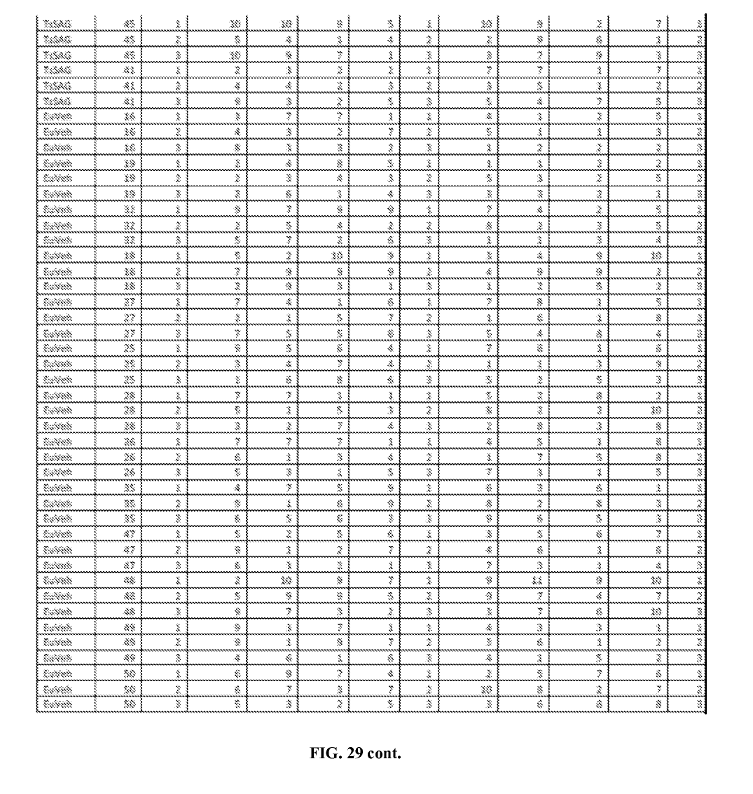

We evaluated the strategy used to find the platform based on an analysis of trajectory and latency as described (Supplementary Notes regarding behavior studies, [FIGS. 19], B and C, and [FIG. 29]). These parameters provide a detailed picture of spatial learning in the MWM that is not obtained from distance traveled alone. TsSAG mice used the same successful strategies as EuVeh, whereas the greatly increased latency for TsVeh mice was correlated with inefficient strategies. The strategy scores were highly correlated with latency in all three groups (Spearman's rho>0.80), indicating that time taken to find the platform was strongly related to the strategy.

Hippocampal physiology is partially normalized by SAG treatment

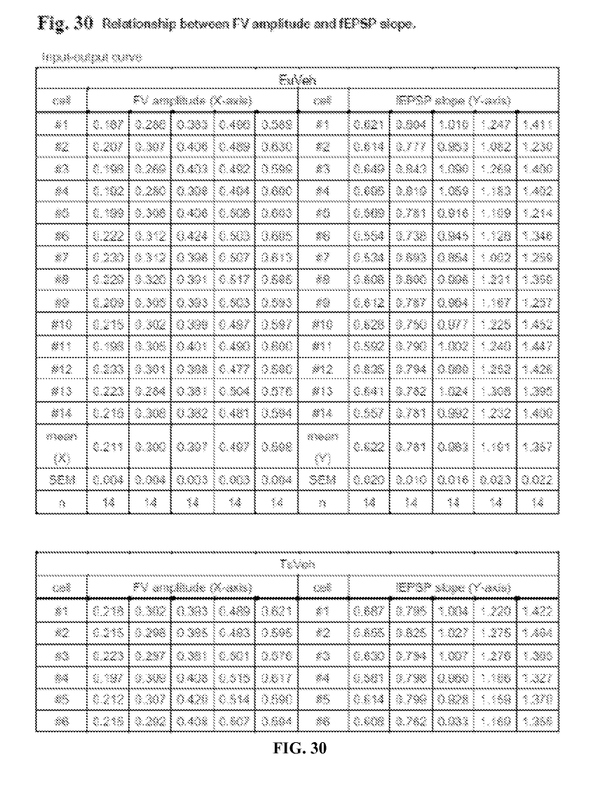

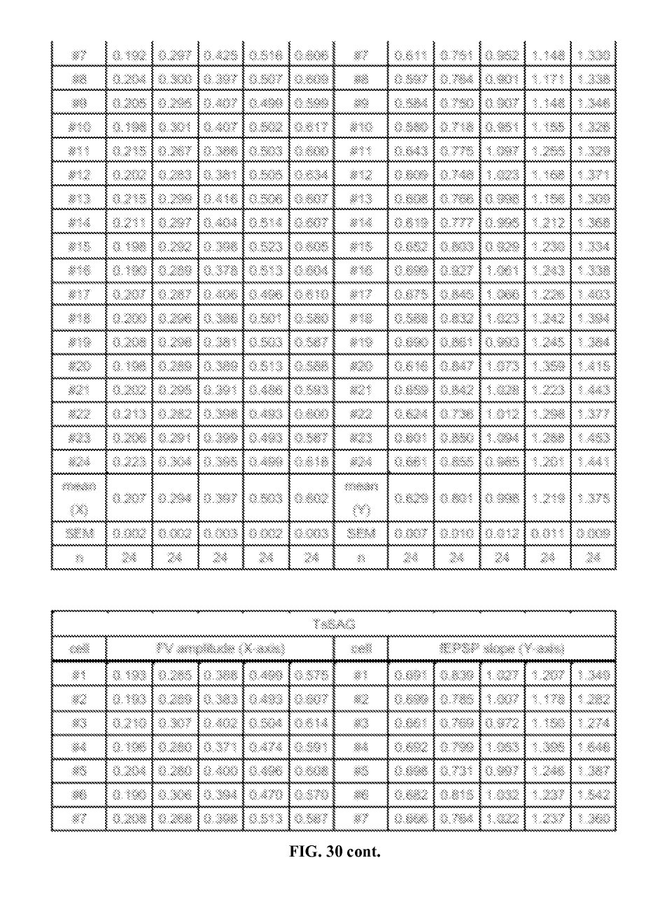

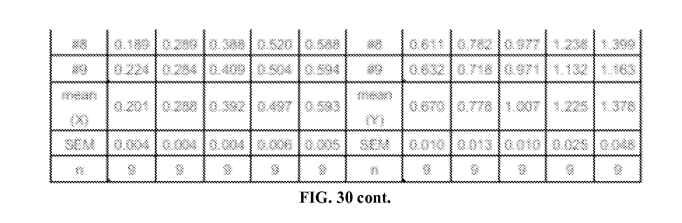

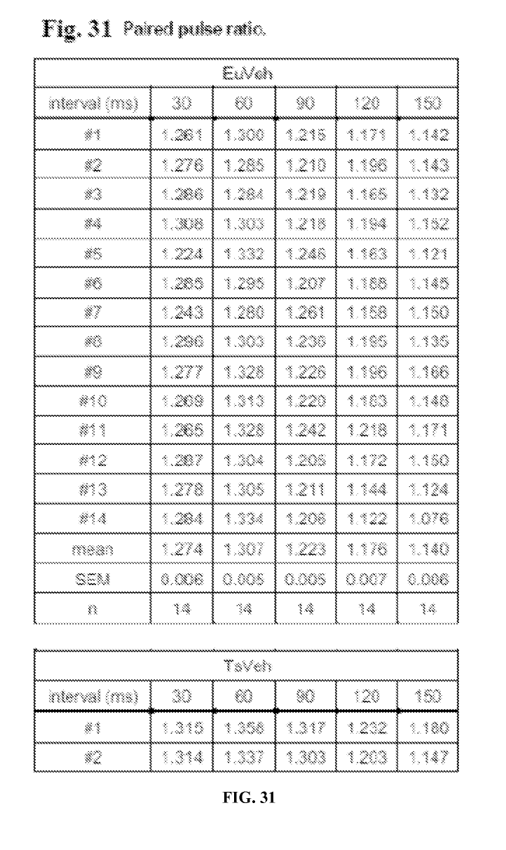

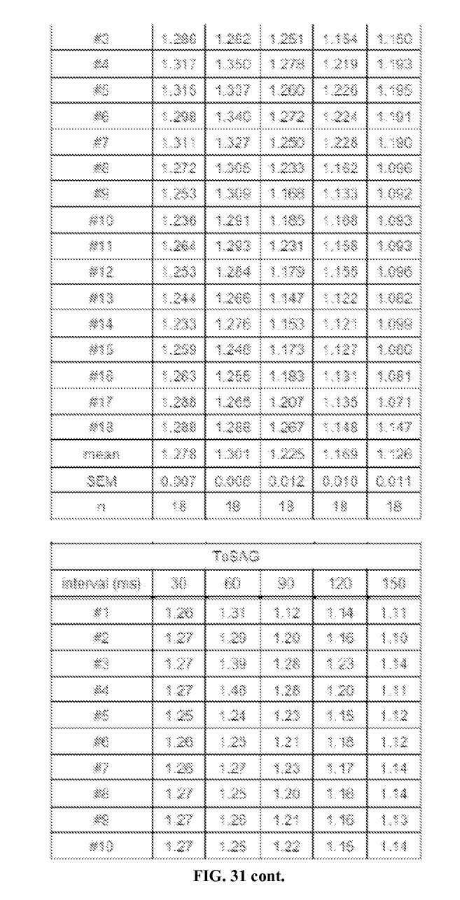

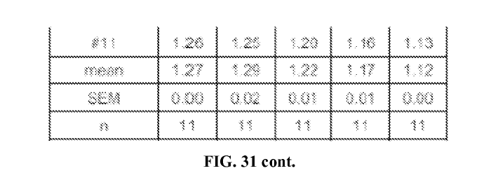

To determine whether improvements in MWM reflect physiological changes in the hippocampus, we used two different measures to characterize basal synaptic transmission. The first was to derive an index of synaptic strength by varying stimulus strength, thereby constructing an input-output plot relating presynaptic fiber volley (FV) amplitude to the onset slope of the field excitatory postsynaptic potential (fEPSP) (FIG. [13]A and [FIG. 30]). Second, we estimated the probability of neurotransmitter release by application of pulse pairs delivered at intervals ranging from 30 to 150 ms. The PPR serves as an index of release probability in a synapse (FIG. [13]B and [FIG. 31]). Both of these measures revealed similar basal synaptic properties in EuVeh, TsVeh, and TsSAG mice.

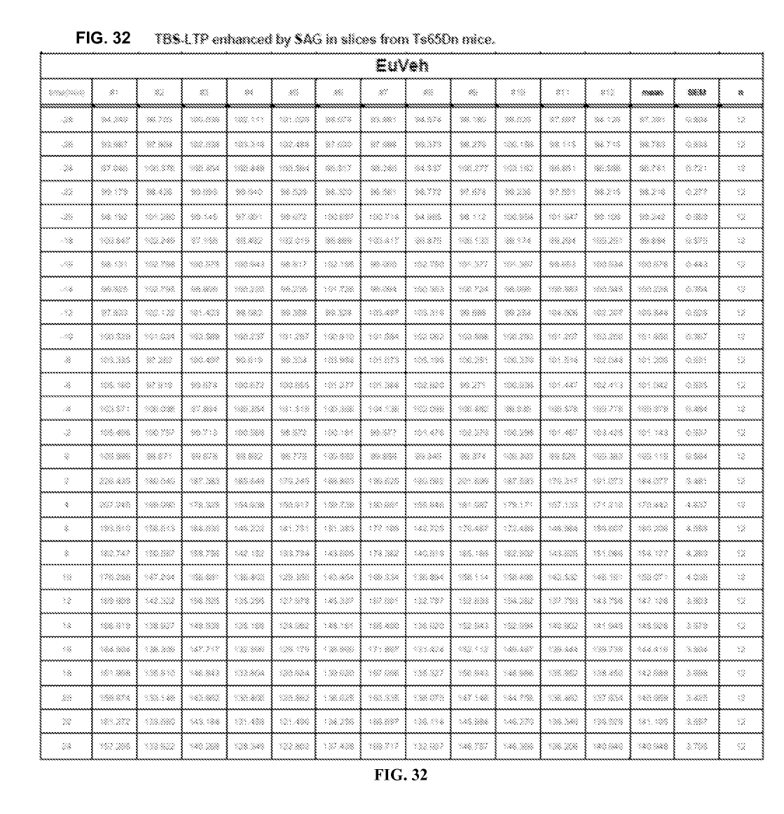

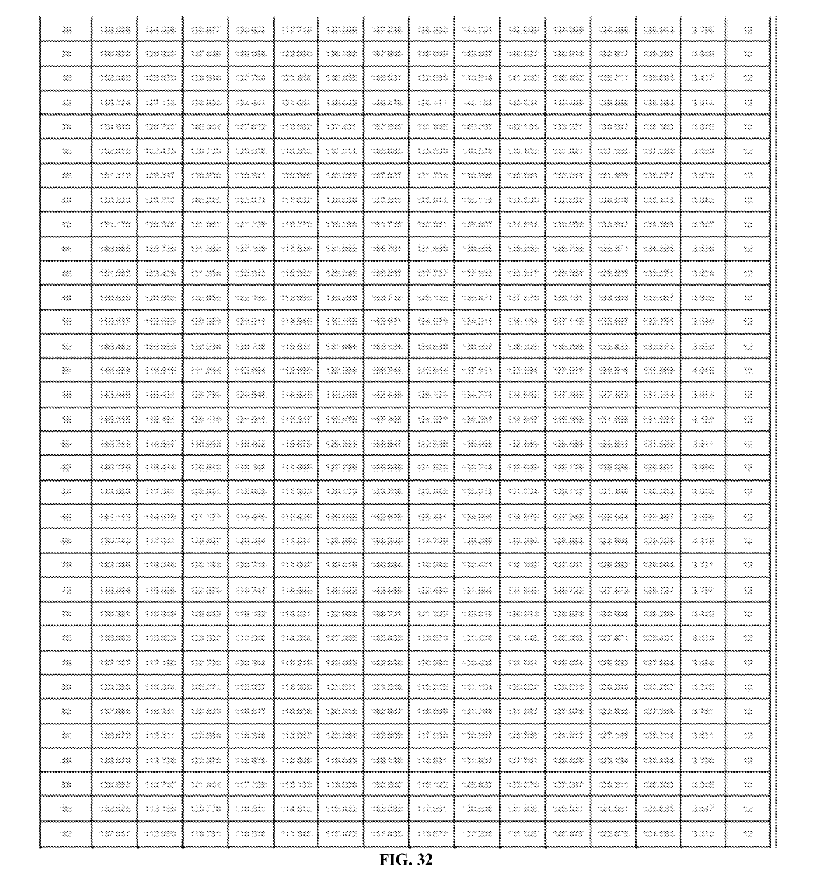

LTP evoked by theta burst stimulation (TBS) results in a rapid and sustained increase of AMPA receptor (AMPAR)-mediated responses in Schaffer collateral-CA1 synapses. In hippocampal slices derived from EuVeh mice, fEPSP was increased to 138.6±3.4% (n=12) of baseline at t=30 min after stimulation and sustained at the level of 127.3±3.7% of baseline at t=80 min (FIG. [13]C and [FIG. 32]). TBS induced LTP in TsVeh was significantly reduced (123.6±2.3% of baseline at t=30 min, P=0.001) and continued to decay more rapidly than in euploid control mice (117.4±2.7% of baseline at t=80 min, P=0.038), consistent with previous findings. However, in acute hippocampal slices derived from TsSAG mice, the magnitude of TBS induced LTP was significantly increased compared to TsVeh (132.9±2.2% of baseline at t=30 min, P=0.006) and not different from euploid (127.4±2.0% of baseline at t=80 min, P=0.97).

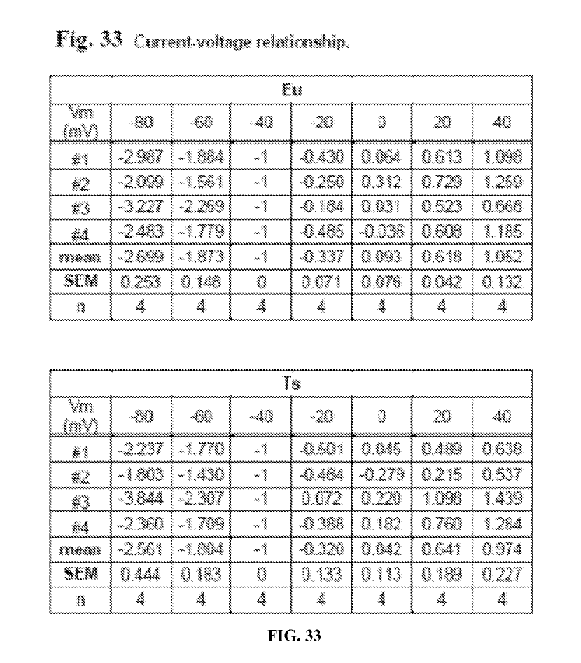

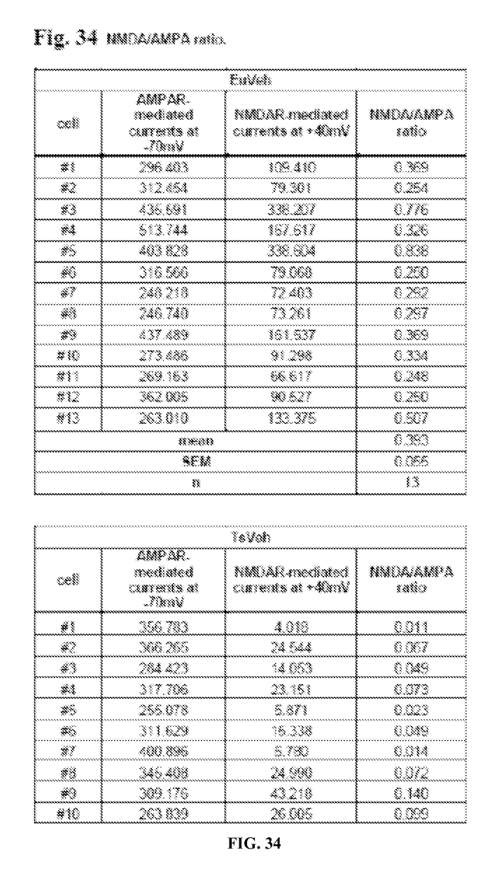

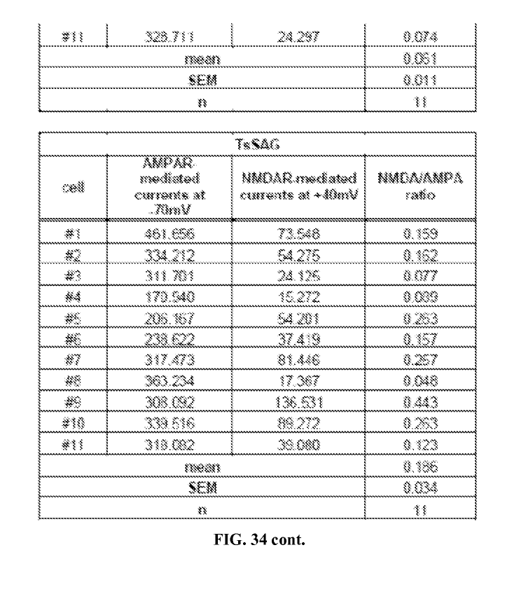

We examined synaptic properties that might underlie reduced LTP in Ts65Dn. The current-voltage relationship of evoked EPSCs in Schaffer collateral-CA1 synapses was similarly linear in euploid and Ts65Dn mice (FIG. [14]A and [FIG. 33]), indicating no difference in calcium permeable AMPARs. Next, we monitored the amplitude of evoked EPSCs using conditions that separately reveal AMPAR- and N-methyl-Daspartate receptor (NMDAR)-dependent responses. The ratio of the NMDAR/AMPAR-dependent responses in Ts65Dn mice was markedly reduced compared to that in the euploid mice (FIG. [14]B and [FIG. 34]) (P=0.06±0.011 in Ts65Dn, n=11; P=0.39±0.059, n=13 in euploid; P=0.00002). The NMDA/AMPA ratio in TsSAG (0.19±0.034, n=11) was significantly increased compared to that in TsVehmice (P=0.003) but was not restored to euploid levels (P=0.006). This is consistent with the reduction of NMDAR-dependent LTP in Ts65Dn mice and enhancement of NMDAR dependent LTP in SAG-treated animals.

### Discussion

GCPs in cerebellum of newborn Ts65Dn mice demonstrate a short lag in the initiation of the burst of proliferation relative to euploid. Acute SAG treatment stimulates the division of trisomic cells, and here, a single treatment on the day of birth was sufficient to overcome the transient proliferation deficit and normalize cerebellar structure in adult Ts65Dn mice. Ts65Dn mice do not show behavioral measures of cerebellar dysfunction in typical assays such as the accelerating rotarod. Accordingly, our analysis focused on electrophysiological measures and revealed that SAG treatment is linked to a modest increase of LTD in lobule III (but not in lobule IX) of Ts65Dn. In contrast, a single treatment with SAG resulted in robust improvement in learning and memory behavior in assays that are sensitive to hippocampal function, and to improved NMDAR function and synaptic plasticity. The persistence of these improvements is striking and invites comparison with reports of persistent improvements of cognition and LTP after pharmacological treatment of adolescent Ts65Dn mice with g-aminobutyric acid type A (GABAA) antagonists. Understanding the basis for these long-term therapeutic effects may have implications for treating DS, and the current results should encourage further exploration of a possible role for Shh in perinatal programming of hippocampus.

SAG penetrates the blood-brain barrier and can be anticipated to activate Shh signaling in cerebellar and hippocampal neurons. We did not see compensation of the small deficit in the number of replicating cells in DG of Ts65Dn by perinatal SAG treatment. From this result, it appears that normalization of DG cell number is not a necessary condition for normalizing the several behavioral and physiological outcomes in Ts65Dn mice that were assessed in this study. Normalization of cerebellar morphology might contribute to improved behavioral outcomes in the MWM. The cerebellum plays an important role in spatial learning, where it is involved in the acquisition of optimal strategies in tasks in which memory is a component, including the MWM hidden platform. Here, improved learning and memory were correlated with normalization of cerebellar morphology after SAG treatment. These results are consistent with role of cerebellum in spatial learning and suggest that the marked cerebellar hypoplasia in DS may contribute to some cognitive deficits as well.

Up-regulation of the Shh pathway by SAG has now been shown to be efficacious in several situations. In addition to correction of cerebellar hypoplasia in trisomic models, Shh or SAG can support proliferation of neural precursors in vivo after spinal cord injury in rats. SAG administration can also counter the antiproliferative effects of glucocorticoids on cerebellar GCPs in newborn mice. A number of ciliopathies have pathology related to disruption of hedgehog signaling, and SAG might have a therapeutic role in ameliorating some of these effects.

However, pharmacological stimulation of the Shh pathway in newborn infants as a therapeutic strategy might be problematic. Hedgehog signaling plays a central role in many fundamental aspects of development including axis formation and generation of neural crest, and many of its effects are dosage-sensitive. Shh is also required for stem cell generation and maintenance in differentiated tissues. Chronic Shh pathway stimulation is observed in a number of tumor types and directly linked to an increased incidence of medulloblastoma. SAG-treated mice studied here showed no evidence of tumor formation or obvious complications in the first 4 months of life. Before a clinical application is contemplated in people with DS, however, it will be necessary to better understand the SAG role in hippocampal function and the sensitivity to possible side effects on different genetic backgrounds while refining both the dosage and the route of drug administration. It would be useful to understand why trisomic GCP (and possibly other trisomic cells) has an attenuated response to the mitogenic effects of Shh, which might offer further targets for therapy. We note that there is no evidence in our data and no current theoretical basis for a positive role of Shh pathway stimulation at birth on cognitive ability in euploid adults.

We demonstrated the efficacy of a possible approach to the improvement of learning and memory in a trisomic mouse model. A single injection of a Shh pathway agonist on the day of birth corrected a key developmental deficiency in cerebellum, restoring normal structure in adults. This single treatment evoked a positive and lasting effect on hippocampal-dependent learning and memory, and partially normalized hippocampal synaptic NMDAR function and NMDAR-dependent LTP expression. These observations suggest a possible approach to ameliorate cognitive deficits that occur as a consequence of trisomy 21.

Materials and Methods

Study Design

Our previously published studies show that the Ts65Dn mouse displays and predicts aspects of cerebellar pathology that occur in people with DS, that the cerebellar hypoplasia is substantially due to an attenuated response of gcp to the mitogenic effects of Shh growth factor in the period close to the time of birth, and that stimulation of the Shh pathway with systemic application of SAG at P0 eliminates the gcp deficit at P6. On the basis of these findings, we designed a study to determine whether these effects of SAG might extend beyond the perinatal period. We injected animals at birth with a dose of SAG that produced salutary effects in previous studies from our laboratory and others. One set of animals was prepared for behavior studies on the basis of our previous determination of variation/cohort size required to power a significant analysis of the MWM paradigm, which is robustly affected in Ts65Dn mice. Similarly, sample sizes for the cohorts subjected to the standard electrophysiological paradigms tested here were chosen on the basis of previous experience. Statistical analyses are described in detail below. In all cases, investigators performing tests were blind to genotype and treatment.

Animals

Founder B6EiC3H-a/A-Ts65Dn (Ts65Dn) mice were obtained from the Jackson Laboratory and maintained in our colony as an advanced intercross on a C57BL/6J×C3H/HeJ background. SAG was synthesized as described, dissolved in ethanol or dimethyl sulfoxide, and resuspended in triolein. Activity of this batch of SAG was established by comparison to the amount of GCP proliferation relative to Shh ([FIG. 15]). Each pup in a given litter received a subcutaneous dose of SAG (20 mg/g) or vehicle in 20 ml.

Behavior Testing.

Animals were given a coded ID by someone other than the investigator so that all tests were performed by investigators who were blind to genotype and treatment group. Tests were performed in the following order: open field, Y maze, MWM. The open-field test was conducted in the photobeam activity system (San Diego Instruments) in a novel room to which the mice had not been habituated before the test. Mice were placed in a clear acrylic container [16 inches (W)×16 inches (D)×15 inches (H)] for 90 min in the first phase and 50 min in the second phase. The numbers of movements at the center, movements at the periphery, and rearings were recorded. Normalized activity is defined as the number of beam breaks at the center or periphery divided by the total number of beam breaks by the mouse. This was further categorized as fine motor activity (if the same beam is broken twice sequentially) or ambulatory activity (if contiguous beams are broken). Data shown are from both phases of open-field testing ([FIG. 24]).

For the Y maze, mice were habituated to handling for 3 days. They were released on a randomly chosen arm of a stainless steel Y-shaped apparatus, and movements were tracked for 5 min with the SMART program (San Diego Instruments). An entrance was scored when the head and front two paws were in an arm >0.2 s ([FIG. 23]).

MWM was initiated a week after Y maze. A tank of 120-cm diameter was filled with dilute latex paint at 19° to 22° C. For the visible platform test, the position of a platform submerged about 1 cm below the surface was indicated with a flag. This test was conducted on 1 day with three blocks of trials of four attempts each lasting up to 60 s. The position of the cued platform was changed for each attempt in each trial ([FIG. 26]). The hidden platform test was conducted 10 days later, with the platform always in the same position for three training days. Latency and path were recorded ([FIG. 27]). The following day, the platform was removed for the probe trial, when mice were allowed to swim for 3 min and the time spent in each quadrant was measured ([FIG. 28]). Tracks followed by the mice were extracted with the SMART program (San Diego Instruments) and scored with a modification of the method of Petrosini et al. ([FIG. 18] and [FIG. 29]).

Histological Measurements.

Tissue harvest and histological preparation were performed as described. Relative midline sagittal area of the cerebellum was measured with Image J and normalized to the midline area of the entire brain. Unbiased stereology was performed with Stereologer 1.3 (SPA Inc.) on 30-mm sections of the brain of P6 animals. The optical disector method was used to obtain density, and Cavalieri's principle was used to estimate volume. The frame area of the disectors was 169 mm2, depth was 10 mm, and guard height was 5 mm. Disectors were spaced at intervals of 95 mm. Nuclei were counted at 500×. The coefficient of error within and between samples was ≤10%. The sampling fraction was one in six sections. On average, 13 sections per animal were sampled.

BrdU (250 mg/g) was included in the SAG or vehicle preparations injected at P0. Treated mice were sacrificed at P6. The brain was fixed in 4% paraformaldehyde for 14 hours at 4° C. and then transferred to 20% sucrose solution with one change after 24 hours. Serial coronal sections (50 mm) that contained the hippocampus were cut from lateral 1.94 mm to 4.04 mm bregma, compare the Mouse Brain in Stereotaxic Coordinates. One in five sections was processed for BrdU and NeuN double labeling according to the indirect immunofluorescence method of Coons with the primary mouse anti-NeuN biotin (Chemicon) and rat anti-BrdU (Novus) antibodies, followed by secondary Alexa Flour 488 donkey anti-rat immunoglobulin G (IgG) (Molecular Probes) and Alexa Fluor 594 streptavidin (Molecular Probes) antibodies. The number of BrdU-labeled nuclei in the DG was estimated with the principles of unbiased stereology as described above. Volume (Vref) of the chosen half of the DG was estimated with the total area of the sampled sections (Aref), the average thickness of the sections (t), and the sampling fraction. Each sampled section was imaged with two-photon excitation by the Chameleon Vision II laser (Coherent Inc.) attached to a Zeiss axioscope 710NLO microscope. A low-magnification image was used to determine the section area. A pilot experiment determined the area and density of the optical dissector placement so as to allow counting of about 15 to 20 nuclei per section or 100 to 150 nuclei per animal. On average, eight sections were analyzed per animal. An area of 21 mm×21 mm was found to be acceptable with one disector placed every 0.02 mm2 through the DG. An estimate of the total number of BrdU-labeled nuclei was attained by multiplying the Vref by Nv (observed density) ([FIGS.] [21] and [22]).

Electrophysiology

Cerebellum. Parasagittal slices (250 mm) were obtained from EuVeh (n=9), EuSAG (n=5), TsVeh (n=9), or TsSAG (n=9) mice aged 21 to 28 days with a Leica vibratome in an ice-cold cutting solution containing 225 mM sucrose, 119 mM NaCl, 2.5 mM KCl, 0.1 mM CaCl2, 4.9 mMMgCl2, 26.2 mM NaHCO3, 1 mMNaH2PO4, 1.25 mM glucose, and 3 mM kynurenic acid bubbled with 95% O2 and 5% CO2. Whole-cell recordings were made from Purkinje cells in either lobule III or lobule IX at 70 mV in artificial cerebrospinal fluid (aCSF) containing 124 mM NaCl, 2.5 mM KCl, 2.5 mM CaCl2, 1.3 mMMgCl2, 26.2 mM NaHCO3, 1 mM NaH2PO4, and 20 mM glucose bubbled with 95% O2 and 5% CO2 at room temperature. Gabazine (5 mM) (Sigma) was added to block GABAA receptor currents. Recording electrodes contained a solution composed of 120 mMCs-methanesulfonate, 10 mMCsCl, 10 mMHepes, 0.2 mMEGTA, 4 mMNa2-ATP (adenosine triphosphate), and 0.4 mMNa-GTP (guanosine triphosphate) (pH 7.25). Paired stimulations (50 ms apart) were done with a glass electrode filled with aCSF by passing 20 to 40 mA of current with 0.2-ms duration to evoke EPSCs having around 200-pA amplitude. LTD was induced by a train of 10 stimuli at 100 Hz depolarizing the postsynaptic cells to 0 mV, which was repeated 30 times every 2 s. Currents were filtered at 1 kHz, measured with Multiclamp 700B (Molecular Devices), and acquired with Clampex software (Molecular Devices) at 5 kHz. EPSC amplitudes, rise time, and decay tau were measured offline with Clampfit software (Molecular Devices). Two-way ANOVA was used for statistical analysis ([FIG. 23]).

Hippocampus: Slice preparation. Transverse hippocampal slices (400-mm thick) were prepared at P90 to P120 by cutting on a tissue slicer in ice-cold dissection buffer: 110 mM choline chloride, 2.5 mMKCl, 7 mMMgCl2, 0.5 mMCaCl2, 2.4 mM sodium pyruvate, 1.3 mM sodium L-ascorbate, 1.2 mM NaH2PO4, 25 mMNaHCO3, and 20 mMD-glucose. Slices were recovered for 3 to 6 hours at room temperature in aCSF composed of 124 mM NaCl, 2.5 mM KCl, 1.3 mM MgCl2, 2.5 mM CaCl2, 1 mM NaH2PO4, 26.2 mM NaHCO3, and 20 mM D-glucose and saturated with 95% O2 and 5% CO2. Hemi slices were recorded in an interface chamber, maintained at 32° C. for 1 hour, and perfused continuously with aCSF at a rate of 3 ml/min.

Hippocampus: Field potential recording. fEPSPs were recorded from the stratum radiatum of acute hippocampal slices in response to stimulation of the Schaffer collateral commissural pathway, as described. Stimulus intensity was adjusted to elicit 50 to 60% of the maximal fEPSP slope response. LTP was measured in Schaffer collateral-CA1 synapses. Experimenters were blind to the genotype/treatment throughout the experiments. LTP was induced by TBS (five trains of four pulses; at 100 Hz and 200 ms apart). Evoked responses were stored online and analyzed offline with Clampfit (version 9.2). Time course of LTP was expressed as percentage of the fEPSP slope during the baseline recording ([FIGS.] [30] to [32]).

NMDAR/AMPAR ratio and I-V curves of AMPA-evoked EPSCs

Evoked EPSCs and the peak amplitude were recorded at a holding potential of Vh=−70 mV to access AMPAR-mediated responses. NMDAR-mediated responses were nest-recorded at Vh=+40 mV in the presence of the selective AMPAR antagonist NBQX (10 mM, Tocris). For I-V curves, spermine (100 mM, Sigma) was added to the pipette solution to block GluR2-lacking AMPARs at positive potentials. Evoked AMPAR-mediated responses were recorded from different membrane potentials ranging from 80 to +40 mVin 20-mVsteps. Amplitudes of currents were normalized to the value measured at −40 mV. Whole-cell voltage-clamp recordings of hippocampal CA1 pyramidal neurons were performed in the presence of GABAA receptor antagonist (10 mM Gabazine, Sigma) and NMDAR antagonist (50 mM D-APS, Sigma). The pipette solution contained 90 mM Cs-methane sulfonate, 48.5 mM CsCl, 5 mM EGTA, 2 mM MgCl2, 2 mMNa-ATP, 0.4 mMNa-GTP, 1 mM QX 314 bromide, and 5 mM Hepes (pH 7.2, 290±5 mmol/kg). Statistical comparison was performed by the independent t test and ANOVA for multiple comparisons ([FIGS.] [33] and [34]).

Statistical Analysis

All statistical tests were conducted in SPSS or SigmaStat. All analyses presented here were performed specifically to compare TsVeh, TsSAG, and EuVeh. In all instances where EuSAG data are reported, they are always compared to the EuVeh group in a pairwise analysis with Fisher's LSD. All behavioral tests were performed while the experimenter was blinded to genotype and treatment. Statistical analyses were similarly conducted blinded to genotype and treatment.

All morphological data (cell number, density, and areas) were tested for normality by quantile-quantile plots or Kolmogorov-Smirnov test. MANOVA (Wilk's 1) was carried out for cerebellar GC density and normalized area, followed by pairwise comparisons with Fisher's LSD. For GC number in the P6 DG, pairwise comparisons between the three groups were carried out with Fisher's LSD. Normalized numbers of BrdU-labeled cells in the GC were analyzed with one-way ANOVA followed by Fisher's LSD. Open-field data were tested with MANOVA for normalized number of fine motor and ambulatory movements at the center versus the periphery and for the number of rearings and for spontaneous alternation and number of arm entries in the Y maze. The value of Wilk's 1 was determined, followed by correction for multiple pairwise comparisons with the Bonferroni method. All MWM data were tested for normal distribution with Kolmogorov-Smirnov test or quantile-quantile plots. The hidden platform data were transformed before being tested in parametric tests. The probe test data and strategy scores were analyzed with nonparametric tests. Latency to platform in the MWM was analyzed with two-way repeated-measures ANOVA, with the trials in visible or hidden platform included as the repeated measurement, followed by multiple pairwise comparisons between the three groups. P values were corrected by the Bonferroni method to maintain the family-wise a value at 0.05.

Probe test results in MWM were analyzed with the nonparametric version of one-way ANOVA Kruskal-Wallis rank test, followed by the Mann-Whitney test for pairwise comparisons. Correlation between scores on trajectory and latency was determined with the nonparametric Spearman's rho. The frequency of different scores was compared with the c2 test and Fisher's exact P value.” See Ishita Das et al., “Hedgehog Agonist Therapy Corrects Structural and Cognitive Deficits in a Down Syndrome Mouse Model,” Science Translational Medicine, Vol. 5, Issue 201; p. 201ra120 (2013) (Internal Citations removed).

“Supplementary Notes Regarding Behavior Studies, FIG. [FIG. 15], [FIG. 17] and [FIG. 19]: Granule Cell Precursor Proliferation Assay

The activity of the single batch of SAG used in our experiments was compared to the concentration that caused similar GCP proliferation as 5 nM of dually lipidated Shh (Shh-Np) in an in vitro assay ([FIG. 15]) with isolated GCPs from P6-P7 cerebella. We note that absolute values of GCP responsiveness are influenced by differences in genetic background, age and maturity of the mice and do not necessarily provide a reproducible metric for comparison across laboratories. However, all four previous reports of in vivo SAG response used the same final dose of 20 μg/g or a similar dose to the one administered to the mice used in our experiments.

Morris Water Maze Strategy Choice

As demonstrated here, a single treatment with SAG at P0 normalized performance of adult Ts65Dn mice in the MWM (FIG. [11]). Recent studies have highlighted a role for the cerebellum in spatial learning, proposing that it is necessary in the acquisition of optimal strategies for spatial learning in tasks such as the MWM hidden platform. Accordingly, we analyzed search strategies used by control and SAG treated trisomic mice based on the path to the platform ([FIG. 19]). Scores correlated significantly with the latency for all groups (Spearman's rho>0.80), indicating that reduced latencies for both TsSAG and EuVeh compared to TsVeh were due to the use of improved strategies for platform finding.

TsVeh mice frequently showed a behavior in which they continued to swim against the wall in a single quadrant and did not seem to be making an attempt to look for the platform at all. This behavior is a form of thigmotaxis which has been noted previously in Ts65Dn mice. This behavior was added to the 10 point scale of Petrosini et al. and given a score of 11. Search pattern 11 was seen predominantly in TsVeh animals ([FIG. 19]b). All the scores were based on spatial strategy, except score 11 which could be a mixture of behavior and spatial learning. All trajectories were scored by investigators blinded to genotype and treatment, as well as to trial number (attempt number within a trial was differentiated using color coding) to remove any bias.” See Ishita Das et al., “Hedgehog Agonist Therapy Corrects Structural and Cognitive Deficits in a Down Syndrome Mouse Model,” Science Translational Medicine, Vol. 5, Issue 201; p. 201ra120; Supplemental Materials (2013) (Internal Citations removed).

### EXAMPLES

### Example 1. SHH in Patients with Hyposmia

Objective:

To determine the presence of SHH in human nasal mucus in normal subjects and in patients with smell loss (hyposmia).

Methods:

SHH was measured in 14 normal subjects and in 44 untreated patients with smell loss (hyposmia) of several causes and in 30 of these patients after treatment with oral theophylline using sensitive spectrophotometric ELISA assay.

Results:

SHH was present in nasal mucus in both normal subjects and in patients with hyposmia. However, SHH levels in hyposmic patients were significantly lower than in normal subjects. After treatment with oral theophylline, SHH levels in nasal mucus increased significantly to over 300 times higher than in the untreated state associated. 60% of patients exhibited improved smell function.

Conclusion:

SHH may act as a cell signaling moiety to stimulate stem cells in olfactory epithelium; its diminution in hyposmic patients compared to normals suggests that SHH serves as a biochemical marker for smell loss and acts as a growth factor to maintain normal olfactory function.

Introduction

Members of the hedgehog signaling pathway belong to a family of extracellular signaling molecules involved in the regulation of multiple physiological processes including invertebrate and vertebrate embryo development. Vertebrate organisms express multiple forms of hedgehog; there are three known hedgehogs in mammals—Sonic hedgehog (SHH), Indian hedgehog (IHH) and Desert hedgehog (DHH). SHH plays an important role in several developmental processes involving induction of dopaminergic neurons and cholinergic neurons.

SHH can be synthesized as a 45-kD precursor protein that can be cleaved autocatalytically to yield a 20-kD N-terminal fragment with a cholesterol molecule covalently attached to the C-terminal glycine and a 25-kD C-terminal fragment. Its crystal structure has been determined and it can be structurally homologous to several zinc-dependent hydrolases. The crystal structure of SHH reveals one zinc atom coordinated by two histidines and a glutamate residue. Removal of zinc from SHH inhibits its activity. Increase in activity of cAMP-dependent protein kinase A antagonizes SHH signaling.

Methods

Subjects:

Forty-four patients, aged 10-88 y, 56±3 y (Mean±SEM) took part in this study. Patients were 24 men, aged 12-88 y, 54±4 y, and 20 women, aged 10-84 y, 51±5 y. All patients exhibited smell loss as measured by subjective statement and olfactometry, as previously described. Olfactometry can include determination of detection (DT) and recognition (RT) thresholds and magnitude estimation (ME) for four odors (pyridine, nitrobenzene, thiophene and amyl acetate). Abnormalities of smell function consisted of increased DT or RT above normal (decreased sensitivity) and/or decreased ME (decreased sensitivity) for one or more of the odors presented. Patients exhibited six etiologies related to their smell loss: post-influenza-like hyposmia [(PIHH) 10 patients], allergic rhinitis [15 patients], congenital loss of smell [nine patients], head injury [eight patients], post general anesthesia [one patient] and dysgeusia and oropyrosis [one patient].

Thirty of the hyposmic patients were treated with oral theophylline with a dose range of 200-800 mg taken over a period of 2-10 months. These patients were 17 men, aged 12-78 y, 62±5 y, six with PIHH, nine with allergic rhinitis, one with congenital smell loss and one post anesthesia, and 13 women, aged 12-67 y, 42±6 y, with four with PIHH, one with allergic rhinitis and eight with congenital smell loss. Improvement in smell function consisted of decreased DT or RT (increased sensitivity) and/or increased ME (increased sensitivity) for one or more of the presumed odors.

Normal Subjects:

Fourteen subjects who presented to The Taste or smell Clinic in Washington, D.C. for evaluation of symptoms unrelated to smell loss and other volunteers were a part of this study. Normal subjects were selected in a consecutive manner and included all subjects who exhibited no sensory abnormalities.

Study protocols were previously approved by the Georgetown University Medical Center Institutional Review Board and Chesapeake IRB of Columbia Md. Each participants of the study voluntarily agreed and signed an informal consent participation form.

Procedures:

Patients and subjects were instructed to deposit all the nasal mucus they produced spontaneously over a period of 1-4 days into a 50 ml plastic tube. All samples were refrigerated overnight and collection was longer than 24 hrs.

Each sample was transferred to a 12 ml plastic tube and centrifuged in a refrigerated RC2B Spinco centrifuge at 18,400 rpm for 45-55 min. The supernatant was transferred to PCR tubes and stored at −20° C. until analyzed.

Each sample was analyzed by using a specific spectrophotometric ELISA technique obtained from Abcam Inc. (Cambridge, Mass.). Analysis of duplicate samples agreed within 5%. All analyses were made independent of the knowledge of the status of any subject. Only after all samples were analyzed and results tabulated were samples codified in relationship to clinical diagnosis. Results were analyzed such that Mean±SEM levels in each category were obtained and results compared using Student t tests with p<0.05 considered significant.

Results

SHH was measured in the nasal mucus of all participants (Table 1). Levels of SHH in patients were less than 2% of the levels of SHH found in normal subjects (Table 1).

Mean SHH levels in women were 1.5 times higher than in men (Table 2).

Mean SHH levels in patients having a wide range of etiologies for the cause of their smell loss, varied widely (Table 3). Patients with general anesthesia exhibited the lowest levels of any patient group followed in rank order by patients with allergic rhinitis, congenital smell loss, the patient with dysgeusia and oropyrosis, head injury and PIHH. Mean SHH levels of each patient group were significantly lower than in normal subjects.

Treatment with oral theophylline significantly increased SHH levels by over 330 times among patients (Table 3). Theophylline levels increased significantly above normal levels in both men and women with an increase of 320 times in men but only 17 times in women (Table 3). However, prior to theophylline treatment SHH levels in women were significantly higher than in men (p<0.001).

Categorized by etiology each patient group studied exhibited a significant increase in nasal mucus SHH (Table 4). Patients with allergic rhinitis increased the greatest amount (by over 719 times the untreated state), next the patient post anesthesia (by 48 times), by patients with PIHH (by 46 times) and least by patients with congenital smell loss (by over 21 times).

Oral theophylline treatment in PIHH patients increased SHH to levels significantly above levels in all patients before theophylline treatment (Table 4). However, levels in treated patients with allergic rhinitis and congenital smell loss and following general anesthesia did not exhibit as much change in SHH levels and were below the mean of all treated patients.

Improvement in smell function after oral theophylline treatment occurred in 19 of 31 patients or an overall improvement in 61%.

TABLE 1
SONIC HEDGEHOG IN NASAL MUCUS IN NORMAL SUBJECTS
AND IN PATIENTS WITH HYPOSMIA

| SUBJECTS | SONIC HEDGEHOG* |
|---|---|
| PATIENTS (44) | 149 ± 2^{+,a} |
| NORMALS (14) | 7538 ± 1105 |

( ) Subject number
*in pg/ml
⁺Mean ± SEM
With respect to normals
^{a}p < 0.001

TABLE 2
SONIC HEDGEHOG IN NASAL MUCUS IN PATIENTS WITH
HYPOSMIA CLASSIFIED BY ETIOLOGY OF SMELL LOSS

| SUBJECTS | SONIC HEDGEHOG* |
|---|---|
| ALL PATIENTS (44) | 149 ± 2^{+,a} |
| PIHH (10) | 1527 ± 159^{a} |
| ALLERGIC RHINITIS (15) | 34 ± 2^{a} |
| CONGENITAL (9) | 180 ± 12^{a} |
| HEAD INJURY (8) | 1396 ± 252^{a} |
| DYSGEUSIA WITH OROPYROSIS (1) | 226 |
| POST GENERAL ANESTHESIA (1) | 1.3 |
| NORMALS (14) | 7538 ± 1105 |

( ) Subject number
*in pg/ml
⁺Mean ± SEM
With respect to normals
^{a}p < 0.001

TABLE 3
SONIC HEDGEHOG IN PATIENTS WITH HYPOSMIA UNTREATED
AND AFTER TREATMENT WITH ORAL THEOPHYLLINE

CONDITION

| | | Treatment With |
|---|---|---|
| SUBJECTS | Untreated | Oral Theophylline‡ |
| NORMALS | 7538 ± 1105 (14) | |
| MEN | 150 ± 6^{a1} (24) | 47952 ± 3085^{a,a1} (17) |
| WOMEN | 229 ± 8^{a1} (20) | 3859 ± 303^{a,a1} (13) |
| ALL PATIENTS | 149 ± 2^{+,a1} (44) | 49191 ± 1710^{c,a1} (30) |

( ) Subject number
⁺Mean ± SEM of sonic hedgehog concentration (in pg/ml)
‡Oral theophylline (400-800 mg daily for 2-10 months)
With respect to untreated patients
^{a}p < 0.001
^{b}
^{c}p < 0.05
With respect to normals
^{a1}p < 0.001

TABLE 4
SONIC HEDGEHOG IN NASAL MUCUS IN PATIENTS CLASSIFIED BY ETIOLOGY
UNTREATED AND TREATED WITH ORAL THEOPHYLLINE

| | | | SMELL |
|---|---|---|---|
| | CONDITION* | | IMPROVEMENT |
| | | Treatment With | Patient Number |
| PATIENTS | Untreated | Oral Theophylline | (%) |
| ALL PATIENTS | 149 ± 2 (44) | 49191 ± 1710^{a} (31) | 19 (61) |
| PIHH | 1537 ± 159^{+,a2} (10) | 70735 ± 5751^{a,a1} (10) | 8 (80) |
| ALLERGIC RHINITIS | 34 ± 2^{a2} (15) | 24460 ± 2610^{a,a1} (11) | 5 (45) |
| CONGENITAL | 180 ± 12^{a2} (9) | 3825 ± 474^{a,a1} (9) | 5 (56) |
| HEAD INJURY | 1396 ± 252^{a2} (8) | — | |
| DYSGEUSIA WITH | 226 (1) | — | |
| OROPYROSIS | | | |
| POST GENERAL | 1.3 (1) | 57 (1) | 1 (100) |
| ANESTHESIA | | | |
| NORMALS | 7538 ± 1105 (14) | — | |

( ) Patient number
*Sonic hedgehog concentration (in pg/ml)
⁺Mean ± SEM
With respect to untreated patients
^{a}p < 0.001
With respect to treated patients
^{a1}p < 0.001
With respect to untreated patients
^{a2}p < 0.001

Discussion

This study indicates that SHH can be present in the nasal mucus in both normal subjects and in untreated patients with hyposmia. However, levels in untreated hyposmic patients were significantly lower than in normal subjects similar to results previously demonstrated for levels of nasal mucus cAMP and cGMP which were also significantly lower than in normal subjects. Treatment with oral theophylline significantly increased SHH levels in nasal mucus of patients with hyposmia over those measured in the untreated state consistent with results previously demonstrated for levels of nasal mucus cAMP and cGMP. Prior treatment among prior hyposmic patients with oral theophylline resulted in smell improvement in slightly over 50% of patients whereas in this study 60% of patients exhibited improvement in smell function. Among prior theophylline treated hyposmic patients some exhibited resistance to oral theophylline treatment, a result which also may have occurred among patients in this study.

### Example 2. Sonic Hedgehog in Human Taste Function

Purpose:

To determine the role of sonic hedgehog (Shh) in human taste function.

Background:

Shh is a 20 kD NH₂ terminal protein involved with signaling in multiple cellular systems. We hypothesized that Shh should be found in saliva. Thus, we attempted to measure Shh in saliva in both normal subjects and in patients with taste dysfunction.

Methods:

Shh was measured in parotid saliva of both normal subjects and patients with taste dysfunction of multiple etiologies by use of sensitive spectrophotometric ELISA assay. Taste dysfunction was defined clinically by both subjective inhibition of taste function (including acuity loss) and impaired gustometry.

Results:

Shh was found in parotid saliva in each normal subject. It was also found in each patient with taste dysfunction but at levels significantly lower than in normal subjects. Patients expressed subjective loss of taste function. Impaired gustometry was also measured.

Methods

Subjects

Normal Subjects.

Twenty-six volunteers, aged 22-84 y, 54±5 y (Mean±SEM) with normal taste function were studied. These volunteers were either patients who were presented to The Taste and Smell Clinic in Washington, D.C. for evaluation of symptoms unrelated to taste loss or who were employees of The Taste and Smell Clinic who volunteered for the study. Subjects were selected in a consecutive manner and included all subjects who volunteered for the study.

Patients.

Sixty-four patients, aged 10-88 y, 56±3 y who presented to The Taste and Smell Clinic in Washington, D.C. for evaluation and treatment of taste and smell loss were also studied. Patients were selected consecutively from patients evaluated at The Clinic from 2012-2013. Patients were 12 men, aged 12-88 y, 54±4 y and 14 women, aged 10-84 y, 51±5 y. Taste dysfunction was caused by seven pathological events including post-influenza-like hypogeusia [(PIHH) 17 patients], allergic rhinitis [26 patients], congenital loss of smell with associated hypogeusia [10 patients], head injury [12 patients], post general anesthesia [two patients], dysgeusia and oropyrosis [one patient] and post systemic radiation [one patient]. All patients exhibited taste dysfunction as measured by subjective statement of acuity loss and by impaired gustometry.

Subjective statements of acuity loss were quantitated by use of a scale from 0-100 with 100 reflecting total loss of taste function, 0 reflecting no loss and a number between 0-100 reflecting appropriate degree of loss. Mean±SEM of loss degree was measured among all patients and each pathology initiating taste dysfunction.

Gustometry measurements included measurements of detection (DT) and recognition (RT) thresholds and magnitude estimation (ME) for four tastants [NaCl (salt), sucrose (sweet), HCl (sour) and urea (bitter)]. Abnormalities of taste function were measured by increased DT or RT above normal (decreased sensitivity) and/or decreased ME (decreased sensitivity) for one or more of the tastants presented.

Study protocol was consistent with studies previously approved by the both Institutional Review Board of the Georgetown University Medical Center and Chesapeake IRB of Columbia Md. Each patient and subject agreed to participate in the study and signed an informed consent participation form.

Methods

Patients and volunteers collected saliva by placement of a Lashley cup over Stensen's duct of one parotid gland with saliva stimulated by lingual placement of concentrated lemon juice. Saliva was collected in plastic tubes in ice for timed periods of 8-10 min, as previously described. Flow rate was measured by mean flow over a four minute time period, as previously described. Samples were stored at −20° C. until analyzed.

Each sample was analyzed by use of a sensitive spectrophotometric ELISA technique obtained from Abcam Inc. (Cambridge, Mass.). Analysis of duplicate samples agreed within 5%. All analyses were made independent of the knowledge of the status of any subject. Only after all samples were analyzed were results tabulated and samples classified in relationship to subject status.

Results were analyzed such that mean±SEM levels in each category were obtained and results compared using Student t tests with p<0.05 considered significant.

Results

Mean Shh was present in saliva in each normal volunteer and patient studied (Table 5). Levels in patients were significantly lower than those measured in normal subjects (Table 5).

Shh in saliva did not differ in men or women patients (Table 6).

Shh in saliva demonstrated a pattern of increasing with age with the highest levels demonstrated in the oldest patients studied (Table 7).

Mean Shh levels in patients with various etiologies related to the cause of their taste dysfunction varied widely (Table 8). The lowest level was demonstrated in the patient with dysgeusia and oropyrosis, the highest levels in patients with PIHH (Table 8). While the mean level in all patients was significantly lower than in normal subjects levels in patients with head injury were significantly lower than patients with PIHH.

Subjective loss of taste acuity was present in each patient with taste dysfunction with a mean loss of 41±3%. Subjective loss of flavor perception was present in each patient with a loss of 28±3%. Impaired gustometry were demonstrated in the patients with measurements of increased DT (decreased sensitivity), increased RT (decreased sensitivity) and decreased ME (decreased sensitivity) compared to similar results in normal subjects (Table 9).

Discussion

Results of this study indicate that Shh is present in saliva in both normal subjects and in patients with taste dysfunction. Its presence in human saliva is herein reported for the first time.

As patients aged there was an increase in saliva Shh with the highest levels demonstrated in the oldest patients.

Shh levels in untreated patients with taste dysfunction were significantly lower than in normal subjects similar to results previously demonstrated for levels of saliva cAMP and which have previously been demonstrated to be significantly lower than in normal subjects.

Salivary Shh levels were lower than normal in patients in all diagnostic categories studied. This result suggests that lower than normal levels of salivary Shh may serve as a general diagnostic value for taste dysfunction in patients with these symptoms.

TABLE 5
SONIC HEDGEHOG IN SALIVA IN NORMAL SUBJECTS AND
IN PATIENTS WITH TASTE DYSFUNCTION

| SUBJECTS | SONIC HEDGEHOG* |
|---|---|
| NORMALS (26) | 215 ± 7⁺ |
| PATIENTS (64) | 63 ± 6^{a} |

( ) Subject number
*in pmol/ml
⁺Mean ± SEM
With respect to normals
^{a}p < 0.001

TABLE 6
SONIC HEDGEHOG IN SALIVA IN PATIENTS WITH TASTE
DYSFUNCTION CHARACTERIZED BY GENDER

| PATIENTS | AGE (y) | SONIC HEDGEHOG* |
|---|---|---|
| MEN (37) | 56 ± 3 | 61 ± 7⁺ |
| WOMEN (30) | 55 ± 4 | 62 ± 10 |

( ) Subject number
*in pmol/ml
⁺Mean ± SEM

TABLE 7
SONIC HEDGEHOG IN SALIVA IN PATIENTS WITH TASTE
DYSFUNCTION CHARACTERIZED BY AGE

| PATIENTS | | SONIC HEDGEHOG* |
|---|---|---|
| <30 | (11) | 66 ± 8⁺ |
| 31-40 | (4) | 80 ± 24 |
| 41-50 | (7) | 63 ± 14 |
| 51-60 | (9) | 62 ± 10 |
| 61-70 | (16) | 90 ± 13 |
| 71-80 | (14) | 96 ± 13 |
| >81 | (6) | 104 ± 35 |

( ) Subject number
*in pmol/ml
⁺Mean ± SEM

TABLE 8
SONIC HEDGEHOG IN SALIVA OF PATIENTS WITH
TASTE DYSFUNCTION

| | SONIC |
|---|---|
| CONDITION | HEDGEHOG* |
| PIHH (17) | 104 ± 16⁺ |
| ALLERGIC RHINITIS (26) | 76 ± 6 |
| CONGENITAL (10) | 70 ± 11 |
| HEAD INJURY (12) | 54 ± 11 |
| DYSGEUSIA WITH OROPYROSIS (1) | 29 |
| POST GENERAL ANESTHESIA (2) | 77 |
| POST RADIATION (1) | 115 |

( ) Subject number
*in pmol/ml
⁺Mean ± SEM
With respect to PIHH
b p < 0.005

TABLE 9
TASTE FUNCTION IN PATIENTS COMPARED TO NORMAL SUBJECTS

| | NaCl | | | SUCROSE | | | HCl | | | UREA | | |
|---|---|---|---|---|---|---|---|---|---|---|---|---|
| | DT | RT | ME | DT | RT | ME | DT | RT | ME | DT | RT | ME |
| PATIENTS | 3.9 ± 0.3^{+,a} | 4.8 ± 0.6^{d} | 52 ± 7 | 3.8 ± 0.3^{a} | 3.9 ± 0.2^{a} | 44 ± 6^{b} | 3.8 ± 0.3 | 4.9 ± 0.7 | 49 ± 7^{d} | 4.3 ± 0.5^{e} | 5.3 ± 0.7^{d} | 44 ± 7^{c} |
| (18) | | | | | | | | | | | | |
| NORMALS | 2.3 ± 0.1 | 3.1 ± 0.2 | 68 ± 4 | 2.5 ± 0.1 | 3.2 ± 0.1 | 69 ± 4 | 3.1 ± 0.2 | 3.5 ± 0.1 | 68 ± 4 | 3.2 ± 0.1 | 3.4 ± 0.1 | 68 ± 4 |
| (55) | | | | | | | | | | | | |

RT, Recognition Threshold
DT, Detection Threshold
ME, Magnitude Estimation
( ) Subject number
⁺Mean ± SEM
With respect to normals
^{a}p < 0.001
^{b}p < 0.005
^{c}p < 0.01
^{d}p < 0.02
^{e}p < 0.05

Additional subjects were examined and the data reanalyzed in view of new data.

Methods:

Shh was measured in parotid saliva of both normal subjects and in patients with taste dysfunction of multiple etiologies by use of a sensitive spectrophotometric ELISA assay. Taste dysfunction was defined clinically by both subjective changes of taste acuity and flavor perception and by impaired gustometry. Patients were treated with oral theophylline 200-800 mg daily for 2-10 months with saliva Shh and taste function measured at intervals of 2-8 months.

Results:

Shh was found in parotid saliva in both normal subjects and in patients with taste dysfunction but levels were significantly lower in patients than in normal subjects. Both subjective loss of taste acuity and flavor perception and impaired gustometry was measured in each patient. Theophylline treatment increased saliva Shh and improved both subjective taste function and gustometry.

Conclusions:

This is the first demonstration of Shh in saliva. Decreased saliva Shh secretion can be considered a marker for taste dysfunction in patients with multiple etiologies. Theophylline acts to increase Shh in saliva and thereby improve human taste dysfunction as its increase in nasal mucus improved human smell dysfunction.

Methods

Subjects

Normal Subjects.

Twenty-six volunteers, aged 22-84 y, 54±5 y (Mean±SEM) with normal taste function were studied. These volunteers were either patients who presented to The Taste and Smell Clinic in Washington, D.C. for evaluation of symptoms unrelated to taste loss or who were employees of The Taste and Smell Clinic who volunteered for the study. Subjects were selected in a consecutive manner and included all subjects who volunteered for the study.

Patients.

Eighty-one patients, aged 10-88 y, 56±3 y who presented to The Taste and Smell Clinic in Washington, D.C. for evaluation and treatment of taste and smell loss were also studied. Patients were selected consecutively from patients evaluated at The Clinic from 2012-2013. Patients were 58 men, aged 12-88 y, 54±4 y and 56 women, aged 10-84 y, 51±5 y. Taste dysfunction was caused by seven pathological events including post-influenza-like hypogeusia [(PIHH) 20 patients], allergic rhinitis [31 patients], congenital loss of smell with associated hypogeusia [9 patients], head injury [14 patients], post general anesthesia [three patients], dysgeusia with oropyrosis [one patient] and post systemic radiation [one patient]. All patients exhibited taste dysfunction as measured by subjective statement of taste acuity loss and loss of flavor perception and by impaired gustometry.

Subjective statements of taste acuity loss and loss of flavor perception were quantitated by use of a scale from 0-100 with 100 reflecting total loss of taste acuity or flavor perception, 0 reflecting no loss and a number between 0-100 reflecting appropriate degree of loss. Some of these patients also exhibited taste distortions but these results were not the subject matter for this study and are not included in this study. Mean±SEM of loss degree was measured among all patients and each pathology initiating taste dysfunction.

Gustometry measurements included measurements of detection (DT) and recognition (RT) thresholds and magnitude estimation (ME) for four tastants [NaCl (salt), sucrose (sweet), HCl (sour) and urea (bitter)]. Abnormalities of taste function were measured by increased DT or RT above normal (decreased sensitivity) and/or decreased ME (decreased sensitivity) for one or more of the tastants presented.

Treatment with oral theophylline was administered to 79 of these patients, aged 12-86 y, 41 men and 38 women at doses of 200-1000 mg for periods of 2-10 months. Saliva Shh and measurements of taste function by use of subjective responses of acuity and flavor perception and in olfactometry was measured at intervals of 2-6 months in these patients.

Study protocol was consistent with studies previously approved by the Institutional Review Board of the Georgetown University Medical Center. Each patient and subject agreed to participate in the study and signed an informed consent participation form. All subjects under age 18 y entered into the study after a parent gave informed consent.

Methods

Parotid saliva was collected in patients and normal volunteers by placement of a Lashley cup over Stensen's duct of one parotid gland with saliva stimulated by lingual, timed placement of concentrated lemon juice. Saliva was collected in plastic tubes in ice for timed periods of 8-10 min, as previously described. Flow rate was measured by mean flow over a four minute time period, as previously described. Samples were stored at −20° C. until analyzed.

Each sample was analyzed by use of a sensitive spectrophotometric ELISA technique obtained from Abcam Inc. (Cambridge, Mass.). Analysis of duplicate samples agreed within 5%. All analyses were made independent of the knowledge of the status of any subject. Only after all samples were analyzed were results tabulated and samples classified in relationship to subject status.

Results were analyzed such that mean±SEM levels in each category were obtained and results compared using Student t tests with p<0.05 considered significant.

Results

Shh was present in parotid saliva in each normal volunteer and in each untreated patient with hypogeusia (Table 10). Levels in patients were significantly lower than those measured in normal subjects (Table 10).

Shh in saliva did not differ in untreated men or women patients (Table 11).

Shh in saliva demonstrated a varying pattern with age (Table 12).

Mean Shh levels in patients with various etiologies related to the cause of their taste dysfunction varied widely (Table 13). The lowest level was present in one patient with dysgeusia (distorted taste sensation) and oropyrosis, the highest levels in patients post anesthesia (Table 13). The mean level in each patient category was significantly lower than the mean level in normal subjects.

Subjective loss of taste acuity and flavor perception was present in each patient before treatment with oral theophylline. Impaired gustometry were demonstrated in patients with measurements of increased DT (decreased sensitivity), increased RT (decreased sensitivity) and decreased ME (decreased sensitivity) compared to similar results in normal subjects (Table 14).

After treatment with oral theophylline Shh increased in parotid saliva to levels above those in normal subjects or in untreated patients (Table 15). There was improvement in both subjective taste acuity and flavor perception in about 60% of patients (Table 16). Degree of return of acute acuity and flavor perception was greater in women than in men. Improvement in olfactometry also occurred (data not shown).

Discussion

Results of this study indicate that Shh is present in saliva in both normal subjects and in patients with taste dysfunction. Its presence in human saliva is herein reported for the first time.

Salivary Shh levels were lower than normal in patients in all diagnostic categories studied. This result suggests that lower than normal levels of salivary Shh may serve as a general diagnostic marker for taste dysfunction in patients with these symptoms.

TABLE 10
SONIC HEDGEHOG IN PAROTID SALIVA IN NORMAL SUBJECTS
AND IN PATIENTS WITH TASTE DYSFUNCTION

| SUBJECTS | SONIC HEDGEHOG* |
|---|---|
| NORMALS (26) | 184 ± 12⁺ |
| PATIENTS (81) | 64 ± 6^{a} |

( ) Subject number
* in pmol/ml
+Mean ± SEM
With respect to normals
^{a}p < 0.001

TABLE 11
SONIC HEDGEHOG IN PAROTID SALIVA IN NORMAL SUBJECTS
AND IN UNTREATED PATIENTS WITH TASTE DYSFUNCTION
CHARACTERIZED BY GENDER

| | AGE (y) | SONIC HEDGEHOG* |
|---|---|---|
| NORMAL | | |
| SUBJECTS | | |
| MEN (10) | 70 ± 6 | 186 ± 16⁺ |
| WOMEN (17) | 61 ± 4 | 180 ± 12 |
| PATIENTS | | |
| MEN (41) | 56 ± 3 | 76 ± 7 |
| WOMEN (40) | 55 ± 4 | 66 ± 6 |

( ) Subject number
*in pmol/ml
⁺Mean ± SEM

TABLE 12
SONIC HEDGEHOG IN SALIVA IN PATIENTS WITH TASTE
DYSFUNCTION CHARACTERIZED BY AGE

| PATIENTS‡ | | SONIC HEDGEHOG* |
|---|---|---|
| <30 | (11) | 62 ± 9⁺ |
| 31-40 | (5) | 76 ± 19 |
| 41-50 | (5) | 56 ± 12 |
| 51-60 | (11) | 57 ± 9 |
| 61-70 | (22) | 76 ± 8 |
| 71-80 | (15) | 93 ± 13 |
| >81 | (7) | 62 ± 24 |

‡Age (in years)
( ) Patient number
*in pmol/ml
⁺Mean ± SEM

TABLE 13
SONIC HEDGEHOG IN SALIVA OF UNTREATED PATIENTS WITH
TASTE DYSFUNCTION

| | SONIC |
|---|---|
| CONDITION | HEDGEHOG* |
| PIHH (27) | ⁷58 ± 10^{+a} |
| ALLERGIC RHINITIS (26) | 70 ± 6^{a} |
| CONGENITAL (10) | 76 ± 11^{a} |
| HEAD INJURY (14) | 49 ± 10^{a} |
| DYSGEUSIA WITH OROPYROSIS (1) | 29 |
| POST GENERAL ANESTHESIA (2) | 82 |
| POST RADIATION (1) | 145 |

( ) Patient number
*in pmol/ml
⁺Mean ± SEM
With respect to normals
^{a}p < 0.001

TABLE 14
TASTE FUNCTION IN UNTREATED HYPOGEUSIC PATIENTS COMPARED TO NORMAL SUBJECTS
BY USE OF GUSTOMETRY

| | NaCl | | | SUCROSE | | | HCl | | | UREA | | |
|---|---|---|---|---|---|---|---|---|---|---|---|---|
| | DT | RT | ME | DT | RT | ME | DT | RT | ME | DT | RT | ME |
| PATIENTS | 3.9 ± 0.3^{+,a} | 4.8 ± 0.6^{d} | 52 ± 7 | 3.8 ± 0.3^{a} | 3.9 ± 0.2^{a} | 44 ± 6^{b} | 3.8 ± 0.3 | 4.9 ± 0.7 | 49 ± 7^{d} | 4.3 ± 0.5^{e} | 5.3 ± 0.7^{d} | 44 ± 7^{c} |
| (64) | | | | | | | | | | | | |
| NORMALS | 2.3 ± 0.1 | 3.1 ± 0.2 | 68 ± 4 | 2.5 ± 0.1 | 3.2 ± 0.1 | 69 ± 4 | 3.1 ± 0.2 | 3.5 ± 0.1 | 68 ± 4 | 3.2 ± 0.1 | 3.4 ± 0.1 | 68 ± 4 |
| (26) | | | | | | | | | | | | |

RT, Recognition Threshold
DT, Detection Threshold
ME, Magnitude Estimation
( ) Subject number
⁺Mean ± SEM
With respect to normals
^{a}p < 0.001
^{b}p < 0.005
^{c}p < 0.01
^{d}p < 0.02
^{e}p < 0.05

TABLE 15
SALIVA SONIC HEDGEHOG IN HYPOGEUSIC PATIENTS BEFORE
AND AFTER TREATMENT WITH ORAL THEOPHYLLINE

| | SALIVA |
|---|---|
| PATIENTS | SONIC HEDGEHOG* |
| BEFORE TREATMENT (66) | 71 ± 4^{+a} |
| AFTER TREATMENT (66) | 199 ± 20 |
| MEN (61) | 180 ± 15 |
| WOMEN (58) | 213 ± 33 |

( ) Patient number
*in pmol/ml
⁺Mean ± SEM
With respect to pretreatment
^{a}p < 0.001

TABLE 16
CHANGES IN TASTE FUNCTION IN HYPOGEUSIC PATIENTS
AFTER TREATMENT WITH ORAL THEOPHYLLINE

| | PATIENT | IMPROVEMENT | DEGREE OF |
|---|---|---|---|
| PATIENTS | NUMBER | NUMBER | IMPROVEMENT (%) |
| TASTE ACUITY | 79 | 45 (63) | 28 ± 4⁺ |
| MEN | 38 | 21 (55) | 20 ± 5 |
| WOMEN | 41 | 24 (59) | 35 ± 6 |
| FLAVOR | 79 | 45 (63) | 24 ± 4 |
| PERCEPTION | | | |
| MEN | 38 | 22 (58) | 18 ± 4 |
| WOMEN | 41 | 23 (54) | 36 ± 6 |

( ) Percent improved
⁺Mean ± SEM

### Example 3. Sonic Hedgehog in Patients with Taste Dysfunction: Before and after Treatment with Oral Theophylline

Methods:

After treatment with oral theophylline, Shh was measured in parotid saliva of patients with taste dysfunction of multiple etiologies by use of sensitive spectrophotometric ELISA assay. Taste dysfunction was defined clinically by impaired gustometry.

Results:

Shh was found in parotid saliva in each subject, but was significantly lower in patients with taste dysfunction. Tables 5, 10, and 17. Patients treated with oral theophylline improved subjectively in taste function.

Methods

Subjects

Normal Subjects.

Forty-three patients were treated with oral theophylline at doses of 200-800 mg for periods of 2-10 months, at which time their smell function was evaluated by olfactometry and by SHH measurements in nasal mucus by use of a sensitive spectrophotometric ELISA assay.

These volunteers were either patients who were presented to The Taste and Smell Clinic in Washington, D.C. for evaluation of symptoms unrelated to taste loss or who were employees of The Taste and Smell Clinic who volunteered for the study. Subjects were selected in a consecutive manner and included all subjects who volunteered for the study.

Patients.

Forty-three patients were presented to The Taste and Smell Clinic in Washington, D.C. for evaluation and treatment of taste and smell loss. Taste dysfunction was caused by seven pathological events including post-influenza-like hypogeusia [(PIHH) 13 patients], allergic rhinitis [15 patients], congenital loss of smell with associated hypogeusia [8 patients], head injury [4 patients], post general anesthesia [two patients], and post systemic radiation [one patient]. All patients exhibited a decreased Shh level.

Gustometry measurements included measurements of detection (DT) and recognition (RT) thresholds and magnitude estimation (ME) for four tastants [NaCl (salt), sucrose (sweet), HCl (sour) and urea (bitter)]. Abnormalities of taste function were measured by increased DT or RT above normal (decreased sensitivity) and/or decreased ME (decreased sensitivity) for one or more of the tastants presented.

Study protocol was consistent with studies previously approved by the Institutional Review Board of the Georgetown University Medical Center. Each patient and subject agreed to participate in the study and signed an informed consent participation form.

Methods

Patients and volunteers collected saliva by placement of a Lashley cup over Stensen's duct of one parotid gland with saliva stimulated by lingual placement of concentrated lemon juice. Saliva was collected in plastic tubes in ice for timed periods of 8-10 min, as previously described. Flow rate was measured by mean flow over a four minute time period, as previously described. Samples were stored at −20° C. until analyzed.

Each sample was analyzed by use of a sensitive spectrophotometric ELISA technique obtained from Abcam Inc. (Cambridge, Mass.). Analysis of duplicate samples agreed within 5%. All analyses were made independent of the knowledge of the status of any subject. Only after all samples were analyzed were results tabulated and samples classified in relationship to subject status.

Results were analyzed such that mean±SEM levels in each category were obtained and results compared using Student t tests with p<0.05 considered significant.

Results

Shh was present in saliva in each patient studied however various etiologies related to the cause of their taste dysfunction varied widely (Table 17). Levels in patients were significantly lower than those measured in normal subjects (Tables 5 and 10).

The lowest level was demonstrated in the patient with congenital, the highest levels in patients with Anesthesia Induced (Table 17).

Impaired gustometry were demonstrated in the patients with measurements of increased DT (decreased sensitivity), increased RT (decreased sensitivity) and decreased ME (decreased sensitivity) compared to similar results in normal subjects (Tables 5, 10, and 18). Surprisingly, treatment with theophylline increased levels of Shh by almost 2 fold while improving gustometric functions (Table 18).

### Discussion

Results of this study indicate that Shh is increased in saliva in patients with taste dysfunction as a result of theophylline treatment. Theophylline also improved gustometric functions of treated patients.

TABLE 17
SALIVA SONIC HEDGEHOG IN PATIENTS
AFTER ORAL THEOPHYLLINE

| | SALIVA |
|---|---|
| | SONIC HEDGEHOG |
| CONDITION | (pmol/ml) |
| ANESTHESIA INDUCED (2) | 151 |
| ALLERGIC RHINITIS (26) | 212 ± 14⁺ |
| CONGENITAL (10) | 132 ± 23 |
| HEAD INJURY (14) | 115 ± 15 |
| PIHH (27) | 210 ± 12 |
| POST RADIATION (1) | 145 |

( ) Patient number
⁺Mean ± SEM

TABLE 18
TASTE FUNCTION AND SONIC HEDGEHOG IN SALIVA IN PATIENTS WITH TASTE
DYSFUNCTION BEFORE AND AFTER TREATMENT WITH THEOPHYLLINE

| | NaCl | | | SUCROSE | | | HCl |
|---|---|---|---|---|---|---|---|
| CONDITION | DT | RT | ME | DT | RT | ME | DT |
| UNTREATED | 3.9 ± 0.3⁺ | 4.8 ± 0.6 | 52 ± 7 | 3.8 ± 0.3 | 3.9 ± 0.2 | 44 ± 6 | 3.8 ± 0.3 |
| (81) | | | | | | | |
| THEOPHYLLINE | 3.4 ± 0.2 | 3.6 ± 0.2 | 58 ± 5 | 3.3 ± 0.2 | 3.6 ± 0.2 | 49 ± 5 | 3.6 ± 0.2 |
| TREATED (81) | | | | | | | |

| | | | | | | SALIVA |
|---|---|---|---|---|---|---|
| | | | | | | SONIC |
| | HCl | | UREA | | | HEDGEHOG |
| CONDITION | RT | ME | DT | RT | ME | (pmol/ml) |
| UNTREATED | 4.9 ± 0.7 | 49 ± 7 | 4.3 ± 0.5 | 5.3 ± 0.7 | 44 ± 7 | 63 ± 6 |
| (81) | | | | | | |
| THEOPHYLLINE | 3.7 ± 0.3 | 54 ± 5 | 3.9 ± 0.3 | 3.9 ± 0.3 | 54 ± 5 | 102 ± 10 |
| TREATED (81) | | | | | | |

RT, Recognition Threshold
DT, Detection Threshold
ME, Magnitude Estimation
( ) Patient number
⁺Mean ± SEM

### Example 4. Diagnosing Patients with Chromosomal Abnormalities and/or One or More Symptoms Associated with Chromosomal Abnormalities

In this example, bodily fluids can be used to measure the levels of one or more members of the hedgehog signaling pathway. For example, a whole blood sample, a serum sample, a plasma sample, a urine sample, a saliva sample, a mucus sample, a perspiration sample, or a combination thereof; can be extracted from patients and subjects. Known methods can be used to prepare the sample for diagnosis. Levels of SHH, DHH, and IHH, can be subsequently measured by, for example, antibody-based methods, including but not limited to, an immunostain, an immunoprecipitation, an immunoelectrophoresis, an immunoblot, and a western blot. Other methods can be used as well, including but not limited to, a spectrophotometry assay.

Typically, the levels of members of the hedgehog signaling pathway in patients exhibiting chromosomal abnormalities and/or one or more symptoms associated with chromosomal abnormalities, can be lower than normal controls. For example, in patients with chromosomal abnormalities and/or one or more symptoms associated with chromosomal abnormalities, (a) the level of SHH can be or about: 0 pg/mL, greater than 0 pg/mL to less than less than 1 pg/mL, 1 pg/mL to 25 pg/mL, 15 pg/mL to 30 pg/mL, 20 pg/mL to 40 pg/mL; 35 pg/mL to 50 pg/mL; 45 pg/mL to 100 pg/mL; 75 pg/mL to 150 pg/mL, 125 pg/mL to 1000 pg/mL, 900 pg/mL to 2500 pg/mL, 2000 pg/mL to 5000 pg/mL, 4000 pg/mL to 7500 pg/mL, 6000 pg/mL to 10,000 pg/mL, greater than 0 pg/mL to less than 25 pg/mL, greater than 0 pg/mL to less than 30 pg/mL, greater than 0 pg/mL to less than 40 pg/mL, greater than 0 pg/mL to less than 50 pg/mL, greater than 0 pg/mL to less than 100 pg/mL, greater than 0 pg/mL to less than 150 pg/mL, greater than 0 pg/mL to less than 1000 pg/mL, greater than 0 pg/mL to less than 2500 pg/mL, greater than 0 pg/mL to less than 5000 pg/mL, greater than 0 pg/mL to less than 7500 pg/mL; greater than 0 pg/mL to less than 10,000 pg/mL, 1 pg/mL to 10,000 pg/mL, 15 pg/mL to 10,000 pg/mL, 20 pg/mL to 10,000 pg/mL, 20 pg/mL to 10,000 pg/mL, 35 pg/mL to 10,000 pg/mL, 45 pg/mL to 10,000 pg/mL, 75 pg/mL to 10,000 pg/mL, 125 pg/mL to 10,000 pg/mL, 900 pg/mL to 10,000 pg/mL, 2000 pg/mL to 10,000 pg/mL, 4000 pg/mL to 10,000 pg/mL, and 5000 pg/mL to 10,000 pg/mL; (b) the level of IHH can be or about: 0 pg/mL, greater than 0 pg/mL to 0.1 pg/mL, 0.05 pg/mL to 0.15 pg/mL, 0.125 pg/mL to 0.2 pg/mL, 0.15 pg/mL to 0.30 pg/mL, 0.25 pg/mL to 0.5 pg/mL, 0.4 pg/mL to 0.7 pg/mL, 0.6 pg/mL to 0.75 pg/mL, 0.725 pg/mL to 0.9 pg/mL, 0.8 pg/mL to 1.0 pg/mL, less than 1.0 pg/mL, greater than 0 pg/mL to 0.15 pg/mL, greater than 0 pg/mL to 0.2 pg/mL, greater than 0 pg/mL to 0.3 pg/mL, greater than 0 pg/mL to 0.5 pg/mL, greater than 0 pg/mL to 0.7 pg/mL, greater than 0 pg/mL to 0.75 pg/mL, greater than 0 pg/mL to 0.9 pg/mL, greater than 0 pg/mL to 1.0 pg/mL, 0.05 pg/mL to 1.0 pg/mL, 0.125 pg/mL to 1.0 pg/mL, 0.15 pg/mL to 1.0 pg/mL, 0.25 pg/mL to 1.0 pg/mL, 0.4 pg/mL to 1.0 pg/mL, 0.6 pg/mL to 1.0 pg/mL, 0.725 pg/mL to 1.0 pg/mL, 0.9 pg/mL to 1.0 pg/mL; (c) the level of DHH can be or about: 0 pg/mL, greater than 0 pg/mL to 0.1 pg/mL, 0.05 pg/mL to 0.15 pg/mL, 0.125 pg/mL to 0.2 pg/mL, 0.15 pg/mL to 0.30 pg/mL, 0.25 pg/mL to 0.5 pg/mL, 0.4 pg/mL to 0.7 pg/mL, 0.6 pg/mL to 0.75 pg/mL, 0.725 pg/mL to 0.9 pg/mL, 0.8 pg/mL to 1.0 pg/mL, 0.9 pg/mL to 1.1 pg/mL, 1.0 pg/mL to 1.3 pg/mL, 1.2 pg/mL to 1.5 pg/mL, 1.4 pg/mL to 2.0 pg/mL, 1.9 pg/mL to 2.5 pg/mL, 2.4 pg/mL to 3.0 pg/mL, 2.9 pg/mL to 3.5 pg/mL, 3.4 pg/mL to 3.8 pg/mL, 3.7 pg/mL to 3.9 pg/mL, 3.85 pg/mL to 5.0 pg/mL, less than 5.0 pg/mL, greater than 0 pg/mL to 0.15 pg/mL, greater than 0 pg/mL to 0.2 pg/mL, greater than 0 pg/mL to 0.3 pg/mL, greater than 0 pg/mL to 0.5 pg/mL, greater than 0 pg/mL to 0.7 pg/mL, greater than 0 pg/mL to 0.75 pg/mL, greater than 0 pg/mL to 0.9 pg/mL, greater than 0 pg/mL to 1.0 pg/mL, greater than 0 pg/mL to 1.1 pg/mL, greater than 0 pg/mL to 1.3 pg/mL, greater than 0 pg/mL to 1.5 pg/mL, greater than 0 pg/mL to 2.0 pg/mL, greater than 0 pg/mL to 2.5 pg/mL, greater than 0 pg/mL to 3.0 pg/mL, greater than 0 pg/mL to 3.5 pg/mL, greater than 0 pg/mL to 3.8 pg/mL, greater than 0 pg/mL to 3.9 pg/mL, greater than 0 pg/mL to 5.0 pg/mL, 0.1 pg/mL to 5.0 pg/mL, 0.05 pg/mL to 5.0 pg/mL, 0.125 pg/mL to 5.0 pg/mL, 0.2 pg/mL to 5.0 pg/mL, 0.15 pg/mL to 5.0 pg/mL, 0.25 pg/mL to 5.0 pg/mL, 0.4 pg/mL to 5.0 pg/mL, 0.6 pg/mL to 5.0 pg/mL, 0.725 pg/mL to 5.0 pg/mL, 0.8 pg/mL to 5.0 pg/mL, 0.9 pg/mL to 5.0 pg/mL, 1.0 pg/mL to 5.0 pg/mL, 1.2 pg/mL to 5.0 pg/mL, 1.4 pg/mL to 5.0 pg/mL, 1.9 pg/mL to 5.0 pg/mL, 2.4 pg/mL to 5.0 pg/mL, 2.9 pg/mL to 5.0 pg/mL, 3.4 pg/mL to 5.0 pg/mL, 3.7 pg/mL to 5.0 pg/mL, 3.85 pg/mL to 5.0 pg/mL, and 4.0 pg/mL to 5.0 pg/mL. There may be some inter-patient variability because the levels of the different members of the hedgehog signaling pathway vary based on the person. In normal controls, the levels of SHH, IHH, and DHH, can be higher, and can be significantly higher, than the levels of patients with one or more chromosomal abnormalities and/or one or more symptoms of chromosomal abnormalities. In most cases, the threshold level can be an average level for one or more members of the hedgehog signaling pathway as measured in a control population comprising subjects with no chromosomal abnormalities. In another example, the level of one or more members of the hedgehog signaling pathway can be at least one order of magnitude lower than said threshold level.

In some cases, the diagnosed result, e.g., one or more chromosomal abnormalities and/or one or more symptoms of chromosomal abnormalities, can be transferred via a communication medium. Exemplary types of communication media can include, but are not limited to written, printed, and electronic types of media.

In other cases, a computer can implement the diagnosis of one or more chromosomal abnormalities and/or one or more symptoms of chromosomal abnormalities. The computer may be a specialty computer, designed specifically for the task at hand.

### Example 5: Treating Patients with Chromosomal Abnormalities and/or One or More Symptoms Associated with Chromosomal Abnormalities

Patients diagnosed with one or more symptoms associated with chromosomal abnormalities, can be treated using a variety of methods. For example, patients can be treated with a PDE inhibitor. Some of the patients can be given a non-selective PDE inhibitor, a PDE-1 selective inhibitor, a PDE-2 selective inhibitor, a PDE-3 selective inhibitor, a PDE-4 selective inhibitor, a PDE-5 selective inhibitor, a PDE-10 selective inhibitor, or a combination thereof.

To treat one or more symptoms associated with chromosomal abnormalities, some of the patients receiving a non-selective PDE inhibitor, can be given a methylxanthine derivative, including but not limited to caffeine, theophylline, doxophylline, cipamphylline, neuphylline, pentoxiphylline, or diprophylline. Some patients receiving a PDE 1 inhibitor can be given vinpocetine. Some patients receiving a PDE 2 inhibitor can be given EHNA. Some patients receiving a PDE 3 inhibitor can be given inamrinone, anagrelide, or cilostazol. Some patients receiving a PDE 4 inhibitor can be given mesembrine, rolipram, ibudilast, piclamilast, luteolin, drotaverine, or roflumilast. Some patients received a PDE 5 inhibitor can be given sildenafil, tadalafil, vardenafil, udenafil, avanafil, or dipyridamole. Other patients receiving a PDE 10 inhibitor can be given papaverine, OMS824 (from Omeros Corporation), and/or PF-2545920 (from Pfizer).

In some cases, patients can be given forskolin to treat one or more symptoms associated with chromosomal abnormalities. In other cases, patients can be given theophylline to treat one or more symptoms associated with chromosomal abnormalities. Because different patients react differently to forskolin and/or theophylline, patients can be given an optimal amount of the respective drugs. For example, forskolin can be given, e.g., intranasally, and/or present in a positive amount selected from a group consisting of: less than 500 mg to 450 mg, 475 mg to 425 mg, 435 mg to 400 mg, 415 mg to 300 mg, 325 mg to 250 mg, 275 mg to 150 mg, 200 mg to 100 mg, 135 mg to 80 mg, 95 mg to 65 mg, 75 mg to 50 mg, 60 mg to 40 mg, 45 mg to 25 mg, 30 mg to 20 mg, 15 mg to 5 mg, 10 mg to 2.5 mg, 3.5 mg to 1 mg, 2 mg to greater than 0 mg, less than about 500 mg to about 450 mg, about 475 mg to about 425 mg, about 435 mg to about 400 mg, about 415 mg to about 300 mg, about 325 mg to about 250 mg, about 275 mg to about 150 mg, about 200 mg to about 100 mg, about 135 mg to about 80 mg, about 95 mg to about 65 mg, about 75 mg to about 50 mg, about 60 mg to about 40 mg, about 45 mg to about 25 mg, about 30 mg to about 20 mg, about 15 mg to about 5 mg, about 10 mg to about 2.5 mg, about 3.5 mg to about 1 mg, about 2 mg to greater than 0 mg, 500 mg, less than 500 mg to 450 mg, less than 500 mg to 425 mg, less than 500 mg to 400 mg, less than 500 mg to 300 mg, less than 500 mg to 250 mg, less than 500 mg to 150 mg, less than 500 mg to 100 mg, less than 500 mg to 80 mg, less than 500 mg to 65 mg, less than 500 mg to 50 mg, less than 500 mg to 40 mg, less than 500 mg to 25 mg, less than 500 mg to 20 mg, less than 500 mg to 5 mg, less than 500 mg to 2.5 mg, less than 500 mg to 1 mg, less than 500 mg to greater than 0 mg, about 500 mg, less than about 500 mg to about 450 mg, less than about 500 mg to about 425 mg, less than about 500 mg to about 400 mg, less than about 500 mg to about 300 mg, less than about 500 mg to about 250 mg, less than about 500 mg to about 150 mg, less than about 500 mg to about 100 mg, less than about 500 mg to about 80 mg, less than about 500 mg to about 65 mg, less than about 500 mg to about 50 mg, less than about 500 mg to about 40 mg, less than about 500 mg to about 25 mg, less than about 500 mg to about 20 mg, less than about 500 mg to about 5 mg, less than about 500 mg to about 2.5 mg, less than about 500 mg to about 1 mg, less than about 500 mg to greater than about 0 mg, greater than 0 mg to 450 mg, greater than 0 mg to 425 mg, greater than 0 mg to 400 mg, greater than 0 mg to 300 mg, greater than 0 mg to 250 mg, greater than 0 mg to 150 mg, greater than 0 mg to 100 mg, greater than 0 mg to 80 mg, greater than 0 mg to 65 mg, greater than 0 mg to 50 mg, greater than 0 mg to 40 mg, greater than 0 mg to 25 mg, greater than 0 mg to 20 mg, greater than 0 mg to 5 mg, greater than 0 mg to 2.5 mg, greater than 0 mg to 1 mg, greater than 0 mg to about 450 mg, greater than 0 mg to about 425 mg, greater than 0 mg to about 400 mg, greater than 0 mg to about 300 mg, greater than 0 mg to about 250 mg, greater than 0 mg to about 150 mg, greater than 0 mg to about 100 mg, greater than 0 mg to about 80 mg, greater than 0 mg to about 65 mg, greater than 0 mg to about 50 mg, greater than 0 mg to about 40 mg, greater than 0 mg to about 25 mg, greater than 0 mg to about 20 mg, greater than 0 mg to about 5 mg, greater than 0 mg to about 2.5 mg, and greater than 0 mg to about 1 mg.

In other cases, Theophylline can be given, e.g., intranasally, and/or present in a positive amount selected from a group consisting of: less than 45 mg, 30 mg, 15 mg, 10 mg, 5 mg, 1 mg, 500 μg, 250 μg, 120 μg, 80 μg, 40 μg, or 20 μg and less than about 45 mg, about 30 mg, about 15 mg, about 10 mg, about 5 mg, about 1 mg, about 500 μg, about 250 μg, about 120 μg, about 80 μg, about 40 μg, or about 20 μg, greater than 0 μg to 20 μg, 10 μg to 40 μg, 30 μg to 80 μg, 70 μg to 120 μg, 100 μg to 250 μg, 200 μg to 500 μg, 400 μg to 1 mg, 900 μg to 5 mg, 4 mg to 10 mg, 9 mg to 15 mg, 14 mg to 30 mg, 25 mg to 45 mg, greater than 0 μg to about 20 μg, about 10 μg to about 40 μg, about 30 μg to about 80 μg, about 70 μg to about 120 μg, about 100 μg to about 250 μg, about 200 μg to about 500 μg, about 400 μg to about 1 mg, about 900 μg to about 5 mg, about 4 mg to about 10 mg, about 9 mg to about 15 mg, about 14 mg to about 30 mg, about 25 mg to about 45 mg, greater than 0 μg to 40 μg, greater than 0 μg to 80 μg, greater than 0 μg to 120 μg, greater than 0 μg to 250 μg, greater than 0 μg to 500 μg, greater than 0 μg to 1 mg, greater than 0 μg to 5 mg, greater than 0 μg to 10 mg, greater than 0 μg to 15 mg, greater than 0 μg to 30 mg, greater than 0 μg to 45 mg, greater than 0 μg to about 40 μg, greater than 0 μg to about 80 μg, greater than 0 μg to about 120 μg, greater than 0 μg to about 250 μg, greater than 0 μg to about 500 μg, greater than 0 μg to about 1 mg, greater than 0 μg to about 5 mg, greater than 0 μg to about 10 mg, greater than 0 μg to about 15 mg, greater than 0 μg to about 30 mg, greater than 0 μg to about 45 mg, greater than 0 μg to 45 mg, 10 μg to 45 mg, 30 μg to 45 mg, 70 μg to 45 mg, 100 μg to 45 mg, 200 μg to 45 mg, 400 μg to 45 mg, 900 μg to 45 mg, 4 mg to 45 mg, 9 mg to 45 mg, 14 mg to 45 mg, 35 mg to 45 mg, greater than 0 μg to about 45 mg, about 10 μg to about 45 mg, about 30 μg to about 45 mg, about 70 μg to about 45 mg, about 100 μg to about 45 mg, about 200 μg to about 45 mg, about 400 μg to about 45 mg, about 900 μg to about 45 mg, about 4 mg to about 5 mg, about 9 mg to about 45 mg, about 14 mg to about 45 mg, about 35 mg to about 45 mg.

In some cases, patients can be given riociguat. In many case, low levels of riociguat can be given to patients. For example, riociguat can be given, e.g., intranasally, and/or present in a positive amount selected from a group consisting of: greater than 0.0 μg to 1 μg, 0.5 μg to 2 μg, 1.5 μg to 3.0 μg, 2.5 μg to 10 μg, 5 μg to 15 μg, 12.5 μg to 30 μg, 25 μg to 50 μg, 40 μg to 80 μg, 60 μg to 100 μg, 90 μg to 120 μg, 110 μg to 130 μg, 125 μg to 150 μg, 140 μg to 180 μg, 170 μg to 200 μg, 200 μg to 230 μg, 215 μg to 240 μg, 235 μg to less than 250 μg, less than 250 μg, greater than about 0.0 μg to about 1 μg, about 0.5 μg to about 2 μg, about 1.5 μg to about 3.0 μg, about 2.5 μg to about 10 μg, about 5 μg to about 15 μg, about 12.5 μg to about 30 μg, about 25 μg to about 50 μg, about 40 μg to about 80 μg, about 60 μg to about 100 μg, about 90 μg to about 120 μg, about 110 μg to about 130 μg, about 125 μg to about 150 μg, about 140 μg to about 180 μg, about 170 μg to about 200 μg, about 200 μg to about 230 μg, about 215 μg to about 240 μg, about 235 μg to less than 250 μg greater than 0.0 μg to 2 μg, greater than 0.0 μg to 3 μg, greater than 0.0 μg to 10 μg, greater than 0.0 μg to 15 μg, greater than 0.0 μg to 30 μg, greater than 0.0 μg to 50 μg, greater than 0.0 μg to 80 μg, greater than 0.0 μg to 100 μg, greater than 0.0 μg to 120 μg, greater than 0.0 μg to 130 μg, greater than 0.0 μg to 150 μg, greater than 0.0 μg is to 180 μg, greater than 0.0 μg to 200 μg, greater than 0.0 μg to 230 μg, greater than 0.0 μg to 240 μg, greater than 0.0 μg to 250 μg, greater than 0.0 μg to about 2 μg, greater than 0.0 μg to about 3 μg, greater than 0.0 μg to about 10 μg, greater than 0.0 μg to about 15 μg, greater than 0.0 μg to about 30 μg, greater than 0.0 μg to about 50 μg, greater than 0.0 μg to about 80 μg, greater than 0.0 μg to about 100 μg, greater than 0.0 μg to about 120 μg, greater than 0.0 μg to about 130 μg, greater than 0.0 μg to about 150 μg, greater than 0.0 μg to about 180 μg, greater than 0.0 μg to about 200 μg, greater than 0.0 μg to about 230 μg, greater than 0.0 μg to about 240 μg, greater than 0.0 μg to about 250 μg, 0 μg to less than 250 μg, 0.5 μg to less than 250 μg, 1.5 μg to less than 250 μg, 2.5 μg to less than 250 μg, 5 μg to less than 250 μg, 12.5 μg to less than 250 μg, 25 μg to less than 250 μg, 40 μg to less than 250 μg, 60 μg to less than 250 μg, 90 μg to less than 250 μg, 110 μg to less than 250 μg, 125 μg to less than 250 μg, 140 μg to less than 250 μg, 170 μg to less than 250 μg, 200 μg to less than 250 μg, 215 μg to less than 250 μg, 0 μg to less than about 250 μg, about 0.5 μg to less than about 250 μg, about 1.5 μg to less than about 250 μg, about 2.5 μg to less than about 250 μg, about 5 μg to less than about 250 μg, about 12.5 μg to less than about 250 μg, about 25 μg to less than about 250 μg, about 40 μg to less than about 250 μg, about 60 μg to less than about 250 μg, about 90 μg to less than about 250 μg, about 110 μg to less than about 250 μg, about 125 μg to less than about 250 μg, about 140 μg to less than about 250 μg, about 170 μg to less than about 250 μg, about 200 μg to less than about 250 μg, and about 215 μg to less about than 250 μg.

In some cases, patients can be given cilastazol. In many case, low levels of cilastazol can be given to patients. For example, cilastazol can be given, e.g., intranasally, and/or present in a positive amount selected from a group consisting of: less than 500 mg to 450 mg, 475 mg to 425 mg, 435 mg to 400 mg, 415 mg to 300 mg, 325 mg to 250 mg, 275 mg to 150 mg, 200 mg to 100 mg, 135 mg to 80 mg, 95 mg to 65 mg, 75 mg to 50 mg, 60 mg to 40 mg, 45 mg to 25 mg, 30 mg to 20 mg, 15 mg to 5 mg, 10 mg to 2.5 mg, 3.5 mg to 1 mg, 2 mg to greater than 0 mg, less than about 500 mg to about 450 mg, about 475 mg to about 425 mg, about 435 mg to about 400 mg, about 415 mg to about 300 mg, about 325 mg to about 250 mg, about 275 mg to about 150 mg, about 200 mg to about 100 mg, about 135 mg to about 80 mg, about 95 mg to about 65 mg, about 75 mg to about 50 mg, about 60 mg to about 40 mg, about 45 mg to about 25 mg, about 30 mg to about 20 mg, about 15 mg to about 5 mg, about 10 mg to about 2.5 mg, about 3.5 mg to about 1 mg, about 2 mg to greater than 0 mg, 500 mg, less than 500 mg to 450 mg, less than 500 mg to 425 mg, less than 500 mg to 400 mg, less than 500 mg to 300 mg, less than 500 mg to 250 mg, less than 500 mg to 150 mg, less than 500 mg to 100 mg, less than 500 mg to 80 mg, less than 500 mg to 65 mg, less than 500 mg to 50 mg, less than 500 mg to 40 mg, less than 500 mg to 25 mg, less than 500 mg to 20 mg, less than 500 mg to 5 mg, less than 500 mg to 2.5 mg, less than 500 mg to 1 mg, less than 500 mg to greater than 0 mg, about 500 mg, less than about 500 mg to about 450 mg, less than about 500 mg to about 425 mg, less than about 500 mg to about 400 mg, less than about 500 mg to about 300 mg, less than about 500 mg to about 250 mg, less than about 500 mg to about 150 mg, less than about 500 mg to about 100 mg, less than about 500 mg to about 80 mg, less than about 500 mg to about 65 mg, less than about 500 mg to about 50 mg, less than about 500 mg to about 40 mg, less than about 500 mg to about 25 mg, less than about 500 mg to about 20 mg, less than about 500 mg to about 5 mg, less than about 500 mg to about 2.5 mg, less than about 500 mg to about 1 mg, less than about 500 mg to greater than about 0 mg, greater than 0 mg to 450 mg, greater than 0 mg to 425 mg, greater than 0 mg to 400 mg, greater than 0 mg to 300 mg, greater than 0 mg to 250 mg, greater than 0 mg to 150 mg, greater than 0 mg to 100 mg, greater than 0 mg to 80 mg, greater than 0 mg to 65 mg, greater than 0 mg to 50 mg, greater than 0 mg to 40 mg, greater than 0 mg to 25 mg, greater than 0 mg to 20 mg, greater than 0 mg to 5 mg, greater than 0 mg to 2.5 mg, greater than 0 mg to 1 mg, greater than 0 mg to about 450 mg, greater than 0 mg to about 425 mg, greater than 0 mg to about 400 mg, greater than 0 mg to about 300 mg, greater than 0 mg to about 250 mg, greater than 0 mg to about 150 mg, greater than 0 mg to about 100 mg, greater than 0 mg to about 80 mg, greater than 0 mg to about 65 mg, greater than 0 mg to about 50 mg, greater than 0 mg to about 40 mg, greater than 0 mg to about 25 mg, greater than 0 mg to about 20 mg, greater than 0 mg to about 5 mg, greater than 0 mg to about 2.5 mg, and greater than 0 mg to about 1 mg.

In some cases, patients can be given roflumilast. In many case, low levels of roflumilast can be given to patients. For example, roflumilast can be given, e.g., intranasally, and/or present in a positive amount selected from a group consisting of: less than 10 mg, 5 mg, 1 mg, 500 μg, 250 μg, 120 μg, 80 μg, 40 μg, or 20 μg and less than about 10 mg, about 5 mg, about 1 mg, about 500 μg, about 250 μg, about 120 μg, about 80 μg, about 40 μg, or about 20 μg. Roflumilast can be also given, e.g., intranasally, and/or present in an amount selected from a group consisting of: greater than 0 μg to 20 μg, 10 mg to 40 μg, 30 μg to 80 μg, 70 μg to 120 μg, 100 μg to 250 μg, 200 μg to 500 μg, 400 μg to 1 mg, 900 μg to 5 mg, 4 mg to 10 mg, 9 mg to 15 mg, 14 mg to 30 mg, 25 mg to 45 mg, greater than 0 μg to about 20 μg, about 10 μg to about 40 μg, about 30 μg to about 80 μg, about 70 μg to about 120 μg, about 100 μg to about 250 μg, about 200 μg to about 500 μg, about 400 μg to about 1 mg, about 900 μg to about 5 mg, about 4 mg to about 10 mg, greater than 0 μg to 40 μg, greater than 0 μg to 80 μg, greater than 0 μg to 120 μg, greater than 0 μg to 250 μg, greater than 0 μg to 500 μg, greater than 0 μg to 1 mg, greater than 0 μg to 5 mg, greater than 0 μg to 10 mg, greater than 0 μg to 15 mg, greater than 0 μg to 30 mg, greater than 0 μg to 45 mg, greater than 0 μg to about 40 μg, greater than 0 μg to about 80 μg, greater than 0 μg to about 120 μg, greater than 0 μg to about 250 μg, greater than 0 μg to about 500 μg, greater than 0 μg to about 1 mg, greater than 0 μg to about 5 mg, greater than 0 μg to about 10 mg, greater than 0 μg to 10 mg, 10 μg to 10 mg, 30 μg to 10 mg, 70 μg to 10 mg, 100 μs to 10 mg, 200 μg to 10 mg, 400 μg to 10 mg, 900 μg to 10 mg, 4 mg to 10 mg, 9 mg to 10 mg, greater than 0 μg to about 10 mg, about 10 μg to about 10 mg, about 30 μg to about 10 mg, about 70 μg to about 10 mg, about 100 μg to about 10 mg, about 200 μg to about 10 mg, about 400 μg to about 10 mg, about 900 μg to about 10 mg, about 4 mg to about 10 mg, and/or about 9 mg to about 10.

In some cases, patients can be given papaverine. In many case, low levels of papaverine can be given to patients. For example, papaverine can be given, e.g., intranasally, and/or present in a positive amount selected from a group consisting of: less than 500 mg to 450 mg, 475 mg to 425 mg, 435 mg to 400 mg, 415 mg to 300 mg, 325 mg to 250 mg, 275 mg to 150 mg, 200 mg to 100 mg, 135 mg to 80 mg, 95 mg to 65 mg, 75 mg to 50 mg, 60 mg to 40 mg, 45 mg to 25 mg, 30 mg to 20 mg, 15 mg to 5 mg, 10 mg to 2.5 mg, 3.5 mg to 1 mg, 2 mg to greater than 0 mg, less than about 500 mg to about 450 mg, about 475 mg to about 425 mg, about 435 mg to about 400 mg, about 415 mg to about 300 mg, about 325 mg to about 250 mg, about 275 mg to about 150 mg, about 200 mg to about 100 mg, about 135 mg to about 80 mg, about 95 mg to about 65 mg, about 75 mg to about 50 mg, about 60 mg to about 40 mg, about 45 mg to about 25 mg, about 30 mg to about 20 mg, about 15 mg to about 5 mg, about 10 mg to about 2.5 mg, about 3.5 mg to about 1 mg, about 2 mg to greater than 0 mg, 500 mg, less than 500 mg to 450 mg, less than 500 mg to 425 mg, less than 500 mg to 400 mg, less than 500 mg to 300 mg, less than 500 mg to 250 mg, less than 500 mg to 150 mg, less than 500 mg to 100 mg, less than 500 mg to 80 mg, less than 500 mg to 65 mg, less than 500 mg to 50 mg, less than 500 mg to 40 mg, less than 500 mg to 25 mg, less than 500 mg to 20 mg, less than 500 mg to 5 mg, less than 500 mg to 2.5 mg, less than 500 mg to 1 mg, less than 500 mg to greater than 0 mg, about 500 mg, less than about 500 mg to about 450 mg, less than about 500 mg to about 425 mg, less than about 500 mg to about 400 mg, less than about 500 mg to about 300 mg, less than about 500 mg to about 250 mg, less than about 500 mg to about 150 mg, less than about 500 mg to about 100 mg, less than about 500 mg to about 80 mg, less than about 500 mg to about 65 mg, less than about 500 mg to about 50 mg, less than about 500 mg to about 40 mg, less than about 500 mg to about 25 mg, less than about 500 mg to about 20 mg, less than about 500 mg to about 5 mg, less than about 500 mg to about 2.5 mg, less than about 500 mg to about 1 mg, less than about 500 mg to greater than about 0 mg, greater than 0 mg to 450 mg, greater than 0 mg to 425 mg, greater than 0 mg to 400 mg, greater than 0 mg to 300 mg, greater than 0 mg to 250 mg, greater than 0 mg to 150 mg, greater than 0 mg to 100 mg, greater than 0 mg to 80 mg, greater than 0 mg to 65 mg, greater than 0 mg to 50 mg, greater than 0 mg to 40 mg, greater than 0 mg to 25 mg, greater than 0 mg to 20 mg, greater than 0 mg to 5 mg, greater than 0 mg to 2.5 mg, greater than 0 mg to 1 mg, greater than 0 mg to about 450 mg, greater than 0 mg to about 425 mg, greater than 0 mg to about 400 mg, greater than 0 mg to about 300 mg, greater than 0 mg to about 250 mg, greater than 0 mg to about 150 mg, greater than 0 mg to about 100 mg, greater than 0 mg to about 80 mg, greater than 0 mg to about 65 mg, greater than 0 mg to about 50 mg, greater than 0 mg to about 40 mg, greater than 0 mg to about 25 mg, greater than 0 mg to about 20 mg, greater than 0 mg to about 5 mg, greater than 0 mg to about 2.5 mg, and greater than 0 mg to about 1 mg.

Patients can be also given a variety of other therapeutic agents. For examples, cytochrome p450 inhibitors can be given to patients.

Patients can be also given β-adrenergic activators, including but not limited to β₁-adrenergic activators, β₂-adrenergic activators, and uncharacterized β-adrenergic activators. For example, patients can be given a β₁-adrenergic activators selected from a group consisting of dobutamine, isoproterenol, xamoterol, and epinephrine; a β₂-adrenergic activators selected from a group consisting of albuterol, levalbuterol, fenoterol, formoterol, isoproterenol (β₁ and β₂), metaproterenol, salmeterol, terbutaline, clenbuterol, isoetarine, pirbuterol, procaterol, ritodrine, and epinephrine; and/or a uncharacterized β-adrenergic activators selected from a group consisting of arbutamine, befunolol, bromoacetylalprenololmenthane, broxaterol, cimaterol, cirazoline, denopamine, dopexamine, etilefrine, hexoprenaline, higenamine, isoxsuprine, mabuterol, methoxyphenamine, nylidrin, oxyfedrine, prenalterol, ractopamine, reproterol, rimiterol, tretoquinol, tulobuterol, zilpaterol, and zinterol.

In many cases, the therapeutic agents given to the patients can be steroid-free.

Some patients can be treated for one or more symptoms associated with chromosomal abnormalities by altering the levels of members of the hedgehog signaling pathway. For example, patients can be given an effective amount of one or more members of the hedgehog signaling pathway. In some cases, patients can be given an effective amount of the one or more exogenous members of the hedgehog signaling pathway. These members (e.g., RNA or protein) can be made in vitro or in vivo by known methods.

Alternatively, patients can be treated for one or more symptoms associated with chromosomal abnormalities by activating the expression of an effective amount of one or more members of the hedgehog signaling pathway. In some cases, genetic manipulation responsible for the expression of one or more members of the hedgehog signaling pathway can be performed in vitro or in vivo. For example, promoter regions can be activated to increase the expression of one or more members of the hedgehog signaling pathway. This can include, but not limited to methods such as gene therapy. In other cases, activated expression can be effectuated through a therapeutic agent. Additionally, the treatment can directly or indirectly affect levels of one or more members of the hedgehog signaling pathway.

In many cases, the patients can be given a combination treatment. Any of the previously mentioned therapeutic agents and/or methods can be given in combinations of two or more.

With regards to timing, a woman can be treated when she is sexually active, is attempting to conceive, and/or actually conceives. The treating can be performed 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 weeks after conception. The treating can be performed during labor or immediately after birth. In some embodiments, the treating can be performed 1 week after conception. In some embodiments, the treating can be performed 2 weeks after conception. In some embodiments, the treating can be performed 3 weeks after conception. In some embodiments, the treating can be performed 4 weeks after conception. In some embodiments, the treating can be performed 5 weeks after conception. In some embodiments, the treating can be performed 6 weeks after conception. In some embodiments, the treating can be performed 7 weeks after conception. In some embodiments, the treating can be performed 8 weeks after conception. In some embodiments, the treating can be performed 9 weeks after conception. In some embodiments, the treating can be performed 10 weeks after conception. In some embodiments, the treating can be performed 11 weeks after conception. In some embodiments, the treating can be performed 12 weeks after conception. In some embodiments, the treating can be performed 13 weeks after conception. In some embodiments, the treating can be performed 14 weeks after conception. In some embodiments, the treating can be performed 15 weeks after conception. In some embodiments, the treating can be performed 16 weeks after conception. In some embodiments, the treating can be performed 17 weeks after conception. In some embodiments, the treating can be performed 18 weeks after conception. In some embodiments, the treating can be performed 19 weeks after conception. In some embodiments, the treating can be performed 20 weeks after conception. In some embodiments, the treating can be performed 21 weeks after conception. In some embodiments, the treating can be performed 22 weeks after conception. In some embodiments, the treating can be performed 23 weeks after conception. In some embodiments, the treating can be performed 24 weeks after conception. In some embodiments, the treating can be performed 25 weeks after conception. In some embodiments, the treating can be performed 26 weeks after conception. In some embodiments, the treating can be performed 27 weeks after conception. In some embodiments, the treating can be performed 28 weeks after conception. In some embodiments, the treating can be performed 29 weeks after conception. In some embodiments, the treating can be performed 30 weeks after conception. In some embodiments, the treating can be performed 31 weeks after conception. In some embodiments, the treating can be performed 32 weeks after conception. In some embodiments, the treating can be performed 33 weeks after conception. In some embodiments, the treating can be performed 34 weeks after conception. In some embodiments, the treating can be performed 35 weeks after conception. In some embodiments, the treating can be performed 36 weeks after conception. In some embodiments, the treating can be performed 37 weeks after conception. In some embodiments, the treating can be performed 38 weeks after conception. In some embodiments, the treating can be performed 39 weeks after conception. In some embodiments, the treating can be performed 40 weeks after conception.

### Example 6: Treating Disease with cGMP Activators and/or cAMP Activators

To ameliorate one or more symptoms associated with chromosomal abnormalities in patients in need thereof, patients can be given one or more cGMP activators, one or more cAMP activators, or any combination thereof.

The patients can be given a cGMP activators selected from a group consisting of 3-(5′-hydroxymethyl-2′-furyl)-1-benzylindazole (YC-1), YC-1 derivatives, anthranilic acids derivatives, ataciguat (HMR1766), benzydamine analogs, CFM1517, A-350619, nitrovasodilators, molsidomine, nitroxyl (HNO), BAY 41-2272, BAY 41-8543, BAY 58-2667, cinaciguat (BAY 58-2667), and riociguat (BAY 63-2521).

Sometimes the patients can be given a cAMP activators selected from a group consisting of 3-(5′-hydroxymethyl-2′-furyl)-1-benzylindazole (YC-1), glucagon, PDE inhibitors, prostaglandin E1 (PGE1; pharmaceutically known as alprostadil), forskolin, and β-adrenergic activators.

In many cases, giving a patient one or more cAMP activator and/or one or more cAMP activators, will ameliorate one or more symptoms associated with chromosomal abnormalities in patients so diagnosed.

In many cases, the patients can be given a combination treatment. Any of the previously mentioned therapeutic agents and/or methods can be given in combinations of two or more.

### Example 7: Alternative Formulations

The previously described therapeutic agents, individually or in combination, can be formulated so that they can be suitable for administration by a method selected from a group consisting of: oral administration, transmucosal administration, buccal administration, inhalation administration, intranasal administration, parental administration, intravenous administration, subcutaneous administration, intramuscular administration, sublingual administration, transdermal administration, and rectal administration. In this case, for ease of delivery to the target site, the therapeutic agents can be formulated as suitable for intranasal and oral administration.

For example, the route of administration can penetrate the placental barrier and/or the blood/brain barrier. For example, in some embodiments, pregnant mothers can be given a dose of a drug, which will then reach the fetus by crossing the placental barrier. The drug can then enter the fetus and find its way to target sites, e.g., the brain. If the drug reaches the brain, it can cross the blood/brain barrier. In some other embodiments, a breast feeding mother can be given a dose of drug, which will then reach the baby by flowing to the mother's breast milk. The breast milk containing the drug can be then fed to the baby. In some embodiments, the drug can be used to fortify baby's formula and/or milk and then fed to the baby. The drug will eventually find its way to the brain by crossing the blood/brain barrier. In some embodiments, the drug may or may not cross the blood/brain barrier.

Different excipients can be used for the different formulations. For example, sweeter excipients can be used to mask bitterness with while binders can be used to form tablets.

Patients can be given a liquid form of the therapeutic agent, suitable for intranasal and oral administration. The pH of the liquid therapeutic agent can be adjusted because the pH can play a role in efficacy. The pH can be, for example, about: 6.0, 6.1, 6.2, 6.3, 6.4, 6.5, 6.6, 6.7, 6.8, 6.9, 7.0, 7.1, 7.2, 7.3, 7.4, 7.5, or range from, for example, 5.0 to 5.5; 5.1 to 5.6; 5.2 to 5.7; 5.3 to 5.8; 5.4 to 5.9; 5.5 to 6.0; 5.6 to 6.1; 5.7 to 6.2; 5.8 to 6.3; 5.9 to 6.4; 6.0 to 6.5; 6.1 to 6.6; 6.2 to 6.7; 6.3 to 6.8; 6.4 to 6.9; 6.5 to 7.0; 6.6 to 7.1; 6.7 to 7.2; 6.8 to 7.3; 6.9 to 7.4; 7.0 to 7.5; 7.1 to 7.6; 7.2 to 7.7; 7.3 to 7.8; 7.4 to 7.9; 7.5 to 8.0; 7.6 to 8.1; 7.7 to 8.2; 7.8 to 8.3; 7.9 to 8.4; 8.0 to 8.5; 8.1 to 8.6; 8.2 to 8.7; 8.3 to 8.8; 8.4 to 8.9; 8.5 to 9.0; 8.6 to 9.1; 8.7 to 9.2; 8.8 to 9.3; 8.9 to 9.4; 9.0 to 9.5; about 5.0 to about 5.5; about 5.1 to about 5.6; about 5.2 to about 5.7; about 5.3 to about 5.8; about 5.4 to about 5.9; about 5.5 to about 6.0; about 5.6 to about 6.1; about 5.7 to about 6.2; about 5.8 to about 6.3; about 5.9 to about 6.4; about 6.0 to about 6.5; about 6.1 to about 6.6; about 6.2 to about 6.7; about 6.3 to about 6.8; about 6.4 to about 6.9; about 6.5 to about 7.0; about 6.6 to about 7.1; about 6.7 to about 7.2; about 6.8 to about 7.3; about 6.9 to about 7.4; about 7.0 to about 7.5; about 7.1 to about 7.6; about 7.2 to about 7.7; about 7.3 to about 7.8; about 7.4 to about 7.9; about 7.5 to about 8.0; about 7.6 to about 8.1; about 7.7 to about 8.2; about 7.8 to about 8.3; about 7.9 to about 8.4; about 8.0 to about 8.5; about 8.1 to about 8.6; about 8.2 to about 8.7; about 8.3 to about 8.8; about 8.4 to about 8.9; about 8.5 to about 9.0; about 8.6 to about 9.1; about 8.7 to about 9.2; about 8.8 to about 9.3; about 8.9 to about 9.4; and/or about 9.0 to about 9.5.

### Example 8: Treating Chromosomal Abnormalities and/or One or More Symptoms Associated with Chromosomal Abnormalities with Low Levels of Ricociguat

The intranasal formulation contains lower and sometimes significantly lower amounts of riociguat when compared to what can be necessary for oral dosing. For example, patients can be given an intranasal formulation of riociguat, wherein riociguat can be present in a positive amount selected from a group consisting of: greater than 0.0 μg to 1 μg, 0.5 μg to 2 μg, 1.5 μg to 3.0 μg, 2.5 μg to 10 μg, 5 μg to 15 μg, 12.5 μg to 30 μg, 25 μg to 50 μg, 40 μg to 80 μg, 60 μg to 100 μg, 90 μg to 120 μg, 110 μg to 130 μg, 125 μg to 150 μg, 140 μg to 180 μg, 170 μg to 200 μg, 200 μg to 230 μg, 215 μg to 240 μg, 235 μg to less than 250 μg, less than 250 μg, greater than about 0.0 μg to about 1 μg, about 0.5 μg to about 2 μg, about 1.5 μg to about 3.0 μg, about 2.5 μg to about 10 μg, about 5 μg to about 15 μg, about 12.5 μg to about 30 μg, about 25 m to about 50 μg, about 40 m to about 80 μg, about 60 μg to about 100 μg, about 90 μg to about 120 μg, about 110 μg to about 130 μg, about 125 μg to about 150 μg, about 140 μg to about 180 μg, about 170 μg to about 200 μg, about 200 μg to about 230 μg, about 215 μg to about 240 μg, about 235 μg to less than 250 μg greater than 0.0 μg to 2 μg, greater than 0.0 μg to 3 μg, greater than 0.0 μg to 10 μg, greater than 0.0 μg to 15 μg, greater than 0.0 μg to 30 μg, greater than 0.0 μg to 50 μg, greater than 0.0 μg to 80 μg, greater than 0.0 μg to 100 μg, greater than 0.0 μg to 120 μg, greater than 0.0 μg to 130 μg, greater than 0.0 μg to 150 μg, greater than 0.0 μg to 180 μg, greater than 0.0 μg to 200 μg, greater than 0.0 μg to 230 μg, greater than 0.0 m to 240 μg, greater than 0.0 μg to 250 μg, greater than 0.0 μg to about 2 μg, greater than 0.0 μg to about 3 μg, greater than 0.0 μg to about 10 μg, greater than 0.0 μg to about 15 μg, greater than 0.0 μg to about 30 μg, greater than 0.0 μg to about 50 μg, greater than 0.0 μg to about 80 μg, greater than 0.0 lag to about 100 μg, greater than 0.0 μg to about 120 μg, greater than 0.0 μg to about 130 μg, greater than 0.0 μg to about 150 μg, greater than 0.0 μg to about 180 μg, greater than 0.0 μg to about 200 μg, greater than 0.0 μg to about 230 μg, greater than 0.0 μg to about 240 μg, greater than 0.0 μg to about 250 μg, 0 μg to less than 250 μg, 0.5 μg to less than 250 μg, 1.5 μg to less than 250 μg, 2.5 μg to less than 250 μg, 5 μg to less than 250 μg, 12.5 μg to less than 250 μg, 25 μg to less than 250 μg, 40 μg to less than 250 μg, 60 μg to less than 250 μg, 90 μg to less than 250 μg, 110 μg to less than 250 μg, 125 μg to less than 250 μg, 140 μg to less than 250 μg, 170 μg to less than 250 μg, 200 μg to less than 250 μg, 215 μg to less than 250 μg, 0 μg to less than about 250 μg, about 0.5 μg to less than about 250 μg, about 1.5 μg to less than about 250 μg, about 2.5 μg to less than about 250 μg, about 5 μg to less than about 250 μg, about 12.5 μg to less than about 250 μg, about 25 μg to less than about 250 μg, about 40 μg to less than about 250 μg, about 60 μg to less than about 250 μg, about 90 μg to less than about 250 μg, about 110 μg to less than about 250 μg, about 125 μg to less than about 250 μg, about 140 μg to less than about 250 μg, about 170 μg to less than about 250 μg, about 200 μg to less than about 250 μg, and about 215 μg to less about than 250 μg. Other patients can be given an intranasal formulation of riociguat wherein riociguat can be present in a positive amount: less than 250 μg to greater than 0 or about less than 250 μg to greater than 0; less than 200 μg to greater than 0 or about less than 200 μg to greater than 0; less than 150 μg to greater than 0 or about less than 150 μg to greater than 0; less than 100 μg to greater than 0 or about less than 100 μg to greater than 0; less than 50 μg to greater than 0 and/or about less than 50 μg to greater than 0.

Some patients can be also treated with non-intranasal inhalational and/or intravenous formulations of riociguat because the effective dosage of riociguat for inhalational and/or intravenous formulations require significantly lower amounts of riociguat.

In many cases, the patients can be given a combination treatment. Any of the previously mentioned therapeutic agents and/or methods can be given in combinations of two or more.

While preferred embodiments of the present invention have been shown and described herein, it will be obvious to those skilled in the art that such embodiments can be provided by way of example only. Numerous variations, changes, and substitutions will now occur to those skilled in the art without departing from the invention. It should be understood that various alternatives to the embodiments of the invention described herein may be employed in practicing the invention.

### Example 9: Diagnosing and Treating Down Syndrome or Other Chromosomal Abnormalities

Decreased levels of SHH may play an important role in the development of symptoms associated with chromosomal abnormalities, e.g., chromosomal translocations, trisomy 13, trisomy 18, and trisomy 21. To ameliorate one or more symptoms associated with chromosomal defects, a subject can be first diagnosed (in utero or post-birth) with having a chromosomal abnormality, e.g., chromosomal translocations, trisomy 13, trisomy 18, and trisomy 21. For example, prenatal genetic testing can be used to detect the presence or absence of a chromosomal abnormality in utero. Any known method can be used to diagnosis a subject with carrying a fetus and/or embryo with chromosomal abnormalities, e.g., chromosomal translocations, trisomy 13, trisomy 18, and trisomy 21. If an invasive prenatal test is used, any known method can be used, e.g., amniocentesis, chorionic villus sampling, embryoscopy, fetoscopy, and/or percutaneous umbilical cord blood sampling. If a non-invasive prenatal test is used, any known method can be used, e.g., fetal cells in maternal blood, cell-free fetal DNA in maternal blood, preimplantation genetic diagnosis, external examination, ultrasound detection, fetal heartbeat, non-stress test, transcervical retrieval of trophoblast cells, and maternal serum screening. For example, kits that utilize cell free fetal DNA extracted from the maternal blood can be used. Kits such as Harmony Prenatal Test (Ariosa), MaterniT (Sequenom), MaterniT21 (Sequenom), and Panorama (Natera) can be used. If diagnostic tests can be performed post-birth, any known methods such as simply karyotyping, methods using fluorescence hybridization, and/or any other genetic tests can be used.

After a subject is diagnosed with carrying a fetus and/or an embryo with one or more chromosomal abnormalities and/or one or more symptoms of chromosomal abnormalities, the subject can be treated with one or more activators of the hedgehog signaling pathway that activates one or more members of the hedgehog signaling pathway selected from a group consisting of: SHH, DHH, and IHH, and combinations thereof. In some cases, the activators of the hedgehog signaling pathway agonizes SHH. The one or more activators of the hedgehog signaling pathway can be selected from the group consisting of: cyclic adenosine monophosphate activator and/or cyclic guanosine monophosphate activator. The one or more activators of the hedgehog signaling pathway can also be selected from the group consisting of: ELND005, a drug that decreases myo-inositol, RG1662, Picrotoxin, GABA blocked drugs, PTZ, Nicotine, Green tea extract, Nerve growth factors, introducing a XIST gene, theophylline, riociguat, forskolin, phosphodiesterase inhibitor, or combinations thereof. Some subjects can be given theophylline.

In some instances, the one or more activators of the hedgehog signaling can be formulated as at least one composition or dosage unit. These formulations can be steroid-free. These formulations can also be formulated for intranasal administration.

Some subjects having one or more chromosomal abnormalities and/or one or more symptoms associated with chromosomal abnormalities can require continuous, indefinite treatment of the one or more activators of the hedgehog signaling pathway. For example, in some cases, the subject having one or more chromosomal abnormalities and/or one or more symptoms associated with chromosomal abnormalities can be treated in utero by giving the pregnant mother one or more activators of the hedgehog signaling pathway. However, the one or more activators of the hedgehog signaling pathway must be able to cross the fetal-placental barrier and the blood/brain barrier. For instance, theophylline, e.g., oral or nasal dosage forms, can be given to pregnant mothers. In other cases, the subject having one or more chromosomal abnormalities and/or one or more symptoms associated with chromosomal abnormalities can be treated at birth by giving the infant one or more activators of the hedgehog signaling pathway. For example, theophylline can be given orally, e.g., by mixing it with milk/formula or by giving theophylline to a breast-feeding mother, wherein the theophylline can be within the breast milk, or theophylline can be injected directly or given as a nasal dosage to the infant. The same can be done during the period of time known as post-partum, e.g., given theophylline to the infant direct or to a breast feeding mother. The subject having one or more chromosomal abnormalities and/or one or more symptoms of chromosomal abnormalities, can be also treated continuously (in utero and/or at birth), or any duration, including for the rest of its life.

By giving to a subject having one or more chromosomal abnormalities and/or one or more symptoms of chromosomal abnormalities, one or more activators of the hedgehog signaling pathway, one or more symptoms associated with the chromosomal abnormality can be ameliorated. For example, cognitive and/or physical symptoms can be ameliorated. Some cognitive symptoms that can be ameliorated by this method can be intellectual disability, inability to speak, mental illness, autism, depression, anxiety, epileptic seizures, and dementia. Additionally, one or more physical abnormalities can be ameliorated, e.g., stunted growth, umbilical hernia, increased skin on the neck, low muscle tone, narrow roof of mouth, flat head, flexible ligaments, large tongue, abnormal outer ears, flattened nose, separation of first and second toes, abnormal teeth, slanted eyes, shortened hands, short neck, obstructive sleep apnea, bent fifth finger tip, brushfield spots in the iris, cataracts, keratonconus, glaucoma, hearing problems, otitis media with effusion, poor Eustachian tube function, single transverse palmar crease, protruding tongue, congenital heart disease, strabismus, congenital hypothyroidism, diabetes, duodenal atresia, pyloric stenosis, Meckel diverticulum, imperforate anus, celiac disease, gastroesophageal reflux disease, early menopause, infertility, and undescended testicles.

If a patient has for example, down syndrome, treatment with one or more activators of the hedgehog signaling pathway can lead to ameliorating some abnormalities that are common in almost all down syndrome patients, such as facial dysmorphology, a small and hypocellular brain, and/or the histopathology of Alzheimer disease. Other diseases that can be treated by methods disclosed herein, include, but are not limited to, vision problems (such as cataracts, near-sightedness, “crossed” eyes, and rapid, involuntary eye movements), hearing loss, infections, hypothyroidism, blood disorders (such as leukemia, anemia, and ploycythemia), hypotonia, problems with upper part of the spine (such as misshapen bones in the upper part of the spine, underneath the base of the skull), disrupted sleep patterns and sleep disorders (such as sleep apnea), gum disease and dental problems (such as slower developing teeth, developing teeth in a different order, developing fewer teeth, or having misaligned teeth, compared to normal counterparts (e.g., persons without down syndrome), epilepsy, digestive problems, celiac disease (such as intestinal problems when down syndrome persons eat gluten), and/or mental health and emotional problems (such as anxiety, depression, and Attention Deficit Hyperactivity Disorder, repetitive movements, aggression, autism, psychosis, and/or social withdrawal).

Furthermore, down syndrome patients treated with one or more activators of the hedgehog signaling pathway can decrease the risk of certain diseases, for example, congenital heart disease (CHD). The decrease in risk can be lowered, e.g., to levels associated with people with no known chromosomal abnormalities. Some diseases risks that can be lowered by the methods herein, can be e.g., CHD, leukemia, and Hirschsprung.

### Example 10: Sonic Hedgehog Levels During Cilostazol and Roflumilast Treatment

Patients were given oral doses of roflumilast alone and the patients nasal mucus was isolated and measured for sonic hedgehog levels. For example, Patient ID No. 11, was given 500 micrograms of daliresp (roflumilast) orally once per day for 4 months. Sonic hedgehog levels were presented at 6665 μg/mol, which is within the levels measured with theophylline treatment alone.

Patients were also given oral doses of cilostazol alone and then measured for nasal mucosal sonic hedgehog levels. For example, Patient ID No. 7, was given 100 mg of cilostazol one per day orally for 4 months. Sonic hedgehog levels were present at a mean of 769 ng/mol, which is also within levels previously measured for theophylline treatment alone.

Treatment with both oral theophylline and cilostazol levels reveal a sonic hedgehog level of a mean of 520 μg/mol. Similar values were measured with oral theophylline and rofluimlast. As with oral theophylline values varied a great deal but they were all significantly higher than before the drugs were administered.

The following is the raw data from these studies. Regarding the terms in the following data, “AR” can refer to allergic rhinitis. “PIHH” can refer to post-influenza-like hyposmia and hypogeusia. “C” can refer to congenital. “HI” can refer to head injury. “A” can refer to anesthesia-induced. “I” can mean idiopathic. Patient names were blocked out for privacy.

### ShhN Nasal Mucus Vs PDE Inhibitor Comparison

DALIRESP ONLY

| | | | | | | | ShhN | Theo | |
|---|---|---|---|---|---|---|---|---|---|
| | | | | | | | (pg/mol) | Dose | |
| ID | Patient | Date of Visit | Gender | Date of Birth | Age | Diagnosis | Nasal Mucus | (mg) | Date 1* Theo |
| 11 | | Apr. 24, 2014 | 1 | Sep. 20, 1927 | 87 | AR | 6664.912 | — | Apr. 18, 2002-Dec. 1, 2006 |

| | | | Daliresp | | | | |
|---|---|---|---|---|---|---|---|
| | Cilostazol | Date 1º | Dose | Date 1º | | | |
| ID | Dose (mg) | Cilostazol | (mcg) | Daliresp | Other Med | Date 1º Other | Hx Dexa |
| 11 | — | Jul. 16, 2004-Dec. | 500 qd | Apr. 27, 2012 | — | — | Oct. 10, 2002, |
| | | 1, 2006 | | | | | Jul. 13, 2006, |
| | | | | | | | Sep. 7, 2006 |

CILOSTAZOL + DALIRESP ONLY

| | | | | | | | ShhN | Theo | |
|---|---|---|---|---|---|---|---|---|---|
| | | | | | | | (pg/mol) | Dose | |
| ID | Patient | Date of Visit | Gender | Date of Birth | Age | Diagnosis | Nasal Mucus | (mg) | Date 1* Theo |
| 7 | | Jan. 2, 2014 | 0 | Aug. 11, 1931 | 82 | PIHH | 520.278 | — | — |

| | | | Daliresp | | | | |
|---|---|---|---|---|---|---|---|
| | Cilostazol | Date 1º | Dose | Date 1º | | | |
| ID | Dose (mg) | Cilostazol | (mcg) | Daliresp | Other Med | Date 1º Other | Hx Dexa |
| 7 | 200 | Mar. 14, 2013 | 250 qd | Nov. 7, 2013 | — | — | — |

THEOPHYLLINE + DALIRESP ONLY

| | | | | | | | ShhN | Theo | |
|---|---|---|---|---|---|---|---|---|---|
| | | | | | | | (pg/mol) | Dose | |
| ID | Patient | Date of Visit | Gender | Date of Birth | Age | Diagnosis | Nasal Mucus | (mg) | Date 1* Theo |
| 1 | | Mar. 6, 2014 | 0 | Apr. 12, 1944 | 70 | AR | 176.644 | 800 | Apr. 22, 2010 |
| 4 | | Apr. 26, 2012 | 0 | Sep. 25, 1931 | 81 | PIHH | 1604.655 | 600 | Mar. 21, 1996 |
| 5 | | Nov. 29, 2012 | 1 | Nov. 7, 1965 | 47 | Congenital | 97.581 | 300 | Apr. 14, 2011 |
| 9 | | Jun. 8, 2013 | 0 | Mar. 8, 1957 | 56 | AR | 60.592 | 600 | May 19, 2011 |
| | | | | | n = 4 | | n = 4 | | |
| | | | | Average | 63 | Average | 484.87 | | |
| | | | | Std. Error | 7 | Std. Error | 374.05 | | |

| | | | Daliresp | | | | |
|---|---|---|---|---|---|---|---|
| | Cilostazol | Date 1º | Dose | Date 1º | | | |
| ID | Dose (mg) | Cilostazol | (mcg) | Daliresp | Other Med | Date 1º Other | Hx Dexa |
| 1 | — | — | 500 qd | Nov. 7, 2013 | — | — | — |
| 4 | — | — | 250 qod | Mar. 29, 2012 | Zinc | May 3, 2001 | Nov. 1, 2008 |
| 5 | — | — | 250 qd | Mar. 1, 2012 | — | — | |
| 9 | — | — | 500 qd | May 20, 2011 | Zinc | Jun. 24, 2011 | May 19, 2011, |
| | | | | | | | Jun. 6, 2013 |

THEOPHYLLINE + CILOSTAZOL + DALIRESP

| | | | | | | | ShhN | Theo | |
|---|---|---|---|---|---|---|---|---|---|
| | | | | | | | (pg/mol) | Dose | |
| ID | Patient | Date of Visit | Gender | Date of Birth | Age | Diagnosis | Nasal Mucus | (mg) | Date 1* Theo |
| 3 | | Jul. 9, 2012 | 0 | Sep. 26, 1971 | 41 | AR | 57.044 | 400 | Mar. 2, 2006 |
| 8 | | Jan. 9, 2014 | 0 | Aug. 15, 1938 | 75 | PIHH | 665.448 | 800 | Aug. 26, 2004 |
| 12 | | Aug. 23, 2012 | 0 | Nov. 1, 1939 | 73 | AR | 48.084 | 800 | Dec. 7, 2006 |
| 2 | | Mar. 1, 2013 | 0 | Oct. 11, 1945 | 67 | AR | 58.179 | 800 | Jan. 18, 2007 |
| 6 | | Mar. 27, 2014 | 1 | Oct. 26, 1972 | 41 | AR | 811.333 | 800 | Jan. 14, 2010 |
| 10 | | Mar. 28, 2013 | 1 | Nov. 10, 1956 | 56 | HI | 55.868 | 800 | Feb. 22, 2007 |
| | | | | | n = 6 | | n = 6 | | |
| | | | | Average | 59 | Average | 282.66 | | |
| | | | | Std. Error | 6 | Std. Error | 145.35 | | |

| | | | Daliresp | | | | |
|---|---|---|---|---|---|---|---|
| | Cilostazol | Date 1º | Dose | Date 1º | | | |
| ID | Dose (mg) | Cilostazol | (mcg) | Daliresp | Other Med | Date 1º Other | Hx Dexa |
| 3 | 100 | Jun. 19, 2009 | 250 qd | Mar. 15, 2012 | — | — | |
| 8 | 100 | Jun. 2, 2005 | 500 qd | May 17, 2012 | — | — | Jan. 26, 2005 |
| 12 | 100 | Dec. 6, 2007 | 250 qd | Jun. 8, 2012 | — | — | — |
| 2 | 200 | Sep. 10, 2009 | 250 qd | Mar. 8, 2012 | Singulair | Aug. 30, 2010 | Apr. 5, 2007, |
| | | | | | 10 mg | | Sep. 10, 2009 |
| 6 | 200 | Feb. 2, 2011 | 500 qd | Jan. 11, 2013 | — | — | Sep. 16, 2010 |
| 10 | 200 | Jun. 16, 2011 | 500 qd | 3/38/13 | — | — | Feb. 27, 2007, |
| | | | | | | | Nov. 9, 2007, |
| | | | | | | | Mar. 19, 2009, |
| | | | | | | | Jun. 16, 2011 |

CILOSTAZOL ONLY

| | | | | | | | ShhN | Theo | |
|---|---|---|---|---|---|---|---|---|---|
| | | | | | | | (pg/mol) | Dose | |
| ID | Patient | Date of Visit | Gender | Date of Birth | Age | Diagnosis | Nasal Mucus | (mg) | Date 1* Theo |
| 31 | | Nov. 7, 2013 | 0 | Aug. 11, 1931 | 82 | PIHH | 768.949 | — | — |

| | | | Daliresp | | | | |
|---|---|---|---|---|---|---|---|
| | Cilostazol | Date 1º | Dose | Date 1º | | | |
| ID | Dose (mg) | Cilostazol | (mcg) | Daliresp | Other Med | Date 1º Other | Hx Dexa |
| 31 | 200 | Mar. 14, 2013 | — | Nov. 7, 2013 | — | — | — |

THEOPHYLLINE + CILOSTAZOL

| | | | | | | | ShhN | | |
|---|---|---|---|---|---|---|---|---|---|
| | | | | | | | (pg/mol) | Theo Dose | |
| ID | Patient | Date of Visit | Gender | Date of Birth | Age | Diagnosis | Nasal Mucus | (mg) | Date 1* Theo |
| 17 | | May 10, 2012 | 0 | Jun. 7, 1933 | 79 | Anesthesia | 52.872 | 800 | Oct. 26, 2007 |
| | | | | | | induced | | | |
| 14 | | Feb. 3, 2012 | 0 | Oct. 11, 1945 | 66 | AR | 72.95 | 800 | Jan. 18, 2007 |
| 16 | | Feb. 16, 2012 | 0 | Sep. 26, 1971 | 40 | AR | 346.327 | 400 | Mar. 2, 2006 |
| 18 | | Feb. 6, 2014 | 0 | Dec. 3, 1944 | 69 | AR | 288.166 | 800 | Jan. 19, 2012 |
| 19 | | Feb. 20, 2014 | 0 | Jan. 17, 1933 | 81 | AR | 254.448 | 800 | Oct. 25, 2012 |
| 20 | | Mar. 27, 2014 | 0 | Mar. 16, 1985 | 29 | AR | 595.648 | 1000 | Jun. 10, 2010 |
| 24 | | Apr. 17, 2014 | 0 | Jun. 22, 1957 | 57 | AR | | 800 | May 31, 2012 |
| 29 | | Oct. 25, 2012 | 1 | Oct. 26, 1972 | 40 | AR | 75.482 | 800 | Jan. 14, 2010 |
| 34 | | Mar. 7, 2014 | 0 | Mar. 25, 1933 | 81 | AR | 2921.864 | 600 | Mar. 20, 2008 |
| 35 | | Sep. 8, 2006 | 1 | Sep. 20, 1927 | 79 | AR | 311.893 | 200 | Apr. 18, 2002-Dec. 1, 2006 |
| 36 | | Feb. 23, 2012 | 0 | Nov. 1, 1939 | 72 | AR | 143.319 | 800 | Dec. 7, 2006 |
| 25 | | Mar. 4, 2010 | 0 | Aug. 23, 1957 | 53 | AR, PIHH | 80.761 | 800 | Jan. 8, 2007 |
| 13 | | Aug. 30, 2012 | 0 | May 24, 1949 | 63 | HI | 100.518 | 400 | May 30, 1996 |
| 33 | | Mar. 20, 2009 | 1 | Nov. 10, 1956 | 52 | HI | 57.003 | 800 | Feb. 22, 2007 |
| 22 | | May 22, 2014 | 0 | Apr. 17, 1945 | 69 | Idiopathic | 283.519 | 200 | Nov. 29, 2007 |
| 26 | | Mar. 27, 2014 | 1 | Nov. 25, 1950 | 63 | Idiopathic | 3125.654 | 600 | Jun. 7, 2010 |
| 28 | | May 17, 2012 | 1 | Jan. 12, 1940 | 72 | Idiopathic | 197.55 | 200 | Dec. 14, 2000 |
| 21 | | Jun. 5, 2014 | 0 | Sep. 25, 1931 | 83 | PIHH | 4100.101 | 600 | Mar. 21, 1996 |
| 23 | | Feb. 24, 2011 | 1 | Feb. 24, 1951 | 60 | PIHH | 59.375 | 800 | Mar. 19, 2009 |
| 27 | | May 5, 2011 | 0 | Jul. 3, 1953 | 58 | PIHH | 169.563 | 400 | Dec. 11, 2008 |
| [symbol] 2 | | Jan. 5, 2012 | 0 | Aug. 15, 1938 | 73 | PIHH | | 800 | Aug. 26, 2004 |
| [symbol] 5 | | Jun. 5, 2014 | 0 | Nov. 1, 1946 | 68 | PIHH, AR | | 800 | Feb. 19, 2009 |
| [symbol] 0 | | Jun. 5, 2009 | 0 | Jun. 11, 1954 | 55 | PIHH, AR | 775.176 | 400 | Oct. 17, 2002 |
| | | | | | n = 23 | | n = 20 | | |
| | | | | Average | 64 | Average | 808.03 | | |
| | | | | Std. Error | 3 | Std. Error | 282.38 | | |

| | | | Daliresp | | | | |
|---|---|---|---|---|---|---|---|
| | Cilostazol | Date 1º | Dose | Date 1º | | Date 1º | |
| ID | Dose (mg) | Cilostazol | (mcg) | Daliresp | Other Med | Other | Hx Dexa |
| 17 | 200 | Feb. 10, 2011 | — | — | Thioridizine 20 mg, | Jan. 8, 2009, | |
| | | | | | TCMS | Apr. 23, 2007 | |
| 14 | 200 | Sep. 10, 2009 | — | Mar. 8, 2012 | Singulair 10 mg | Aug. 30, 2010 | Apr. 5, 2007, Sep. 10, 2009 |
| 16 | 100 | Jun. 19, 2009 | — | Mar. 15, 2012 | | | |
| 18 | 100 | Sep. 12, 2013 | — | — | — | | — |
| 19 | 100 | Feb. 20, 2014 | — | — | — | | |
| 20 | 100 | Sep. 1, 2011 | — | — | — | | |
| 24 | 100 | Nov. 21, 2013 | — | — | — | | |
| 29 | 200 | Feb. 2, 2011 | — | Jan. 11, 2013 | — | — | Sep. 16, 2010 |
| 34 | 50 | Mar. 7, 2014 | — | — | — | — | |
| 35 | 200 | Jul. 16, 2004-Dec. | — | Apr. 27, 2012 | — | — | Oct. 10, 2002, Jul. 13, 2006, |
| | | 1, 2006 | | | | | Sep. 7, 2006 |
| 36 | 200 | Dec. 6, 2007 | — | Jun. 8, 2012 | — | — | — |
| 25 | 100 | Apr. 25, 2009 | — | | | | |
| 13 | 100 | Apr. 28, 2011 | — | — | Zinc | Mar. 10, 1983 | |
| 33 | 200 | Jun. 16, 2011 | — | 3/38/13 | — | — | Feb. 27, 2007, Nov. 9, 2007, |
| | | | | | | | Mar. 19, 2009, Jun. 16, 2011 |
| 22 | 50 | Jan. 8, 2009 | — | — | — | — | |
| 26 | 200 | Mar. 21, 2013 | — | — | — | — | |
| 28 | 150 | Aug. 19, 2005 | — | May 17, 2012 | Zinc | Nov. 8, 2001 | |
| 21 | 200 | Jul. 25, 2013 | — | Mar. 29, 2012, | Zinc | May 3, 2001 | Nov. 1, 2008 |
| | | | | DC | | | |
| 23 | 200 | Feb. 1, 2010 | | | | | |
| 27 | 200 | Jun. 11, 2009 | | | | | |
| 32 | 100 | Jun. 2, 2005 | — | May 17, 2012 | Thioridazine 10 mg | May 26, 2011 | Jan. 26, 2005 |
| 15 | 100 | Jun. 4, 2011 | — | — | Singulair 10 mg, | Mar. 3, 2011, | |
| | | | | | Thioridazine 10 mg | Sep. 10, 2009 | |
| 30 | 100 | Jun. 6, 2009 | — | — | — | | Aug. 19, 2010, Jun. 4, 2009, |
| | | | | | | | Nov. 11, 2005, Dec. 16, 2005, |
| | | | | | | | Dec. 18, 2003, Mar. 27, 2003 |

[symbol]

indicates data missing or illegible when filed

1.-69. (canceled)

70. A method for ameliorating one or more symptoms associated with a chromosomal abnormality in a subject comprising treating the subject with an amount of one or more phosphodiesterase inhibitors effective to reduce or eliminate the one or more symptoms.

71. The method of claim 70, wherein the chromosomal abnormality comprises trisomy 21, trisomy 18, trisomy 13, or a combination thereof.

72. The method of claim 70, wherein the one or more PDE inhibitors comprise a selective PDE inhibitor, a non-selective PDE inhibitor, or a combination thereof.

73. The method of claim 70, wherein the one or more PDE inhibitors comprise theophylline.

74. The method of claim 70, wherein the subject is treated in utero.

75. The method of claim 74, wherein treating the subject comprises administering the one or more phosphodiesterase inhibitors to the subject's mother.

76. The method of claim 70, wherein the subject is treated after birth.

77. The method of claim 70, wherein the one or more symptoms comprise one or more physical symptoms, on or more cognitive symptoms, or a combination thereof.

78. The method of claim 70, wherein the one or more symptoms comprise one or more structural defects in the brain.

79. The method of claim 78, wherein the one or more structural defects in the brain comprise abnormal cerebellar structure, abnormal hippocampal function, abnormal cerebellar area, abnormal hippocampal area, decreased cellular proliferation within the cerebellum, decreased cellular proliferation within the hippocampus, decreased number of cells within the cerebellum, decreased number of cells within the cerebellum, decreased cerebellar volume, decreased hippocampal volume, decreased cerebellar area, decreased hippocampal area, or a combination thereof.

80. A method of prophylactically reducing a risk that an offspring will develop one or more symptoms associated with a chromosomal abnormality comprising administering an amount of one or more phosphodiesterase inhibitors to a female that is effective to reduce a risk that the offspring will develop the one or more symptoms.

81. The method of claim 80, wherein the chromosomal abnormality comprises trisomy 21, trisomy 18, trisomy 13, or a combination thereof.

82. The method of claim 80, wherein the one or more PDE inhibitors comprise a selective PDE inhibitor, a non-selective PDE inhibitor, or a combination thereof.

83. The method of claim 80, wherein the one or more PDE inhibitors comprise theophylline.

84. The method of claim 80, wherein the one or more symptoms comprise one or more physical symptoms, on or more cognitive symptoms, or a combination thereof.

85. The method of claim 80, wherein the one or more symptoms comprise one or more structural defects in the brain.

86. The method of claim 85, wherein the one or more structural defects in the brain comprise abnormal cerebellar structure, abnormal hippocampal function, abnormal cerebellar area, abnormal hippocampal area, decreased cellular proliferation within the cerebellum, decreased cellular proliferation within the hippocampus, decreased number of cells within the cerebellum, decreased number of cells within the cerebellum, decreased cerebellar volume, decreased hippocampal volume, decreased cerebellar area, decreased hippocampal area, or a combination thereof.

87. The method of claim 80, wherein the female is pregnant.

88. The method of claim 80, wherein the female is planning or attempting to conceive.

89. The method of claim 80, wherein the female is greater than 35 years old.
